(19) United States
(12) Patent Application Publication
(10) Pub. No.: US 2011/0003815 A1
(43) Pub. Date: Jan. 6, 2011

(54) DIPHENYL SUBSTITUTED CYCLOALKANES

(75) Inventors: Anthony Ogawa, Mountainside, NJ (US); Feroze Ujjainwalla, Scotch Plains, NJ (US); Bing Li, Towaco, NJ (US); Lin Chu, Scotch Plains, NJ (US); Minal Patel, Galloway, NJ (US); Helen M. Armstrong, Westfield, NJ (US); Hyun O. Ok, Colonia, NJ (US); Rosemary Sisco, Old Bridge, NJ (US); Dwight MacDonald, L'Ile Bizard (CA); John Hutchinson, La Jolla, CA (US); Helene Perrier, Chatham, NJ (US)

Correspondence Address: MERCK, P O BOX 2000, RAHWAY, NJ, 07065-0907, US

(73) Assignee: MERCK & CO., INC.

(21) Appl. No.: 12/682,150

(22) PCT Filed: Oct. 6, 2008
(86) PCT No.: PCT/US08/11522

§ 371 Date: Sep. 15, 2010

Related U.S. Application Data
(60) Provisional application No. 60/998,449, filed on Oct. 10, 2007.

(51) Int. Cl. A61K 31/5377; C07D 401/14; C07D 413/12; C07D 211/68; C07D 417/12; C07D 403/12; A61K 31/4439; A61K 31/444; A61K 31/501; A61P 9/10

(52) U.S. Cl. 514/236.2; 546/268.4; 546/269.1; 544/138; 546/194; 546/271.4; 546/268.7; 546/272.4; 546/275.4; 546/272.1; 544/238; 514/333; 514/318; 514/252.05

(57) ABSTRACT

The present invention provides compounds of Formula I which are FLAP inhibitors useful as anti-atherosclerotic, anti-asthmatic, anti-allergic, anti-inflammatory and cytoprotective agents.

### FIELD OF THE INVENTION

The instant invention involves compounds that inhibit 5-lipoxygenase activating protein (FLAP), compositions containing such compounds and methods of treatment using such compounds for the treatment and prevention of atherosclerosis and related diseases and conditions.

### BACKGROUND OF THE INVENTION

Inhibition of leukotriene biosynthesis has been an active area of pharmaceutical research for many years. Leukotrienes are potent contractile and inflammatory mediators derived through the oxygenation of arachidonic acid by 5-lipoxygenase.

One class of leukotriene biosynthesis inhibitors are those known to act through inhibition of 5-lipoxygenase (5-LO). In general, 5-LO inhibitors have been sought for the treatment of allergic rhinitis, asthma and inflammatory conditions including arthritis. One example of a 5-LO inhibitor is the marketed drug zileuton, which is indicated for the treatment of asthma. More recently, it has been reported that 5-LO may be an important contributor to the atherogenic process; see Mehrabian, M. et al., Circulation Research, 2002 Jul. 26, 91(2):120-126.

A new class of leukotriene biosynthesis inhibitors (now known as FLAP inhibitors) distinct from 5-LO inhibitors is described in Miller, D. K. et al., “Identification and isolation of a membrane protein necessary for leukotriene production,” Nature, vol. 343, No. 6255, pp. 278-281 (18 Jan. 1990). See also Dixon, R. A. et al, “Requirement of a 5-lipoxygenase-activating protein for leukotriene synthesis,” Nature, vol 343, no. 6255, pp. 282-4 (18 Jan. 1990). 5-LO inhibitor compounds were used to identify and isolate the inner nuclear membrane 18,000 dalton protein 5-lipoxygenase-activating protein (FLAP). These compounds inhibit the formation of cellular leukotrienes but have no direct effect on soluble 5-LO activity. In cells, arachidonic acid is released from membrane phospholipids by the action of cytosolic phospholipase 2. This arachidonic acid is transferred to nuclear membrane bound 5-lipoxygenase by FLAP. The presence of FLAP in cells is essential for the synthesis of leukotrienes. Additionally, based on studies described in Helgadottir, A., et al., Nature Genetics, vol 36, no. 3 (March 2004) pp. 233-239, it is believed that the gene encoding 5-lipoxygenase activating protein confers risk for myocardial infarction and stroke in humans.

Despite significant therapeutic advances in the treatment and prevention of atherosclerosis and ensuing atherosclerotic disease events, such as the improvements that have been achieved with HMG-CoA reductase inhibitors, further treatment options are clearly needed. The instant invention addresses that need by providing compounds, compositions and methods for the treatment or prevention of atherosclerosis as well as related conditions.

### SUMMARY OF THE INVENTION

The instant invention relates to compounds of Formula I which are FLAP inhibitors, methods for their preparation, and methods and pharmaceutical formulations for using these compounds in mammals, especially humans. This invention provides compounds of structural Formula I and the pharmaceutically acceptable salts thereof. This invention also involves the use of compounds described herein to slow or halt atherogenesis. Therefore, one object of the instant invention is to provide a method for treating atherosclerosis, which includes halting or slowing the progression of atherosclerotic disease once it has become clinically evident, comprising administering a therapeutically effective amount of a compound of Formula I to a patient in need of such treatment. Another object is to provide methods for preventing or reducing the risk of developing atherosclerosis and atherosclerotic disease events, comprising administering a prophylactically effective amount of a compound of Formula Ito a patient who is at risk of developing atherosclerosis or having an atherosclerotic disease event.

The compounds of Formula I are also useful as anti-asthmatic, anti-allergic, anti-inflammatory and cytoprotective agents. They are also useful in treating angina, cerebral spasm, glomerular nephritis, hepatitis, endotoxemia, uveitis, and allograft rejection. The instant invention provides methods of treatment comprising administering a therapeutically effective amount of a compound of Formula Ito a patient in need of the above-described treatments.

A further object is to provide the use of FLAP inhibitors of Formula I in combination with other therapeutically effective agents, including other anti-atherosclerotic drugs. These and other objects will be evident from the description contained herein.

### DETAILED DESCRIPTION OF THE INVENTION

The instant invention provides compounds of formula I:

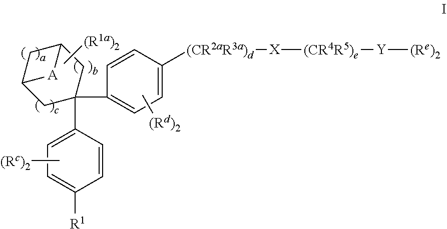

and the pharmaceutically acceptable salts thereof wherein:

a is an integer selected from 0, 1, 2 and 3;

b and c are each integers independently selected from 0, 1 and 2;

A represents a methylene or ethylene group;

each R^{1a} is independently selected from the group consisting of: —H, —F, —C₁₋₆alkyl, —OH, —OC₁₋₆alkyl, -fluoroC₁₋₆alkyl, -fluoro C₁₋₆alkoxy, —N(R^{a})₂ and C₁₋₆alkylN(R^{a})₂,

or one R^{1a} group can represent oxo and the other is as previously defined;

R¹ is selected from the group consisting of:
(a) a 5-membered aromatic or partially unsaturated heterocyclic ring containing 2 to 4 heteroatoms selected from N, S and O, wherein the heterocyclic ring is optionally substituted with one or more of R⁶;
(b) a 6-membered aromatic or partially unsaturated heterocyclic ring containing 1 to 2 heteroatoms selected from N and O, wherein the heterocyclic ring is optionally substituted with one or more of R⁶;
(c) an 8-membered aromatic or partially unsaturated ortho-fused bicyclic ring system containing 3-5 heteroatoms selected from one sulfur and 2-4 of nitrogen wherein one carbon in the ring is optionally substituted with a group selected from ═O, ═S, —SMe, —NH₂, —CF₃, —C₁₋₄alkyl and C₁₋₄alkyl substituted with a group selected from —NH₂, —OH, —OC₁₋₄alkyl, —CN and 1-3 of fluoro;
(d) a 9-membered aromatic or partially unsaturated ortho-fused bicyclic ring system containing 3-4 nitrogen atoms, wherein one carbon in the ring is optionally substituted with a group selected from ═O, ═S, —SMe, —NH₂, —CF₃, —Cl, pyrrolidinyl, tetrahydrofuranyl, —C₁₋₄alkyl and C₁₋₄alkyl substituted with a group selected from —NH₂, —OH, —OC₁₋₄alkyl, —CN, 1-3 of fluoro and piperidinyl;
(e) —C₁₋₆alkyl, —C₂₋₆alkenyl, and —C₂₋₆alkynyl, said alkyl, alkenyl and alkynyl groups being optionally substituted with R¹² and optionally substituted with R¹³;
(f) —C₃₋₆cycloalkyl optionally substituted with 1-3 substituents selected from the group consisting of fluoro, —NH₂, —OH and —C₁₋₃alkyl optionally substituted with 1-3 of fluoro;
(g) —O—R^{6a} wherein R^{6a} is selected from the group consisting of (1) —C₁₋₆alkyl optionally substituted with R¹² and optionally substituted with R¹³, (2) —C₃₋₆cycloalkyl optionally substituted with R¹² and optionally substituted with R¹³ and (3) —C₂₋₆alkyl-R¹⁰; and
(h) —H, —OH, —CN, —CO₂R^{4a}, —C(O)NR⁷R⁸, NR⁷R⁸, —NR^{b}SOₚR^{a}, —NR^{b}C(O)R^{a}, —NR^{b}C(O)NR^{a}R^{b}, —S(O)ₚR^{a}, and —S(O)ₚNR^{a}R^{b};

p is an integer selected from 0, 1 and 2;

X is selected from the group consisting of a bond, —O—, —S— and —C(R¹⁴)₂—;

R^{4a} is selected from the group consisting of —H, —C₁₋₆alkyl and —C₃₋₆cycloalkyl;

R⁶ is selected from the group consisting of (a) —C₁₋₆alkyl optionally substituted with one or more substituents selected from the group consisting of —OH, —NH₂, —N(CH₃)₂, NH(C═O)O^{t}Bu, —CN, fluoro (for example, 1-3 of fluoro), and —O—C₁₋₄alkyl optionally substituted with one or more substituents selected from the group consisting of —OH, phenyl and fluoro, (b) —C₁₋₆alkyl-R¹⁰, (c) —OC₁₋₆alkyl optionally substituted with one or more substituents selected from the group consisting of —OH, —NH₂ and fluoro, (d) —C₃₋₆cycloalkyl optionally substituted with one or more substituents selected from the group consisting of methyl, —OH, —NH₂, —NH(C═O)O^{t}Bu, —CF₃ and fluoro, (e) —NR⁷R⁸, (f) —SO₂C₁₋₃alkyl, (g) —(CH₂)₀₋₃CO₂—R⁸, (h) —OH, (i) ═O (oxo), (j) —SH, (k) ═S, (l) —SMe, (m) —Cl, (n) 1-5 of fluoro, (O) —CF₃, (p) —CN and (q) R¹⁰;

R⁷ is selected from the group consisting of (a) —H, (b) —C₁₋₆alkyl optionally substituted with one or more substituents selected from the group consisting of —F, —CN, —NH₂ and —OH, (c) —C₃₋₆cycloalkyl optionally substituted with one or more substituents selected from the group consisting of methyl, —CF₃, —F, —NH₂ and —OH, (d) —COC₁₋₆alkyl optionally substituted with one or more substituents selected from the group consisting of —F and —OH, (e) —C(O)OC₁₋₆alkyl optionally substituted with one or more substituents selected from the group consisting of methyl, phenyl, —CF₃, —F and —OH, (f) a 4-6 membered saturated heterocyclic ring containing one N and/or one O, wherein the ring is bonded to the nitrogen in —NR⁷R⁸ through a carbon atom in the ring, and wherein the ring is optionally substituted with one or more substituents selected from the group consisting of methyl, —CF₃, —F, CH₂CH₂F, CH₂CHF₂, CH₂CF₃, —NH₂ and —OH, and wherein the ring is optionally bridged by a —CH₂CH₂— group, and (g) a 5-membered heterocyclic ring comprising one, two or three heteroatoms selected from N, O and S, (h) a 5-membered aromatic or partially unsaturated heterocyclic ring containing 2 to 4 heteroatoms selected from N, S and O, wherein the heterocyclic ring is optionally substituted with one or more of R⁶, (i) a 6-membered aromatic or partially unsaturated heterocyclic ring containing 1 to 2 heteroatoms selected from N and O, wherein the heterocyclic ring is optionally substituted with one or more of R⁶;

R⁸ is selected from the group consisting of (a) —H, (b) —C₁₋₆alkyl optionally substituted with one or more substituents selected from the group consisting of —F, —NH₂ and —OH, and (c) —C₃₋₆cycloalkyl optionally substituted with one or more substituents selected from the group consisting of methyl, —CF₃, —F, —NH₂ and —OH;

R¹⁰ is a heterocyclic ring selected from the group consisting of (a) azetidinyl optionally substituted with one or more of methyl, —F and —OH, (b) pyrrolidinyl optionally substituted with one or more of methyl, —F and —OH, (c) piperidinyl optionally substituted with one or more of methyl, —F and —OH, (d) piperazinyl optionally substituted with ═O, and (e) morpholinyl optionally substituted with one or more of methyl and —F; and

Y is selected from the group consisting of (a) a 5-membered aromatic or partially unsaturated heterocyclic ring containing 1 to 4 heteroatoms selected from 1 to 4 of N and zero to 1 of S, wherein the heterocyclic ring is optionally substituted with R¹¹, (b) a 6-membered aromatic or partially unsaturated heterocyclic ring containing 1 to 2 N heteroatoms, wherein the heterocyclic ring is optionally substituted with R¹¹, (c) a 9-membered bicyclic aromatic or partially unsaturated heterocyclic ring containing 1 to 4 N heteroatoms, wherein the heterocyclic ring is optionally substituted with R¹¹ and (d) a 10-membered bicyclic aromatic or partially unsaturated heterocyclic ring containing 1 to 4 N heteroatoms, wherein the heterocyclic ring is optionally substituted with R¹¹; and

R¹¹ is selected from the group consisting of —F, —NH₂, —OH, —OC₃₋₄cycloalkyl, —C₁₋₃alkyl optionally substituted with 1-3 fluoro, and —OC₁₋₃alkyl optionally substituted with phenyl or 1-3 fluoro.

R¹² is selected from the group consisting of: —CO₂R^{4a}, —C(O)NR⁷R⁸, —N(R^{a})₂, —NR^{b}SOₚR^{a}, —NR^{b}C(O)R^{a}, —NR^{b}C(O)NR^{a}R^{b}, —S(O)ₚNR^{a}R^{b}, —S(O)ₚR^{a}, —F, —CF₃, phenyl, Het and Z¹,

R¹³ is selected from the group consisting of —OH, —NH₂ and 1-5 of —F;

R¹⁴ is selected from the group consisting of —H and —C₁₋₄alkyl optionally substituted with 1-3 fluoro groups;

each R^{a} is independently selected from the group consisting of
a) —H,
b) —C₁₋₆alkyl, —C₂₋₆alkenyl and —C₂₋₆alkynyl, wherein each is optionally substituted with 1-2 substituents selected from the group consisting of: —OH, —OC₁₋₄alkyl, —CN, —NH₂, —NHC₁₋₄alkyl, and —N(C₁₋₄alkyl)₂, and —CF₃, and optionally with 1-3 of fluoro,
c) —C₃₋₆cycloalkyl, optionally substituted by 1-2 substituents selected from the group consisting of: —C₁₋₄alkyl, —OH, —OC₁₋₄alkyl, —CN, —NH₂, —NHC₁₋₄alkyl, and —N(C₁₋₄alkyl)₂, and —CF₃, and optionally with 1-3 of fluoro,
d) Het and Het-C₁₋₄alkylene-, the Het moieties being optionally substituted on carbon with 1-2 substituents selected from the group consisting of —F, —OH, —CO₂H, —C₁₋₄alkyl, —CO₂C₁₋₄alkyl, —OC₁₋₄alkyl, —NH₂, —NHC₁₋₄alkyl, —N(C₁₋₄alkyl)₂, —NHC(O)C₁₋₄alkyl, oxo, —C(O)NHC₁₋₄alkyl and —C(O)N(C₁₋₄alkyl)₂; and optionally substituted on nitrogen when present with a group selected from —C₁₋₄alkyl and —C₁₋₄acyl; and the alkylene portion of Het-C₁₋₄alkylene- being optionally substituted with a member selected from the group consisting of —OH, —CN, —OC₁₋₄alkyl, —NH₂, —NHC₁₋₄alkyl, —N(C₁₋₄alkyl)₂ and 1-3 of fluoro,
e) Z² and Z²—C₁₋₄alkylene-, the alkylene portion of Z²—C₁₋₄alkylene- being optionally substituted with a substituent selected from the group consisting of —OH, —CN, —OC₁₋₄alkyl, —NH₂, —NHC₁₋₄alkyl, —N(C₁₋₄alkyl)₂ and 1-3 of fluoro;

each R^{b} is independently selected from the group consisting of —H and —C₁₋₃alkyl optionally substituted with 1-2 members selected from the group consisting of NH₂, —OH, —F, —CN and —CF₃;

R^{c}, R^{d}, and R^{e} are each independently selected from —H, —F, —Br, —C₁₋₆alkyl, —CN, —OH, —OC₁₋₆alkyl, -fluoroC₁₋₆alkyl, -fluoroC₁₋₆alkoxy, —N(R^{f})₂ or —C₁₋₆alkylN(R^{f})₂, where C₁₋₆alkyl and OC₁₋₆alkyl are optionally substituted by 1-3 of fluoro;

each R^{f} is independently selected from the group consisting of —H and
(a) —C₁₋₁₀a —C₃₋₁₀alkenyl, or —C₃₋₁₀alkynyl, optionally substituted with 1-3 fluoro groups or 1-2 members selected from the group consisting of: —OH, —OC₁₋₆alkyl, —CN, —NH₂, —NHC₁₋₄alkyl, and —N(C₁₋₄alkyl)₂;
(b) Aryl or Ar—C₁₋₆alkylene-, the aryl portions being optionally substituted with 1-2 of —C₁₋₆alkyl, —CN, —OH, —OC₁₋₆alkyl, -fluoroC₁₋₆alkyl, -fluoroC₁₋₆alkoxy, —C₁₋₆alkyl-NH₂, —C₁₋₆alkylNHC₁₋₄alkyl, —C₁₋₆alkylN(C₁₋₄alkyl)₂, —NH₂, —NHC₁₋₄alkyl; —N(C₁₋₄alkyl)₂, —NHC(O)C₁₋₄alkyl, —C(O)NHC₁₋₄alkyl, —C(O)N(C₁₋₄alkyl)₂, —CO₂H and —CO₂C₁₋₆alkyl groups, and 1-3 —F, —Cl or —Br groups; and

the alkylene portion of Ar—C₁₋₆alkylene- being optionally substituted with —OH, —OC₁₋₆alkyl; —NH₂, —NHC₁₋₄-alkyl, —N(C₁₋₄alkyl)₂, and 1-3 fluoro groups;

(c) Hetcy or Hetcy-C₁₋₆alkylene-, each being optionally substituted on carbon with 1-2 members selected from the group consisting of: —F, —OH, —CO₂H, —C₁₋₆alkyl, —CO₂C₁₋₆alkyl, —OC₁₋₆alkyl, —NH₂, —NHC₁₋₄alkyl, —N(C₁₋₄alkyl)₂, —NHC(O)C₁₋₄alkyl, oxo, —C(O)NHC₁₋₄alkyl and —C(O)N(C₁₋₄alkyl)₂; and optionally substituted on nitrogen when present with —C₁₋₆alkyl or —C₁₋₆acyl; and

the alkylene portion of Hetcy-C₁₋₆alkylene- being optionally substituted with 1-2 of: —F, —OH, —OC₁₋₆alkyl, —NH₂, —NHC₁₋₄alkyl and —N(C₁₋₄alkyl)₂;

(d) HAR or HAR-C₁₋₆alkylene-, said HAR and HAR portion of HAR-C₁₋₆alkylene- being substituted with 1-2 members selected from the group consisting of: —F, —Cl, —Br, —C₁₋₆alkyl, —CN, —OH, —OC₁₋₆alkyl, -fluoroC₁₋₆alkyl, -fluoroC₁₋₆alkoxy NH₂, —NHC₁₋₄alkyl, —N(C₁₋₄alkyl)₂, —NHC(O)C₁₋₄alkyl, —C(O)NHC₁₋₄alkyl, —C(O)N(C₁₋₄alkyl)₂, —CO₂H, —CO₂C₁₋₆alkyl; and

the alkylene portion of HAR-C₁₋₆alkylene- being optionally substituted with 1-2 of: —F, —OH, —OC₁₋₆alkyl, —NH₂, —NHC₁₋₄alkyl and —N(C₁₋₄alkyl)₂;

Het is selected from the group consisting of azetidinyl, pyrrolidinyl, piperidinyl, piperazinyl, morpholinyl, tetraydrofuranyl and β-lactamyl, δ-lactamyl, γ-lactamyl and tetrahydropyranyl;

Z¹ is selected from the group consisting of:
a) Z²,
b) an 8-membered aromatic or partially unsaturated ortho-fused bicyclic ring system containing 3-5 heteroatoms selected from one sulfur and 2-4 of nitrogen wherein one carbon in the ring is optionally substituted with a group selected from ═O, ═S, —SMe, —NH₂, —CF₃, —Cl, —C₁₋₄alkyl and C₁₋₄alkyl substituted with a group selected from —NH₂, —OH, —OC₁₋₄alkyl, —CN and 1-3 of fluoro, and
c) a 9-membered aromatic or partially unsaturated ortho-fused bicyclic ring system containing 3-4 nitrogen atoms, wherein one carbon in the ring is optionally substituted with a group selected from ═O, ═S, —SMe, —NH₂, —CF₃, —Cl, —C₁₋₄alkyl and C₁₋₄alkyl substituted with a group selected from —NH₂, —OH, —OC₁₋₄alkyl, —CN and 1-3 of fluoro; and

Z² is selected from the group consisting of:
a) a 5-membered aromatic or partially unsaturated heterocyclic ring containing 2-4 nitrogen atoms, wherein one nitrogen in the ring is optionally substituted with a group selected from —C₁₋₄alkyl and —C₁₋₄alkyl substituted with a group selected from —NH₂, —OH, —CN and 1-3 of fluoro, and one carbon in the ring is optionally substituted with a group selected from ═O, ═S, —SMe, —NH₂, —CF₃, —Cl, —C₁₋₄alkyl, —C₁₋₄alkyl substituted with a group selected from —NH₂, —OH, —OC₁₋₄alkyl, —CN and 1-3 of fluoro, and —OC₁₋₄alkyl optionally substituted by —OH or 1-3 of fluoro;
b) a 5-membered aromatic or partially unsaturated heterocyclic ring containing 2-3 heteroatoms selected from one oxygen or one sulfur and 1-2 of nitrogen, wherein one nitrogen in the ring is optionally substituted with a group selected from C₁₋₄alkyl and C₁₋₄alkyl substituted with a group selected from —NH₂, —OH, —CN and 1-3 of fluoro, and one carbon in the ring is optionally substituted with a group selected from ═O, ═S, —SMe, —NH₂, —CF₃, —Cl, C₁₋₄alkyl optionally substituted with a group selected from —NH₂, —OH, —OC₁₋₄alkyl, —CN and 1-3 of fluoro, and —OC₁₋₄alkyl optionally substituted by —OH or 1-3 of fluoro; and
c) a 6-membered aromatic or partially unsaturated heterocyclic ring containing 1-2 nitrogen atoms, wherein one nitrogen in the ring is optionally substituted with a group selected from —C₁₋₄alkyl and —C₁₋₄alkyl substituted with a group selected from —NH₂, —OH, —CN and 1-3 of fluoro, and one carbon in the ring is optionally substituted with a group selected from ═O, ═S, —SMe, —NH₂, —CF₃, —Cl, —C₁₋₄alkyl, —C₁₋₄alkyl substituted with a group selected from —NH₂, —OH, —OC₁₋₄alkyl, —CN and 1-3 of fluoro, and —OC₁₋₄alkyl optionally substituted by —OH or 1-3 of fluoro;

d and e are each integers independently selected from 0, 1, and 2, such that the sum of d plus e is 0 to 4;

R^{2a}, R^{3a}, R⁴ and R⁵ are each independently selected from the group consisting of —H, —C₁₋₆alkyl, —OC₁₋₆alkyl, —OH, -fluoro, -fluoroC₁₋₆alkyl, -fluoroC₁₋₆alkoxy, —N(R^{f})₂, where R^{f} is as hereinbefore defined, and none or one of CR^{2a}R^{3a} and none or one of CR⁴R⁵ can represent a group selected from carbonyl, thiocarbonyl, C═NR^{f} and a 3- to 7-membered cycloalkyl ring.

In one embodiment of the present invention, “a” is 0 or 2.

In another embodiment of the present invention, b is 0 or 1.

In another embodiment of the present invention, c is 0 or 1.

In another embodiment of the present invention, A is methylene.

In another embodiment of the present invention, R^{1a} is hydrogen, —F, —OH or C₁₋₆alkyl. Preferably, R^{1a} is hydrogen or C₁₋₆alkyl. Most preferably, R^{1a} is hydrogen.

Examples of suitable groups represented by

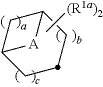

where ∘ marks the point of attachment include cyclobutyl, cyclohexyl and

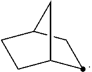

In another embodiment of the present invention, R^{c} is hydrogen, —F, —Cl, —OH or C₁₋₆alkyl. Preferably, R^{c} is hydrogen or —C₁₋₆alkyl. Most preferably, R^{c} is hydrogen.

In another embodiment of the present invention, R^{d} is hydrogen, —F, —Cl, —OH or C₁₋₆alkyl. Preferably, R^{d} is hydrogen or —C₁₋₆alkyl. Most preferably, R^{d} is hydrogen.

In another embodiment of the present invention, R^{e} is hydrogen, —F, —Cl, —OH or C₁₋₆alkyl. Preferably, R^{e} is hydrogen or —C₁₋₆alkyl. Most preferably, R^{e} is hydrogen.

In another embodiment of the present invention, Y is selected from the group consisting of (a) a 5-membered aromatic heterocyclic ring containing 1 to 2 heteroatoms selected from 1 to 2 of N and zero to 1 of S, wherein the heterocyclic ring is optionally substituted by R¹¹ as hereinbefore defined, and (b) a 6-membered heterocyclic ring, containing 1 to 2 N heteroatoms, wherein the heterocyclic ring is optionally substituted by R¹¹. Preferably, Y is selected from thiazolyl, pyridinyl, pyridazinyl and pyrimidinyl, optionally substituted by —F, —OCH₃ and —NH₂. Examples of suitable Y groups include 2-thiazolyl, 2-pyridyl and 2-pyrimidinyl.

In another embodiment of the present invention, X is a bond or —O—. Preferably, X is —O—.

In another embodiment of the present invention, the sum of d plus e is 0, 1 or 2. Preferably, the sum of d plus e is 1 or 2. More preferably, the sum of d plus e is 1. Most preferably, d is zero and e is 1.

In another embodiment of the present invention, —(CR^{2a}R^{3a}) is absent (i.e. d is zero) or —(CR^{2a}R^{3a}) is —C═O, —CH₂, —CHF, —CH(OH), —CH(OCH₃), —CH(OCH₂CH₃), —CH(OCF₃) or —CH(OCH₂phenyl). Preferably, —(CR^{2a}R^{3a}) is absent (i.e. d is zero).

In another embodiment of the present invention, —(CR⁴R⁵) is —CH₂, —CH(CH₃) or —CH(CH₂CH₃), or —(CR⁴R⁵) is absent (i.e. e is zero). Preferably, —(CR⁴R⁵)— is —CH₂—.

In another embodiment of the present invention, R¹ is a 5-membered aromatic or partially unsaturated heterocyclic ring containing 2 to 4 heteroatoms selected from N, S and O, wherein the heterocyclic ring is optionally substituted with R⁶. Examples of suitable R¹ groups that are optionally substituted with R⁶ include:

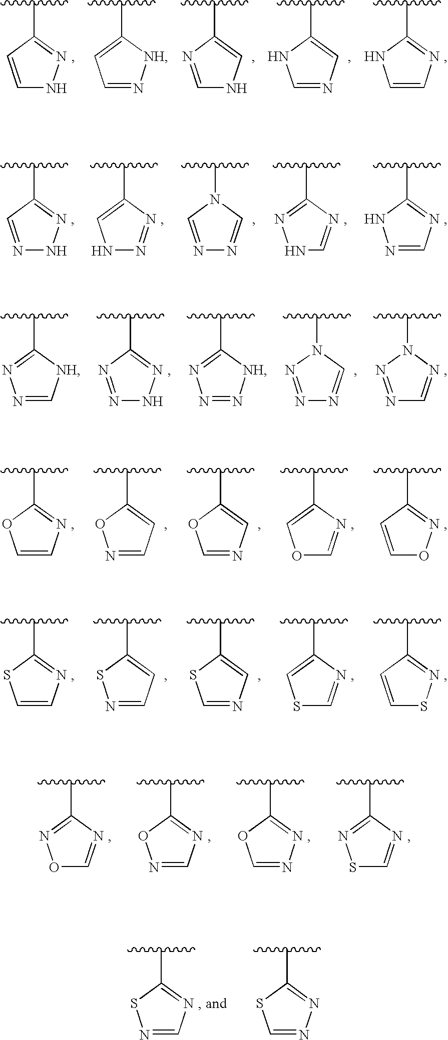

Examples of suitable R⁶ groups include: methyl, ethyl, —NH₂, —CH₂OH, oxo,

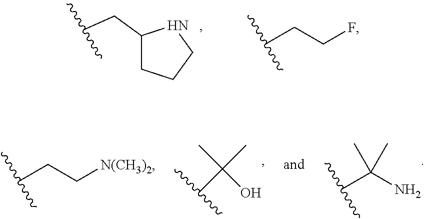

In another embodiment of the present invention, R¹ is a 6-membered aromatic or partially unsaturated heterocyclic ring containing 1 to 2 heteroatoms selected from N and O, wherein the heterocyclic ring is optionally substituted with R⁶. Examples of suitable R¹ groups that are optionally substituted with R⁶ include

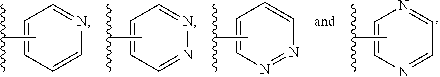

especially

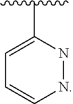

Examples of suitable R⁶ groups include: methyl and —C(OH)(CH₃)₂.

In another embodiment of the present invention, R¹ is —C(O)NR⁷R⁸, where R⁷ and R⁸ are as defined in relation to formula (I). Preferably, R¹ is —CONHR⁷. Examples of suitable R¹ groups include: —C(O)NH-cyclopropyl, —C(O)NHCH₂C(CH₃)₂OH, —C(O)NHCH₂C(CH₃)₂NH₂,

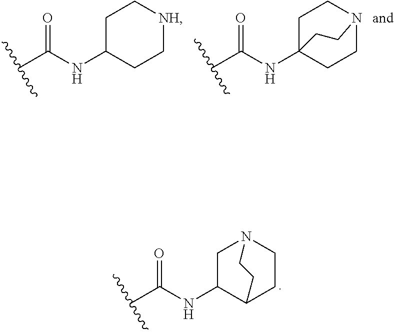

In another embodiment of the present invention, there is provided the compound of formula (Ia):

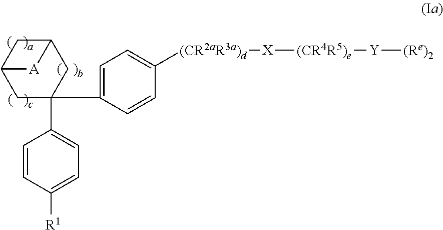

and pharmaceutically acceptable salts thereof, wherein R¹, R^{2a}, R^{3a}, R⁴, R⁵, R^{e}, a, b, c, d, e, A, X and Y are defined in relation to formula (I).

Preferably, the compound of formula (Ia) has the formula (Ia-1):

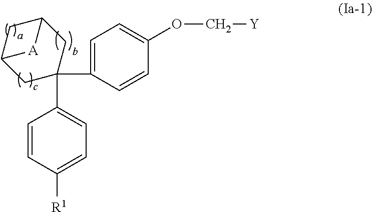

and pharmaceutically acceptable salts thereof, wherein R¹, a, b, c, A and Y are as defined in relation to formula (I). In one subset of formula (Ia-1) R¹ is a 5-membered aromatic or partially unsaturated heterocyclic ring containing 2 to 4 heteroatoms selected from N, S and O, wherein the heterocyclic ring is optionally substituted with one or more of R⁶ and R⁶ is as defined in relation to formula (I). In another subset R¹ is selected from oxazole, isoxazole, oxadiazole, thiadiazole, triazole, pyrazole, and tetrazole, wherein each of these groups includes its corresponding partially saturated derivative (e.g., oxazolinone, dioxazolinone, etc.), and wherein each group is optionally substituted with one or more of R⁶. In another subset R¹ is a 6-membered aromatic or partially unsaturated heterocyclic ring containing 1 to 2 nitrogen atoms, wherein the heterocyclic ring is optionally substituted with one or more of R⁶. In another subset R¹ is pyridyl, pyrazinyl or pyridazinyl optionally substituted with one or more of R⁶. In another subset of formula (Ia-1) Y is selected from pyridyl, pyrimidinyl and thiazolyl, each optionally substituted with R¹¹. In yet another subset Y is selected from pyridyl, pyrimidinyl and thiazolyl each optionally substituted with R¹¹ and R¹ is a 5-membered aromatic or partially unsaturated heterocyclic ring containing 2 to 4 heteroatoms selected from N, S and O, wherein the heterocyclic ring is optionally substituted with one or more of R⁶.

More preferably, the compound of formula (Ia) has the formula (Ia-1a):

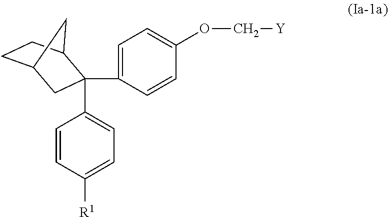

and pharmaceutically acceptable salts thereof, wherein R¹ and Y are defined in relation to formula (I). In one subset of formula (Ia-1a) R¹ is a 5-membered aromatic or partially unsaturated heterocyclic ring containing 2 to 4 heteroatoms selected from N, S and O, wherein the heterocyclic ring is optionally substituted with one or more of R⁶ and R⁶ is as defined in relation to formula (I). In another subset R¹ is selected from oxazole, isoxazole, oxadiazole, thiadiazole, triazole, pyrazole, and tetrazole, wherein each of these groups includes its corresponding partially saturated derivative (e.g., oxazolinone, dioxazolinone, etc.), and wherein each group is optionally substituted with one or more of R⁶. In another subset R¹ is a 6-membered aromatic or partially unsaturated heterocyclic ring containing 1 to 2 nitrogen atoms, wherein the heterocyclic ring is optionally substituted with one or more of R⁶. In another subset R¹ is pyridyl, pyrazinyl or pyridazinyl optionally substituted with one or more of R⁶. In another subset of formula (Ia-1a) Y is selected from pyridyl, pyrimidinyl and thiazolyl, each optionally substituted with R¹¹. In yet another subset Y is selected from pyridyl, pyrimidinyl and thiazolyl each optionally substituted with R¹¹ and R¹ is a 5-membered aromatic or partially unsaturated heterocyclic ring containing 2 to 4 heteroatoms selected from N, S and O, wherein the heterocyclic ring is optionally substituted with one or more of R⁶.

The term “alkyl” means carbon chains which may be linear or branched, or combinations thereof, containing the indicated number of carbon atoms. Examples of alkyl groups include methyl, ethyl, propyl, iso-propyl (i-propyl), butyl, sec- and tert-butyl (s-butyl, t-butyl), pentyl, hexyl, and the like. “Cycloalkyl” is intended to be a cyclized alkyl ring having the indicated number of carbon atoms. Examples of cycloalkyl include cyclopropyl, cyclobutyl, cyclopentyl, and cyclohexyl. The cycloalkyl ring may be substituted on any available carbon which results in the creation of a stable structure, including the ring carbon which serves as the point of attachment to the rest of the molecule. Preferably, cycloalkyl is cyclopropyl or cyclobutyl, and more particularly, when it is substituted with —CH₃ or —CF₃, the substituent is on the ring carbon which serves as the point of attachment to the rest of the molecule.

“Heteroaryl” (HAR) means a mono- or fused aromatic or partially unsaturated ring or ring system containing up to two rings, with each ring containing 5 to 6 atoms, and containing at least one heteroatom selected from O, S and N. Examples include the following:

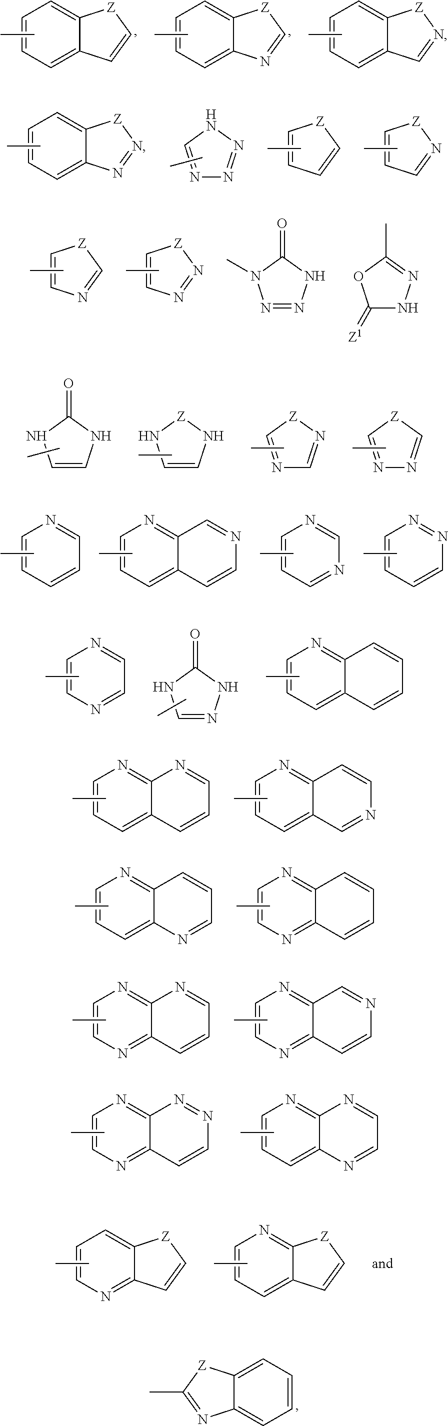

wherein Z represents O, S or NH; and Z¹ represents O or S.

Heteroaryl also includes aromatic heterocyclic groups fused to heterocycles that are non-aromatic or partially aromatic, and aromatic heterocyclic groups fused to cycloalkyl rings. The term also includes partially unsaturated monocyclic rings that are not aromatic, such as 2- or 4-pyridones attached through the nitrogen or N-substituted-(1H,3H)-pyrimidine-2,4-diones (N-substituted uracils). Heteroaryl also includes such groups in charged form, e.g., pyridinium. Substituents, when present, may be on any available carbon in the ring; suitable substituents may also be on available nitrogens in the ring.

The terms “Hetcy” and “heterocycle” and derivatives thereof such as “heterocyclyl” and “heterocyclic ring” mean mono- and bicyclic saturated rings and ring systems containing at least one heteroatom selected from N, S and O, each of said ring having from 3 to 10 atoms in which the point of attachment may be carbon or nitrogen. Examples of “heterocyclyl” include azetidinyl, pyrrolidinyl, piperidinyl, piperazinyl, morpholinyl, γ-lactam, imidazolidinyl, 2,3-dihydrofuro(2,3-b)pyridyl, benzoxazinyl, tetrahydrohydroquinolinyl, tetrahydroisoquinolinyl, dihydroindolyl, and the like. The term also includes partially unsaturated monocyclic rings that are not aromatic, such as 2- or 4-pyridones attached through the nitrogen or N-substituted-(1H,3H)-pyrimidine-2,4-diones (N-substituted uracils). Heterocyclyl moreover includes such moieties in charged form, e.g., piperidinium. Substituents, when present, may be on any available carbon in the ring; suitable substituents may also be on available nitrogens in the ring.

The phrase “optionally substituted with one or more substituents” is intended to mean that the total number of substituents on the optionally substituted moiety overall may be zero, one or more than one, and that each carbon and heteroatom (when present) available for substitution in the given moiety may independently be unsubstituted or mono- or poly-substituted, with one or more substituents that are the same or different at each occurrence and which result in the creation of a stable structure. The term “poly-substituted” is intended to mean two or more substituents, e.g. di-, tri-, tetra-, penta-substitution and higher as appropriate, valence and stability permitting. For example, C₁₋₃alkyl Optionally substituted with one or more of fluoro includes, but is not limited to, —CH₃, —CH₂F, —CHF₂, —CF₃, —CH₂CH₃, —CH₂—CH₂F, —CHF—CH₂F, —CF₂—CF₃, —CH(CF₃)—CH₃, —CF₂—CF₂—CF₃, and the like. In some instances, the number of substituents which may optionally be present on a moiety is specified, for example but not limited to, 1-3 of —F (fluoro). For example, methyl optionally substituted with 1-3 of —F includes —CH₃, —CH₂F, —CHF₂ and —CF₃.

Some of the compounds encompassed herein may exist as tautomers, e.g., keto-enol tautomers. For the purpose of illustration, when R¹ is a 5-membered heterocyclic ring and R⁶ is oxo, the resulting compound may be capable of tautomerism, as exemplified below:

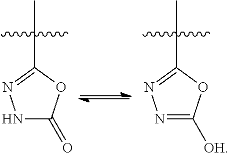

Where compounds of this invention are capable of tautomerization, all individual tautomers as well as mixtures thereof are included in the scope of this invention.

Reference to the compounds of this invention as those of “Formula I” “Formula Ia,” “Formula Ib,” or any other generic structural formulas used herein is intended to encompass compounds falling within the scope of the structural Formula including pharmaceutically acceptable salts, esters and solvates thereof where such forms are possible, unless specified otherwise. The term “pharmaceutically acceptable salts” refers to salts prepared from pharmaceutically acceptable non-toxic bases or acids including inorganic or organic bases and inorganic or organic acids. Salts derived from inorganic bases include aluminum, ammonium, calcium, copper, ferric, ferrous, lithium, magnesium, manganic salts, manganous, potassium, sodium, zinc, and the like. Particularly preferred are the ammonium, calcium, lithium, magnesium, potassium, and sodium salts. Salts derived from pharmaceutically acceptable organic non-toxic bases include salts of primary, secondary, and tertiary amines, substituted amines including naturally occurring substituted amines, cyclic amines, and basic ion exchange resins, such as arginine, betaine, caffeine, choline, N,N′-dibenzylethylenediamine, diethylamine, 2-diethylaminoethanol, 2-dimethylaminoethanol, ethanolamine, ethylenediamine, N-ethylmorpholine, N-ethylpiperidine, glucamine, glucosamine, histidine, hydrabamine, isopropylamine, lysine, methylglucamine, morpholine, piperazine, piperidine, polyamine resins, procaine, purines, theobromine, triethylamine, trimethylamine, tripropylamine, tromethamine, and the like. When the compound of the present invention is basic, salts may be prepared from pharmaceutically acceptable non-toxic acids, including inorganic and organic acids. Such acids include acetic, benzenesulfonic, benzoic, camphorsulfonic, citric, ethanesulfonic, formic, fumaric, gluconic, glutamic, hydrobromic, hydrochloric, isethionic, lactic, maleic, malic, mandelic, methanesulfonic, malonic, mucic, nitric, pamoic, pantothenic, phosphoric, propionic, succinic, sulfuric, tartaric, p-toluenesulfonic acid, trifluoroacetic acid, and the like, and particularly citric, fumaric, hydrobromic, hydrochloric, trifluoroacetic, maleic, phosphoric, sulfuric, and tartaric acids.

Pharmaceutically acceptable esters can optionally be made by esterification of an available carboxylic acid group or by formation of an ester on an available hydroxy group in a compound. Such esterified compounds may serve as pro-drugs which can be hydrolyzed back to their acid or hydroxy form. Examples of pharmaceutically acceptable esters include, but are not limited to, —C₁₋₄alkyl and —C₁₋₄alkyl substituted with phenyl.

The compounds of Formula I may contain one or more asymmetric centers, and can thus occur as racemates, racemic mixtures, single enantiomers, diastereoisomeric mixtures and individual diastereoisomers. The present invention includes all such isomers, as well as salts, esters and solvates of such racemates, mixtures, enantiomers and diastereoisomers. Furthermore, some of the crystalline forms of compounds of the present invention may exist as polymorphs and as such are intended to be included in the present invention. In addition, some of the compounds of the instant invention may form solvates with water or common organic solvents. Such solvates and hydrates are likewise encompassed within the scope of this invention.

Compounds of structural Formula I may be separated into their individual diastereoisomers by, e.g., fractional crystallization from suitable solvents, e.g., DCM/hexanes or EtOAc/hexanes, or via chiral chromatography using an optically active stationary phase. Absolute stereochemistry may be determined by X-ray crystallography of crystalline products or crystalline intermediates which are derivatized, if necessary, with a reagent containing a stereogenic center of known configuration.

The ability of the compounds of this invention to inhibit biosynthesis of the leukotrienes makes them useful for preventing or reversing the symptoms induced by the leukotrienes in a human subject. Accordingly, this invention provides a method for preventing the synthesis, the action, or the release of leukotrienes in a mammal which comprises administering to said mammal a FLAP inhibitory effective amount of a compound of this invention. Such FLAP inhibitory activity can be measured using the FLAP Assay described herein. Since leukotrienes are potent inflammatory mediators, also provided is method of treating an inflammatory condition in a mammal which comprises administering a therapeutically effective amount of a compound of this invention to a mammal in need of such treatment.

The inhibition of the mammalian biosynthesis of leukotrienes also indicates that the compounds and pharmaceutical compositions thereof are useful to treat, prevent or ameliorate atherosclerosis in mammals, and especially in humans. Therefore, the compounds of formula I can be used for the treatment of atherosclerosis comprising administering a therapeutically effective amount of a compound of Formula Ito a patient in need of such treatment. A further aspect of this invention involves a method for preventing or reducing the risk of developing atherosclerosis, comprising administering a prophylactically effective amount of a compound of formula Ito a patient in need of such treatment, for example, a patient who is at risk of developing atherosclerosis.

Atherosclerosis is characterized by the deposition of atheromatous plaques containing cholesterol and lipids on the innermost layer of the walls of large and medium-sized arteries. Atherosclerosis encompasses vascular diseases and conditions that are recognized and understood by physicians practicing in the relevant fields of medicine. Atherosclerotic cardiovascular disease including restenosis following revascularization procedures, coronary heart disease (also known as coronary artery disease or ischemic heart disease), cerebrovascular disease including multi-infarct dementia, and peripheral vessel disease including erectile dysfunction, are all clinical manifestations of atherosclerosis and are therefore encompassed by the terms “atherosclerosis” and “atherosclerotic disease.”

A FLAP inhibitor may be administered to prevent or reduce the risk of occurrence, or recurrence where the potential exists, of a coronary heart disease (CHD) event, a cerebrovascular event, and/or intermittent claudication. Coronary heart disease events are intended to include CHD death, myocardial infarction (i.e., a heart attack), and coronary revascularization procedures. Cerebrovascular events are intended to include ischemic or hemorrhagic stroke (also known as cerebrovascular accidents) and transient ischemic attacks. Intermittent claudication is a clinical manifestation of peripheral vessel disease. The term “atherosclerotic disease event” as used herein is intended to encompass coronary heart disease events, cerebrovascular events, and intermittent claudication. It is intended that persons who have previously experienced one or more non-fatal atherosclerotic disease events are those for whom the potential for recurrence of such an event exists.

Accordingly, the instant invention also provides a method for preventing or reducing the risk of a first or subsequent occurrence of an atherosclerotic disease event comprising the administration of a prophylactically effective amount of a FLAP inhibitor to a patient at risk for such an event. The patient may already have atherosclerotic disease at the time of administration, or may be at risk for developing it.

The method of this invention particularly serves to prevent or slow new atherosclerotic lesion or plaque formation, and to prevent or slow progression of existing lesions or plaques, as well as to cause regression of existing lesions or plaques. Accordingly, one aspect of this invention encompassed within the scope of treatment of atherosclerosis involves a method for halting or slowing the progression of atherosclerosis, including halting or slowing atherosclerotic plaque progression, comprising administering a therapeutically effective amount of a FLAP inhibitor to a patient in need of such treatment. This method also includes halting or slowing progression of atherosclerotic plaques existing at the time the instant treatment is begun (i.e., “existing atherosclerotic plaques”), as well as halting or slowing formation of new atherosclerotic plaques in patients with atherosclerosis.

Another aspect of this invention encompassed within the scope of treatment of atherosclerosis involves a method for regression of atherosclerosis, including regression of atherosclerotic plaques existing at the time the instant treatment is begun, comprising administering a therapeutically effective amount of a FLAP inhibitor to a patient in need of such treatment. Another aspect of this invention involves a method for preventing or reducing the risk of atherosclerotic plaque rupture comprising administering a prophylactically effective amount of a FLAP inhibitor to a patient in need of such treatment.

The ability of the compounds of Formula Ito inhibit biosynthesis of the leukotrienes makes them useful for preventing or reversing the symptoms induced by the leukotrienes in a human subject. This inhibition of the mammalian biosynthesis of leukotrienes indicates that the compounds and pharmaceutical compositions thereof are useful to prevent or reduce the risk for, treat or ameliorate in mammals and especially in humans: 1) pulmonary disorders including diseases such as asthma, chronic bronchitis, and related obstructive airway diseases, 2) allergies and allergic reactions such as allergic rhinitis, contact dermatitis, allergic conjunctivitis, and the like, 3) inflammation such as arthritis or inflammatory bowel disease, 4) pain, 5) skin disorders such as atopic eczema, and the like, 6) cardiovascular disorders such as angina, formation of atherosclerotic plaques, myocardial ischemia, hypertension, platelet aggregation and the like, 7) renal insufficiency arising from ischaemia induced by immunological or chemical (cyclosporin) etiology and 8) migraine or cluster headache, 9) ocular conditions such as uveitis, 10) hepatitis resulting from chemical, immunological or infectious stimuli, 11) trauma or shock states such as burn injuries, endotoxemia and the like, 12) allograft rejection, 13) prevention of side effects associated with therapeutic administration of cytokines such as Interleukin II and tumor necrosis factor, 14) chronic lung diseases such as cystic fibrosis, bronchitis and other small- and large-airway diseases, 15) cholecystitis, 16) multiple sclerosis, 17) proliferation of myoblastic leukemia cells, and 18) acne.

Thus, the compounds of the present invention may also be used to treat or prevent mammalian (especially, human) disease states such as erosive gastritis; erosive esophagitis; diarrhea; cerebral spasm; premature labor; spontaneous abortion; dysmenorrhea; ischemia; noxious agent-induced damage or necrosis of hepatic, pancreatic, renal, or myocardial tissue; liver parenchymal damage caused by hepatoxic agents such as CCl₄ and D-galactosamine; ischemic renal failure; disease-induced hepatic damage; bile salt induced pancreatic or gastric damage; trauma- or stress-induced cell damage; and glycerol-induced renal failure. The compounds also act as inhibitors of tumor metastasis and exhibit cytoprotective action.

The FLAP inhibitors of this invention can also be administered for prevention, amelioration and treatment of glomerulonephritis (see Guasch A., Zayas C. F., Badr K F. (1999), “MK-591 acutely restores glomerular size selectivity and reduces proteinuria in human glomerulonephritis,” Kidney Int., 56:261-267); and also for and prevention, amelioration and treatment of kidney damage resulting from diabetes complications (see Valdivielso J M, Montero A., Badr K F., Munger K A. (2003), “Inhibition of FLAP decreases proteinuria in diabetic rats,” J. Nephrol., 16(1):85-940.)

In addition, the compounds of this invention can also be used for the treatment of chronic obstructive pulmonary disease (COPD). As described in S. Kilfeather, Chest, 2002, vol 121, 197, airway neutrophilia in COPD patients is believed to be a contributing source of inflammation and is associated with airway remodeling. The presence of neutrophils is mediated in part by LTB₄, and treatment with the instant compounds could be used to reduce neutrophilic inflammation in patients with COPD.

The cytoprotective activity of a compound may be observed in both animals and man by noting the increased resistance of the gastrointestinal mucosa to the noxious effects of strong irritants, for example, the ulcerogenic effects of aspirin or indomethacin. In addition to lessening the effect of non-steroidal anti-inflammatory drugs on the gastrointestinal tract, animal studies show that cytoprotective compounds will prevent gastric lesions induced by oral administration of strong acids, strong bases, ethanol, hypertonic saline solutions, and the like. Two assays can be used to measure cytoprotective ability. These assays are: (A) an ethanol-induced lesion assay and (B) an indomethacin-induced ulcer assay and are described in EP 140,684.

In particular, the compounds of the invention would be useful to reduce the gastric erosion caused by co-administration of a cyclooxygenase-2 selective inhibitor and low-dose aspirin. Cyclooxygenase-2 selective inhibitors are widely used as effective anti-inflammatory drugs with less potential for gastrointestinal complications as compared to traditional, non-selective non-steroidal anti-inflammatory drugs. However, the combined use of a cyclooxygenase-2 selective inhibitor with low-dose aspirin for cardio protection may compromise the gastrointestinal safety of this class of compounds. By virtue of its activity as a 5-lipoxygenase inhibitor, the compounds of the invention would be expected to be gastric protective in this regard. See Fiorucci, et al. FASEB J. 17:1171-1173, 2003. Cyclooxygenase-2 selective inhibitors for use with the invention include but are not limited to etoricoxib (ARCOXIA™) and celecoxib (CELEBREX®). A compound of this invention in combination with a cyclooxygenase-2 selective inhibitor could be administered in unit dosage form or separately to a patient on low-dose aspirin therapy. Alternatively, the cyclooxygenase-2 inhibitor could be administered in unit dosage form with low-dose aspirin, in which case a compound of this invention would be administered separately. All three active ingredients in unit dosage form is also encompassed. Conventional dosage amounts of the cyclooxygenase-2 selective inhibitor and aspirin (for cardio protection) may be utilized. Aspirin could be administered at 81 mg once daily.

The term “patient” includes mammals, especially humans, who use the instant active agents for the prevention or treatment of a medical condition. Administering of the drug to the patient includes both self-administration and administration to the patient by another person. The patient may be in need of treatment for an existing disease or medical condition, or may desire prophylactic treatment to prevent or reduce the risk of onset of atherosclerosis.

The term “therapeutically effective amount” is intended to mean that amount of a drug or pharmaceutical agent that will elicit the biological or medical response of a tissue, a system, animal or human that is being sought by a researcher, veterinarian, medical doctor or other clinician. The term “prophylactically effective amount” is intended to mean that amount of a pharmaceutical drug that will prevent or reduce the risk of occurrence of the biological or medical event that is sought to be prevented in a tissue, a system, animal or human by a researcher, veterinarian, medical doctor or other clinician. It is understood that a specific daily dosage amount can simultaneously be both a therapeutically effective amount, e.g., for treatment to slow progression of existing atherosclerosis, and a prophylactically effective amount, e.g., for prevention of an atherosclerotic disease event or formation of new lesions.

In general, FLAP inhibitors can be identified as those compounds which have an IC₅₀ in the “FLAP Binding Assay” that is less than or equal to 1 μM, and preferably 500 nM or less, more preferably 100 nM or less, and most preferably 25 nM or less.

An effective amount of a FLAP inhibitor in the method of this invention is in the range of about 0.01 mg/kg to about 30 mg/kg of body weight per day, preferably 0.1 mg to about 15 mg per kg, and most preferably 0.5 to 7.5 mg per kg, in single or divided doses. A single daily dose is preferred but not necessary. For an average body weight of 70 kg, the dosage level is therefore from about 1 mg to about 2000 mg of drug per day, e.g. 10 mg, 25 mg, 50 mg, 75 mg, 100 mg, 125 mg, 150 mg, 175 mg, 200 mg, 250 mg or 500 mg per day, preferably given as a single daily dose or in divided doses two to four times a day, or in sustained release form. It will be understood, however, that the specific dose level for any particular patient will depend upon a variety of factors including the age, body weight, general health, sex, diet, time of administration, route of administration, rate of excretion, drug combination and the severity of the patient's condition. A consideration of these factors is well within the purview of the ordinarily skilled clinician for the purpose of determining the therapeutically effective or prophylactically effective dosage amount needed to prevent, counter, or arrest the progress of the condition. It is expected that the FLAP inhibitor will administered chronically on a daily basis for a length of time appropriate to treat or prevent the medical condition relevant to the patient, including a course of therapy lasting months, years or the life of the patient.

One or more additional active agents may be administered with a compound of Formula I. The term “additional active agent (or agents)” is intended to mean a pharmaceutically active agent (or agents) different from the compound of formula I. In a broad embodiment, any suitable additional active agent or agents, including but not limited to anti-atherosclerotic agents such as a lipid modifying compound, anti-diabetic agents and/or anti-obesity agents, may be used in combination with the compound of formula I in a single dosage formulation, or may be administered to the patient in a separate dosage formulation, which allows for concurrent or sequential administration of the active agents. The additional active agent or agents may have more than one pharmaceutical activity, for example it may have both lipid-modifying effects and anti-diabetic activity. Examples of additional active agents which may be employed include but are not limited to HMG-CoA reductase inhibitors, which include statins in their lactonized or dihydroxy open acid forms and pharmaceutically acceptable salts and esters thereof, including but not limited to lovastatin (see U.S. Pat. No. 4,342,767), simvastatin (see U.S. Pat. No. 4,444,784), pravastatin, particularly the sodium salt thereof (see U.S. Pat. No. 4,346,227), fluvastatin particularly the sodium salt thereof (see U.S. Pat. No. 5,354,772), atorvastatin, particularly the calcium salt thereof (see U.S. Pat. No. 5,273,995), pitavastatin also referred to as NK-104 (see PCT international publication number WO 97/23200) and rosuvastatin (CRESTOR®; see U.S. Pat. No. 5,260,440); 5-lipoxygenase inhibitors; cholesterol ester transfer protein (CETP) inhibitors, for example JTT-705 and torcetrapib, also known as CP529,414; HMG-CoA synthase inhibitors; squalene epoxidase inhibitors; squalene synthetase inhibitors (also known as squalene synthase inhibitors), acyl-coenzyme A: cholesterol acyltransferase (ACAT) inhibitors including selective inhibitors of ACAT-1 or ACAT-2 as well as dual inhibitors of ACAT-1 and -2; microsomal triglyceride transfer protein (MTP) inhibitors; niacin; niacin receptor agonists such as acipimox and acifran, as well as niacin receptor partial agonists; bile acid sequestrants; LDL (low density lipoprotein) receptor inducers; platelet aggregation inhibitors, for example glycoprotein IIb/IIIa fibrinogen receptor antagonists and aspirin; human peroxisome proliferator activated receptor gamma (PPARγ) agonists including the compounds commonly referred to as glitazones for example pioglitazone and rosiglitazone and, including those compounds included within the structural class known as thiazolidinediones as well as those PPARγ agonists outside the thiazolidinedione structural class; PPARα agonists such as clofibrate, fenofibrate including micronized fenofibrate, and gemfibrozil; PPAR dual α/γ agonists; vitamin B₆ (also known as pyridoxine) and the pharmaceutically acceptable salts thereof such as the HCl salt; vitamin B₁₂ (also known as cyanocobalamin); folic acid or a pharmaceutically acceptable salt or ester thereof such as the sodium salt and the methylglucamine salt; anti-oxidant vitamins such as vitamin C and E and beta carotene; beta-blockers; angiotensin II antagonists such as losartan; angiotensin converting enzyme inhibitors such as enalapril and captopril; calcium channel blockers such as nifedipine and diltiazam; endothelian antagonists; agents that enhance ABCA1 gene expression; FXR and LXR ligands including both inhibitors and agonists; bisphosphonate compounds such as alendronate sodium; and cyclooxygenase-2 inhibitors such as etoricoxib, celecoxib and valdecoxib.

Still another type of agent that can be used in combination with the compounds of this invention are cholesterol absorption inhibitors. Cholesterol absorption inhibitors block the movement of cholesterol from the intestinal lumen into enterocytes of the small intestinal wall. This blockade is their primary mode of action in reducing serum cholesterol levels. These compounds are distinct from compounds which reduce serum cholesterol levels primarily by mechanisms of action such as acyl coenzyme A—cholesterol acyl transferase (ACAT) inhibition, inhibition of triglyceride synthesis, MTP inhibition, bile acid sequestration, and transcription modulation such as agonists or antagonists of nuclear hormones. Cholesterol absorption inhibitors include but are not limited to those described in U.S. Pat. No. 5,846,966, U.S. Pat. No. 5,631,365, U.S. Pat. No. 5,767,115, U.S. Pat. No. 6,133,001, U.S. Pat. No. 5,886,171, U.S. Pat. No. 5,856,473, U.S. Pat. No. 5,756,470, U.S. Pat. No. 5,739,321, U.S. Pat. No. 5,919,672, U.S. Pat. No. 6,498,156, US2004/0082561, US2004/0067913, US2004/0063929, US2002-0137689, WO 05/047248, WO 05/021497, WO 05/021495, WO 05/000353, WO 04/005247, WO 00/63703, WO 00/60107, WO 00/38725, WO 00/34240, WO 00/20623, WO 97/45406, WO 97/16424, WO 97/16455, and WO 95/08532. An exemplary cholesterol absorption inhibitor is ezetimibe, marketed in the U.S. under the tradename ZETIA® described in U.S. Pat. No. Re 37721 and the Physician's Desk Reference.

This and other cholesterol absorption inhibitors can be identified according to the assay of hypolipidemic compounds using the hyperlipidemic hamster described in U.S. Pat. No. Re 37721, beginning in column 20, in which hamsters are fed a controlled cholesterol diet and dosed with test compounds for seven days. Plasma lipid analysis is conducted and data is reported as percent reduction of lipid versus control.

Therapeutically effective amounts of cholesterol absorption inhibitors include dosages of from about 0.01 mg/kg to about 30 mg/kg of body weight per day, preferably about 0.1 mg/kg to about 15 mg/kg. For an average body weight of 70 kg, the dosage level is therefore from about 0.7 mg to about 2100 mg of drug per day, e.g. 10, 20, 40, 100 or 200 mg per day, preferably given as a single daily dose or in divided doses two to six times a day, or in sustained release form. This dosage regimen may be adjusted to provide the optimal therapeutic response when the cholesterol absorption inhibitor is used in combination with a compound of the instant invention.

In the method of treatment of this invention, the FLAP inhibitors may be administered via any suitable route of administration such as orally, parenterally, or rectally in dosage unit formulations containing conventional non-toxic pharmaceutically acceptable carriers, adjuvants and vehicles. The term parenteral as used herein includes subcutaneous injections, intravenous, intramuscular, intrasternal injection or infusion techniques. Oral formulations are preferred.

For oral use, the pharmaceutical compositions of this invention containing the active ingredient may be in forms such as tablets, troches, lozenges, aqueous or oily suspensions, dispersible powders or granules, emulsions, hard or soft capsules, or syrups or elixirs. Compositions intended for oral use may be prepared according to any method known to the art for the manufacture of pharmaceutical compositions and such compositions may contain one or more agents selected from the group consisting of sweetening agents, flavoring agents, coloring agents and preserving agents in order to provide pharmaceutically elegant and palatable preparations. Tablets contain the active ingredient in admixture with non-toxic pharmaceutically acceptable excipients, which are suitable for the manufacture of tablets. These excipients may be for example, inert diluents, such as calcium carbonate, sodium carbonate, lactose, calcium phosphate or sodium phosphate; granulating and disintegrating agents, for example, corn starch, or alginic acid; binding agents, for example starch, gelatin or acacia, and lubricating agents, for example, magnesium stearate, stearic acid or talc.

Oral immediate-release and time-controlled release dosage forms may be employed, as well as enterically coated oral dosage forms. Tablets may be uncoated or they may be coated by known techniques to delay disintegration and absorption in the gastrointestinal tract and thereby provide a sustained action over a longer period. For example, a time delay material such as glyceryl monostearate or glyceryl distearate may be employed. One example of a time-controlled release device is described in U.S. Pat. No. 5,366,738. They may also be coated by the technique described in U.S. Pat. Nos. 4,256,108; 4,166,452; and 4,265,874 to form osmotic therapeutic tablets for controlled release.

Formulations for oral use may also be presented as hard gelatin capsules wherein the active ingredient is mixed with an inert solid diluent, for example, calcium carbonate, calcium phosphate or kaolin, or as soft gelatin capsules wherein the active ingredients is mixed with water or miscible solvents such as propylene glycol, PEGs and ethanol, or an oil medium, for example peanut oil, liquid paraffin, or olive oil.

Aqueous suspensions contain the active material in admixture with excipients suitable for the manufacture of aqueous suspensions. Such excipients are suspending agents, for example sodium carboxymethylcellulose, methylcellulose, hydroxy-propylmethycellulose, sodium alginate, polyvinyl-pyrrolidone, gum tragacanth and gum acacia; dispersing or wetting agents may be a naturally-occurring phosphatide, for example lecithin, or condensation products of an alkylene oxide with fatty acids, for example polyoxyethylene stearate, or condensation products of ethylene oxide with long chain aliphatic alcohols, for example heptadecaethyleneoxycetanol, or condensation products of ethylene oxide with partial esters derived from fatty acids and a hexitol such as polyoxyethylene sorbitol monooleate, or condensation products of ethylene oxide with partial esters derived from fatty acids and hexitol anhydrides, for example polyethylene sorbitan monooleate. The aqueous suspensions may also contain one or more preservatives, for example ethyl, or n-propyl, p-hydroxybenzoate, one or more colouring agents, one or more flavouring agents, and one or more sweetening agents, such as sucrose, saccharin or aspartame.

Oily suspensions may be formulated by suspending the active ingredient in a vegetable oil, for example arachis oil, olive oil, sesame oil or coconut oil, or in mineral oil such as liquid paraffin. The oily suspensions may contain a thickening agent, for example beeswax, hard paraffin or cetyl alcohol. Sweetening agents such as those set forth above, and flavoring agents may be added to provide a palatable oral preparation. These compositions may be preserved by the addition of an anti-oxidant such as ascorbic acid.

Dispersible powders and granules suitable for preparation of an aqueous suspension by the addition of water provide the active ingredient in admixture with a dispersing or wetting agent, suspending agent and one or more preservatives. Suitable dispersing or wetting agents and suspending agents are exemplified by those already mentioned above. Additional excipients, for example sweetening, flavoring and coloring agents, may also be present.

The pharmaceutical compositions of the invention may also be in the form of an oil-in-water emulsion. The oily phase may be a vegetable oil, for example olive oil or arachis oil, or a mineral oil, for example liquid paraffin or mixtures of these. Suitable emulsifying agents may be naturally-occurring phosphatides, for example soy bean, lecithin, and esters or partial esters derived from fatty acids and hexitol anhydrides, for example sorbitan monooleate, and condensation products of the said partial esters with ethylene oxide, for example polyoxyethylene sorbitan monooleate. The emulsions may also contain sweetening and flavouring agents.

Syrups and elixirs may be formulated with sweetening agents, for example glycerol, propylene glycol, sorbitol or sucrose. Such formulations may also contain a demulcent, a preservative and flavoring and coloring agents. The pharmaceutical compositions may be in the form of a sterile injectable aqueous or oleagenous suspension. This suspension may be formulated according to the known art using those suitable dispersing or wetting agents and suspending agents which have been mentioned above. The sterile injectable preparation may also be a sterile injectable solution or suspension in a non-toxic parenterally-acceptable diluent or solvent, for example as a solution in 1,3-butanediol. Among the acceptable vehicles and solvents that may be employed are water, Ringer's solution and isotonic sodium chloride solution. Cosolvents such as ethanol, propylene glycol or polyethylene glycols may also be used. In addition, sterile, fixed oils are conventionally employed as a solvent or suspending medium. For this purpose any bland fixed oil may be employed including synthetic mono- or diglycerides. In addition, fatty acids such as oleic acid find use in the preparation of injectables.

The instant invention also encompasses a process for preparing a pharmaceutical composition comprising combining a compound of Formula I with a pharmaceutically acceptable carrier. Also encompassed is the pharmaceutical composition which is made by combining a compound of Formula I with a pharmaceutically acceptable carrier.

A therapeutically effective amount of a compound of Formula I can be used for the preparation of a medicament useful for treating or preventing any of the medical conditions described herein, in dosage amounts described herein. For example, a compound of Formula I can be used for the preparation of a medicament useful for preventing or reducing the risk of developing atherosclerotic disease, halting or slowing the progression of atherosclerotic disease once it has become clinically manifest, and preventing or reducing the risk of a first or subsequent occurrence of an atherosclerotic disease event. Additionally, the medicament may be useful for the treatment of asthma, allergies and allergic conditions, inflammation, COPD or erosive gastritis. The medicament comprised of a compound of Formula I may also be prepared with one or more additional active agents, such as those described herein.

The compounds of structural formula I of the present invention can be prepared according to the procedures of the following Schemes and Examples, using appropriate materials and are further exemplified by the specific examples which follow. Moreover, by utilizing the procedures described herein, one of ordinary skill in the art can readily prepare additional compounds of the present invention claimed herein. The compounds illustrated in the examples are not, however, to be construed as forming the only genus that is considered as the invention. All temperatures are degrees Celsius unless otherwise noted. Mass spectra (MS) were measured by electron-spray ion-mass spectroscopy (ES-MS).

The instant compounds are generally isolated in a pharmaceutically accepteable form which can either be the free base or an appropriate salt derivative, such as those described above. The free amine bases corresponding to the isolated salts can be generated by neutralization with a suitable base, such as aqueous sodium hydrogencarbonate, sodium carbonate, sodium hydroxide, or potassium hydroxide, and extraction of the liberated amine free base into an organic solvent followed by evaporation. The amine free base isolated in this manner can be further converted into another pharmaceutically acceptable salt by dissolution in an organic solvent followed by addition of the appropriate acid and subsequent evaporation, precipitation, or crystallization.

Some abbreviations used herein are as follows:

ABCA1 is adenosyltriphosphate-binding cassette-family A1; Ac is acetyl; AcOH is acetic acid; AIBN is 2,2′-azobis(2-methylpropionitrile); aq. is aqueous; Ar is Aryl; atm is normal atmospheric pressure; Bn is benzyl; Boc is tert-butylcarbamoyl; br is broad; Bu is butyl; ^{t}Bu is tert-butyl; celite is Celite® diatomaceous earth; conc. is concentrated (for HCl, conc. is a 12 M aq. solution); cpm is counts per minute; δ is chemical shift; DAST is diethylaminosulfur trifluoride; DBU is 1,8-diazabicyclo[5.4.0]undec-7-ene; DCM is dichloromethane; d is doublet; DEAD is diethylazodicarboxylate; DIAD is diisopropylazodicarboxylate; DIBAL-H is diisobutylaluminum hydride; DIPEA is diisopropylethylamine; DMAP is 4-dimethylaminopyridine; DMF is N,N-dimethylformamide; DMPU is 1,3-dimethyl-3,4,5,6-tetrahydro-2(1H)-pyrimidinone; dppf is 1,1′-bis(diphenylphosphino)ferrocene; DMSO is dimethyl sulfoxide; EDC is N-(3-dimethylaminopropyl)-N′-ethylcarbodiimide hydrochloride; EDTA is ethylendiamine tetraacetic acid; equiv. is equivalent(s); ES-MS is electrospray ion-mass spectroscopy; Et is ethyl; Et₂O is diethyl ether; EtOH is ethanol, EtOAc is ethyl acetate; FXR is farnesoid X receptor; g is gram; h is hours; HATU is O-(7-azabenzotriazol-1-yl)-N,N,N′,N′-tetramethyluronium hexafluorophosphate; HetAr or HAR is Heteroaryl; HMG-CoA is 3-hydroxy-3-methylglutaryl coenzyme A; ¹HNMR is proton nuclear magnetic resonance; HOAt is 1-hydroxy-7-azabenzotriazole; HOBt is 1-hydroxybenzotriazole; HPLC is high performance liquid chromatography; Hz is hertz; i is Iso; IC₅₀ is concentration at which 50% inhibition exists; J is internuclear coupling constant; kg is kilogram; LDA is lithium diisopropylamide; LG is leaving group; LHMDS is lithium bis(trimethylsilyl)amide; LTB₄ is leukotriene B₄; LXR is liver X receptor; m is multiplet; M is molar; Me is methyl; m.p. is melting point; mg is milligram; μg is microgram; MeOH is methanol; MHz is megahertz; min is minute; mL is milliliter; mm is millimeter; μL is microliter; mM is milimolar; μM is micromolar; mmol is milimoles; Ms is methanesulfonyl; MS is mass spectrum, and a mass spectrum obtained by ES-MS may be denoted herein by “ES”; m/z is mass to charge ratio; n is normal; N is normal; nm is nanometer; nM is nanomolar; NMM is N-methylmorpholine; NMO is N-methylmorpholine-N-oxide; NMP is N-methylpyrrolidin-2-one; ⁿPr is n-propyl; p is pentet; p is para; PEG is polyethylene glycol; Ph is phenyl; Phth is phthalimidoyl; PPARα is peroxisome proliferator activated receptor alpha; Pr is propyl; ⁱPr is isopropyl; psi is pounds per square inch of pressure; p-TSA is para-toluenesulfonic acid; PyBOP is benzotriaxole-1-yl-oxy-tris-pyrrolidino-phosphonium hexafluorophosphate; q is quartet; rt is room temperature; s is singlet; sec is secondary; t is triplet; ^{t}BuOH is tert-butanol; tert is tertiary; Tf is trifluoromethanesulfonyl; TFA is trifluoroacetic acid; and THF is tetrahydrofuran; TLC is thin layer chromatography; Ts is tosyl; UV is ultraviolet; W is watts; wt. % is percentage by weight; ×g is times gravity; ° C. is degrees Celsius; % w/v is percentage in weight of the former agent relative to the volume of the latter agent.

In the Schemes, all substituents are as defined above unless indicated otherwise.

Reaction schemes A-T illustrate the methods employed in the synthesis of the compounds of the present invention of structural Formula I. All abbreviations are as defined above unless indicated otherwise.

Reaction scheme A illustrates a general method for the synthesis of compounds of type 5. In this method, a ketone of type 1 can be arylated twice in an electrophilic aromatic substitution process called the Friedel-Crafts reaction. Typical conditions for effecting such an arylation include initial addition of one aromatic-coupling partner of type 2 to the ketone 1 to afford an intermediary alcohol of type 3, subsequent generation of an intermediate carbocation of type 4, derived from 3, followed by in situ trapping with a second aromatic-coupling partner of type 2 which may or may not be the same as the first aromatic coupling partner. Formation of 4 may occur spontaneously in solution or it may be promoted with a reagent capable of ionizing 3, like a protic acid such as p-TSA, or concentrated hydrochloric acid or a suitable Lewis acid. In certain cases, it may be preferable to conduct the reaction in the presence of a free radical scavenger such as 3-mercaptopropionic acid or the like. The reaction is conducted typically in an inert organic solvent, at temperatures between −20° C. and the boiling temperature of the solvent. The product is a compound of type 5, which can be elaborated to compounds of the present invention (I) as described in the subsequent schemes.

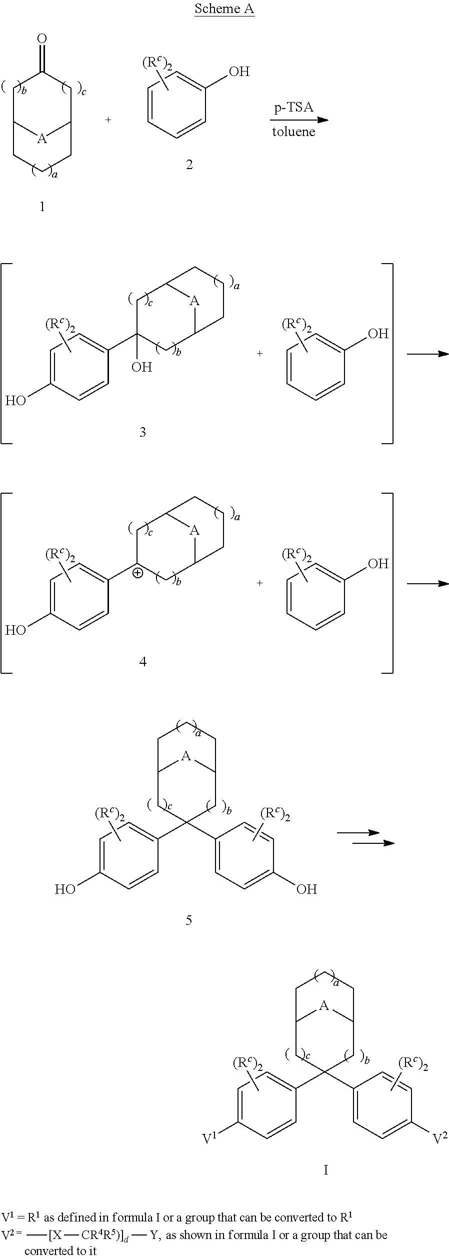

Reaction scheme B illustrates a method of synthesis of a compound type 8. In this method, each of the aromatic coupling partners are introduced sequentially, but in separate chemical manipulations. For example, a ketone of type 1 is treated with an organometallic reagent of type 6, capable of transferring an aryl group, to afford a compound of type 7. Preferred organometallic reagents for effecting this transformation include organolithium (6, M=Li) and organomagnesium (6, M=Mg; Grignard) compounds. When organolithium reagents are employed, the reaction can be conducted in a variety of solvents, such as hexanes or diethyl ether or the like, at temperatures between −78° C. and rt. When Grignard reagents are employed, it is customary to conduct the reaction in a suitable ethereal solvent such as THF or diethyl ether, or mixtures thereof, at temperatures between −78° C. and the boiling temperature of the solvent. The organolithium and Grignard reagents are commonly purchased from commercial sources, but can be prepared synthetically according to known methods of organic synthesis. The resulting alcohol 7 can be reacted applying the aforementioned Friedel Crafts arylation methodologies. The product is a compound of type 8, which can be elaborated to compounds of the present invention (I) as described in the subsequent schemes.

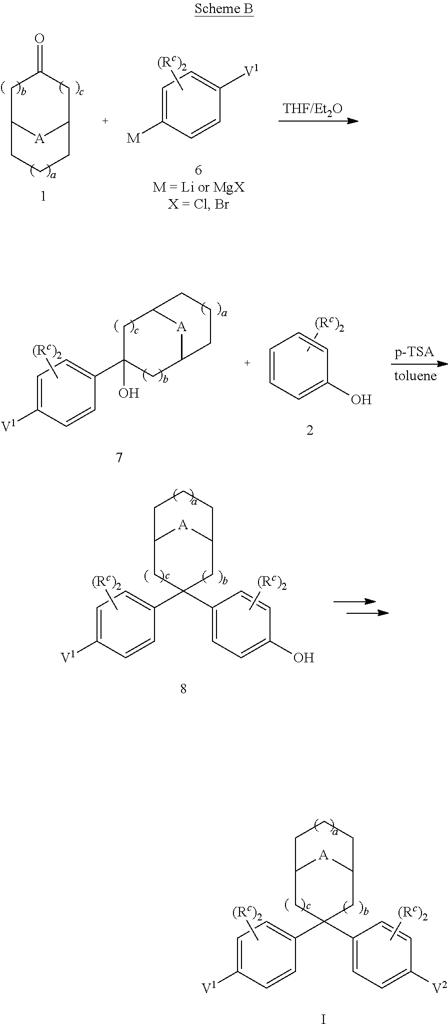

Reaction scheme C illustrates a method of synthesis of compound 16. In this method, an acid chloride derivative of type 9, often generated from the respective carboxylic acid precursor using methods known to those skilled in the art of organic synthesis, is treated with an organometallic reagent of type 10 or type 11 to afford a product of type 12. Preferred organometallic reagents for effecting this transformation include organomagnesium (Grignard) and organozinc compounds. When Grignard reagents (10) are employed, the preferred conditions are similar to those described in scheme B. When organozinc reagents (11) are employed, the reaction is generally conducted in the presence of a suitable organotransition metal catalyst such as bis(triphenylphosphino)palladium(II) dichloride or copper(I) chloride or the like, in a variety of solvents such as THF or diethyl ether, at temperatures between −20° C. and rt (Chem. Rev. 1993, 93, 2117-2188). The Grignard and organozinc reagents are commonly purchased from commercial sources, but can be prepared synthetically according to known methods of organic synthesis. The resulting benzophenone of type 12 is treated with a phosphonium ylide of the type 14 to afford an olefin of type 15. Ylide 14 was generated from a phosphonium salt of type 13 that was reacted with a suitable base, such as lithium bis(trimethylsilyl)amide, LDA, or similar bases possessing alternate counterions, such as sodium or potassium, typically in an ethereal solvent such as diethyl ether or THF, at temperatures between −78° C. and the solvent boiling temperature. The resulting olefin of type 15 is cyclopropanated in the presence of a metallocarbene to afford a cyclopropane of type 16. An example of this reaction is known as the Simmons-Smith reaction. Typical conditions for effecting such a cyclopropanation include the generation of a reactive organozinc intermediate by reacting diiodomethane with zinc-copper couple, and the like, typically in an ethereal solvent, such as diethyl ether, at temperatures between 0° C. and room temperature. Since the reaction mixture is heterogeneous, conditions that favor reagent mixing, such as the use of a sonicating water bath, may be employed. An alternative method for generating compound 16 involves reacting olefin 15 with diiodomethane in the presence of a suitable organometallic reagent, such as diethyl zinc, typically in halogenated solvents, such as dichloroethane, at temperatures between 0° C. and room temperature. Compounds of type 16 can be elaborated to compounds of the present invention (I) as described in the subsequent schemes.

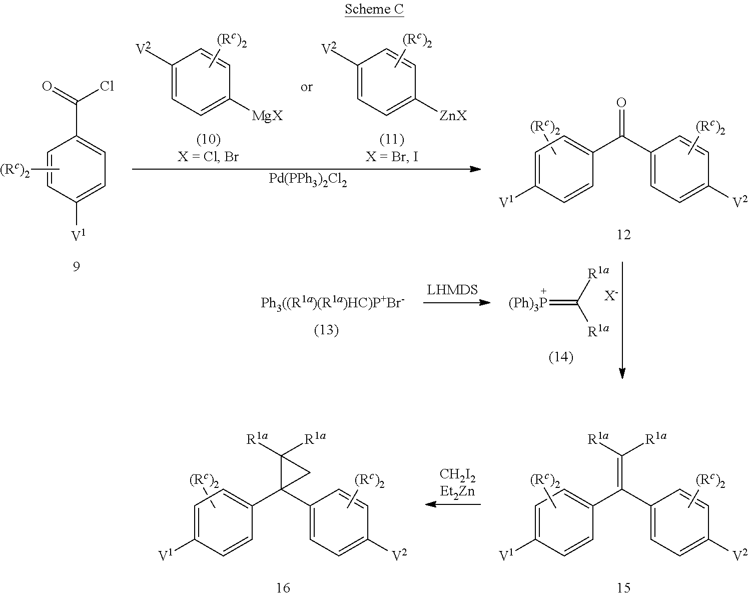

Reaction scheme D illustrates a method for generating compounds of structural formula 20. In this method, olefin 17 can be converted to a cyclobutanone of type 18 in a [2+2]cycloaddition process involving ketene or a ketene equivalent. Since ketene is a highly poisonous gas, it is generally more convenient to use a ketene equivalent generated in situ. Convenient methods for the generation of ketenes include dehydrohalogenation of acyl chlorides or dehalogenation of α-halo acyl chlorides. Accordingly, sonication of trichloroacetyl chloride with zinc dust generates dichloroketene which participates in a [2+2] cycloaddition reaction with 17 to afford the cycloaddition product 18. The reaction is usually conducted in an ethereal solvent like diethyl ether, or THF, at room temperature, for 12-24 hours. Dehalogenation of 18 can be achieved in the presence of zinc dust and a mild protic acid such as acetic acid, at temperatures between 50-100° C., for 6-12 hours. The resulting ketone 19, which is formally a cycloaddition product between 17 and ketene, is then transformed to 20, as removal of the carbonyl functionality of 19 can be achieved using a variety of methods known in the chemical literature, such as the Wolff-Kishner reduction. In this method, hydrazine hydrate is allowed to react with 19, in the presence of base, typically potassium hydroxide, at elevated temperatures up to 200° C., in a solvent such as diethylene glycol. Compounds of type 20 can be elaborated to compounds of the present invention (I) as described in the subsequent schemes.

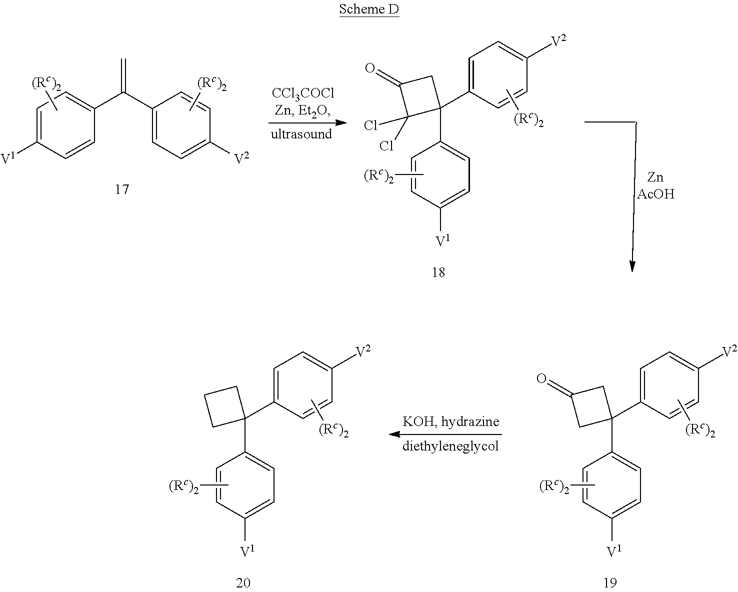

Reaction scheme E illustrates the conversion of compounds of type 19 to compounds of structural formula 21, 22, and 23. In this method, a single ring homologation of cyclobutanone 19 affords a cyclopentanone of type 21, which after a second subsequent ring homologation, furnishes a mixture of regiosomeric cyclohexanones of type 22 and type 23. Conditions for effecting the ring expansion include the method of Yamamoto (K. Maruoka, A. B. Concepcion and H. Yamamoto, Synthesis 1994, 1283-1290) in which the ketone derivative is treated with diazomethane in the presence of an organoaluminum reagent such as trimethylaluminum or methyl aluminum bis(2,6-di-tert-butyl-4-methylphenoxide) (MAD) or the like. The reaction is usually conducted in an inert organic solvent like DCM, and at low temperature, preferably −78° C., for periods of 1-3 hours. Reduction of 21 according to the aforementioned Wolff-Kishner method affords 24 (c=1) while analogous reduction of either/both 22 and 23 affords 24 (c=2).

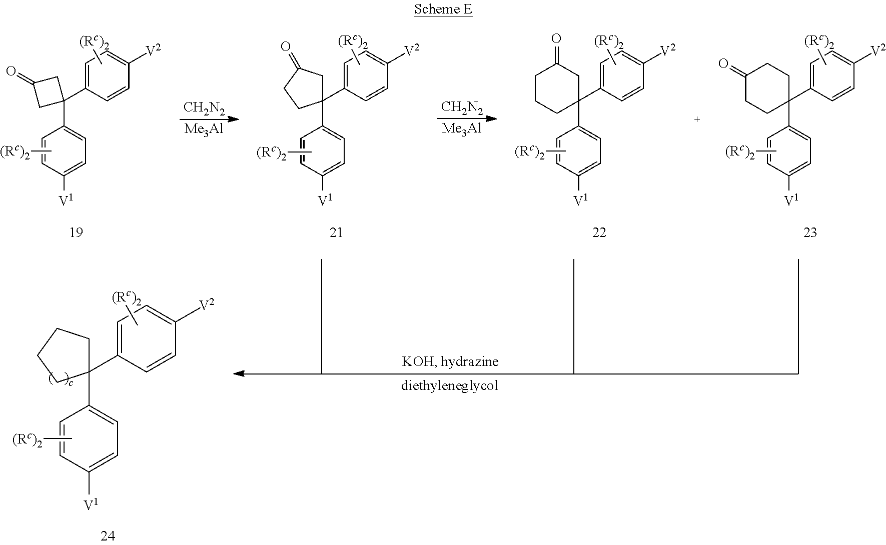

Reaction scheme F illustrates a method for the elaboration of a compound of type 25 to afford a compound of type 26. In this method, 25 is treated with methanol in the presence of a suitable palladium catalyst, such as [1,1′-bis(diphenylphosphino)-ferrocene]dichloropalladium(II), or the like, and a tertiary amine base, such as triethylamine, or diisopropylethylamine, or the like, in an inert organic solvent like dimethylformamide. The reaction is usually conducted at elevated temperature, typically between 50° C. and 100° C., for periods of 3-24 h, under an atmosphere of carbon monoxide (J. Org. Chem. 1974, 39, 3318-3326). In certain cases, it may be preferable to use elevated pressures of carbon monoxide, or an additive, such as lithium chloride, to promote or accelerate the reaction. The product of the reaction is an ester of structural formula 26, which can be elaborated to compounds of the present invention (I) as described in the subsequent schemes.

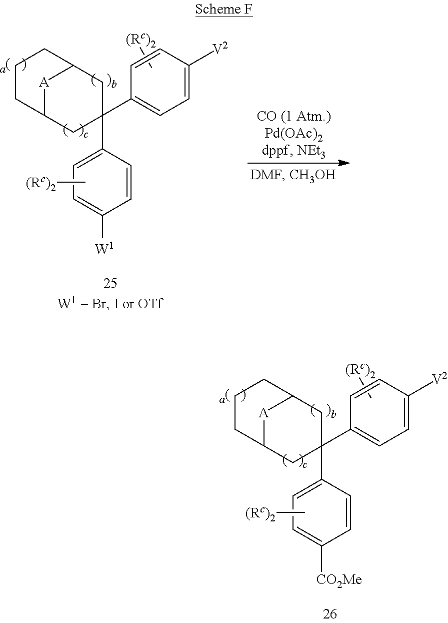

Reaction scheme G illustrates a method for the elaboration of a compound of type 25 to afford a compound of type 27. In this method, 25 is reacted with potassium cyanide, or a similar cyanide source, such as trimethylsilylcyanide, or the like, in the presence of a suitable palladium catalyst/ligand system. It may be preferable to use an inorganic additive, such as copper(I) iodide, and/or a mild base, such as triethylamine, to accelerate or promote the reaction. The reaction is usually performed in a suitable degassed inert organic solvent, preferably a polar aprotic solvent, such as acetonitrile, DMF or NMP, at elevated temperatures, generally between 50-140° C., for a period of 3-24 h. The product of the reaction is a nitrile of structural formula 27, which can be elaborated to compounds of the present invention (I) as described in the subsequent schemes.

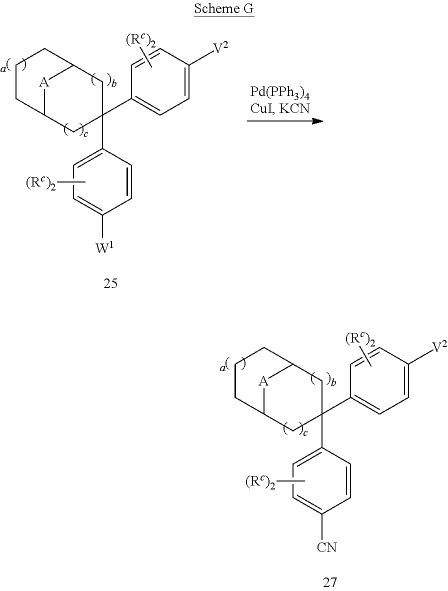

Reaction scheme H illustrates a method of synthesis of compounds of type 28. In this method, a compound of type 26 can be hydrolyzed to carboxylic acids of type 28 using a variety of methods known to those skilled in organic synthesis. The product carboxylic acid of structural formula 28 can be used as a coupling partner in reaction Scheme I or synthetically modified using a variety of methods known in organic synthesis to afford compounds of the present invention (I).

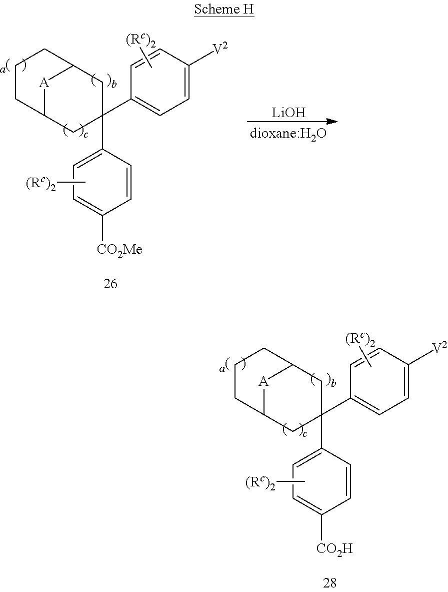

Reaction scheme I illustrates a method of synthesis of compounds of structural formula 29, 30 and 31. In this method, 25 is treated with either allyltributylstannane or vinyltributylstannane in the presence of a suitable palladium catalyst such as [1,1′-bis-(diphenylphosphino)-ferrocene]dichloropalladium(II), in an inert organic solvent like DMF or NMP. The reaction is usually conducted at elevated temperatures, typically between 50-120° C., for periods of 2-24 hours. In certain cases, it may be necessary to use an additive such as lithium chloride to promote the reaction. Often, the reaction times can be significantly reduced if the reaction is conducted under microwave irradiation. The product of the reaction is an alkene of structural formula 29 which can be synthetically elaborated, using a variety of methods known in organic synthesis. For example, 29 can be oxidatively cleaved to afford an aldehyde of type 30, which can be further oxidized to a carboxylic acid derivative of structural formula 31. A method for the oxidative cleavage reaction is the two-step process shown in reaction scheme I. Alkene 29 is first oxidized to a vicinal diol using catalytic osmium tetraoxide in the presence of a stoichiometric reoxidant such as NMO, in a solvent system such as acetone-water. The intermediate vicinal diol which forms is generally not isolated, but is in turn subjected to cleavage with sodium periodate in a suitable mixed solvent system like THF-water to afford 30. Both steps in the oxidative cleavage sequence are generally completed during periods of several minutes to a few hours, at temperatures between 0° C. and room temperature. Alternatively, the oxidative cleavage of 29 may also be accomplished using ozone, or by other methods known to those skilled in the art. Aldehyde 30 can then be further oxidized to 31 using a buffered chlorite oxidation system. In this method, 30 is treated with sodium chlorite and monobasic sodium phosphate in the presence of a chlorine scavenger, such as 2-methyl-2-butene. The reaction is conducted typically in a solvent system like n-butanol-water, for periods of 1-6 hours, at temperatures between 0° C. and room temperature. In certain cases, 29 can be directly converted to 31 using the sodium periodate/ruthenium trichloride reagent system. Both 30 and 31 can be elaborated in numerous ways known in organic synthesis to furnish other compounds of the present invention (I).

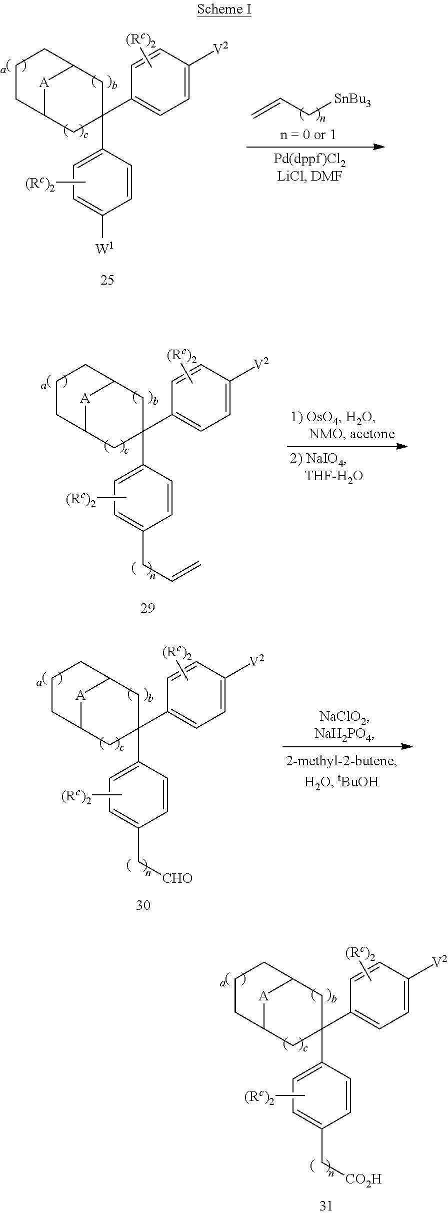

Reaction scheme J illustrates a method of synthesis of a compound of type 32. In this method, compounds of type 25 can be reduced by treatment with an appropriate reducing agent, such as a trialkylammonium formate, or ammonium formate, or triethylsilane, or the like, in the presence of a suitable homogeneous palladium catalyst, such as [1,1′-bis(diphenylphosphino)ferrocene]-dichloropalladium(II) in an inert organic solvent, preferably a polar aprotic solvent, such as DMF, or NMP. The reaction is usually run at elevated temperatures, typically between 50-90° C., to afford an aryl compound of type 32.

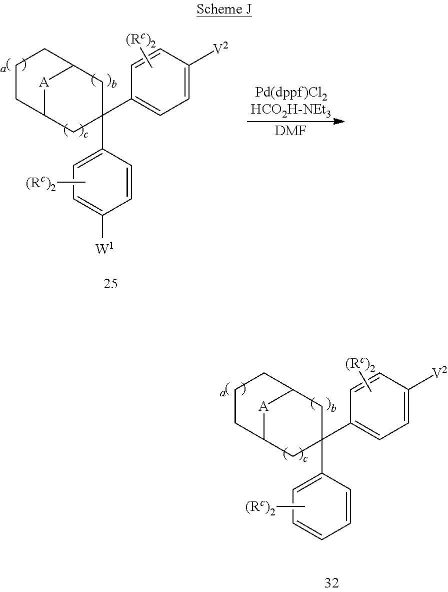

Reaction scheme K illustrates a method of synthesis of compounds of structural formula 34. In the most general case, 31 is treated with an amine of type 33 to afford an amide of type 34. The amide bond coupling reaction illustrated in reaction scheme K is conducted in an appropriate inert solvent such as DMF, DCM or the like and may be performed with a variety of reagents suitable for amide coupling reactions such as HATU, EDC or PyBOP. Preferred conditions for the amide bond coupling reaction shown in reaction Scheme K are known to those skilled in organic synthesis. Such modifications may include, but are not limited to, the use of basic reagents such as triethylamine, DIPEA, or NMM, or the addition of an additive such as HOAt or HOBt. Alternatively, 33 may be treated with an activated ester or acid chloride derivative of 31, to afford 34. The amide bond coupling shown in reaction Scheme K is usually conducted at temperatures between 0° C. and room temperature, occasionally at elevated temperatures, and the coupling reaction is typically conducted for periods of 1 to 24 hours.

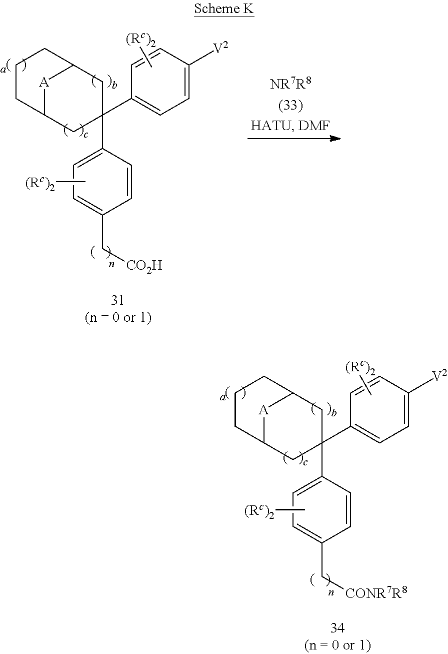

Reaction scheme L illustrates a method for the synthesis of a compound of type 36. In this method, 31 is subjected to the Curtius reaction to afford the N-Boc protected amine of structural formula 35. The reaction is performed by reacting 31 with diphenylphosphoryl azide in the presence of a tertiary amine such as triethylamine or DIPEA in a solvent such as toluene. The initial product is generally accepted to be the acyl azide, which is rearranged to the isocyanate in a thermal process analogous to the Wolff rearrangement of acyl carbenes. The rearrangment is conducted typically at the reflux temperature of the solvent, for instance 110° C., and the rearrangement is usually completed in periods of 1-5 hours. The intermediate isocyanate which forms is generally not isolated, but is in turn subjected to in situ reaction with a suitable alcohol such as tert-butyl alcohol to afford carbamate 35. The N-Boc group can be removed by a suitable deprotection method such as treatment with hydrogen chloride in EtOAc or TFA in DCM. The deprotection is conducted typically at temperatures between 0° C. and room temperature, and the reaction is usually complete in 0.5-3 hours. The product amine of structural formula 36 can be used as a coupling partner using a variety of methods known in organic synthesis to afford compounds of the present invention (I).

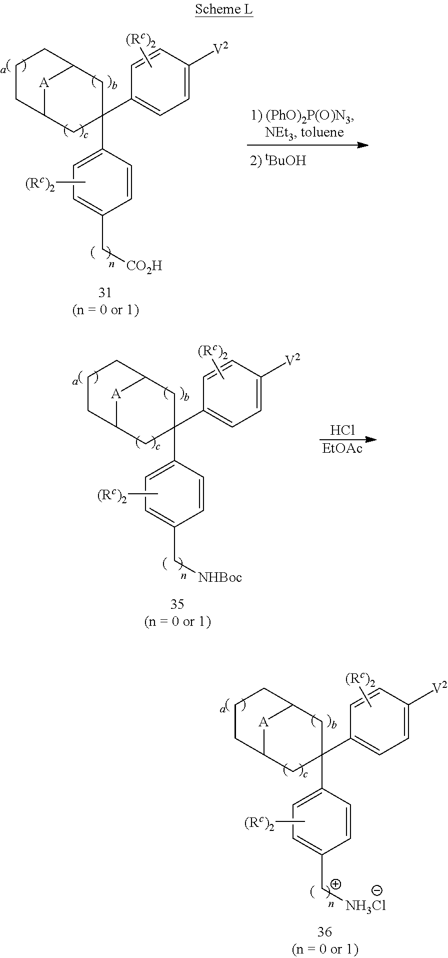

Reaction scheme M illustrates methods for the syntheses of compounds of type 39. For example, 36 can participate in amide bond coupling reactions with a carboxylic acid of type 37 to afford an amide structural formula 39, using the reagents and conditions described for the generalized amide coupling protocol shown in reaction Scheme K in the presence of a suitable tertiary amine base, such as triethylamine, or diisopropylethylamine, or the like. Alternatively, 36 may also be treated with an activated ester or acid chloride derivative of type 38, to afford 39. Typical conditions for effecting such a transformation include treatment of 36 with acid chloride 38 in the presence of excess tertiary amine base such as triethylamine. It is customary to perform the reaction in an inert organic solvent such as DMF or DCM, at temperatures between 0° C. and the reflux temperature of the solvent, frequently at room temperature and for periods of 1-24 hours.

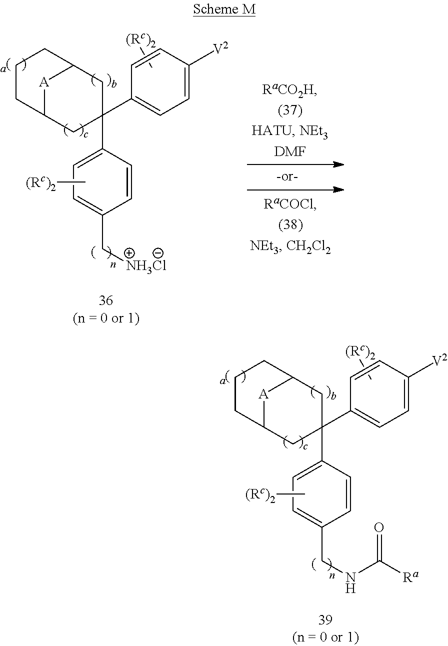

As shown in reaction scheme N, 36 can also be elaborated using the Fukuyama modification of the Mitsunobu reaction (Fukuyama, T.; Jow, C.-K.; Cheung, M. Tetrahedron Lett. 1995, 36, 6373-74). For example, 36 may be reacted with an arylsulfonyl chloride such as 2-nitrobenzenesulfonyl chloride, 4-nitrobenzenesulfonyl chloride or 2,4-dinitrobenzenesulfonyl chloride and a tertiary amine base such as 2,4,6-collidine or 2,6-lutidine in an inert organic solvent such as DCM. Alternatively, the reaction can also be performed under the classical Schotten-Baumann conditions as shown in scheme N, in which 36 and the arylsulfonyl chloride are allowed to react in aqueous alkaline solution. The product of this reaction is the sulfonamide of type 40, which can be further modified by reaction with an alcohol of type 41 in the presence of triphenylphosphine and an activating agent such as DEAD, DIAD, or the like. The reaction is performed in a suitable inert organic solvent such as benzene, toluene, THF or mixtures thereof, typically at room temperature, and the reaction is generally complete in 0.5-3 hours. The product of this reaction is the dialkylsulfonamide of type 42, which can be desulfonylated by treatment with either a nucleophilic amine like n-propylamine, in a solvent such as DCM, or with mercaptoacetic acid and triethylamine in DCM. In either case, the reaction is conducted typically at room temperature, for periods of 5 minutes to 1 hour. When a 2- or 4-nitrobenzenesulfonyl derivative is employed, the cleavage of the sulfonamide is accomplished with either the combination of thiophenol and potassium carbonate in a solvent like DMF, or with mercaptoacetic acid and lithium hydroxide in DMF. In either case, the reaction is conducted at room temperature, for periods of 1-3 hours. The secondary amine product of type 43 can be modified further using a variety of methods known in organic synthesis to provide other compounds of the present invention. For example, 43 may be subjected to a reductive amination reaction with an aldehyde or ketone of type 44 using the conditions described in the bottom of reaction Scheme N to afford compounds of type 45.

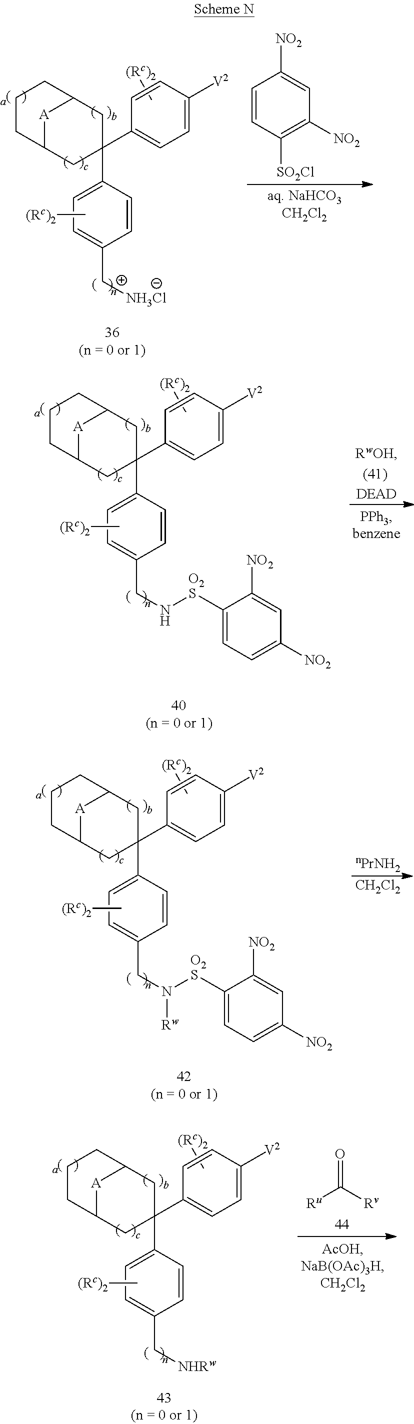

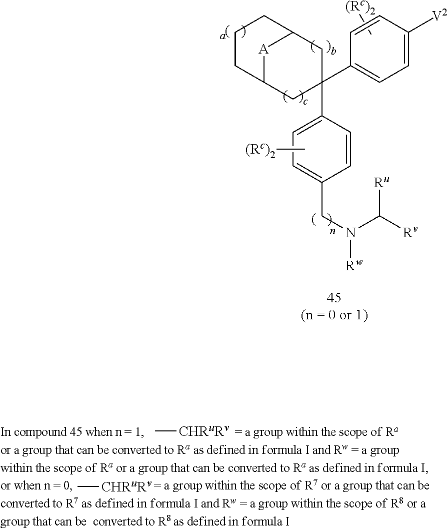

Reaction scheme O illustrates a method of synthesis of compounds of structural formula 48. In this method, commonly referred to as the Suzuki reaction, a compound of type 25 can be treated with an aryl- or heteroaryl-boronic acid of type 46, or alternatively, an aryl- or heteroaryl-boronate of type 47, in the presence of a suitable palladium catalyst, such as [1,1′-bis(diphenylphosphino)ferrocene]-dichloropalladium(II), or tetrakis(triphenylphosphine) palladium (0), or the like, and a mild base, such as sodium carbonate, sodium phosphate tribasic, or the like (Pure Appl. Chem. 1991, 63, 419-422). The reaction is usually performed in a suitable degassed aqueous mixture of inert organic solvents, such as toluene, ethanol or dioxane, at elevated temperatures, generally between 70° C. and the boiling temperature of the solvent mixture, for a period of 3-24 h. Recently, conditions suitable for performing Suzuki reactions at room temperature have been published (for example, see: J. Am. Chem. Soc. 2000, 122, 4020-4028, and references therein).

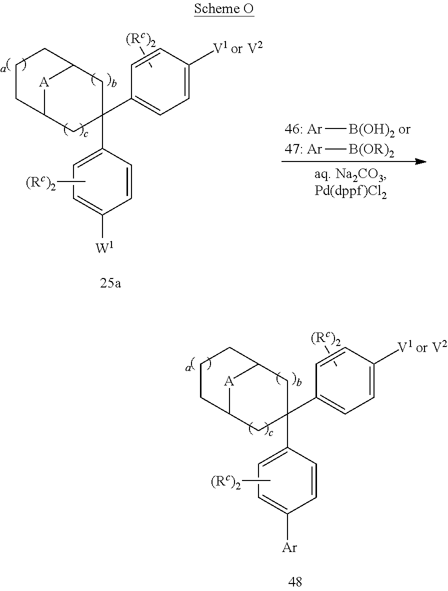

Reaction scheme P illustrates an alternate method of synthesis of compounds of structural formula 48. In this method, a compound of type 25 is treated with bis(pinacolato)diboron in the presence of a suitable palladium catalyst, such as [1,1′-bis(diphenylphosphino)ferrocene]-dichloropalladium(II), and an activating reagent, such as potassium acetate, or the like. The reaction is usually performed in a suitable degassed inert organic solvent, such as dimethyl sulfoxide or dioxane, or the like, at elevated temperatures, generally between 70° C. and 100° C., for a period of 1-24 h (J. Org. Chem. 1995, 60, 7508-7510). The product of this reaction is an intermediate boronate of type 49, which can employ a reagent of type 50 and participate in organotransition metal catalyzed cross-coupling reactions, such as the Suzuki reaction (Scheme O), to afford compounds of the present invention (I).

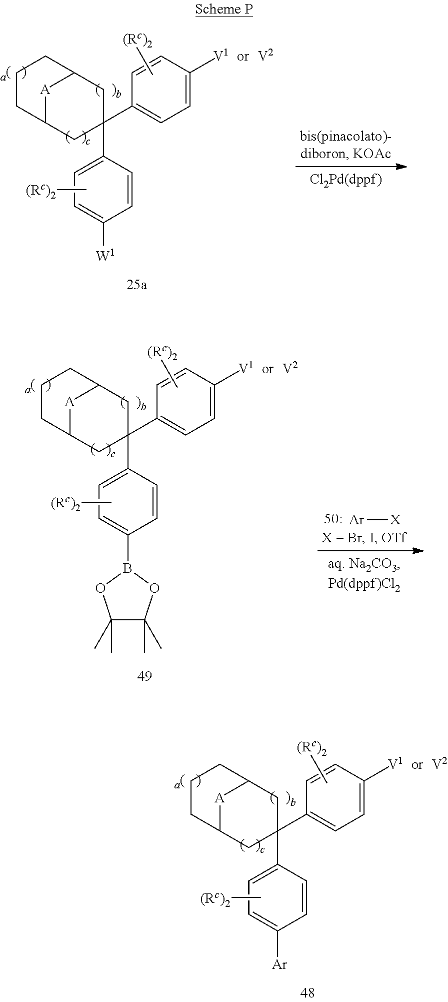

Reaction scheme Q illustrates a method of synthesis of compounds of structural formula 55 and 56, in which group X of formula I of the present invention is a carbon atom. In this method, 51 is treated with a triflating agent, such as trifluoromethansulfonic anhydride or 2-(N,N,-bis(trifluoromethansulfonyl)amino pyridine, or the like, in the presence of a tertiary amine base, such as triethylamine or diisopropylethylamine, to afford an intermediate compound of type 52. The triflating reaction is typically performed in aprotic organic solvents, such as DCM or THF, at temperatures that range from −78° C. to room temperature. Compounds of type 52 can be treated with a terminal alkyne of type 53 in an organotransition metal catalyzed cross-coupling process commonly referred to as the Sonogashira reaction. The reaction is performed in the presence of a suitable palladium catalyst and a copper(I) co-catalyst, such as copper(I) iodide, and typically employs an excess of an amine base, such as triethylamine and diethylamine. The reaction is conducted in an inert organic solvent such as DMF, at temperatures ranging from ambient temperature to about 100° C., for a period of 3-24 hours. The product of the reaction is an alkyne of type 54 which can then be converted into an alkene derivative of type 55 or a saturated alkane derivative of type 56. If 55 is desired, preferred conditions for performing the partial reduction of 54 involve the use of a Lindlar catalyst reagent system under an atmospheric or elevated pressure of hydrogen. The reaction is usually conducted in an inert organic solvent, such as EtOH and EtOAc, or combinations thereof, and at room temperature for a period of 3-15 hours. If 56 is desired, then the reduction of 54 is performed with any one of a variety of palladium-on-carbon catalysts, at either atmospheric or elevated pressure of hydrogen.

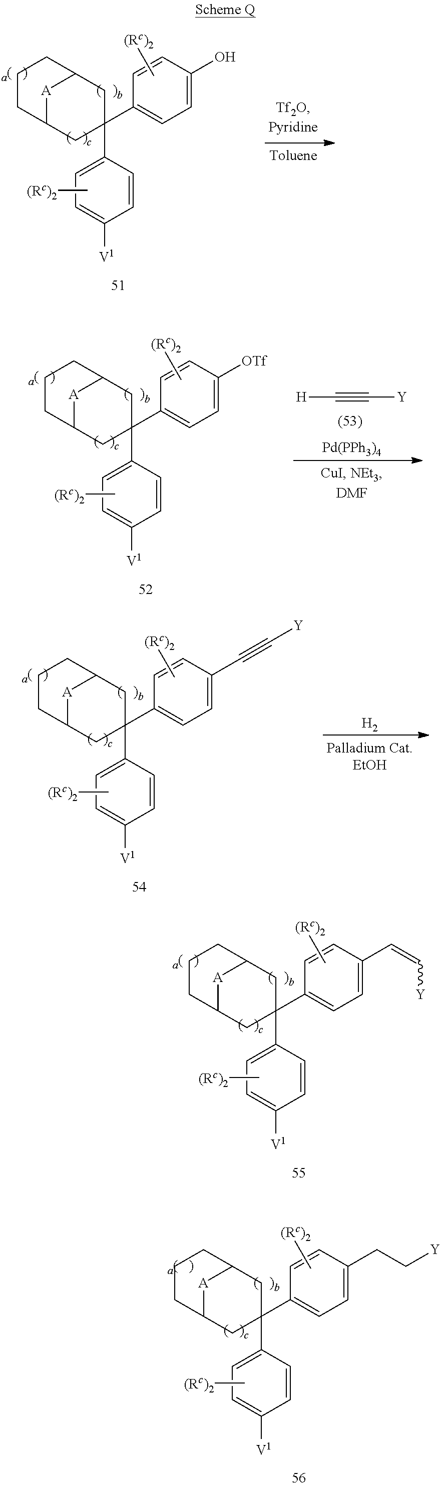

Scheme R illustrates a method of synthesis for compounds of type 60, in which group X of formula I in the present invention is a carbon atom. In this method, which is a modification of the method commonly referred to as the Kucherov reaction, an alkyne of type 54 is treated with a concentrated acid, such as sulfuric acid, or the like, in water or an alternate protic solvent, at temperatures that range between 0° C. and room temperature. This method can also be performed using mercuric sulfate, as a substitute for concentrated acid, to promote the alkyne hydration. The product of this reaction is a ketone of type 57, which can be treated with a reducing agent, such as sodium borohydride or lithium borohydride, under a variety of conditions known to those skilled in the art. In addition, several methods exist for effecting stereoselective reduction of 57 to either antipode of alcohol 58. For example, the application of sub-stoichiometric amounts of chiral oxazaborolidine reagents in conjunction with a stoichiometric reducing agent, such as borane-dimethylsulfide, effects the aforementioned stereoselective reduction of 57 (Angew. Chem. Int. Ed. 1998, 37, p. 1986-2012, and references therein). Alcohol 58 can be treated with an electrophile of type 59 in the presence of a suitable base, such as sodium hydride to afford compounds of type 60. It is customary to conduct the alkylation reaction in a polar aprotic solvent, such as THF, DMF or N-methyl-2-pyrrolidinone, or the like, at temperatures generally between −20° C. and room temperature.

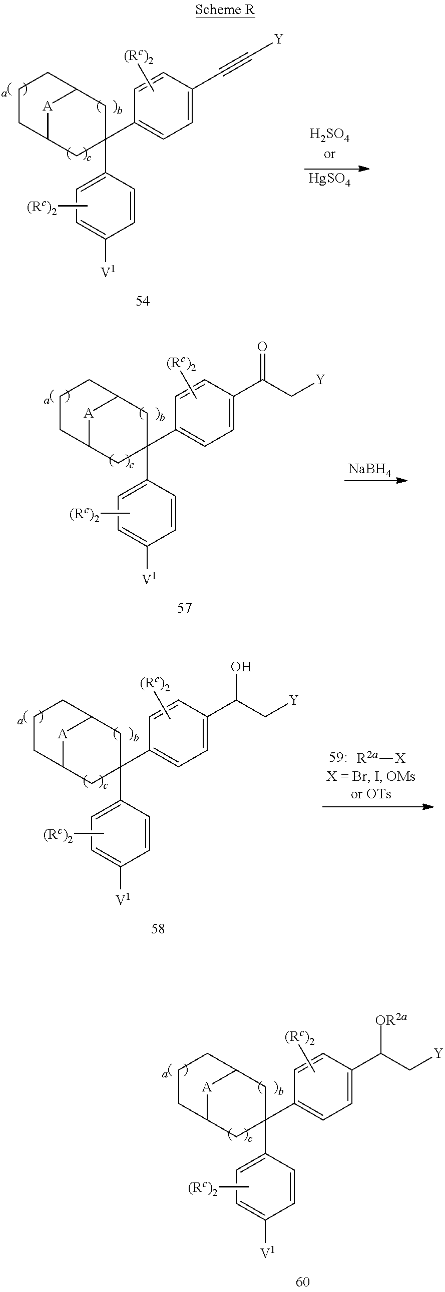

Scheme S illustrates that compounds of structural formula 61 can be elaborated to a variety of heterocyclic (HAR) derivatives of structural formula 62 using known methods in organic synthesis. Specific examples of such transformations are shown in the Examples section. Leading references for effecting such transformations include:
1) Joule, J. A; Mills, K. and Smith, G. F. Heterocyclic Chemistry, Chapman & Hall, 1995, 3rd Edn., and references cited therein;
2) Katrittzky, A. R.; Rees, C. W. (Eds), Comprehensive Heterocyclic Chemistry: The Structure, Reactions, Synthesis, and Uses of Heterocyclic Compounds, Pergamon Press, Oxford, 1984, 8v, and references cited therein; and
3) Comprehensive Heterocyclic Chemistry II: Review of the Literature 1982-1995: The Structure, Reactions, Synthesis and Uses of Heterocyclic Compounds, Pergamon Press, New York, 2nd Edn., 1996, 11v, and references cited therein.

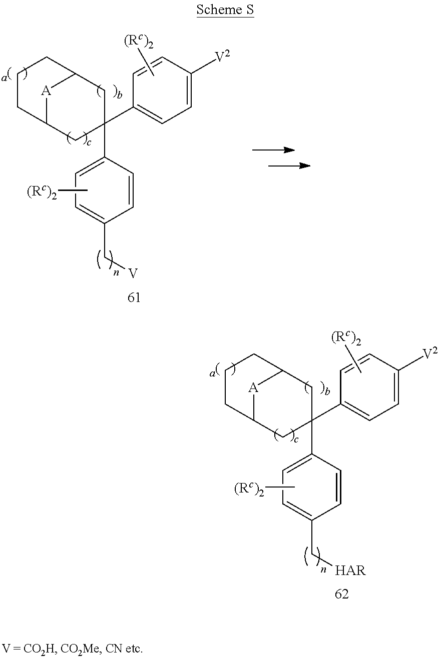

Scheme T illustrates a method for the resolution of a compound of structural formula 63 in which the asterisked carbon is a center of chirality. Generally, the latter, or intermediates en route to their preparation, may be resolved to afford enantiomerically pure compounds such as 64 and 65 by chiral stationary phase liquid chromatography techniques or other suitable methods known in organic synthesis.

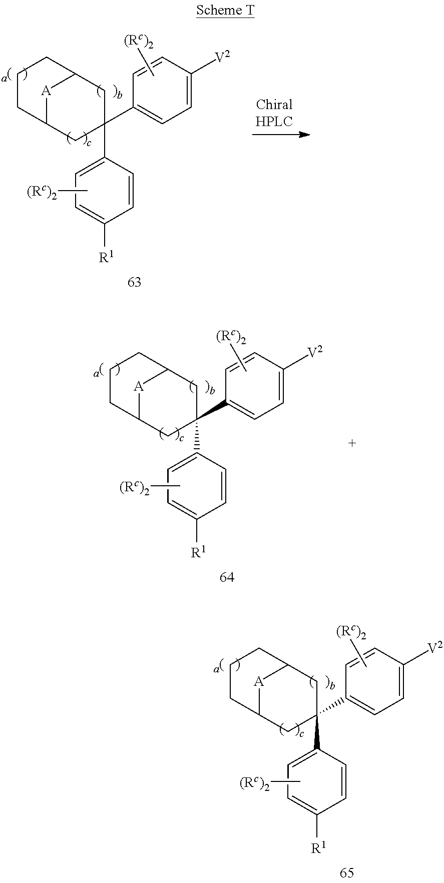

Intermediates used in the synthesis of compounds of this invention and Examples of compounds of this invention can be prepared using the following procedures. In the Tables associated with the following Schemes and Examples, compounds having mass spectral data were synthetically prepared.

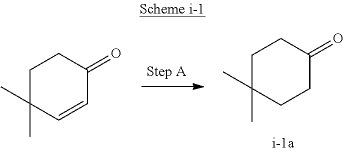

### Step A: Preparation of 4,4-dimethylcyclohexanone (I-1a)

A mixture of 4,4-dimethyl-2-cyclohexen-1-one (4.90 g, 39.5 mmol) and palladium (1.68 g of 5 wt. % on activated carbon) in EtOAc (100 mL) was hydrogenated at atmospheric pressure for 14 h. The resulting mixture was filtered through a short column of Celite®, eluting copiously with EtOAc. The filtrate was concentrated in vacuo to afford the title compound i-1a.

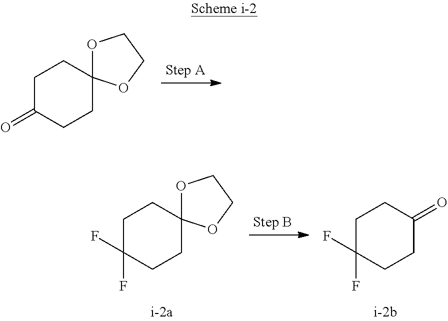

### Step A: Preparation of 8,8-difluoro-1,4-dioxaspiro[4.5]decane (i-2a)

Ethanol (120 μL, 2.00 mmol) was added to a solution of 1,4-dioxaspiro[4.5]-decan-8-one (1.60 g, 10.0 mmol) and Deoxofluor™ (6.20 mL of a 50% solution in toluene) in dichloromethane (50 mL) for 14 h. The reaction mixture was poured into ether and washed successively with saturated aqueous sodium bicarbonate, water and brine. The organics were dried (MgSO₄), filtered and concentrated in vacuo. Purification of the crude residue by flash chromatography on silica gel (gradient elution; 0%-10% EtOAc/hexanes as eluent) afforded the title compound i-2a. ¹HNMR (500 MHz, CDCl₃): δ 3.99 (s, 4H), 2.10 (m, 4H), 1.84 (m, 4H).

### Step B: Preparation of 4,4-difluorocyclohexanone (i-2b)

1.0 M HCl (3 mL) was added to a solution of i-2a (0.430 g, 2.40 mmol) in acetone:water (15 mL of a 4:1 mixture, respectively), and the resulting reaction mixture was heated to 60° C. for 14 h. The reaction mixture was cooled to rt and extracted with Et₂O. The combined organics were washed with brine, dried (MgSO₄), filtered and concentrated in vacuo to afford the title compound i-2b. ¹HNMR (500 MHz, CDCl₃): δ 2.60 (m, 4H), 2.36 (m, 4H).

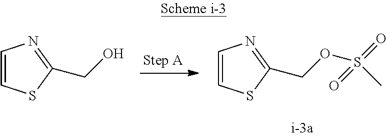

### Step A: Preparation of 1,3-thiaziol-2-ylmethyl methanesulfonate (i-3a)

Methanesulfonic anhydride (282 mg, 1.62 mmol) and triethylamine (300 μL, 2.15 mmol) were added to a solution of 1,3-thiazol-2-ylmethanol (108 mg, 0.935 mmol) in DCM (1 mL) at 0° C. After 10 min, the reaction mixture was partitioned between EtOAc and saturated aqueous sodium bicarbonate solution. The layers were separated, and the aqueous layer washed with EtOAc. The combined organic extracts were dried (Na₂SO₄), filtered, and concentrated in vacuo to afford the title compound i-3a. m/z (ES) 194 (MH)⁺.

Following a similar procedure, i-3b was prepared. m/z (ES) 189 (MH)⁺.

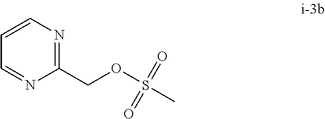

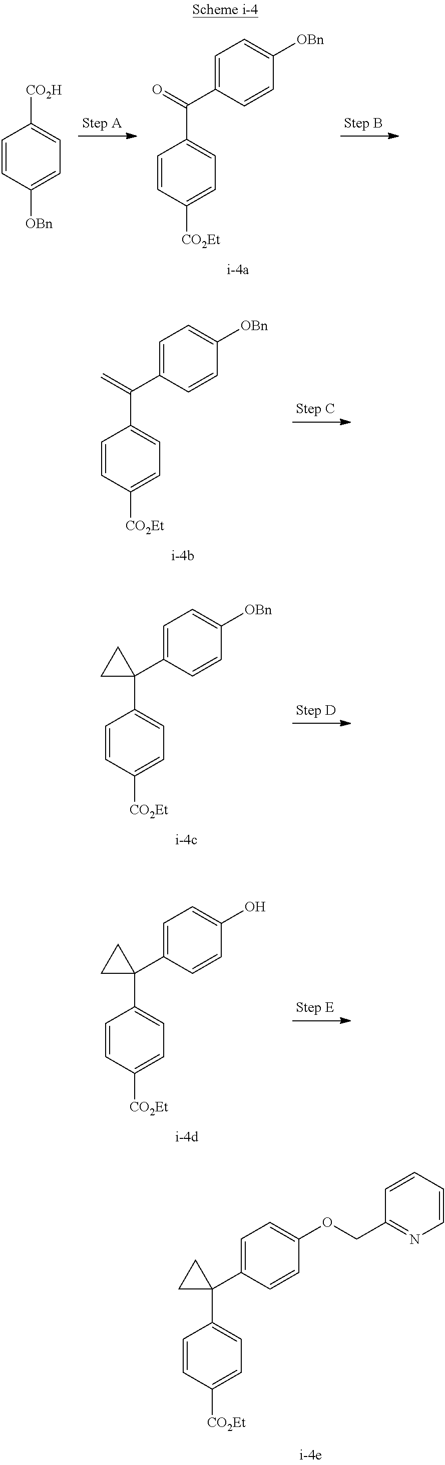

### Step A: Preparation of ethyl 4-[4-(benzyloxy)benzoyl]benzoate (i-4a)

4-Benzyloxybenzoic acid (1.40 g, 5.70 mmol) was dissolved in thionyl chloride (4.0 mL), and the resulting reaction mixture was heated to reflux for 4 h. The reaction mixture was cooled to rt and concentrated in vacuo to afford a crude solid that was added to a stirred solution of dichlorobis(triphenylphosphine) palladium(II) (0.40 g, 0.57 mmol) and 4-ethoxy-carbonylphenylzinc iodide (23 ml of a 0.5 M solution in THF) in THF (23 mL) at 0° C. After 2 h, the reaction mixture was quenched with saturated aqueous ammonium chloride and extracted with EtOAc. The combined organics were dried (Na₂SO₄) and concentrated in vacuo. Purification of the crude residue by flash chromatography on silica gel (gradient elution; 15%-35% EtOAc/hexanes as eluent) afforded the title compound i-4a. m/z (ES) 361 (MH)⁺.

### Step B: Preparation of ethyl 4-{1-[4-(benzyloxy)phenyl]vinyl}benzoate (i-4b)

Potassium bistrimethylsilylamide (8.0 mL of a 0.5 M solution in toluene) was added dropwise over 5 min to a stirred solution of methyltriphenylphosphonium bromide (1.4 g, 4.0 mmol) in THF (20 mL) at 0° C. After 30 min, a solution of i-4a (1.2 g, 3.3 mmol) in THF (15 mL) was added dropwise, and the resulting reaction mixture was allowed to stir at 0° C. for 16 h. The reaction mixture was quenched with saturated aqueous ammonium chloride and extracted with EtOAc. The combined organics were dried (Na₂SO₄) and concentrated in vacuo. Purification of the crude residue by flash chromatography on silica gel (gradient elution; 0%-10% EtOAc/hexanes as eluent) afforded the title compound i-4b. m/z (ES) 359 (MH)⁺.

### Step C: Preparation of ethyl 4-{1-[4-(benzyloxy)phenyl]cyclopropyl}benzoate (i-4c)

Diethylzinc (9.0 mL of a 1.1 M solution in toluene) was added to i-4b (0.59 g, 1.7 mmol) in dichloroethane (5 mL) at 0° C. After 10 min, diiodomethane (1.6 mL, 20 mmol) was added dropwise, and the resulting reaction mixture was allowed to warm to rt. After 16 h, the reaction mixture was quenched with 1.0 M HCl and extracted with EtOAc. The combined organics were dried (Na₂SO₄) and concentrated in vacuo. Purification of the crude residue by flash chromatography on silica gel (gradient elution; 0%-10% EtOAc/hexanes as eluent) afforded the title compound i-4c. m/z (ES) 354 (MH)⁺.

### Step D: Preparation of ethyl 4-[1-(4-hydroxyphenyl)cyclopropyl]benzoate (i-4d)

Iron (III) chloride (0.26 g, 1.6 mmol) was added to i-4c (0.55 g, 1.5 mmol) in dichloromethane (30 mL) at 0° C., and the resulting reaction mixture was allowed to warm slowly to rt. After 5 h, the reaction mixture was quenched with 1.0 M HCl and extracted with DCM. The organics were washed successively with water and brine, dried (Na₂SO₄) and concentrated in vacuo. Purification of the crude residue by flash chromatography on silica gel (gradient elution; 0%-20% EtOAc/hexanes as eluent) afforded the title compound i-4d. m/z (ES) 283 (MH)⁺.

### Step E: Preparation of ethyl 4-{1-[4-(pyridin-2-ylmethoxy)phenyl]cyclopropyl}benzoate (i-4e)

2-Picolyl chloride-hydrochloride (71 mg, 0.31 mmol) was added to a solution of i-4d (87 mg, 0.31 mmol), cesium carbonate (0.10 g, 0.31 mmol) and potassium iodide (52 mg, 0.31 mmol) in DMF (3 mL), and the resulting reaction mixture was allowed to stir at rt for 16 h. The reaction mixture was quenched with saturated aqueous ammonium chloride and extracted with EtOAc. The combined organics were dried (Na₂SO₄) and concentrated in vacuo. Purification of the crude residue by flash chromatography on silica gel (gradient elution; 30%-80% EtOAc/hexanes as eluent) afforded the title compound i-4e. ¹HNMR (500 MHz, CDCl₃): δ 8.78 (d, 1H, J=5.0 Hz), 8.10 (dd, 1H, J=7.5, 8.0 Hz), 7.93 (d, 2H, J=8.2 Hz), 7.87 (d, 1H, J=8.0 Hz), 7.57 (t, 1H, J=6.4 Hz), 7.23 (d, 2H, J=8.9 Hz), 7.21 (d, 2H, J=8.5 Hz), 6.95 (d, 2H, J=8.7 Hz), 5.41 (s, 2H), 4.37 (q, 2H, J=7.1 Hz), 1.38 (t, 3H, J=7.1 Hz), 1.33 (m, 4H). m/z (ES) 374 (MH)⁺.

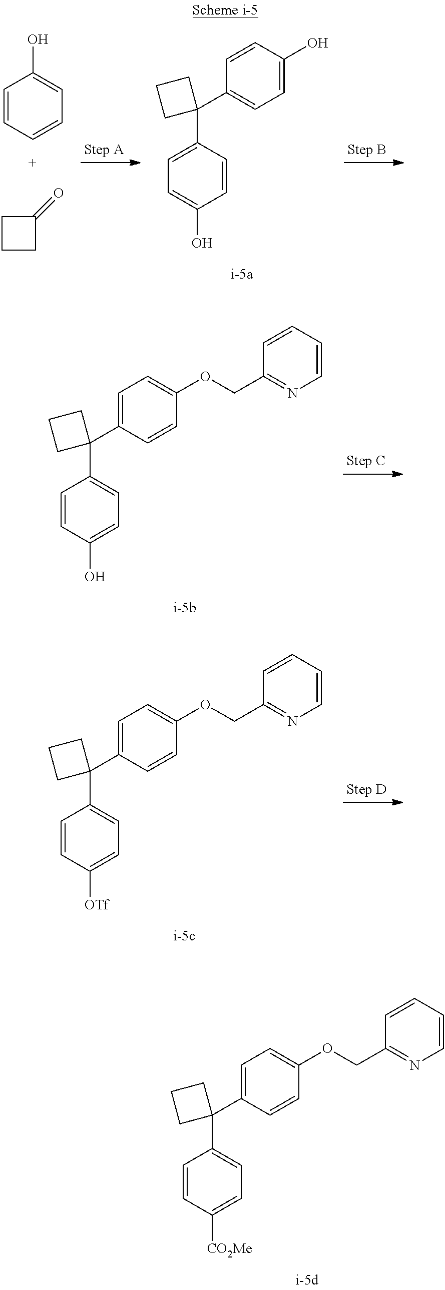

### Step A: Preparation of 4,4′-cyclobutane-1,1-diyldiphenol (i-5a)

Chlorotrimethylsilane (1.3 mL, 10 mmol) was added to a solution of cyclobutanone (0.59 g, 8.4 mmol), phenol (2.4 g, 25 mmol) and 3-mercaptopropionic acid (20 μL). The resulting reaction mixture was heated to 80° C. for 1 h. The reaction mixture was cooled to rt, diluted with DCM and filtered to afford the title compound i-5a.

### Step B: Preparation of 4-{1-[4-(pyridin-2-ylmethoxy)phenyl]cyclobutyl}phenol (i-5b)

A solution of i-5a (0.87 g, 3.4 mmol) in DMF (7 mL) was added to a solution of 2-picolyl chloride-hydrochloride (0.56 g, 3.4 mmol) and potassium carbonate (0.94 g, 6.8 mmol) in DMF (15.0 mL), and the resulting mixture was stirred at rt for 20 h. The reaction mixture was poured into EtOAc and washed successively with water and brine. The organics were dried (Na₂SO₄) and concentrated in vacuo. Purification of the crude residue by flash chromatography on silica gel (gradient elution; 10%-20% EtOAc/hexanes as eluent) afforded the title compound i-5b.

### Step C: Preparation of 4-{1-[4-(pyridin-2-ylmethoxy)phenyl]cyclobutyl}phenyl trifluoromethanesulfonate (i-5c)

Lithium bis(trimethylsilyl)amide (1.1 mL of a 1.0 M solution in THF) was added to a solution of i-5b (0.29 g, 0.87 mmol) in THF (5 mL) at 0° C. After 5 min, phenyl trifluoromethansulfonate (0.37 g, 1.0 mmol) was added, and the resulting reaction mixture was allowed to stir at 0° C. for 1.5 h. The reaction mixture was poured into water and extracted with EtOAc. The organics were dried (Na₂SO₄) and concentrated in vacuo. Purification of the crude residue by flash chromatography on silica gel (isocratic elution; 10% EtOAc/hexanes as eluent) afforded the title compound i-5c, which was carried on immediately to the next step.

### Step D: Preparation of methyl 4-{1-[4-(pyridin-2-ylmethoxy)phenyl]cyclobutyl}benzoate (i-5d)

Palladium (II) acetate (17 mg, 0.074 mmol) and 1,1′-bis(diphenylphosphino)ferrocene (61 mg, 0.11 mmol) were added successively to a solution of i-5c (0.34 g, 0.74 mmol) in triethylamine:DMF:methanol (10 mL of a 1:10:10 mixture, respectively). The reaction mixture was saturated with carbon monoxide and then heated to 80° C. under a carbon monoxide atmosphere (1 atm) for 16 h. After cooling to rt, the reaction mixture was filtered through a pad of Celite®. The filter cake was rinsed with EtOAc, and the combined organics were washed successively with water and brine, dried (Na₂SO₄) and concentrated in vacuo. Purification of the crude residue by flash chromatography on silica gel (isocratic elution; 10% EtOAc/hexanes as eluent) afforded the title compound i-5d. m/z (ES) 374 (MH)⁺. ¹HNMR (500 MHz, CDCl₃): δ 8.63 (d, 1H, J=4.5 Hz), 7.99 (dd, 1H, J=1.8, 6.8 Hz), 7.75 (dt, 1H, J=1.5, 7.5 Hz), 7.56 (d, 1H, J=7.5 Hz), 7.38 (d, 2H, J=8.5 Hz), 7.28 (m, 4H), 6.96 (d, 2H, J=8.5 Hz), 5.22 (s, 2H), 3.93 (s, 3H), 2.77 (t, 4H, J=7.8 Hz), 2.01 (m, 2H).

Following procedures similar to those described above, the following compounds in Table i-5 can be prepared:

TABLE i-5
i-5A

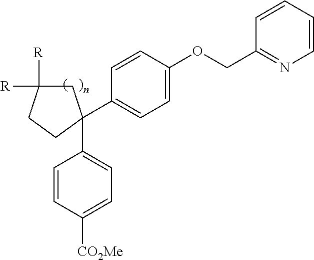

i-5B

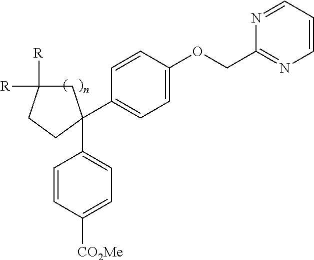

i-5C

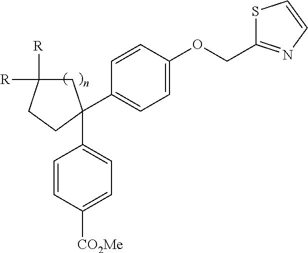

| Ex. i-5A | Ex. i-5B | Ex. i-5C | n | R |
|---|---|---|---|---|
| a | a | a | 1 | H |
| b | b | b | 2 | H |
| c | c | c | 2 | Me |
| d | d | d | 2 | F |

Table i-5. Parent Ion m/z (MH)⁺ data for compounds.
For i-5Aa: methyl 4-{1-[4-(pyridin-2-ylmethoxy)phenyl]cyclopentyl}benzoate: m/z (ES)=388 (MH)⁺
For i-5Ab: methyl 4-{1-[4-(pyridin-2-ylmethoxy)phenyl]cyclohexyl}benzoate: m/z (ES)=402 (MH)⁺
For i-5Ac: methyl 4-{4,4-dimethyl-1-[4-(pyridin-2-ylmethoxy)phenyl]cyclohexyl}benzoate: m/z (ES)=430 (MH)⁺
For i-5Ad: methyl 4-{4,4-difluoro-1-[4-(pyridin-2-ylmethoxy)phenyl]cyclohexyl}benzoate: m/z (ES)=438 (MH)⁺

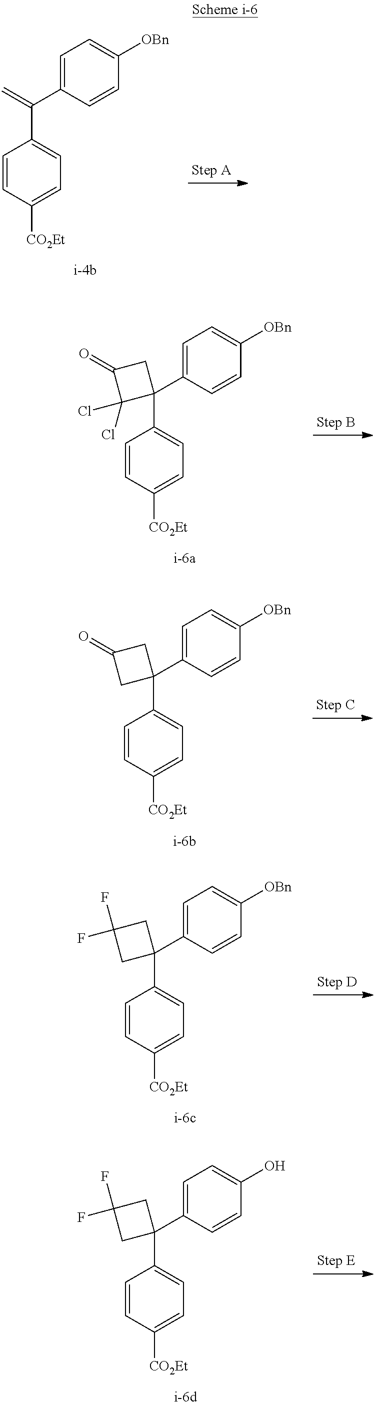

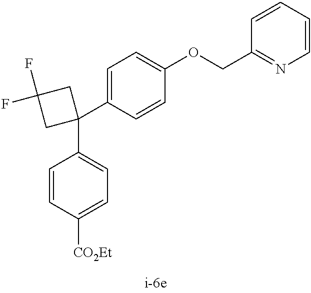

### Step A: Preparation of ethyl 4-{1-[4-(benzyloxy)phenyl]-2,2-dichloro-3-oxocyclobutyl}benzoate (i-6a)

Trichloroacetyl chloride (0.40 mL, 3.6 mmol) was added dropwise to a solution of i-4b (1.3 g, 3.6 mmol) and zinc dust (0.24 g, 3.6 mmol) in ethyl ether:THF (20 mL of a 1:1 mixture), and the resulting reaction mixture was placed in a sonicator bath at rt. Additional portions of trichloroacetyl chloride (2.4 mL, 22 mmol) and zinc dust (0.69 g, 11 mmol) were added over 6 h with constant sonication, and the reaction mixture was allowed to stand overnight at rt. The reaction mixture was quenched with saturated aqueous sodium bicarbonate and extracted with EtOAc. The combined organics were washed successively with water and brine, dried (Na₂SO₄) and concentrated in vacuo. Purification of the crude residue by flash chromatography on silica gel (gradient elution; 0%-10% EtOAc/hexanes as eluent) afforded the title compound i-6a. m/z (ES) 471 (MH)⁺.

### Step B: Preparation of ethyl 4-{1-[4-(benzyloxy)phenyl]-3-oxocyclobutyl}benzoate (i-6b)

Zinc dust (1.8 g, 28 mmol) was added to i-6a (2.2 g, 4.7 mmol) in acetic acid (35 mL), and the resulting reaction mixture was heated to 70° C. for 3 h. The reaction mixture was concentrated and partitioned between EtOAc and saturated aqueous sodium bicarbonate. The layers were separated and the aqueous layer was extracted with EtOAc. The combined organics were washed successively with water and brine, dried (Na₂SO₄) and concentrated in vacuo. Purification of the crude residue by flash chromatography on silica gel (gradient elution; 0%-20% EtOAc/hexanes as eluent) afforded the title compound i-6b. m/z (ES) 401 (MH)⁺.

### Step C: Preparation of ethyl 4-{1-[4-(benzyloxy)phenyl]-3,3-difluorocyclobutyl}benzoate (i-6c)

Ethanol (12 μL, 0.20 mmol) was added to i-6b (0.42 g, 1.1 mmol) and (diethylamino)sulfur trifluoride (0.55 mL, 4.2 mmol) in DCM (10 mL), and the resulting reaction mixture was allowed to stir at rt for 18 h. The reaction mixture was carefully quenched with NaHSO₄ (10% w/v in water) and extracted with EtOAc. The combined organics were washed successively with water and brine, dried (Na₂SO₄) and concentrated in vacuo. Purification of the crude residue by flash chromatography on silica gel (gradient elution; 0%-15% EtOAc/hexanes as eluent) afforded the title compound i-6c. m/z (ES) 423 (MH)⁺.

### Step D: Preparation of ethyl 4-[3,3-difluoro-1-(4-hydroxyphenyl)cyclobutyl]benzoate (i-6d)

Intermediate i-6d was prepared following procedures as described for i-4d. m/z (ES) 333 (MH)⁺.

### Step E: Preparation of ethyl 4-{3,3-difluoro-1-[4-(pyridin-2-ylmethoxy)phenyl]-cyclobutyl}benzoate (i-6e)

Intermediate i-6e was prepared following procedures as described for i-4e. m/z (ES) 424 (MH)⁺.

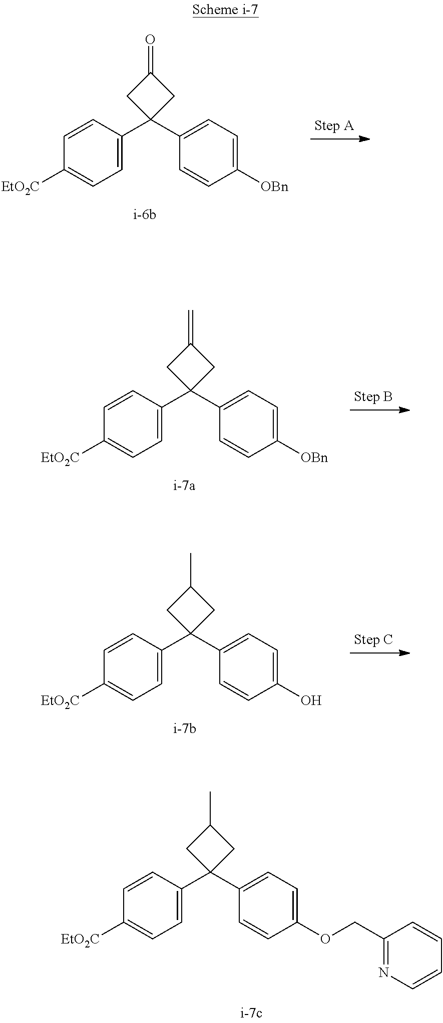

### Step A: Preparation of ethyl 4-{1-[4-(benzyloxy)phenyl]-3-methylenecyclobutyl}benzoate (i-7a)

Potassium bis(trimethylsilyl)amide (3.0 mL of a 0.5 M solution in toluene) was added dropwise to a solution of methyl triphenylphosphonium bromide (0.56 g, 1.6 mmol) in THF (10 mL) at 0° C. After 30 min, a solution of i-6b (0.42 g, 1.1 mmol) in THF (5 mL) was added dropwise, and the resulting reaction mixture was warmed to rt. After 1 h, the reaction mixture was quenched with saturated aqueous ammonium chloride, extracted three times with EtOAc. The combined organics were washed successively with water and brine, dried (Na₂SO₄) and concentrated in vacuo. Purification of the crude residue by flash chromatography on silica gel (gradient elution; 0%-10% EtOAc/hexanes as eluent) afforded the title compound i-7a. m/z (ES) 399 (MH)⁺.

### Step B: Preparation of ethyl 4-[1-(4-hydroxyphenyl)-3-methylcyclobutyl]benzoate (i-7b)

A mixture of i-7a (55 mg, 0.14 mmol) and palladium hydroxide (10 wt. % on activated carbon) in ethanol (3 mL) was hydrogenated at atmospheric pressure for 2 h. The resulting mixture was filtered through a short column of Celite®, eluting copiously with EtOAc. The filtrate was concentrated in vacuo to afford the title compound i-7b as a 1:1 mixture of stereoisomers. m/z (ES) 311 (MH)⁺.

### Step C: Preparation of ethyl 4-{3-methyl-1-[4-(pyridin-2-ylmethoxy)phenyl]cyclobutyl}-benzoate (i-7c)

Intermediate i-7c was prepared as a 1:1 mixture of stereoisomers following procedures as described for i-4e. m/z (ES) 402 (MH)⁺.

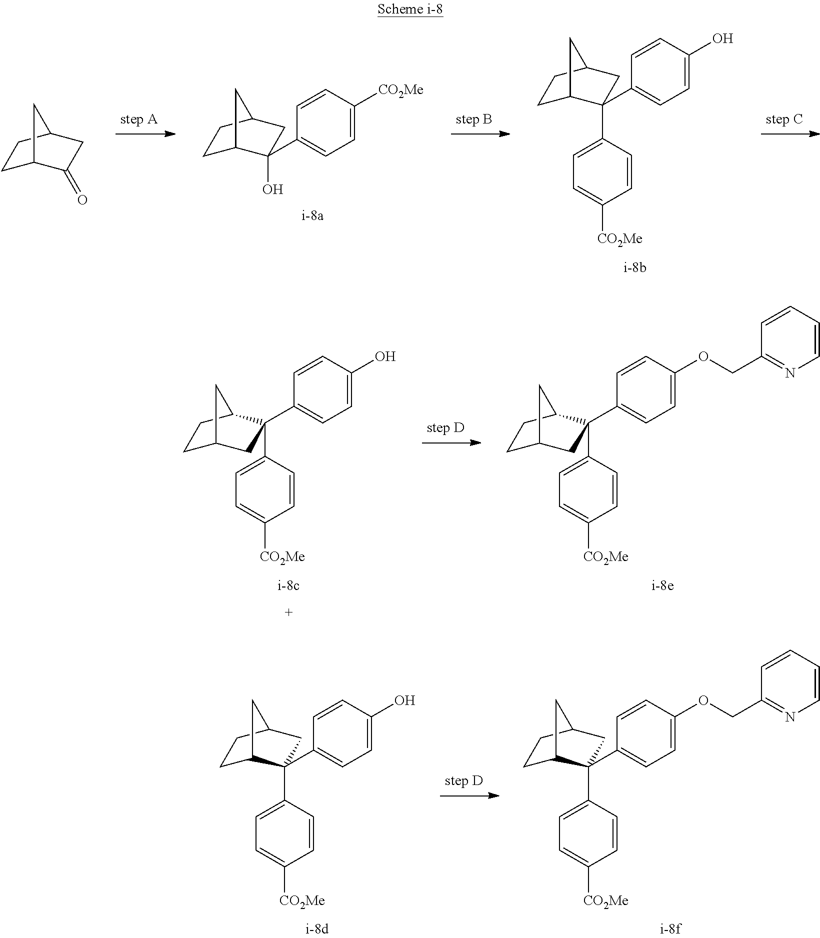

### Step A: Preparation of methyl 4-(2-hydroxybicyclo[2.2.1]hept-2-yl)benzoate (i-8a)

Isopropyl magnesium chloride (48.0 mL of a 2.0 M solution in THF, 0.0970 mol) was added dropwise to a stirred solution of methyl 4-iodobenzoate (23.1 g, 0.088 mol) in THF (200 mL) at −47° C. The reaction mixture was stirred at −47° C. for 1 h and cannulated into a solution of norcamphor (11.6 g, 0.105 mol) in THF (100 mL) at −47° C. The reaction mixture was stirred at −40° C. overnight, and upon warming to −20° C., was quenched by addition of saturated aqueous ammonium chloride. The resulting mixture was extracted with EtOAc, and the combined organics were washed with water and brine, dried (MgSO₄), filtered, and concentrated in vacuo. Purification of the crude residue by flash chromatography on silica gel (gradient elution; 0-30% EtOAc/hexanes as eluent) to afforded the title compound i-8a. m/z (ES) 229 (M−OH)⁺. ¹HNMR (500 MHz, CDCl₃): δ 8.02 (d, 2H, J=10.0 Hz), 7.61 (d, 2H, J=10.0 Hz), 3.94 (s, 3H), 2.61 (bs, 1H), 2.36 (m, 1H), 2.31 (m, 1H), 2.20 (m, 1H), 1.77 (S, 1H), 1.69 (m, 1H), 1.52 (m, 4H), 1.40 (m, 1H).

### Step B: Preparation of methyl 4[2-(4-hydroxyphenyl)bicyclo[2.2.1]hept-2-yl]benzoate (i-8b)

A mixture of phenol (7.80 g, 82.8 mmol) and p-TSA (2.48 g, 13.0 mmol) in toluene (160 mL) were heated at reflux for 10 min in a 2-neck round bottom flask equipped with a Dean-Stark apparatus. A solution of i-8a (10.2 g, 41.4 mmol) in toluene (40 mL) was added over 10 min via an addition funnel, and the resulting mixture was heated at reflux for 14 h. The reaction mixture was cooled to rt and diluted with EtOAc. The resulting mixture was washed with 1.0 N sodium hydroxide, water and brine, dried (MgSO₄), filtered, and concentrated in vacuo. Purification of the crude residue by flash chromatography on silica gel (gradient elution; 0-20% EtOAc/hexanes as eluent) afforded the title compound i-8b. m/z (ES) 323 (MH)⁺.

### Step C: Preparation of i-8c and i-8d

Enantiomers i-8c and i-8d were separated using preparative normal phase chiral HPLC (Chiralpak AD column, 15% EtOH/heptane as eluent). The eluants were concentrated to provide the enantiomers i-8c and i-8d.

For i-8c: (+) CD deflection (Chiralpak AD column (4.6×250 mm; 10 micron, flow rate=1 mL/min, λ=220 nm UV detection), retention time=9.18 min). m/z (ES) 323 (MH)⁺. ¹HNMR (500 MHz, CDCl₃): δ 7.91 (d, 2H, J=8.4 Hz), 7.36 (d, 2H, J=8.5 Hz), 7.18 (d, 2H, J=8.9 Hz), 6.71 (d, 2H, J=8.7 Hz), 4.68 (s, 1H), 3.89 (s, 3H), 3.19 (bs, 1H), 2.42 (bs, 1H), 2.34 (m, 2H), 1.74 (bd, 1H, J=9.6 Hz), 1.52 (m, 2H), 1.41 (dd, 1H, J=1.6, 9.6 Hz), 1.17 (m, 2H).
For i-8d: (−) CD deflection (Chiralpak AD column (4.6×250 mm; 10 micron, flow rate=1 mL/min, λ=220 nm UV detection), retention time=14.95 min). m/z (ES) 323 (MH)⁺. ¹HNMR (500 MHz, CDCl₃): δ 7.91 (d, 2H, J=8.5 Hz), 7.36 (d, 2H, J=8.5 Hz), 7.18 (d, 2H, J=8.7 Hz), 6.71 (d, 2H, J=8.7 Hz), 4.89 (s, 1H), 3.90 (s, 3H), 3.19 (bs, 1H), 2.42 (bs, 1H), 2.34 (m, 2H), 1.75 (bd, 1H, J=9.6 Hz), 1.52 (m, 2H), 1.41 (dd, 1H, J=1.6, 9.6 Hz), 1.17 (m, 2H).

### Step D: Preparation of methyl 4-{2-[4-(pyridin-2-ylmethoxy)phenyl]bicyclo[2.2.1]hept-2-yl}benzoate (i-8e)

Intermediate i-8e was prepared following procedures as described for i-4e. m/z (ES) 414 (MH)⁺. ¹HNMR (500 MHz, CDCl₃): δ 8.60 (bd, 1H, J=4.8 Hz), 7.91 (d, 2H, J=8.4 Hz), 7.72 (dt, 1H, J=7.8, 1.8 Hz), 7.51 (d, 1H, J=8.0 Hz), 7.36 (d, 2H, J=8.5 Hz), 7.24 (d, 2H, J=8.7 Hz), 6.86 (d, 2H, J=8.9 Hz), 5.16 (s, 2H), 3.89 (s, 3H), 3.20 (bs, 1H), 2.42 (bs, 1H), 2.34 (m, 2H), 1.74 (bd, 1H, J=9.4 Hz), 1.61 (b, 1H), 1.52 (m, 2H), 1.41 (dd, 1H, J=9.6, 1.4 Hz), 1.17 (m, 2H).

Compounds i-8g and i-8h can be prepared following procedures as described for i-4e.

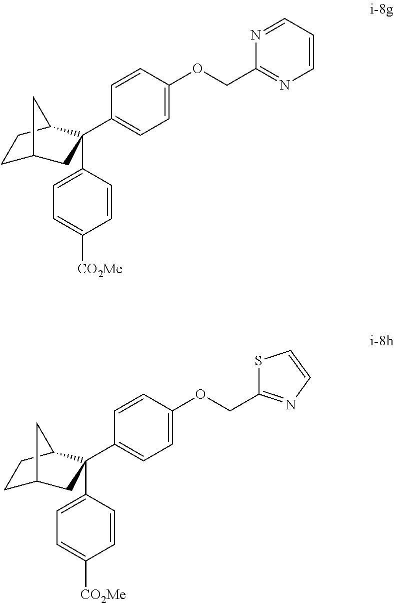

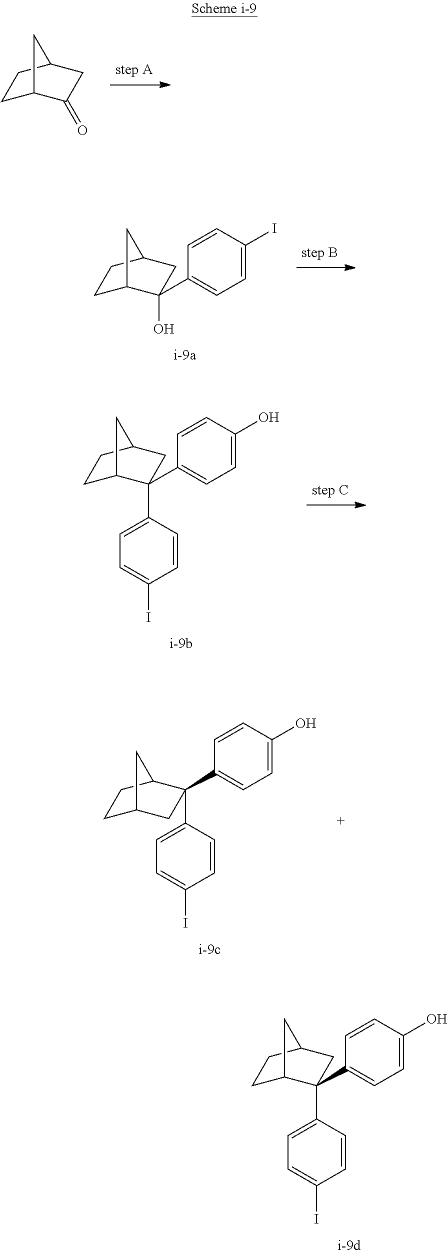

### Step A: Preparation of 2-(4-iodophenyl)bicyclo[2.2.1]heptan-2-ol (i-9a)

n-Butyllithium (98.0 mL of a 2.5 M solution in THF, 245 mmol) was added to a solution of diiodobenzene (80.2 g, 243 mmol) in THF (75 mL) with an addition rate that maintained an the internal temperature below −70° C. The resulting mixture was allowed to stir at −78° C. for 15 min, at which time, a solution of norcamphor (26.7 g, 243 mmol) in THF (15 mL) was added via cannula at such a rate that maintained an internal temperature below −70° C. The reaction mixture was stirred at −78° C. for 15 min, at which point, the cooling bath was removed, and the mixture was warmed to rt over 30 min. The reaction was quenched with a minimum volume of saturated aqueous ammonium chloride solution, dried (MgSO₄), and concentrated in vacuo to afford the title compound i-9a.

### Step B: Preparation of methyl 4[2-(4-iodophenyl)bicyclo[2.2.1]hept-2-yl]phenol (i-9b)

Following a similar procedure, as described for intermediate i-8b, intermediate i-9b was prepared. ¹HNMR (500 MHz, CDCl₃): δ 7.54 (d, 2H, J=8.4 Hz), 7.16 (d, 2H, J=8.7 Hz), 7.03 (d, 2H, J=8.5 Hz), 6.70 (d, 2H, J=8.5 Hz), 4.52 (s, 1H), 3.12 (d, 1H, J=2.5 Hz), 2.40 (bs, 1H), 2.26 (bs, 2H), 1.72 (bd, 1H, J=9.4 Hz), 1.50 (m, 2H), 1.40 (bd, 1H, J=9.6 Hz), 1.19 (m, 2H).

### Step C: Preparation of i-9c and i-9d

Enantiomers i-9c and i-9d were separated using preparative normal phase chiral HPLC. A solution of i-9b in methanol was injected onto a ChiralCel® OD-H (available from Chiral Technologies, Inc., Exton, Pa.) semi-preparative (250×20 mm) HPLC column (eluting with 40% methanol/CO₂ with a column temperature of 40° C. at 50 mL/min with UV detection at 220 nm). The enantiomers were separated with the faster eluting enantiomer i-9c having a retention time of 6.32 min and (+)-CD deflection, and the slower eluting enantiomer i-9d having a retention time of 8.32 min and (−)-CD deflection. The eluants were concentrated to provide the enantiomers i-9c and i-9d.

Compounds i-9e and i-9f can be prepared following the procedures similar to those described above.

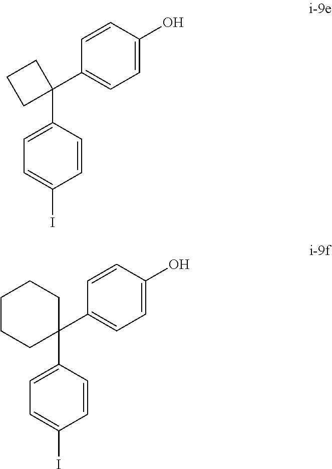

Compounds i-9g through i-9o can be prepared following procedures as described for i-4e.

| | 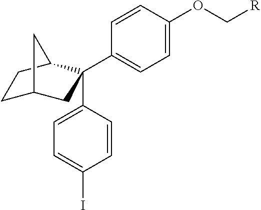 | 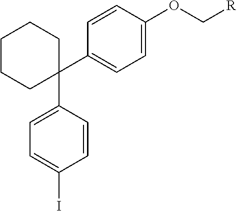 | 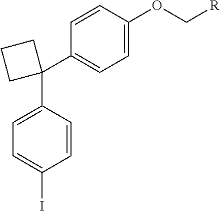 |
|---|---|---|---|
| R = 2-pyridyl | i-9g | i-9j | i-9m |
| R = 2-pyrimidinyl | i-9h | i-9k | i-9n |
| R = 2-thiazolyl | i-9i | i-9l | i-9o |

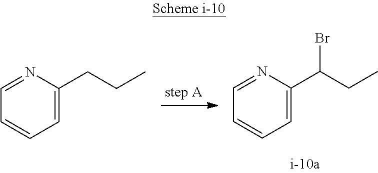

### Step A: Preparation of 2-(1-bromopropyl)pyridine (i-10a)

N-Bromosuccinimide (1.62 g, 9.09 mmol), and 2,2′-azobisisobutyronitrile (0.5 g, 3.05 mmol) was added to a solution of 2-propylpyridine (1.0 g, 8.26 mmol) in carbon tetrachloride (20 mL), and the resulting mixture stirred under a sun lamp overnight. The reaction mixture was filtered through a pad of Celite®, and solid layer was rinsed with DCM. The combined filtrate was concentrated in vacuo, and the resulting crude residue was purified by flash chromatography on silica gel (gradient elution; 2.5-5% EtOAc/hexanes as eluent) to afford the title compound i-10a. m/z (ES) 201 (MH)⁺.

Intermediate i-10b can be prepared following similar procedures as described for i-10a.

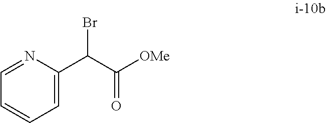

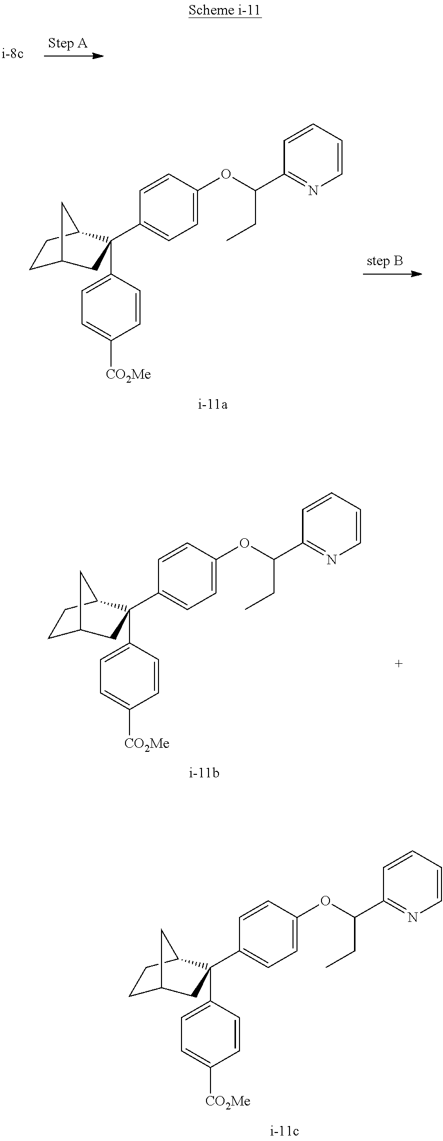

### Step A: Preparation of methyl 4-{2-[4-(1-pyridin-2ylpropoxy)phenyl]bicyclo[2.2.1]hept-2-yl}benzoate (i-11a)

Intermediate i-11a was prepared following the procedure as described for i-4e. m/z (ES) 442 (MH)⁺.

### Step B: Preparation of i-11b and i-11c

Diastereomers i-11b and i-11c were separated using preparative normal phase chiral HPLC. A solution of i-11a in methanol was injected onto a ChiralCel® OJ-H (available from Chiral Technologies, Inc., Exton, Pa.) semi-preparative (250×20 mm) HPLC column (eluting with 20% methanol/CO₂ with a column temperature of 40° C. at 50 mL/min with UV detection at 220 nm). The isomers were separated with the faster eluting enantiomer i-11b having a retention time of 6.09 min, and the slower eluting enantiomer i-11c having a retention time of 7.08 min. The eluants were concentrated to provide the enantiomers i-11b and i-11c.

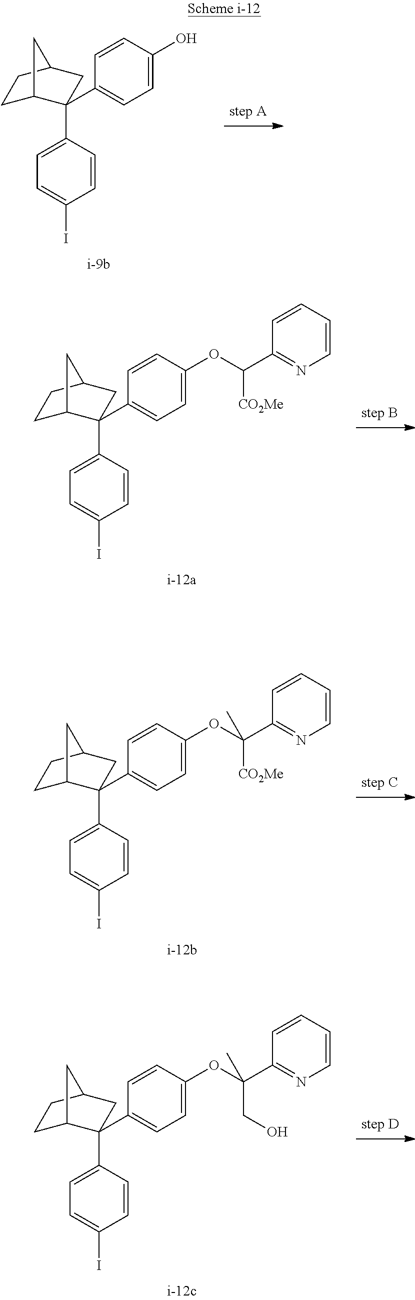

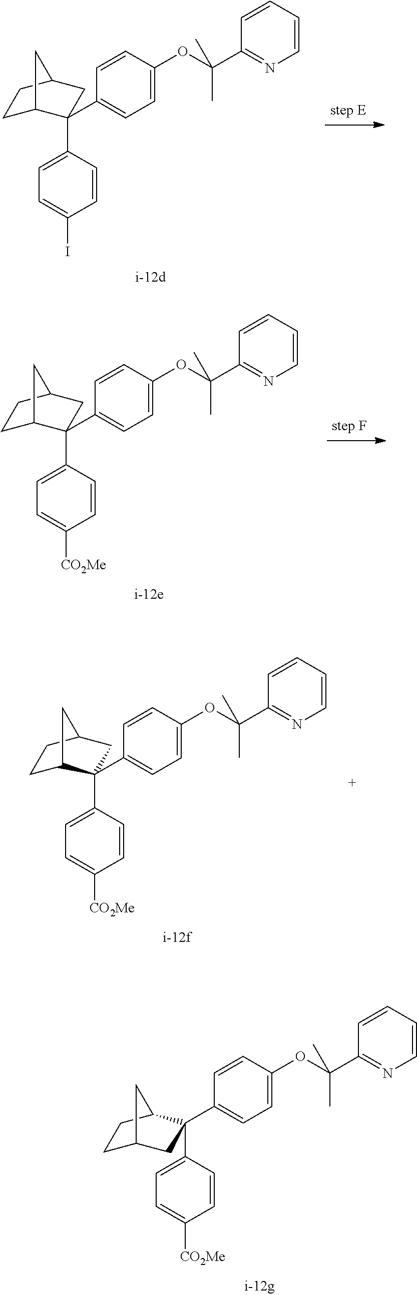

### Step A: Preparation of methyl {4-[2-(4-iodophenyl)bicyclo[2.2.1]hept-2-yl]phenoxy}(pyridin-2-yl)acetate (i-12a)

Intermediate i-12a was prepared utilizing i-9b following the procedure as described for i-4e.

### Step B: Preparation of methyl 2-{4-[2-(4-iodophenyl)bicyclo[2.2.1]hept-2-yl]phenoxy}-2-pyridin-2-ylpropanoate (i-12b)

Lithium diisopropylamide (10.4 mL of a 1.5 M solution in toluene; 15.6 mmol) was added dropwise to a solution of i-12a (6.42 g, 11.9 mmol) in THF (100 mL) at −78° C., and the resulting mixture was warmed to rt over 15 min, then recooled to −78° C. DMPU (2.00 mL) and methyl iodide (2.03 g, 14.3 mmol) were added, successively, and the reaction mixture was stirred at −78° C. for 1 h, then warmed to rt and allowed to stir for 1 h. The reaction was quenched with saturated aqueous ammonium chloride and poured into EtOAc. The layers were separated, and the organic layer was washed with saturated aqueous sodium bicarbonate and brine, dried (Na₂SO₄), filtered, and concentrated in vacuo. Purification of the crude residue by flash chromatography on silica gel (gradient elution; 15-40% EtOAc/hexanes as eluent) afforded the title compound i-12b.

### Step C: Preparation of 2-{4-[2-(4-iodophenyl)bicyclo[2.2.1]hept-2-yl]phenoxy}-2-pyridin-2-ylpropan-1-ol (i-12c)

Lithium borohydride (100 μL of a 2.0 M solution in THF, 0.200 mmol) was added to a solution of i-12b (100 mg, 0.181 mmol) in THF (2 mL). After 16 h, the reaction was quenched with 2.0 M HCl, neutralized with saturated aqueous sodium bicarbonate, and poured into EtOAc. The layers were separated, and the organic layer was washed with saturated aqueous sodium bicarbonate and brine, dried (Na₂SO₄), filtered, and concentrated in vacuo. Purification of the crude residue by flash chromatography on silica gel (gradient elution; 20-50% EtOAc/hexanes as eluent) afforded the title compound i-12c.

### Step F: Preparation of 2-(1-{4-[2-(4-iodophenyl)bicyclo[2.2.1]hept-2-yl]phenoxy}-1-methylethyl)pyridine (i-12d)

A solution of trifluoromethylsulfonic anhydride (160 μL, 0.950 mmol) in DCM (3 mL) was added into a solution of triphenylphosphine oxide (530 mg, 1.90 mmol) in DCM (5 mL) at 0° C. After 15 min, a solution of i-12c (500 mg, 0.95 mmol) in DCM (2.5 mL) was added and allowed to stir for 15 min. Sodium borohydride (140 mg, 3.80 mmol) was then added in one portion, and the resulting mixture was warmed to rt and allowed to stir overnight. The reaction was quenched with 2.0 M HCl, neutralized with saturated aqueous sodium bicarbonate, and poured into EtOAc. The layers were separated, and the organic layer was washed with saturated aqueous sodium bicarbonate and brine, dried (Na₂SO₄), filtered, and concentrated in vacuo. Purification of the crude residue by flash chromatography on silica gel (gradient elution; 0-50% EtOAc/hexanes as eluent) afforded the title compound i-12d.

### Step G: Preparation of methyl 4-{2-[4(1-methyl-1-pyridin-2-ylethoxy)phenyl]bicyclo-[2.2.1]hept-2-yl}benzoate (i-12e)

Intermediate i-12e was prepared following the procedure as described for preparation of i-5d. m/z (ES) 442 (MH)⁺.

### Step H: Preparation of i-12f and i-12g

Enantiomers i-12f and i-12g were separated using preparative normal phase chiral HPLC (Chiralpak AD column, 12% EtOH/heptane as eluent). The eluants were concentrated to provide the enantiomers i-12f and i-12g.

For i-12f: retention time=7.83 min on analytical Chiralpak AD column (4.6×250 mm; 10 micron, flow rate=1 mL/min, λ=220 nm UV detection).
For i-12g: retention time=11.24 min on analytical Chiralpak AD column (4.6×250 mm; 10 micron, flow rate=1 mL/min, λ=220 nm UV detection).

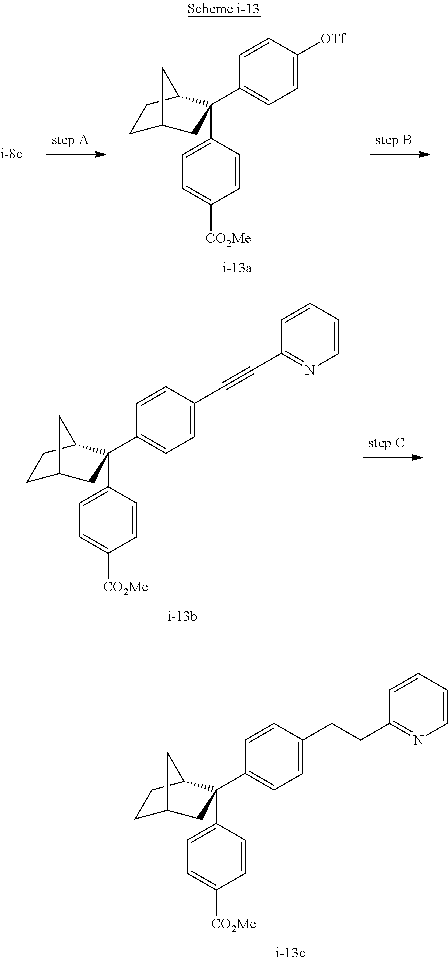

### Preparation of i-13c

### Step A: Preparation of methyl 4-[2-(4-{[(trifluoromethyl)sulfonyl]oxy}phenyl) bicyclo[2.2.1]hept-2-yl]benzoate (i-13a)

Trifluoromethylsulfonic anhydride (301 μL, 3.41 mmol) was added to a solution of pyridine (1.25 mL, 15.5 mmol) and i-8c (1.00 g, 3.10 mmol) in DCM (10.0 mL) at 0° C. The cooling bath was removed, and the reaction mixture was warmed to rt over 30 min. The reaction was partitioned between EtOAc and saturated aqueous sodium bicarbonate. The layers were separated, and the organic layer was washed with brine, dried (Na₂SO₄), and concentrated in vacuo. Purification of the crude residue by flash chromatography on silica gel (gradient elution; 5-20% EtOAc/hexanes as eluent) afforded the title compound i-13a. ¹HNMR (500 MHz, CDCl₃): δ 7.93 (d, 2H, J=8.4 Hz), 7.38 (d, 2H, J=8.9 Hz), 7.34 (d, 2H, J=8.5 Hz), 7.13 (d, 2H, J=8.9 Hz), 3.89 (s, 3H), 3.21 (bs, 1H), 2.45 (bs, 1H), 2.43 (dd, 1H, J=2.1, 13.0 Hz), 2.26 (m, 1H), 1.69 (bd, 1H, J=9.6 Hz), 1.54 (m, 2H), 1.46 (dd, 1H, J=1.5, 9.7 Hz), 1.18 (m, 2H).

### Step B: Preparation of methyl 4-{2-[4-(pyridin-2-ylethynyl)phenyl]bicyclo[2.2.1]heptyl}benzoate (i-13b)

Tetrakis(triphenylphosphine) palladium (250 mg, 0.22 mmol) was added to a solution of i-13a (200 mg, 0.440 mmol), 2-ethynylpyridine (136 mg, 1.32 mmol), tetrabutylammonium iodide (160 mg, 0.440 mmol), triethylamine (310 mg, 3.08 mmol), and copper(I) iodide (25 mg, 0.27 mmol) in DMF (5.00 mL). The resulting mixture was degassed and heated to 80° C. overnight. The reaction mixture was diluted with EtOAc, washed with saturated aqueous sodium bicarbonate and brine, dried (Na₂SO₄), and concentrated in vacuo. Purification of the crude residue by flash chromatography on silica gel (gradient elution; 20-50% EtOAc/hexanes as eluent) afforded the title compound i-13b. m/z (ES) 408 (MH)⁺. ¹HNMR (500 MHz, CDCl₃): δ 8.59 (d, 1H, J=4.8 Hz), 7.90 (d, 2H, J=8.4 Hz), 7.63 (dd, 1H, J=1.6, 7.8 Hz), 7.45 (m, 3H), 7.35 (d, 2H, J=8.2 Hz), 7.31 (d, 2H, J=8.5 Hz), 7.20 (m, 1H), 3.87 (s, 3H), 3.22 (bs, 1H), 2.41 (bs, 1H), 2.39 (d, 1H, J=13.5 Hz), 2.29 (dd, 1H, J=3.4, 13.0 Hz), 1.69 (bd, 1H, J=9.6 Hz), 1.51 (m, 2H), 1.42 (d, 1H, J=9.6 Hz), 1.16 (m, 2H).

### Step C: Preparation of methyl 4-{2-[4-(2-pyridin-2-ylethyl)phenyl]bicyclo[2.2.1]hept-2-yl benzoate (i-13c)

Palladium on carbon (100 mg, 10 wt. % on activated carbon) was added to a solution of i-13b (250 mg, 0.613 mmol) in EtOH:EtOAc (5.00 mL of a 1:1 mixture), and the resulting mixture was hydrogenated (40 psi) for 4 h. The reaction mixture was filtered through a pad of Celite, and the solid layer was rinsed with EtOAc. The combined filtrate was concentrated in vacuo to afford the title compound i-13c. m/z (ES) 412 (MH)⁺. ¹HNMR (500 MHz, CDCl₃): δ 8.56 (d, 1H, J=4.1 Hz), 7.90 (d, 2H, J=8.4 Hz), 7.58 (dt, 1H, J=1.6, 7.6 Hz), 7.36 (d, 2H, J=8.2 Hz), 7.23 (d, 2H, J=8.5 Hz), 7.14 (m, 1H), 7.08 (d, 2H, J=8.2 Hz), 3.88 (s, 3H), 3.23 (bs, 1H), 3.05 (m, 2H), 2.98 (m, 2H), 2.41 (bs, 1H), 2.36 (m, 2H), 1.74 (bd, 1H, J=9.8 Hz), 1.52 (m, 2H), 1.41 (dd, 1H, J=1.4, 9.6 Hz), 1.17 (m, 2H).

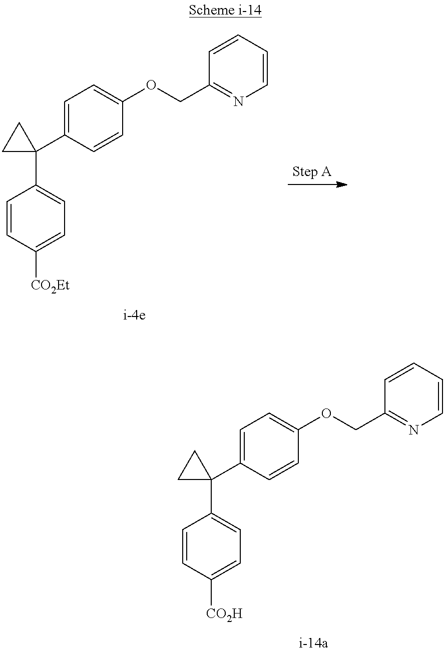

### Step A: Preparation of 4-{1-[4-(pyridin-2-ylmethoxy)phenyl]cyclopropyl}benzoic acid (i-14a)

Lithium hydroxide (31.0 mg, 0.730 mmol) was added to a solution of i-4e (68 mg, 0.18 mmol) in dioxane:water (3.00 mL of a 3:1 mixture), and the resulting reaction mixture was warmed to 55° C. After 1.5 h, the reaction mixture was cooled to rt, quenched with 0.5 M HCl, and extracted with EtOAc. The combined organics were washed successively with water and brine, dried (Na₂SO₄) and concentrated in vacuo to afford the title compound i-14a. m/z (ES) 346 (MH)⁺.

Compounds i-14b-i-14d can be prepared following procedures similar to those described above.

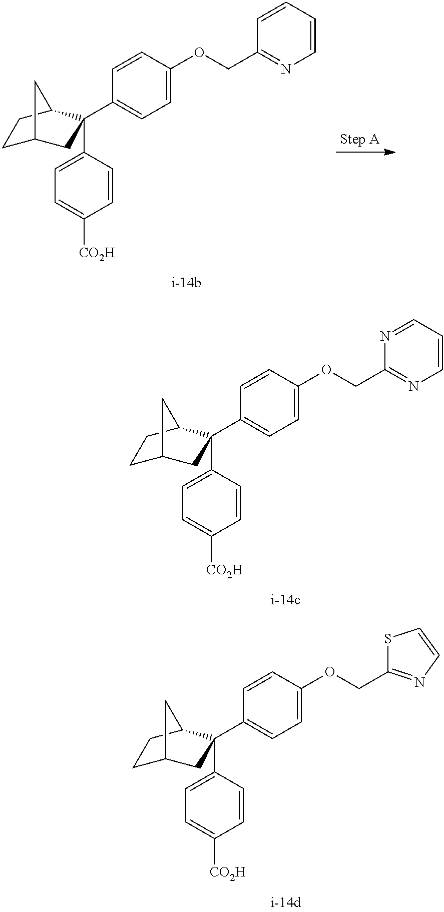

For i-14b: 4-{2-[4-(pyridin-2-ylmethoxy)phenyl]bicyclo[2.2.1]hept-2-yl}benzoic acid: m/z (ES)=400 (MH)⁺

Following procedures similar to those described above, the following compounds in Table i-14 can be prepared:

Table i-14
i-14A

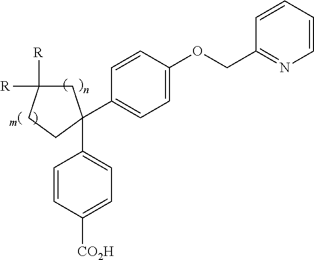

i-14B

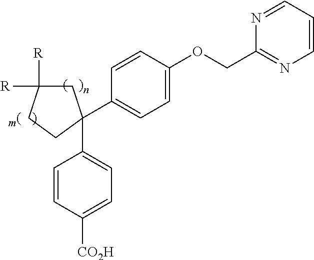

i-14C

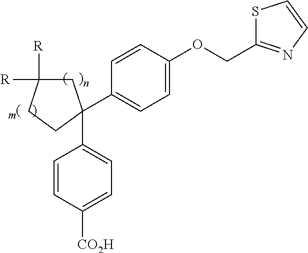

| Ex. i-14A | Ex. i-14B | Ex. i-14C | m | n | R |
|---|---|---|---|---|---|
| a | a | a | 0 | 1 | H |
| b | b | b | 1 | 1 | H |
| c | c | c | 1 | 2 | H |
| d | d | d | 0 | 1 | F |
| e | e | e | 0 | 1 | Me, H |
| f | f | f | 0 | 1 | Me |
| g | g | g | 1 | 2 | F |
| h | h | h | 1 | 2 | Me |

Table i-14. Parent Ion m/z (MH)⁺ data for compounds.
For i-14Aa: 4-{1-[4-(pyridin-2-ylmethoxy)phenyl]cyclobutyl}benzoic acid: m/z (ES)=360 (MH)⁺
For i-14Ab: 4-{1-[4-(pyridin-2-ylmethoxy)phenyl]cyclopentyl}benzoic acid: m/z (ES)=374 (MH)⁺
For i-14Ac: 4-{1-[4-(pyridin-2-ylmethoxy)phenyl]cyclohexyl}benzoic acid: m/z (ES)=388 (MH)⁺
For i-14Ad: 4-{3,3-difluoro-1-[4-(pyridin-2-ylmethoxy)phenyl]cyclobutyl}benzoic acid: m/z (ES)=396 (MH)⁺
For i-14Ae: 4-{3-methyl-1-[4-(pyridin-2-ylmethoxy)phenyl]cyclobutyl}benzoic acid: m/z (ES)=374 (MH)⁺

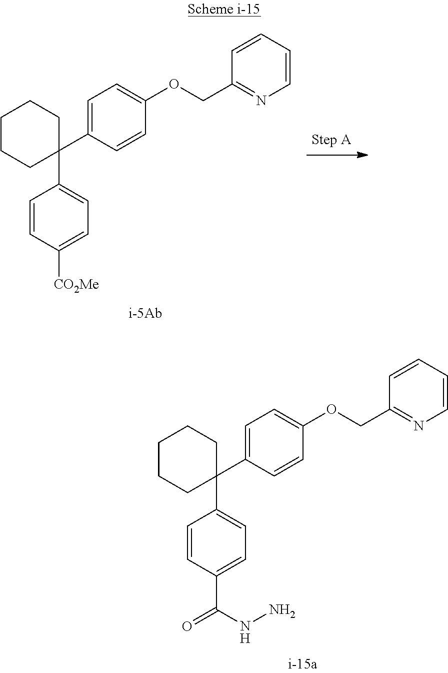

### Preparation of i-15a

### Step A: Preparation of 4-{1-[4-(pyridin-2-ylmethoxy)phenyl]cyclohexyl}benzohydrazide (i-15a)

Hydrazine monohydrate (>10 equiv.) is added to a solution of i-5Ab (1.0 eq) in ethanol, and the resulting mixture is heated to reflux. After 4 h, the reaction mixture is cooled to rt, and diluted with EtOAc and washed three times with water. The combined organics are washed with brine, dried (Na₂SO₄) and concentrated in vacuo to afford the title compound i-15a.

Compounds i-15b through i-15f can be prepared following procedures as described above.

| | 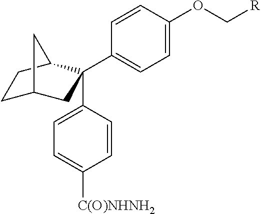 | 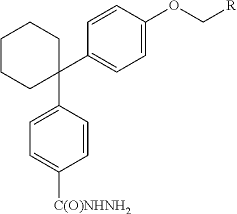 |
|---|---|---|
| R = 2-pyridyl | 1-15b | — |
| R = 2-pyrimidinyl | i-15c | i-15e |
| R = 2-thiazolyl | i-15d | i-15f |

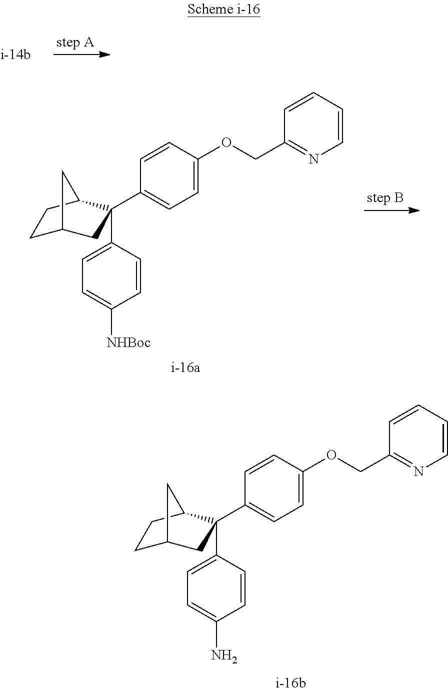

### Preparation of i-16b

### Step A: Preparation of tert-butyl (4-{2-[4-(pyridine-2-ylmethoxy)phenyl]bicyclo[2.2.1]hept-2-yl}phenyl)carbamate (i-16a)

Isobutyl chloroformate (370 μL, 2.80 mmol) was added to a suspension of triethylamine (910 μL, 6.53 mmol) and i-14b (806 mg, 1.85 mmol) in acetone (14.0 mL) at rt. After 1.2 h, the reaction mixture was cooled to 0° C., and a solution of sodium azide (755 mg, 11.6 mmol) in water (5.00 mL) and acetone (5.00 mL) was added. The resulting mixture was stirred at 0° C. for 1 h, and partitioned between toluene and cold saturated aqueous sodium bicarbonate. The layers were separated, and the organic layer was washed with saturated aqueous sodium bicarbonate, dried (Na₂SO₄), and partially concentrated in vacuo. Care should be taken not to concentrate the potentially unstable intermediate acyl azide to dryness. ^{t}BuOH was added to the acyl azide solution, and the resulting mixture was heated to reflux for 4 h. After cooling to rt, the reaction mixture was concentrated in vacuo, and purification of the resulting crude residue by flash chromatography on silica gel (gradient elution; 0-100% (4:5:1) hexanes:DCM:EtOAc/hexanes as eluent) afforded the title compound i-16a.

### Step B: Preparation of 4-{2-[4-(pyridin-2-ylmethoxy)phenyl]bicyclo[2.2.1]hept-2-yl}aniline (i-16b)

A solution of HCl in dioxane (7.00 mL of a 4.0 M solution) and water (350 μL) was added to a solution of i-16a in dioxane (3.00 mL) and water (150 μL) at 0° C. After 40 min, the reaction mixture was warmed to rt and allowed to stir for 35 min. The reaction was quenched with saturated aqueous sodium bicarbonate and extracted with EtOAc. The layers were separated, and the organic layer was washed with saturated aqueous sodium bicarbonate solution, dried (Na₂SO₄), and concentrated in vacuo to afford the title compound i-16b.

Compounds i-16c through i-16g can be prepared following procedures as described above.

| | 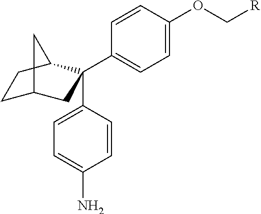 | 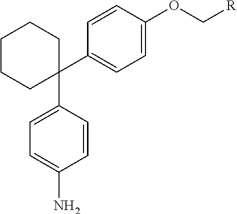 |
|---|---|---|
| R = 2-pyridyl | — | i-16e |
| R = 2-pyrimidinyl | i-16c | i-16f |
| R = 2-thiazolyl | i-16d | i-16g |

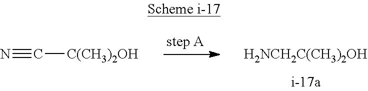

### Step A: Preparation of 1-amino-2-methylpropan-2-ol (i-17a)

Palladium on carbon (53 mg, 10 wt. % on activated carbon) was added to a solution of acetone cyanohydrin (105 mg, 1.23 mmol) in MeOH (9.00 mL) and glacial acetic acid (9.00 mL). The resulting mixture was hydrogenated (1 atm) overnight. The reaction mixture was filtered through a pad of Celite® and the solid layer was rinsed with MeOH. The combined filtrate was partially concentrated in vacuo, and the resulting mixture was treated with conc. HCl (1.00 mL). The mixture was azeotroped with toluene to afford the title compound i-17a. m/z (ES) 90 (MH)⁺.

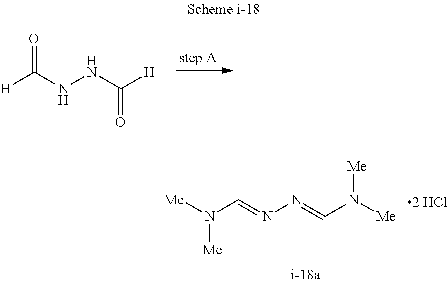

### Step A: Preparation of N′-[(1E)-(dimethylamino)methylene]-N,N-dimethylhydrazono formamide dihydrochloride (i-18a)

Thionyl chloride (2.00 mL, 28.4 mmol) was added into a chilled solution of diformylhydrazine (1.02 g, 11.4 mmol) in DMF (22 mL) at 0° C. After 10 min, the yellow mixture was warmed to rt and allowed to stir overnight. The resulting precipitate was collected and rinsed with DMF, followed by Et₂O to afford the title compound i-18a. m/z (ES) 143 (MH)⁺.

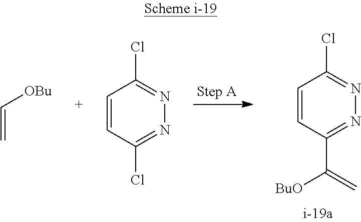

### Step A: Preparation of 3-(1-butoxyvinyl)-6-chloropyridazine (i-19a)

THF (24.0 mL) was added rapidly dropwise to tert-butyllithium (150 mL of a 1.7 M solution in pentane) at −78° C. After 15 min, n-butyl vinyl ether (14.0 mL, 109.4 mmol) was added, and the resulting mixture was warmed to −30° C., at which point modest gas evolution was observed. As gas evolution ceased, a second portion of n-butyl vinyl ether (14.0 mL, 109.4 mmol) was added, maintaining the reaction temperature at −30° C. After gas evolution had ceased, the reaction mixture was cooled to −78° C., and a solution of zinc chloride (29.8 g, 219 mmol) in THF (250 mL) was added rapidly dropwise. After 15 min, the reaction was warmed to −10° C. and transferred via cannula to a stirred solution of 3,6-dichloropyridazine (32.6 g, 219 mmol) and [1,1′-bis(diphenylphosphino)ferrocene]dichloropalladium(II) (16.0 g, 21.9 mmol) in THF (200 mL) at 0° C. After 1 h at 0° C., the reaction mixture was diluted with EtOAc and filtered through a short column of Celite®, eluting with EtOAc. The filtrate was washed with water and brine, dried (sodium sulfate), and concentrated in vacuo. The crude residue was purified by flash chromatography on silica gel (gradient elution; 0%-15% EtOAc/hexanes as eluent) to afford the title compound i-19a. ¹HNMR (500 MHz, CDCl₃): δ 7.80 (d, 1H, J=8.9 Hz), 7.52 (d, 1H, J=8.9 Hz), 5.76 (d, 1H, J=2.5 Hz), 4.55 (d, 1H, J=2.5 Hz), 3.97 (t, 2H, J=6.4 Hz), 1.83 (m, 2H), 1.57 (m, 2H), 1.02 (t, 3H, J=7.5 Hz).

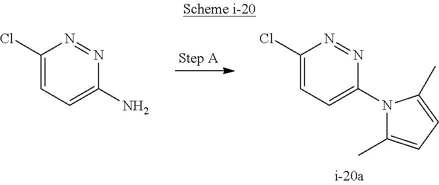

### Preparation of 3-chloro-6-(2,5-dimethyl-1H-pyrrol-1-yl)pyridazine (i-20a)

A mixture of p-TSA (117 mg, 0.618 mmol), 2,5-hexanedione (4.36 mL, 37.1 mmol) and 3-amino-6-chloropyridazine (4.00 g, 30.9 mmol) in toluene (150 mL) was heated at 140° C. for 5 h in a round bottom flask equipped with a condenser and Dean-Stark apparatus. The reaction mixture was cooled to rt and charcoal was added. The mixture was filtered through Celite® and concentrated in vacuo to afford the title compound i-20a. m/z (ES) 208 (MH)⁺.

Compound i-20b was prepared following the procedures described above. m/z (ES) 252 (MH)⁺.

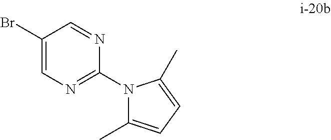

### Example 1

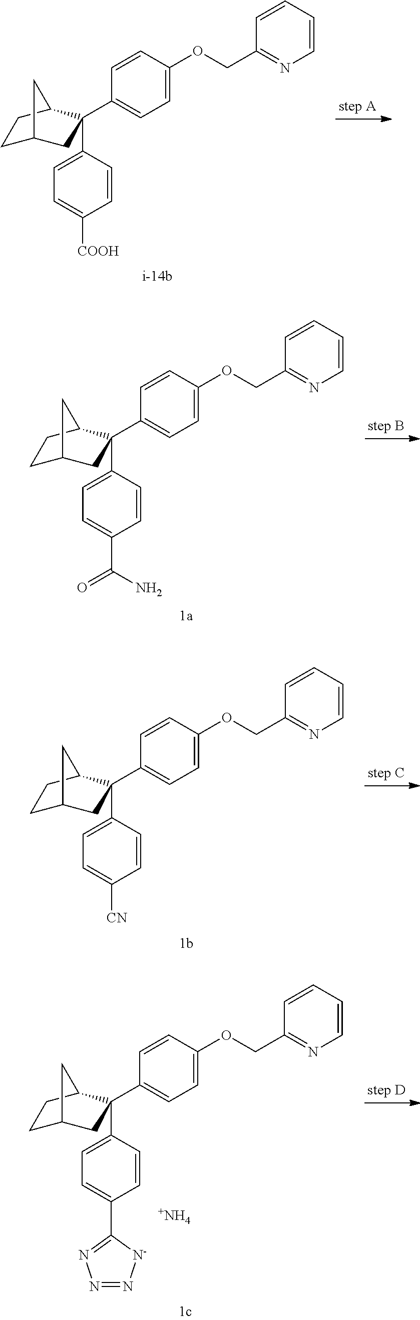

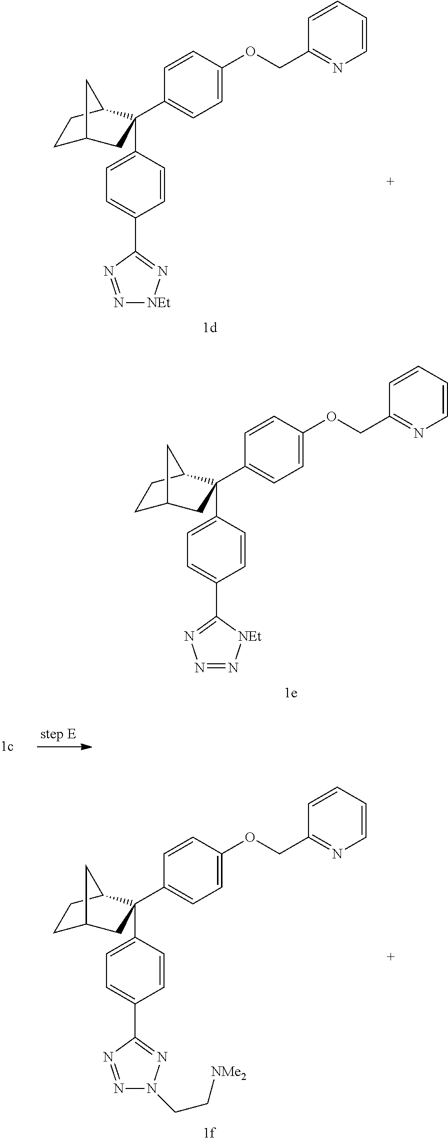

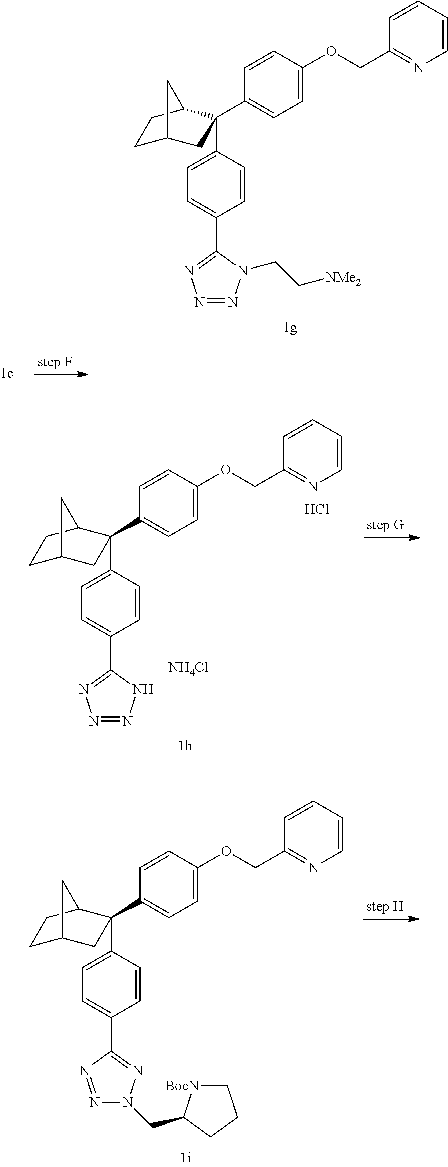

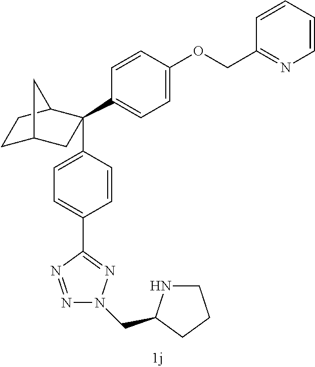

### Step A: Preparation of 4-{2-[4-(pyridin-2-ylmethoxy)phenyl]bicyclo[2.2.1]hept-2-yl}benzamide (1a)

HATU (1.36 g, 3.58 mmol) and DIPEA (6.50 ml, 37.3 mmol) were added to a stirred suspension of i-14b (1.00 g, 2.30 mmol) and ammonium chloride (1.90 g, 35.5 mmol) in DMF (15.0 ml), and the resulting yellow suspension was stirred at rt. After 2 d, the reaction mixture was quenched with saturated aqueous sodium bicarbonate and extracted with EtOAc. The organic extracts were washed with water, dried (Na₂SO₄), and concentrated in vacuo. The crude residue was purified by flash chromatography on silica gel (gradient elution; 0-80% EtOAc/hexane as eluent) to afford the title compound 1a as a white foam. m/z (ES) 399 (MH)⁺.

### Step B: Preparation of 4-{2-[4-(pyridin-2-ylmethoxy)phenyl]bicyclo[2.2.1]hept-2-yl}benzonitrile (1b)

Cyanuric chloride (447 mg, 2.42 mmol) was added to a stirred suspension of 1a (787 mg, 1.97 mmol) in DMF (12.0 ml) at 0° C., and the resulting mixture was warmed to rt. After 40 min, the reaction mixture was cooled to 0° C., quenched with aqueous sodium bicarbonate solution, and extracted with EtOAc. The organic extracts were washed with saturated aqueous sodium bicarbonate and water, dried (Na₂SO₄), and concentrated in vacuo. The crude residue was purified by flash chromatography on silica gel (gradient elution; 0-30% EtOAc/hexane as eluent) to afford the title compound 1b as a white foam. m/z (ES) 381 (MH)⁺.

### Step C: Preparation of ammonium 5-(4-{2-[4-(pyridin-2-ylmethoxy)phenyl]-bicyclo[2.2.1]hept-2-yl}phenyl)tetrazol-1-ide (1c)

Azidotrimethyltin (856 mg, 4.16 mmol) was added to a stirred solution of 1b (782 mg, 2.06 mmol) in toluene (11 ml), and the resulting mixture was heated to 105° C. After 15 min, an additional portion of azidotrimethyltin (1.03 g, 5.00 mmol) was added. After 19 h, the reaction mixture was cooled to rt and quenched with a solution of 1.0 M HCl in ethanol (11 ml) and allowed to stir at rt. After 30 min, the mixture was concentrated in vacuo, and the crude residue was purified by flash chromatography on silica gel (gradient elution; 0-15% MeOH/DCM (with 1% ammonium hydroxide as modifier) as eluent) to afford the title compound 1c as a white solid. m/z (ES) 424 (MH)⁺.

### Step D: Preparation of 2-[(4-{2-[4-(2-ethyl-2H-tetrazol-5-yl)phenyl]bicyclo[2.2.1]hept-2-yl}phenoxy)methyl]pyridine (1d) and 2-[(4-{2-[4-(1-ethyl-1H-tetrazol-5-yl)phenyl]bicyclo[2.2.1]hept-2-yl}phenoxy)methyl]pyridine (1e)

Ethyl iodide (38.0 μL, 0.479 mmol) and powdered potassium carbonate (80.0 mg, 0.583 mmol) were added to a solution of 1c (51.0 mg, 0.116 mmol) in DMF (1.0 mL) at 0° C., and after 10 min, the reaction mixture was warmed to rt. After 1.5 h, the reaction mixture was quenched with saturated aqueous sodium bicarbonate, and extracted with EtOAc. The organic extracts were washed with saturated aqueous sodium bicarbonate and water, dried (Na₂SO₄), and concentrated in vacuo. The crude residue was purified by flash chromatography on silica gel (gradient elution; 0-50% EtOAc/hexanes as eluent) to afford the title compounds 1d (faster eluding isomer, m/z (ES) 452 (MH)⁺) and 1e (slower eluding isomer, m/z (ES) 452 (MH)⁺).

### Step E: Preparation of N,N-dimethyl-2-[5-(4-{2-[4-(pyridin-2-ylmethoxy)phenyl]-bicyclo[2.2.1]hept-2-yl}phenyl)-2H-tetrazol-2-yl]ethanamine (1f) and N,N-dimethyl-2-[5-(4-{2-[4-(pyridin-2-ylmethoxy)phenyl]bicyclo[2.2.1]hept-2-yl}phenyl)-1H-tetrazol-1-yl]ethanamine (1g)

Diethyl azodicarboxylate (28.0 μL, 0.18 mmol) was added to a stirred solution of 1c (44.0 mg, 0.10 mmol), N,N-dimethylethanolamine (15.0 μL, 0.15 mmol), and triphenylphosphine (39.0 mg, 0.15 mmol) in DCM (500 μL), and the resulting mixture was allowed to stir at rt. After 18 h, the reaction mixture was purified directly by flash chromatography on silica gel (gradient elution; 0-50% EtOAc/hexanes as eluent) to afford the title compounds 1f and 1g. The regioisomeric mixture was separated by preparative reversed phase HPLC on YMC Pack Pro C18 stationary phase (CH₃CN/H₂O as eluent, 0.05% TFA as modifier), and lyophilization of the purified fractions afforded the title compounds 1f (slower eluding isomer, m/z (ES) 495 (MH)⁺) and 1g (faster eluding isomer, m/z (ES) 495 (MH)⁺).

### Step F: Preparation of 2-[(4-{2-[4-(1H-tetrazol-5-yl)phenyl]bicyclo[2.2.1]hept-2-yl}phenoxy)methyl]pyridine hydrochloride (1h)

HCl in EtOH (990 μL of a 1.0 M EtOH solution, 0.99 mmol) was added to a stirred solution of 1c (174 mg, 0.395 mmol) in MeOH (5.0 mL), and the resulting mixture was allowed to stir at rt. After 2 min, the reaction mixture was concentrated in vacuo and azeotroped with toluene twice to afford 1 h as an off-white solid.

### Step G: Preparation of tert-butyl (2S)-2-{[5-(4-{2-[4-(pyridin-2-ylmethoxy)phenyl]-bicyclo[2.2.1]hept-2-yl}phenyl)-2H-tetrazol-2-yl]methyl}pyrrolidine-1-carboxylate (1i)

Diethyl azodicarboxylate (35.0 μL, 0.223 mmol) was added dropwise to a stirred suspension of 1 h (49.0 mg, 0.095 mmol), (S)-(−)-1-(tert-butoxycarbonyl)-2-pyrrolidinemethanol (42.0 mg, 0.206 mmol), and triphenylphosphine (52.0 mg, 0.199 mmol) in DCM (1.0 mL), and the resulting mixture was allowed to stir at it overnight. The reaction mixture was purified directly by flash chromatography on silica gel (gradient elution; 0-50% EtOAc/hexanes as eluent) to afford the title compound 1i as a mixture with diethyl hydrazine-1,2-dicarboxylate, a by-product in the reaction.

### Step H: Preparation of 2-({4-[2-(4-{2-[(2S)-pyrrolidin-2-ylmethyl]-2H-tetrazol-5-yl}pheyl)bicyclo[2.2.1]hept-2-yl]phenoxy}methyl)pyridine (1j)

1i (as a mixture with diethyl hydrazine-1,2-dicarboxylate) was dissolved in a solution of hydrochloric acid (4.0 M solution in dioxane, 4 mL) and water (200 μL) at 5° C., and the resulting mixture was allowed to warm to rt. After 30 min, the reaction mixture was concentrated in vacuo, azeotroped with toluene, and the crude residue was purified by flash chromatography on silica gel (gradient elution; 0-100% of 5% MeOH (with 1% ammonium hydroxide as modifier) in DCM/EtOAc as eluent) to afford the title compound 1j as a white solid. m/z (ES) 507 (MH)⁺.

Following the procedures similar to those described above, the following compounds in Tables 1-1 and 1-2 can be prepared:

TABLE 1-1
1A

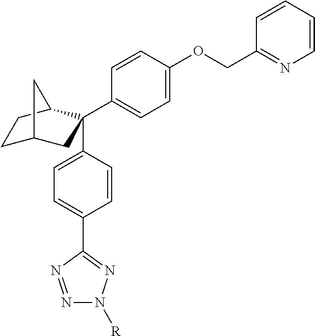

1B

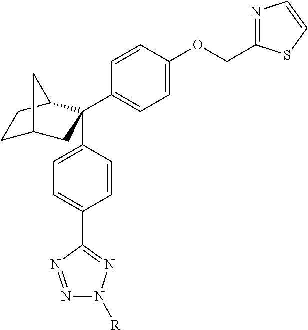

1C

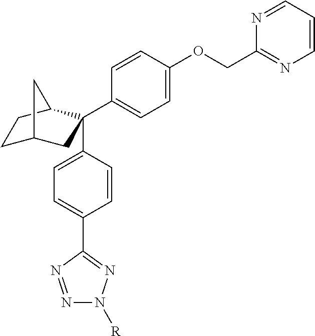

1D

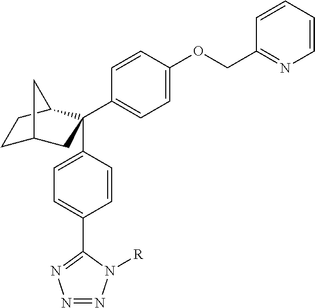

1E

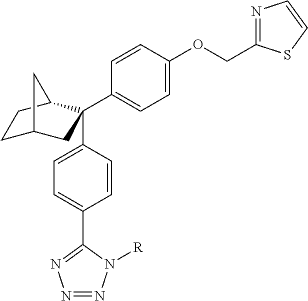

1F

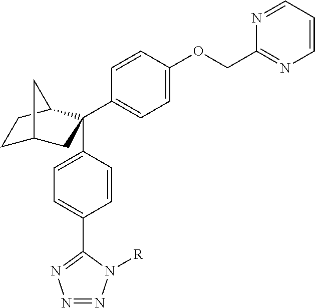

| Ex. | Ex. | Ex. | Ex. | Ex. | Ex. | |
|---|---|---|---|---|---|---|
| 1A | 1B | 1C | 1D | 1E | 1F | R |
| a | a | a | a | a | a | Me |
| — | b | b | — | b | b | Et |
| c | c | c | c | c | c | i-Pr |
| d | d | d | d | d | d | CF₂H |
| — | e | e | — | e | e | H |
| f | f | f | f | f | f | 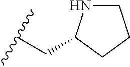 |
| g | g | g | g | g | g | 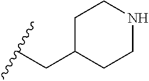 |
| h | h | h | h | h | h | 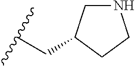 |
| i | i | i | i | i | i | 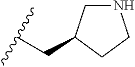 |

Table 1-1. Parent Ion m/z (MH)⁺ data for compounds
For 1Aa: 2-[(4-{(1S,2S,4R)-2-[4-(2-methyl-2H-tetrazol-5-yl)phenyl]bicyclo[2.2.1]hept-2-yl}phenoxy)methyl]pyridine: m/z (ES) 438 (MH)⁺
For 1Ac: 2-[(4-{(1S,2S,4R)-2-[4-(2-isopropyl-2H-tetrazol-5-yl)phenyl]bicyclo[2.2.1]hept-2-yl}phenoxy)methyl]pyridine: m/z (ES) 466 (MH)⁺
For 1Ad: 2-[(4-{(1S,2S,4R)-2-[4-(2-(difluoromethyl)-2H-tetrazol-5-yl)phenyl]bicyclo[2.2.1]-hept-2-yl}phenoxy)methyl]pyridine: m/z (ES) 474 (MH)⁺
For 1Af: 2-({4-[(1S,2S,4R)-2-(4-{2-[(2R)-pyrrolidin-2-ylmethyl]-2H-tetrazol-5-yl}phenyl)bicyclo[2.2.1]hept-2-yl]phenoxy}methyl)pyridine: m/z (ES) 438 (MH)⁺
For 1Ba: 2-methyl-5-(4-{(1S,2S,4R)-2-[4-(1,3-thiazol-2-ylmethoxy)phenyl]bicyclo[2.2.1]hept-2-yl}phenyl)-2H-tetrazole: m/z (ES) 444 (MH)⁺
For 1Bb: 2-ethyl-5-(4-{(1S,2S,4R)-2-[4-(1,3-thiazol-2-ylmethoxy)phenyl]bicyclo[2.2.1]hept-2-yl}phenyl)-2H-tetrazole: m/z (ES) 458 (MH)⁺
For 1Be: 5-(4-{(1S,2S,4R)-2-[4-(1,3-thiazol-2-ylmethoxy)phenyl]bicyclo[2.2.1]hept-2-yl}-phenyl)-2H-tetrazole: m/z (ES) 430 (MH)⁺
For 1Ca: 2-[(4-{(1S,2S,4R)-2-[4-(2-methyl-2H-tetrazol-5-yl)phenyl]bicyclo[2.2.1]hept-2-yl}-phenoxy)methyl]pyramidine: m/z (ES) 439 (MH)⁺
For 1Cb: 2-[(4-{(1S,2S,4R)-2-[4-(2-ethyl-2H-tetrazol-5-yl)phenyl]bicyclo[2.2.1]hept-2-yl}-phenoxy)methyl]pyramidine: m/z (ES) 453 (MH)⁺
For 1Ce: 2-[(4-{(1S,2S,4R)-2-[4-(2H-tetrazol-5-yl)phenyl]bicyclo[2.2.1]hept-2-yl}phenoxy)methyl]pyramidine: m/z (ES) 453 (MH)⁺
For 1Da: 2-[(4-{(1S,2S,4R)-2-[4-(1-methyl-1H-tetrazol-5-yl)phenyl]bicyclo[2.2.1]hept-2-yl}-phenoxy)methyl]pyridine: m/z (ES) 438 (MH)⁺
For 1Dc: 2-[(4-{(1S,2S,4R)-2-[4-(1-isopropyl-1H-tetrazol-5-yl)phenyl]bicyclo[2.2.1]hept-2-yl}phenoxy)methyl]pyridine: m/z (ES) 466 (MH)⁺
For 1Dd: 2-[(4-{(1S,2S,4R)-2-[4-(1-(difluoromethyl)-1H-tetrazol-5-yl)phenyl]bicyclo[2.2.1]-hept-2-yl}phenoxy)methyl]pyridine: m/z (ES) 474 (MH)⁺
For 1Df: 2-({-4-[(1S,2S,4R)-2-(4-{1-[(2R)-pyrrolidin-2-ylmethyl]-1H-tetrazol-5-yl}phenyl)bicyclo[2.2.1]hept-2-yl]phenoxy}methyl)pyridine: m/z (ES) 438 (MH)⁺
For 1Ea: 1-methyl-5-(4-{(1S,2S,4R)-2-[4-(1,3-thiazol-2-ylmethoxy)phenyl]bicyclo[2.2.1]hept-2-yl}phenyl)-1H-tetrazole: m/z (ES) 444 (MH)⁺
For 1Eb: 1-ethyl-5-(4-{(1S,2S,4R)-2-[4-(1,3-thiazol-2-ylmethoxy)phenyl]bicyclo[2.2.1]hept-2-yl}phenyl)-1H-tetrazole: m/z (ES) 458 (MH)⁺
For 1Fa: 2-[(4-{(1S,2S,4R)-2-[4-(1-methyl-1H-tetrazol-5-yl)phenyl]bicyclo[2.2.1]hept-2-yl}-phenoxy)methyl]pyramidine: m/z (ES) 439 (MH)⁺
For 1Fb: 2-[(4-{(1S,2S,4R)-2-[4-(1-ethyl-1H-tetrazol-5-yl)phenyl]bicyclo[2.2.1]hept-2-yl}-phenoxy)methyl]pyramidine: m/z (ES) 453 (MH)⁺

TABLE 1-2
1G

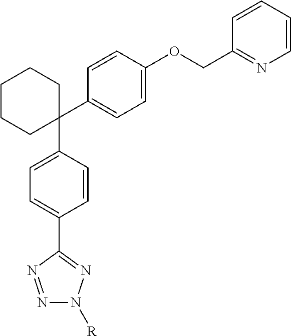

1H

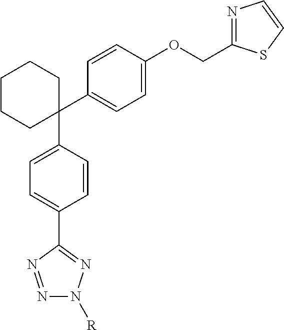

1I

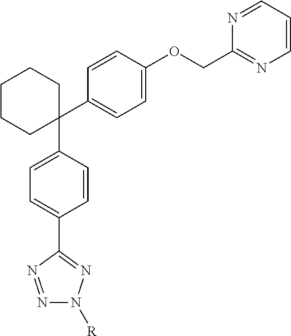

1J

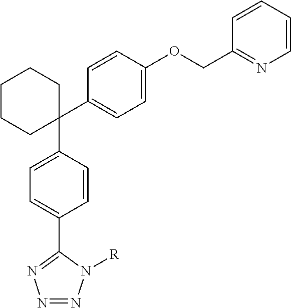

1K

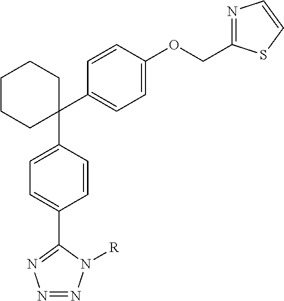

1L

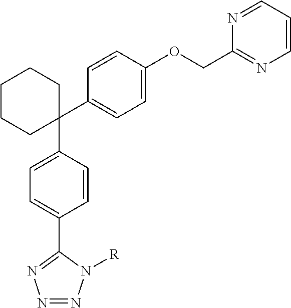

| Ex. | Ex. | Ex. | Ex. | Ex. | Ex. | |
|---|---|---|---|---|---|---|
| 1G | 1H | 1I | 1J | 1K | 1L | R |
| a | a | a | a | a | a | Me |
| b | b | b | b | b | b | Et |
| c | c | c | c | c | c | i-Pr |
| d | d | d | d | d | d | CF₂H |
| e | e | e | e | e | e | H |
| f | f | f | f | f | f | 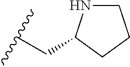 |
| g | g | g | g | g | g | 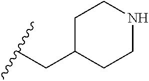 |
| h | h | h | h | h | h | 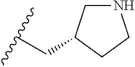 |
| i | i | i | i | i | i | 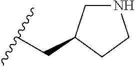 |

### Example 2

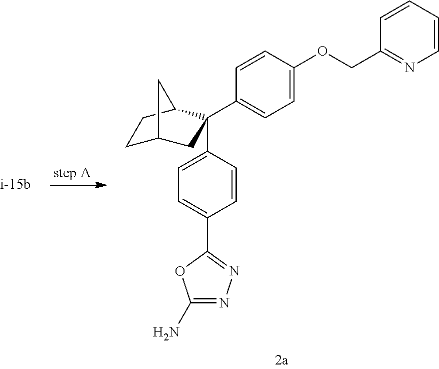

### Step A: Preparation of 5-(4-{2-[4-(pyridin-2-ylmethoxy)phenyl]bicyclo[2.2.1]hept-2-yl}phenyl)-1,3,4-oxadiazol-2-amine (2a)

A solution of cyanogen bromide (12.0 mg, 0.145 mmol) in dioxane (0.1 mL) was added dropwise to a stirred solution of i-15b (50.0 mg, 0.121 mmol) and aqueous sodium bicarbonate (15.0 mg, 0.145 mmol in 0.3 mL of water) in dioxane (0.1 mL), and the resulting mixture was allowed to stir at rt for 1.5 h. The reaction mixture was poured into saturated aqueous sodium bicarbonate and extracted three times with EtOAc. The combined organic extracts were washed with brine, dried (Na₂SO₄) and concentrated in vacuo. Purification of the crude residue by preparative reversed phase HPLC on YMC Pack Pro C18 stationary phase (CH₃CN/H₂O as eluent, 0.05% TFA as modifier), followed by lyophilization of the purified fractions afforded the title compound 2a. m/z (ES) 439 (MH)⁺.

Following the procedures similar to those described above, the following compounds in Table 2 can be prepared:

TABLE 2

| R | 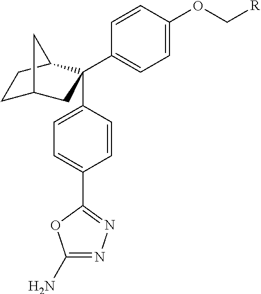 | 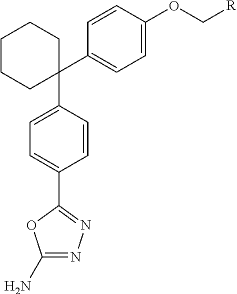 |
|---|---|---|
| 2-pyridyl | — | 2E |
| 2-pyrimidinyl | 2A | 2F |
| 2-thiazolyl | 2B | 2G |
| 5-F-2-pyridyl | 2C | — |
| 3-F-2-pyridyl | 2D | — |

Table 2. Parent Ion m/z (MH)⁺ data for compounds
For 2A: 5-(4-{(1S,2S,4R)-2-[4-(pyrimidin-2-ylmethoxy)phenyl]bicyclo[2.2.1]hept-2-yl}phenyl)-1,3,4-oxadiazol-2-amine: m/z (ES) 440 (MH)⁺
For 2B: 5-(4-{(1S,2S,4R)-2-[4-(1,3-thiazol-2-ylmethoxy)phenyl]bicyclo[2.2.1]hept-2-yl}-phenyl)-1,3,4-oxadiazol-2-amine: m/z (ES) 445 (MH)⁺
For 2C: 5-[4-((1S,2S,4R)-2-{4-[(5-fluoropyridin-2-yl)methoxy]phenyl}bicyclo[2.2.1]hept-2-yl)phenyl]-1,3,4-oxadiazol-2-amine: m/z (ES) 457 (MH)⁺
For 2D: 5-[4-((1S,2S,4R)-2-{4-[(3-fluoropyridin-2-yl)methoxy]phenyl}bicyclo[2.2.1]hept-2-yl)phenyl]-1,3,4-oxadiazol-2-amine: m/z (ES) 457 (MH)⁺
For 2E: 5 5-(4-{1-[4-(pyridin-2-ylmethoxy)phenyl]cyclohexyl}phenyl)-1,3,4-oxadiazol-2-amine: m/z (ES) 427 (MH)⁺

### Example 3

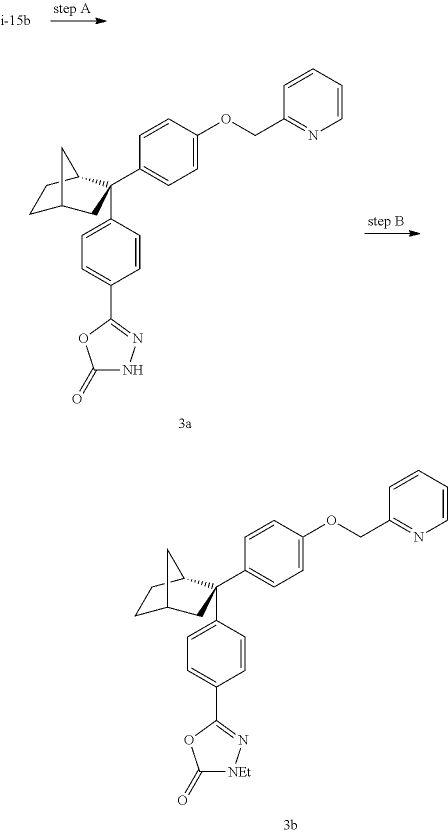

### Step A: Preparation of 5-(4-{2-[4-(pyridin-2-ylmethoxy)phenyl]bicyclo[2.2.1]hept-2-yl}phenyl)-1,3,4-oxadiazol-2-(3H)-one (3a)

Phosgene (115 μL of a 20% solution in toluene, 0.218 mmol) was added dropwise to a stirred solution of i-15b (50.0 mg, 0.121 mmol) in THF (1.0 mL) at −78° C. The resulting mixture was allowed to stir at −78° C. for 15 min, quenched with saturated aqueous sodium bicarbonate solution and extracted three times with DCM. The combined organic extracts were washed with brine, dried (Na₂SO₄), concentrated in vacuo. Purification by preparative reversed phase HPLC on YMC Pack Pro C18 stationary phase (CH₃CN/H₂O as eluent, 0.05% TFA as modifier), followed by lyophilization of the purified fractions afforded the title compound 3a. m/z (ES) 440 (MH)⁺.

### Step B: Preparation of 3-ethyl-5-(4-{2-[4-(pyridin-2-ylmethoxy)phenyl]bicyclo[2.2.1]-hept-2-yl}phenyl)-1,3,4-oxadiazol-2-(3H)-one (3b)

Sodium hydride (60% dispersion in mineral oil, 6.0 mg, 0.15 mmol) was added to a stirred solution of 3a (33.0 mg, 0.075 mmol) in DMF (300 μL) at 0° C. After 10 min, ethyl iodide (9.0 μL, 0.113 mmol) was added, and the resulting mixture was allowed to warm slowly to rt over 2.5 h. The reaction mixture was cooled to 0° C., quenched with saturated aqueous ammonium chloride solution, and extracted three times with EtOAc. The combined organic extracts were washed with brine, dried (Na₂SO₄), concentrated in vacuo, and purified by flash chromatography on silica gel (gradient elution; 0-55% EtOAc/hexanes as eluent) to afford the title compound 3b. m/z (ES) 468 (MH)⁺.

Following the procedures similar to those described above, the following compounds in Table 3 can be prepared:

TABLE 3
3A

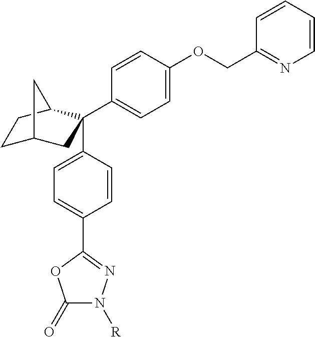

3B

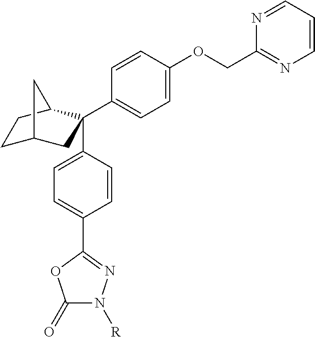

3C

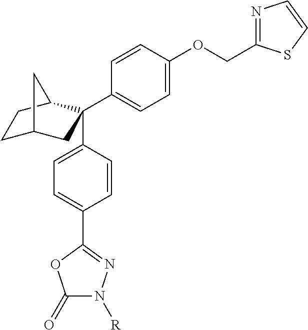

3D

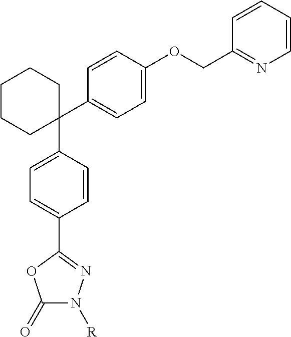

3E

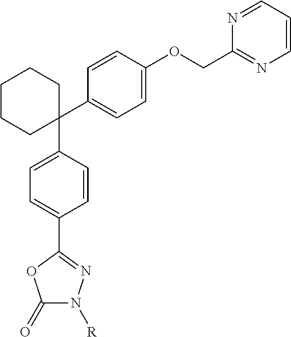

3F

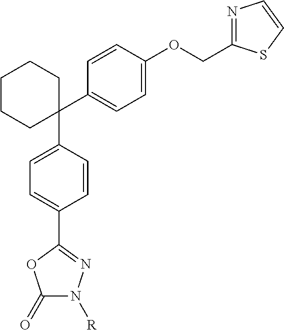

| Ex. | Ex. | Ex. | Ex. | Ex. | Ex. | |
|---|---|---|---|---|---|---|
| 3A | 3B | 3C | 3D | 3E | 3F | R |
| — | a | a | a | a | a | H |
| b | b | b | b | b | b | Me |
| — | c | c | c | c | c | Et |
| d | d | d | d | d | d | i-Pr |
| e | e | e | e | e | e | —CH₂CH₂F |

Table 3. Parent Ion m/z (MH)⁺ data for compounds
For 3Ab: 3-methyl-5-(4-{(1S,2S,4R)-2-[4-(pyridin-2-ylmethoxy)phenyl]bicyclo[2.2.1]hept-2-yl}phenyl)-1,3,4-oxadiazol-2(3H)-one: m/z (ES) 454 (MH)⁺
For 3Ad: 3-isopropyl-5-(4-{(1S,2S,4R)-2-[4-(pyridin-2-ylmethoxy)phenyl]bicyclo[2.2.1]hept-2-yl}phenyl)-1,3,4-oxadiazol-2(3H)-one: m/z (ES) 482 (MH)⁺
For 3Ae: 3-(2-fluoroethyl)-5-(4-{(1S,2S,4R)-2-[4-(pyridin-2-ylmethoxy)phenyl]bicyclo[2.2.1]-hept-2-yl}phenyl)-1,3,4-oxadiazol-2(3H)-one: m/z (ES) 486 (MH)⁺
For 3Da: 5-(4-{1-[4-(pyridin-2-ylmethoxy)phenyl]cyclohexyl}phenyl)-1,3,4-oxadiazol-2(3H)-one: m/z (ES) 428 (MH)⁺
   cyclobutylEx.#3af m/z (ES) 400 (MH)⁺

### Example 4

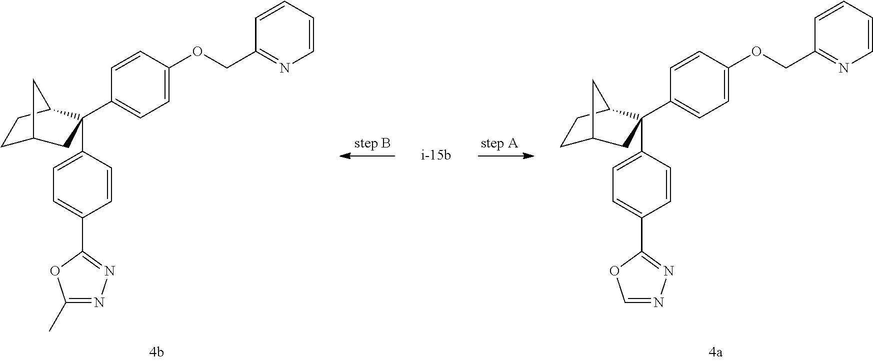

### Step A: Preparation of 2-[(4-{2-[4-(1,3,4-oxaxiazol-2-yl)phenyl]bicyclo[2.2.1]hept-2-yl}phenoxy)methyl]pyridine (4a)

p-TSA in dioxane (6.0 mg in 1.0 mL) was added in two portions to a stirred suspension of i-15b (49.0 mg, 0.118 mmol) in triethylorthoformate (600 μL), and the resulting mixture was allowed to stir at rt overnight. The reaction mixture was diluted with 1M HCl (600 μL), and after 30 min, extracted with EtOAc. The organic extracts were washed with saturated aqueous sodium bicarbonate solution, brine, dried (Na₂SO₄), and concentrated in vacuo. Purification of the crude residue by preparative reversed phase HPLC on YMC Pack Pro C18 stationary phase (CH₃CN/H₂O as eluent, 0.05% TFA as modifier), followed by lyophilization of the purified fractions afforded the title compound 4a. m/z (ES) 424 (MH)⁺.

### Step B: Preparation of 2-[(4-{2-[4-(5-methyl-1,3,4-oxaxiazol-2-yl)phenyl]bicyclo[2.2.1]-hept-2-yl}phenoxy)methyl]pyridine (4b)

A solution of acetyl chloride in DCM (150 μL of an 11% solution, 0.157 mmol) was added in two portions to a stirred solution of i-15b (50.0 mg, 0.121 mmol) and DIPEA (53.0 μL, 0.302 mmol) in DCM (1.0 mL) at rt over 3.5 h, and the resulting mixture was allowed to stir at rt for an additional 1 h. The reaction mixture was diluted with DCM, washed with saturated aqueous sodium bicarbonate solution, brine, dried (Na₂SO₄), and concentrated in vacuo. The crude acetyl hydrazide intermediate was dissolved in thionyl chloride (1.0 mL) and heated to 50° C. for 16 h. After cooling to rt, the reaction mixture was slowly quenched into saturated aqueous sodium bicarbonate and extracted three times with DCM. The combined organic extracts were washed with water, brine, dried (Na₂SO₄), concentrated in vacuo, and purified by flash chromatography on silica gel (gradient elution; 0-75% EtOAc/hexanes as eluent) to afford the title compound 4b. m/z (ES) 438 (MH)⁺.

Following procedures similar to those described above, the following compounds in Table 4 can also be prepared:

TABLE 4
4A

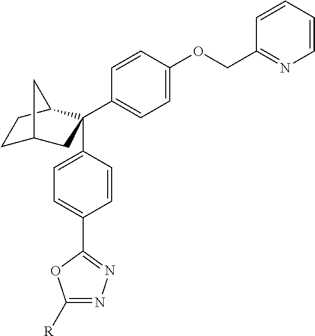

4B

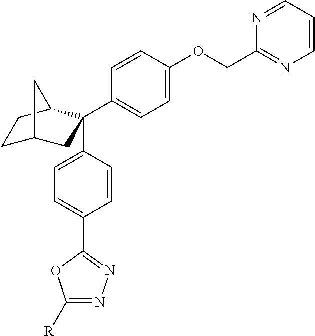

4C

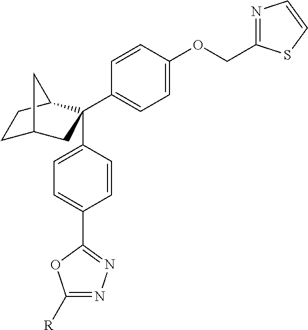

4D

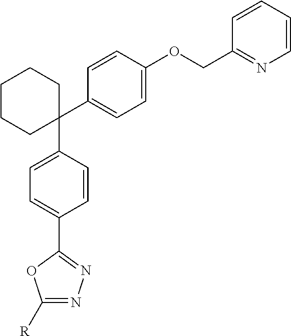

4E

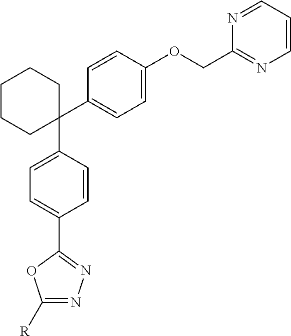

4F

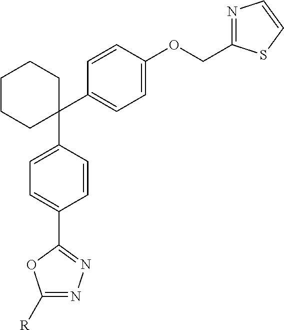

| Ex. | Ex. | Ex. | Ex. | Ex. | Ex. | |
|---|---|---|---|---|---|---|
| 4A | 4B | 4C | 4D | 4E | 4F | R |
| — | a | a | a | a | a | H |
| — | b | b | b | b | b | Me |
| c | c | c | c | c | c | Et |
| d | d | d | d | d | d | Pr |
| e | e | e | e | e | e | CF₃ |
| f | f | f | f | f | f | cyclopropyl |

Table 4. Parent Ion m/z (MH)⁺ data for compounds
For 4Ae: 2-{[4-((1S,2S,4R)-2-{4-[5-(trifluoromethyl)-1,3,4-oxadiazol-2-yl]phenyl}-bicyclo[2.2.1]hept-2-yl)phenoxy]methyl}pyridine: m/z (ES) 492 (MH)⁺
For 4Af: 2-{[4-((1S,2S,4R)-2-{4-[5-cyclopropyl-1,3,4-oxadiazol-2-yl]phenyl}bicyclo[2.2.1]hept-2-yl)phenoxy]methyl}pyridine: m/z (ES) 464 (MH)⁺

### Example 5

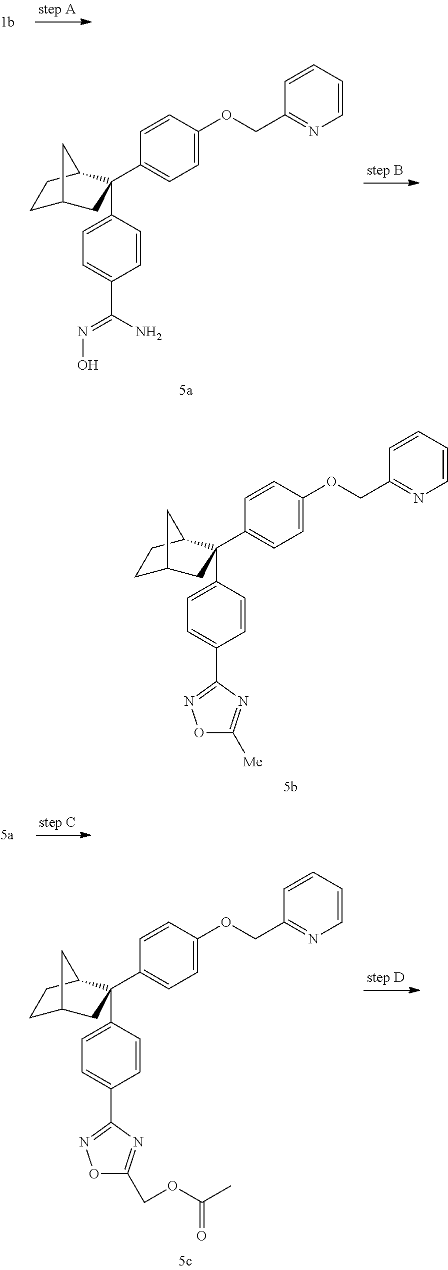

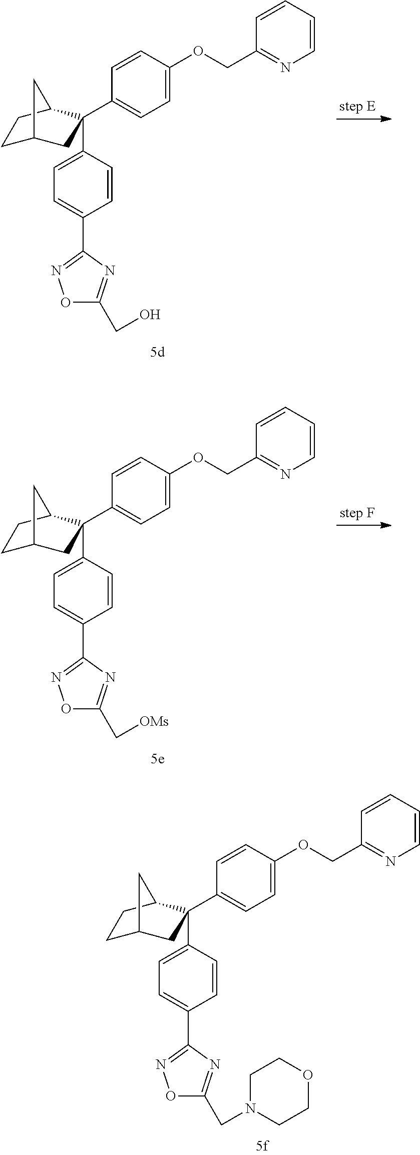

### Step A: Preparation of N′-hydroxy-4-{2-[4-(pyridin-2-ylmethoxy)phenyl]bicyclo-[2.2.1]hept-2-yl}benzenecarboximidamide (5a)

Hydroxylamine (146 μL of a 50% aqueous solution, 2.23 mmol) was added to a stirred solution of 1b (283 mg, 0.743 mmol) in EtOH (3 mL), and the resulting mixture was heated to reflux overnight. After cooling to rt, the reaction mixture was concentrated in vacuo and purified by flash chromatography on silica gel (gradient elution; 0-10% MeOH/DCM as eluent) to afford the title compound 5a. m/z (ES) 414 (MH)⁺.

### Step B: Preparation of 2-[(4-{2-[4-(5-methyl-1,2,4-oxadiazol-3-yl)phenyl]bicyclo[2.2.1]-hept-2-yl}phenoxy)methyl]pyridine (5b)

Acetic anhydride (16.0 μL, 0.169 mmol) was added to a stirred solution of 5a (35.0 mg, 0.0846 mmol) in pyridine (500 μL), and the resulting mixture was heated to 50° C. for 3 d. Additional aliquots of acetic anhydride (16.0 μL, 0.170 mmol) were added during this time to facilitate the reaction. After cooling down to rt, the reaction mixture was concentrated in vacuo and purified by preparative reversed phase HPLC on YMC Pack Pro C18 stationary phase (CH₃CN/H₂O as eluent, 0.05% TFA as modifier) to afford the title compound 5b. m/z (ES) 438 (MH)⁺.

### Step C: Preparation of [3-(4-{2-[4-(pyridin-2-ylmethoxy)phenyl]bicyclo[2.2.1]hept-2-yl}phenyl)-1,2,4-oxadiazol-5-yl]methyl acetate (5c)

5a (1.0 equiv.) is added to a stirred solution of acetoxyacetic acid (1.2 equiv.), EDC (1.2 equiv.), and HOBT (1.3 equiv.) in DCM (0.1 M final concentration), and the resulting mixture is allowed to stir at rt until the reaction is deemed complete. The reaction is quenched with saturated aqueous sodium bicarbonate and extracted with EtOAc. The combined organic extracts are washed with water and brine, dried (Na₂SO₄), and concentrated in vacuo. The crude is dissolved in xylene (0.15 M final concentration) and heated to 110° C. until the reaction is deemed complete. The reaction mixture is concentrated in vacuo to afford the title compound 5c.

### Step D: Preparation of [3-(4-{2-[4-(pyridin-2-ylmethoxy)phenyl]bicyclo[2.2.1]hept-2-yl}phenyl)-1,2,4-oxadiazol-5-yl]methanol (5d)

Potassium carbonate (6.0 equiv. of a 5 M aqueous solution) is added to a solution of 5c (1.0 equiv.) in MeOH (0.3 M final concentration) at 0° C., and the resulting mixture is allowed to stir at 0° C. until the reaction is deemed complete. The reaction mixture is diluted with EtOAc, washed with water and brine, dried (Na₂SO₄), and concentrated in vacuo. The resulting crude residue can be purified by flash chromatography on silica gel to afford the title compound 5d.

### Step E: Preparation of [3-(4-{2-[4-(pyridin-2-ylmethoxy)phenyl]bicyclo[2.2.1]hept-2-yl}phenyl)-1,2,4-oxadiazol-5-yl]methyl methanesulfonate (5e)

Methanesulfonyl chloride (1.5 equiv.) is added to a stirred solution of 5d (1.0 equiv.) and DIPEA (2.5 equiv) in DCM (0.1 M final concentration) at 0° C., and the resulting mixture is warmed slowly to rt and allowed to stir at rt until the reaction is deemed complete. The reaction mixture is diluted with EtOAc, and the organics are washed with water and brine, dried (Na₂SO₄), and concentrated in vacuo to afford the title compound 5e.

### Step F: Preparation of 4-{[3-(4-{2-[4-(pyridin-2-ylmethoxy)phenyl]bicyclo[2.2.1]hept-2-yl}phenyl)-1,2,4-oxadiazol-5-yl]methyl}morpholine (5f)

Morpholine (10 equiv.) is added to a stirred solution of 5e (1.0 equiv.) in DMF (0.1 M final concentration) at rt, and the resulting mixture is allowed to stir at rt until the reaction is deemed complete. The reaction mixture is diluted with EtOAc, and the organics are washed with water and brine, dried (Na₂SO₄), and concentrated in vacuo. The resulting crude residue can be purified by flash chromatography on silica gel to afford the title compound 5f

Following procedures similar to those described above, the following compounds in Table 5 can be prepared:

TABLE 5
5A

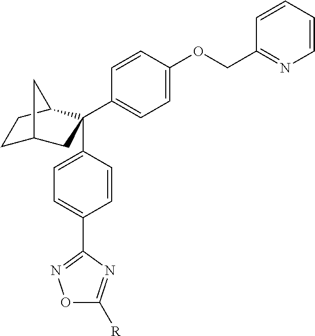

5B

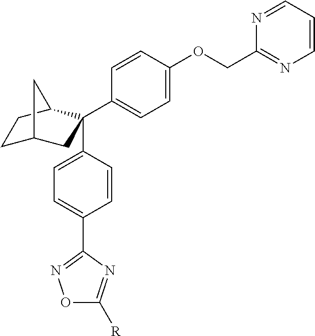

5C

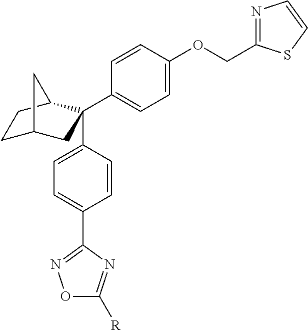

5D

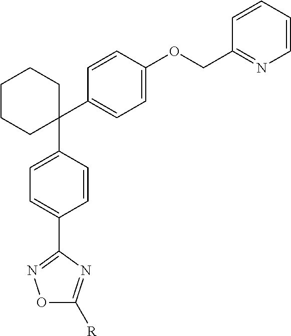

5E

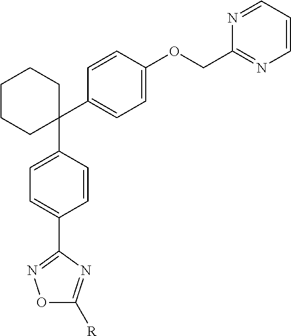

5F

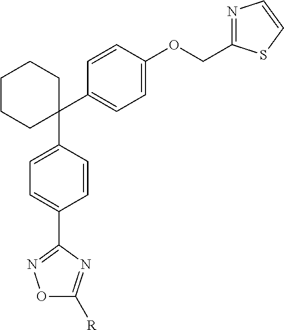

| Ex. | Ex. | Ex. | Ex. | Ex. | Ex. | |
|---|---|---|---|---|---|---|
| 5A | 5B | 5C | 5D | 5E | 5F | R |
| — | a | a | a | a | a | Me |
| b | b | b | b | b | b | Et |
| c | c | c | c | c | c | i-Pr |
| d | d | d | d | d | d | CH₂F |
| e | e | e | e | e | e | CF₂H |
| f | f | f | f | f | f | CH₂OBn |
| g | g | g | g | g | g | cyclopropyl |
| h | h | h | h | h | h | —C(CH₃)₂OH |
| i | i | i | i | i | i | 1-hydroxycyclopropyl |
| j | j | j | j | j | j | —C(OH)(CH₂CH₃)(CF₃) |
| k | k | k | k | k | k | 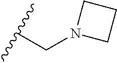 |
| l | l | l | l | l | l | 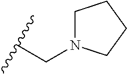 |
| m | m | m | m | m | m | 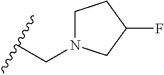 |

Table 5. Parent Ion m/z (MH)⁺ data for compounds
For 5Ab: 2-[(4-{(1S,2S,4R)-2-[4-(5-ethyl-1,2,4-oxadiazol-3-yl)phenyl]bicyclo[2.2.1]hept-2-yl}phenoxy)methyl]pyridine: m/z (ES) 452 (MH)⁺
For 5Ac: 2-[(4-{(1S,2S,4R)-2-[4-(5-isopropyl-1,2,4-oxadiazol-3-yl)phenyl]bicyclo[2.2.1]hept-2-yl}phenoxy)methyl]pyridine: m/z (ES) 466 (MH)⁺
For 5Ad: 2-[(4-{(1S,2S,4R)-2-[4-(5-fluoromethyl-1,2,4-oxadiazol-3-yl)phenyl]bicyclo[2.2.1]-hept-2-yl}phenoxy)methyl]pyridine: m/z (ES) 456 (MH)⁺
For 5Ae: 2-[(4-{(1S,2S,4R)-2-[4-(5-difluoromethyl-1,2,4-oxadiazol-3-yl)phenyl]bicyclo[2.2.1]-hept-2-yl}phenoxy)methyl]pyridine: m/z (ES) 474 (MH)⁺
For 5Af: 2-({4-[(1S,2S,4R)-2-(4-{5-[(benzyloxy)methyl]-1,2,4-oxadiazol-3-yl}phenyl)bicyclo[2.2.1]hept-2-yl]phenoxy}methyl)pyridine: m/z (ES) 544 (MH)⁺
For 5Ag: 2-[(4-{(1S,2S,4R)-2-[4-(5-cyclopropyl-1,2,4-oxadiazol-3-yl)phenyl]bicyclo[2.2.1]hept-2-yl}phenoxy)methyl]pyridine: m/z (ES) 474 (MH)⁺
For 5Ah: 2-[3-(4-{(1S,2S,4R)-2-[4-(pyridin-2-ylmethoxy)phenyl]bicyclo[2.2.1]hept-2-yl}-phenyl)-1,2,4-oxadiazol-5-yl]propan-2-ol: m/z (ES) 482 (MH)⁺
For 5Ai: 1-[3-(4-{(1S,2S,4R)-2-[4-(pyridin-2-ylmethoxy)phenyl]bicyclo[2.2.1]hept-2-yl}phenyl)-1,2,4-oxadiazol-5-yl]cyclopropanol: m/z (ES) 480 (MH)⁺
For 5Aj: 1,1,1-trifluoro-2-[3-(4-{(1S,2S,4R)-2-[4-(pyridin-2-ylmethoxy)phenyl]bicyclo[2.2.1]-hept-2-yl}phenyl)-1,2,4-oxadiazol-5-yl]butan-2-ol: m/z (ES) 550 (MH)⁺
For 5Al: 2-{[4-((1S,2S,4R)-2-{4-[5-(pyrrolidin-1-ylmethyl)-1,2,4-oxadiazol-3-yl]phenyl}-bicyclo[2.2.1]hept-2-yl)phenoxy]methyl}pyridine: m/z (ES) 507 (MH)⁺

### Example 6

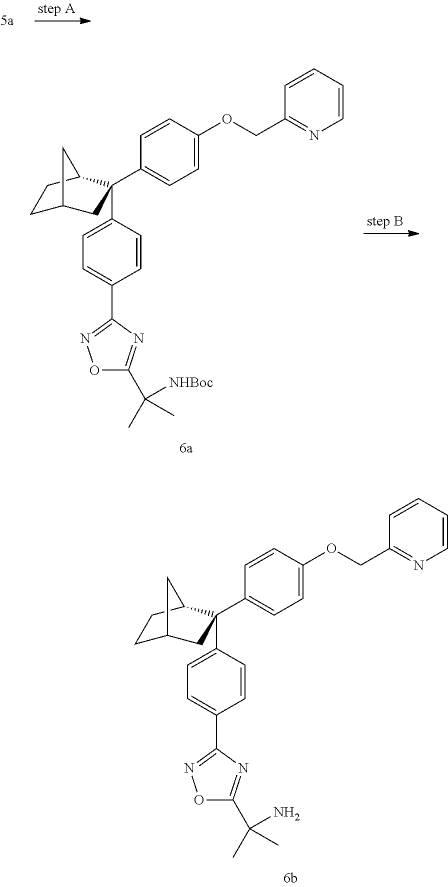

### Step A: Preparation of tert-butyl {1-methyl-1-[3-(4-{2-[4-(pyridin-2-ylmethoxy)phenyl]-bicyclo[2.2.1]hept-2-yl}phenyl)-1,2,4-oxadiazol-5-yl]ethyl}carbamate (6a)

A mixture of 5a (100 mg, 0.242 mmol), Boc-aminoisobutyric acid (148 mg, 0.726 mmol), HATU (184 mg, 0.484 mmol), and DIPEA (295 μL, 1.69 mmol) in DMF (1.2 mL) was allowed to stir at rt for 2 h. The reaction was diluted with EtOAc, and the organics were washed with water and brine, dried (Na₂SO₄), and concentrated in vacuo. The resulting crude residue was purified by flash chromatography on silica gel (gradient elution; 0-85% EtOAc/hexanes as eluent) to afford an acyclic intermediate (m/z (ES) 599 (MH)⁺), a portion of which (33 mg, 0.0584 mmol) was dissolved in anhydrous diglyme (400 μL) and heated to 100° C. for 2.5 h. After cooling to rt, the reaction mixture was concentrated in vacuo, and the resulting residue was purified by flash chromatography on silica gel (gradient elution; 0-50% EtOAc/hexanes as eluent) to afford the title compound 6a. m/z (ES) 581 (MH)⁺.

### Step B: Preparation of 2-[3-(4-{2-[4-(pyridin-2-ylmethoxy)phenyl]bicyclo[2.2.1]hept-2-yl}phenyl)-1,2,4-oxadiazol-5-yl]propan-2-amine (6b)

HCl (400 μL of a 4.0 M (19:1) dioxane:water solution) was added to a stirred solution of 6a (19.0 mg, 0.033 mmol) in dioxane (200 μL) at 10° C., and the resulting mixture was warmed to rt and allowed to stir for 40 min. The reaction mixture was concentrated in vacuo, partitioned into saturated aqueous sodium bicarbonate, and extracted three times with EtOAc. The combined organic extracts were washed with water and brine, dried (Na₂SO₄), concentrated in vacuo. The resulting crude residue was purified by preparative reversed phase HPLC on YMC Pack Pro C18 stationary phase (CH₃CN/H₂O as eluent, 0.05% TFA as modifier), and lyophilization of the purified fractions afforded the title compound 6b. m/z (ES) 481 (MH)⁺.

Following procedures similar to those described above, the following compounds in Table 6 can be prepared:

TABLE 6
6A

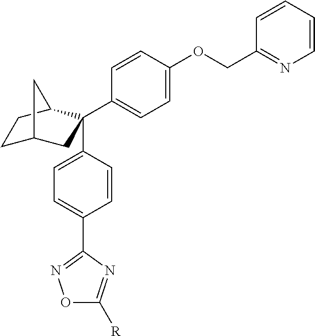

6B

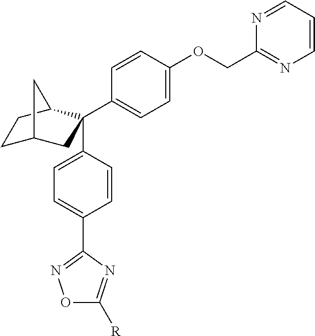

6C

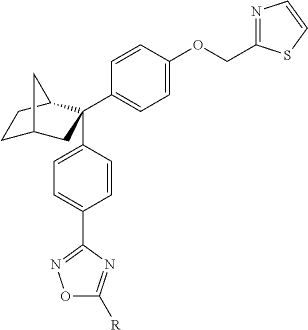

6D

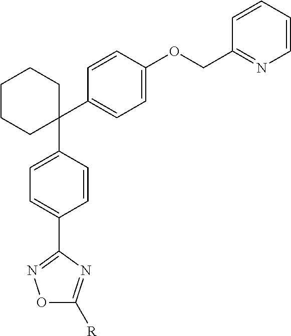

6E

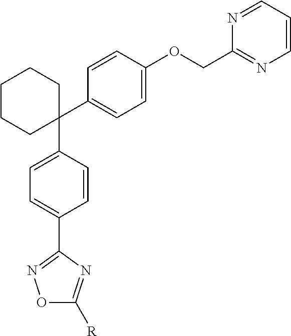

6F

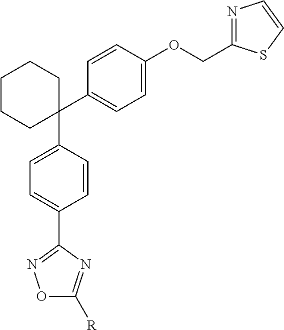

| Ex. | Ex. | Ex. | Ex. | Ex. | Ex. | |
|---|---|---|---|---|---|---|
| 6A | 6B | 6C | 6D | 6E | 6F | R |
| a | a | a | a | a | a | aminomethyl |
| b | b | b | b | b | b | 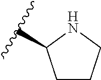 |
| c | c | c | c | c | c | 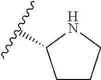 |

Table 6. Parent Ion m/z (MH)⁺ data for compounds
For 6Aa: 2-[(4-{(1S,2S,4R)-2-[4-(5-aminomethyl-1,2,4-oxadiazol-3-yl)phenyl]bicyclo[2.2.1]-hept-2-yl}phenoxy)methyl]pyridine: m/z (ES) 453 (MH)⁺
For 6Ab: 2-({4-[(1S,2S,4R)-2-(4-{5-[(2S)-pyrrolidin-2-yl]-1,2,4-oxadiazol-3-yl}phenyl)bicyclo[2.2.1]hept-2-yl]phenoxy}methyl)pyridine: m/z (ES) 493 (MH)⁺
For 6Ac: 22-({4-[(1S,2S,4R)-2-(4-{5-[(2R)-pyrrolidin-2-yl]-1,2,4-oxadiazol-3-yl}phenyl)bicyclo[2.2.1]hept-2-yl]phenoxy}methyl)pyridine: m/z (ES) 493 (MH)⁺

### Example 7

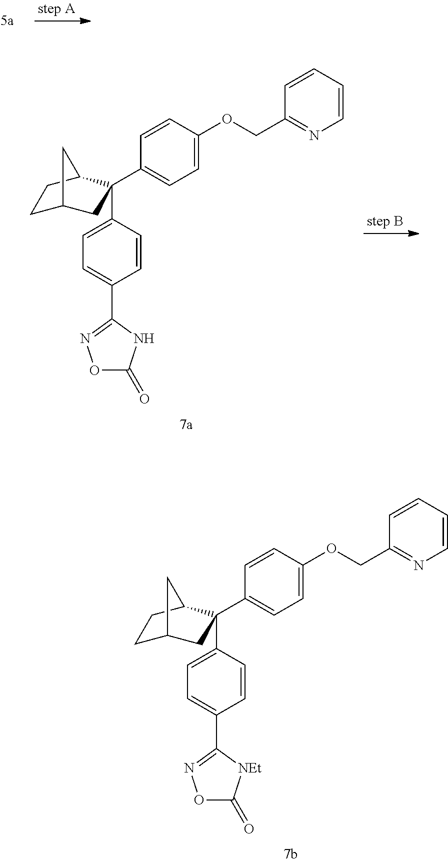

### Step A: Preparation of 3-(4-{2-[4-(pyridin-2-ylmethoxy)phenyl]-bicyclo[2.2.1]hept-2-yl}phenyl)-1,2,4-oxadiazol-5(4H)-one (7a)

Phosgene (72.0 μL, of a 20% toluene solution, 0.137 mmol) was added dropwise to a stirred solution of 5a (31.5 mg, 0.076 mmol) in DCM (1.8 mL) at −78° C., and the resulting mixture was allowed to stir at −78° C. for 2 h. The reaction mixture was quenched with saturated aqueous sodium bicarbonate and extracted three times with DCM. The combined organic extracts were washed with brine, dried (Na₂SO₄) and concentrated in vacuo. Purification of the crude residue by flash chromatography on silica gel (gradient elution; 0-5% MeOH/DCM as eluent) afforded the title compound 7a. m/z (ES) 440 (MH)⁺.

### Step B: Preparation of 4-ethyl-3-(4-{2-[4-(pyridin-2-ylmethoxy)phenyl]-bicyclo[2.2.1]-hept-2-yl}phenyl)-1,2,4-oxadiazol-5(4H)-one (7b)

Sodium hydride (3.0 equiv., 60 wt. % dispersion in mineral oil) is added to a stirred solution of 7a (1.0 equiv.) in DMF (0.1 M final concentration) at 0° C. After 15 min, ethyl iodide (2.0 equiv.) is added, and the resulting mixture is warmed to rt and allowed to stir until the reaction is deemed complete. The reaction is quenched with saturated aqueous ammonium chloride and extracted three times with EtOAc. The combined organic extracts are washed with brine, dried (Na₂SO₄) and concentrated in vacuo. The resulting crude residue can be purified by flash chromatography on silica gel to afford the title compound 7b.

Following procedures similar to those described above, the following compounds in Table 7 can also be prepared:

TABLE 7
7A

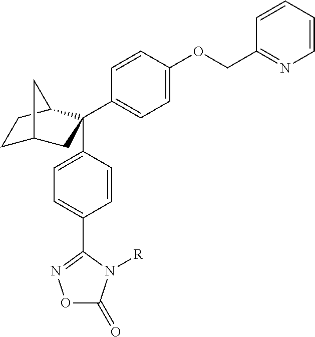

7B

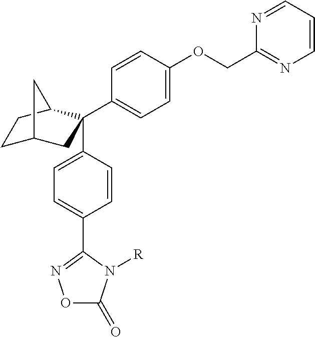

7C

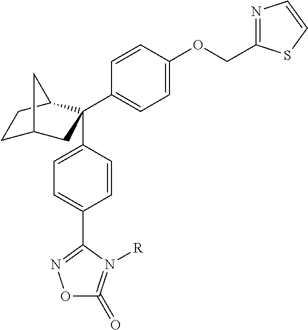

7D

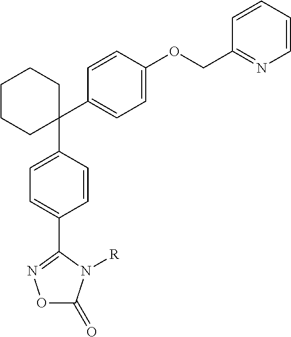

7E

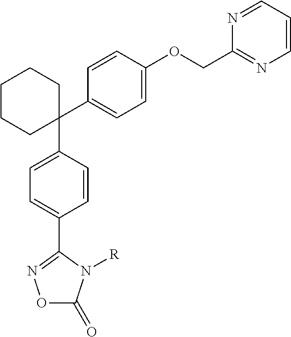

7F

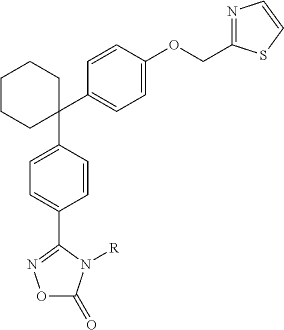

| Ex. | Ex. | Ex. | Ex. | Ex. | Ex. | |
|---|---|---|---|---|---|---|
| 7A | 7B | 7C | 7D | 7E | 7F | R |
| a | a | a | a | a | a | Me |
| — | b | b | b | b | b | Et |
| c | c | c | c | c | c | i-Pr |
| d | d | d | d | d | d | CH₂F |
| e | e | e | e | e | e | —CH₂CH₂F |

### Example 8

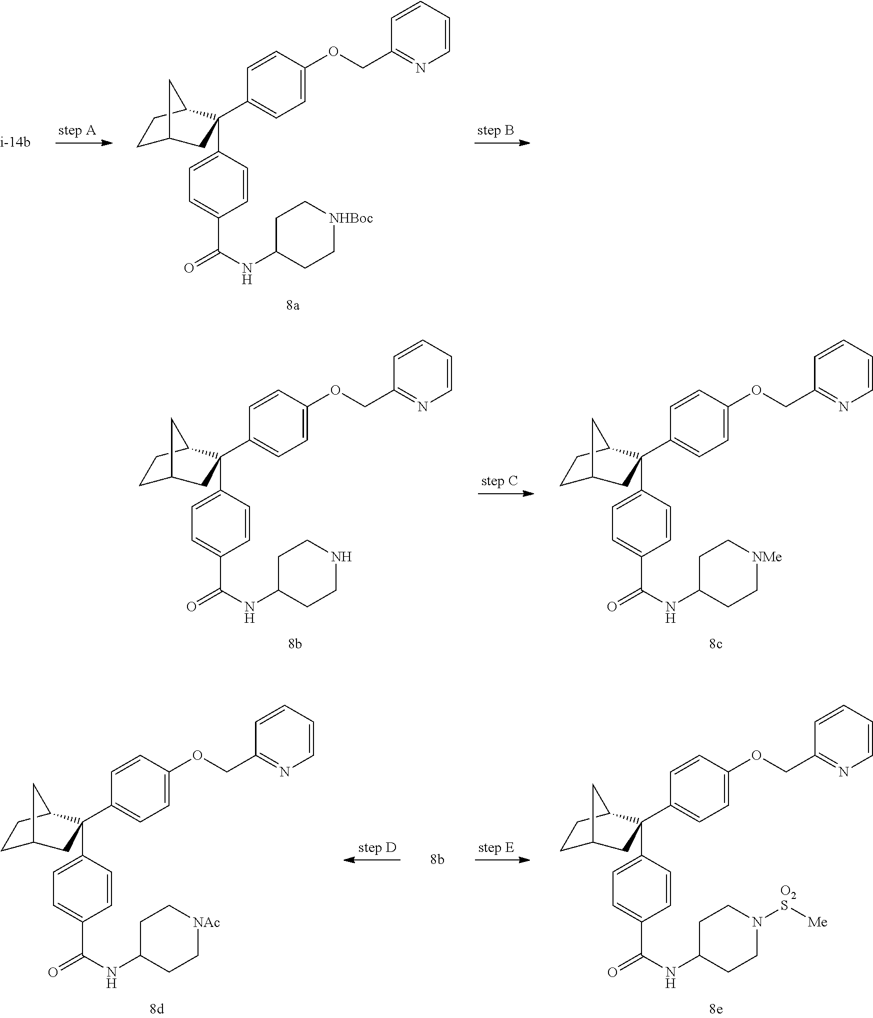

### Step A: Preparation of tert-butyl 4-[(4-{2-[4-(pyridin-2-ylmethoxy)phenyl]bicyclo-[2.2.1]hept-2-yl}benzoyl)amino]piperidine-1-carboxylate (8a)

DIPEA (673 μL, 3.86 mmol) was added to a stirred solution of i-14b (421 mg, 0.966 mmol), tert-butyl 4-aminopiperidine-1-carboxylate (232 mg, 1.16 mmol), HOAT (171 mg, 1.26 mmol), and HATU (477 mg, 1.26 mmol) in DMF (14.0 mL) at rt, and the resulting yellow solution was allowed to stir at rt overnight. The reaction was quenched with saturated aqueous sodium bicarbonate and extracted three times with EtOAc. The combined organic extracts were washed with water and brine, dried (Na₂SO₄) and concentrated in vacuo. Purification of the crude residue by flash chromatography on silica gel (gradient elution; 0-100% EtOAc/hexanes as eluent) afforded the title compound 8a. m/z (ES) 582 (MH)⁺.

### Step B: Preparation of N-piperidin-4-yl-4-{2-[4-(pyridin-2-ylmethoxy)phenyl]bicyclo-[2.2.1]hept-2-yl}benzamide (8b)

8a (526 mg, 0.904 mmol) was dissolved in a solution of HCl (13.5 mL of a 4.0 M (19:1) dioxane:water solution) at 0° C., and the resulting mixture was stirred at 0° C. for 40 min. The reaction was quenched with saturated aqueous sodium bicarbonate and extracted five times with EtOAc. The combined organic extracts were dried (Na₂SO₄) and concentrated in vacuo, and the resulting crude residue was purified by flash chromatography on silica gel (gradient elution; 1-15% MeOH (with 10% ammonium hydroxide as modifier)/DCM as eluent) to afford the title compound 8b. m/z (ES) 482 (MH)⁺.

### Step C: Preparation of N-(1-methylpiperidin-4-yl)-4-{2-[4-(pyridin-2-ylmethoxy)phenyl]bicyclo[2.2.1]hept-2-yl}benzamide (8c)

Sodium cyanoborohydride (12.0 mg, 0.190 mmol) was added to a stirred solution of formaldehyde (12.0 μL of a 37% w/v aqueous solution), 8b (17.0 mg, 0.0353 mmol), and acetic acid (2.0 μL) in MeOH (1.3 mL). The resulting mixture was allowed to stir at rt for 10 min, quenched with saturated aqueous sodium bicarbonate, and extracted three times with EtOAc. The combined organic extracts were washed with brine, dried (Na₂SO₄) and concentrated in vacuo. The crude residue was purified by flash chromatography on silica gel (gradient elution; 6-10% MeOH (with 10% ammonium hydroxide as modifier)/DCM as eluent) to afford the title compound 8c. m/z (ES) 496 (MH)⁺.

### Step D: Preparation of N-(1-acetylpiperidin-4-yl)-4-{2-[4-(pyridin-2-ylmethoxy)phenyl]bicyclo[2.2.1]hept-2-yl}benzamide (8d)

Acetyl chloride (5.0 μL, 0.081 mmol) was added to a stirred solution of triethylamine (10.0 μL, 0.072 mmol) and 8b (19.6 mg, 0.041 mmol) in DCM (500 μL) at 0° C., the resulting mixture was allowed to stir at 0° C. for 20 min. The reaction was quenched with saturated aqueous sodium bicarbonate and extracted three times with EtOAc. The combined organic extracts were washed with saturated aqueous sodium bicarbonate solution and brine, dried (Na₂SO₄) and concentrated in vacuo. The resulting crude residue was purified by flash chromatography on silica gel (gradient elution; 0-8% MeOH/DCM as eluent) to afford the title compound 8d. m/z (ES) 524 (MH)⁺.

### Step E: Preparation of N-[1-(methylsulfonyl)piperidin-4-yl]-4-{2-[4-(pyridin-2-ylmethoxy)phenyl]bicyclo[2.2.1]hept-2-yl}benzamide (8e)

Methanesulfonic anhydride (13.0 mg, 0.073 mmol) was added to a stirred solution of triethylamine (10.0 μL, 0.072 mmol) and 8b (19.6 mg, 0.041 mmol) in DCM (500 μL) at 0° C., and the resulting mixture was allowed to stir at 0° C. for 15 min. The reaction was quenched with saturated aqueous sodium bicarbonate and extracted three times with EtOAc. The combined organic extracts were washed with saturated aqueous sodium bicarbonate and brine, dried (Na₂SO₄) and concentrated in vacuo. The resulting crude residue was purified by flash chromatography on silica gel (gradient elution; 0-8% MeOH/DCM as eluent) to afford the title compound 8e. m/z (ES) 560 (MH)⁺.

Following procedures similar to those described above, the following compounds in Tables 8-1 and 8-2 can be prepared:

TABLE 8-1
8A

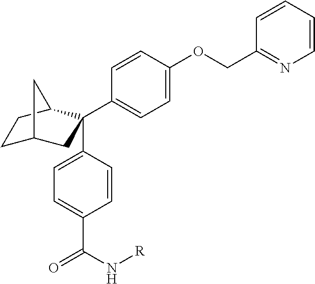

8B

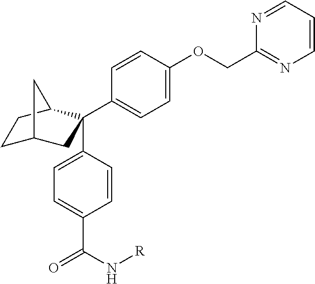

8C

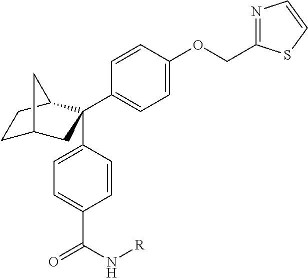

8D

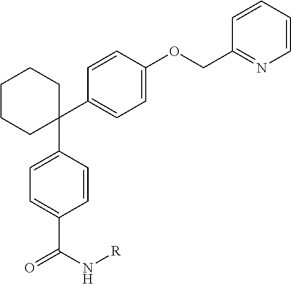

8E

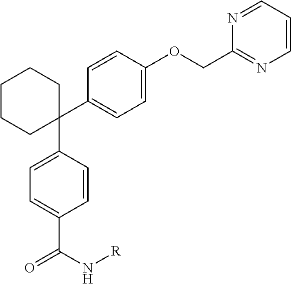

8F

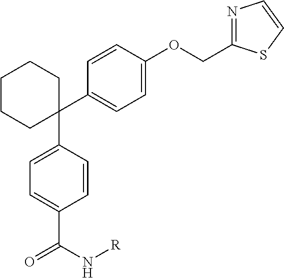

| Ex. | Ex. | Ex. | Ex. | Ex. | Ex. | |
|---|---|---|---|---|---|---|
| 8A | 8B | 8C | 8D | 8E | 8F | R |
| a | a | a | a | a | a | Me |
| b | b | b | b | b | b | Pr |
| c | c | c | c | c | c | i-Pr |
| d | d | d | d | d | d | t-Bu |
| e | e | e | e | e | e | —CH₂CN |
| f | f | f | f | f | f | —CH₂CF₃ |
| g | g | g | g | g | g | cyclopropyl |
| h | h | h | h | h | h | cyclobutyl |
| i | i | i | i | i | i | —CH₂C(CH₃)₂OH |
| j | j | j | j | j | j | 1-hydroxycyclopropyl-methyl |
| k | k | k | k | k | k | —CH₂CH₂OH |
| l | l | l | l | l | l | —CH₂C(CH₃)₂NH₂ |
| m | m | m | m | m | m | 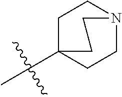 |
| n | n | n | n | n | n | 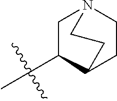 |
| o | o | o | o | o | o | 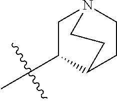 |
| p | p | p | p | p | p | 3-pyridyl |
| q | q | q | q | q | q | 5-pyrimidinyl |
| r | r | r | r | r | r | 4-pyprimidinyl |
| s | s | s | s | s | s | 2-thiazolyl |
| t | t | t | t | t | t | 1,3,4-thiadiazol-2-yl |

Table 8-1. Parent Ion m/z (MH)⁺ data for compounds
For 8Aa: N-methyl-4-{(1S,2S,4R)-2-[4-(pyridin-2-ylmethoxy)phenyl]bicyclo[2.2.1]hept-2-yl}-benzamide: m/z (ES) 413 (MH)⁺
For 8Ab: N-propyl-4-{(1S,2S,4R)-2-[4-(pyridin-2-ylmethoxy)phenyl]bicyclo[2.2.1]hept-2-yl}-benzamide: m/z (ES) 441 (MH)⁺
For 8Ac: N-isopropyl-4-{(1S,2S,4R)-2-[4-(pyridin-2-ylmethoxy)phenyl]bicyclo[2.2.1]hept-2-yl}-benzamide: m/z (ES) 441 (MH)⁺
For 8Ad: N-(tert-butyl)-4-{(1S,2S,4R)-2-[4-(pyridin-2-ylmethoxy)phenyl]bicyclo[2.2.1]hept-2-yl}benzamide: m/z (ES) 455 (MH)⁺
For 8Ae: N-cyanomethyl-4-{(1S,2S,4R)-2-[4-(pyridin-2-ylmethoxy)phenyl]bicyclo[2.2.1]hept-2-yl}benzamide: m/z (ES) 438 (MH)⁺
For 8Af: N-(2,2,2-trifluoroethyl)-4-{(1S,2S,4R)-2-[4-(pyridin-2-ylmethoxy)phenyl]bicyclo-[2.2.1]hept-2-yl}benzamide: m/z (ES) 481 (MH)⁺
For 8Ag: N-cyclopropyl-4-{(1S,2S,4R)-2-[4-(pyridin-2-ylmethoxy)phenyl]bicyclo[2.2.1]hept-2-yl}benzamide: m/z (ES) 439 (MH)⁺
For 8Ah: N-cyclobutyl-4-{(1S,2S,4R)-2-[4-(pyridin-2-ylmethoxy)phenyl]bicyclo[2.2.1]hept-2-yl}benzamide: m/z (ES) 453 (MH)⁺
For 8Ai: N-(2-hydroxy-2-methylpropyl)-4-{(1S,2S,4R)-2-[4-(pyridin-2-ylmethoxy)phenyl]-bicyclo[2.2.1]hept-2-yl}benzamide: m/z (ES) 471 (MH)⁺
For 8Aj: N-[(1-hydroxycyclopropyl)methyl]-4-{(1S,2S,4R)-2-[4-(pyridin-2-ylmethoxy)phenyl]bicyclo[2.2.1]hept-2-yl}benzamide: m/z (ES) 469 (MH)⁺
For 8Al: N-(2-amino-2-methylpropyl)-4-{(1S,2S,4R)-2-[4-(pyridin-2-ylmethoxy)phenyl]-bicyclo[2.2.1]hept-2-yl}benzamide: m/z (ES) 470 (MH)⁺
For 8An: N-[(3R)-1-azabicyclo[2.2.2]oct-3-yl]-4-{(1S,2S,4R)-2-[4-(pyridin-2-ylmethoxy)phenyl]bicyclo[2.2.1]hept-2-yl}benzamide: m/z (ES) 508 (MH)⁺
For 8Ao: N-[(3S)-1-azabicyclo[2.2.2]oct-3-yl]-4-{(1S,2S,4R)-2-[4-(pyridin-2-ylmethoxy)phenyl]-bicyclo[2.2.1]hept-2-yl}benzamide: m/z (ES) 508 (MH)⁺
For 8Ap: N-pyridin-3-yl-4-{(1S,2S,4R)-2-[4-(pyridin-2-ylmethoxy)phenyl]bicyclo[2.2.1]hept-2-yl}benzamide: m/z (ES) 476 (MH)⁺
For 8Aq: N-pyrimidin-5-yl-4-{(1S,2S,4R)-2-[4-(pyridin-2-ylmethoxy)phenyl]bicyclo[2.2.1]hept-2-yl}benzamide: m/z (ES) 477 (MH)⁺
For 8Ar: N-pyrimidin-4-yl-4-{(1S,2S,4R)-2-[4-(pyridin-2-ylmethoxy)phenyl]bicyclo[2.2.1]hept-2-yl}benzamide: m/z (ES) 477 (MH)⁺
For 8As: N-thiazol-2-yl-4-{(1S,2S,4R)-2-[4-(pyridin-2-ylmethoxy)phenyl]bicyclo[2.2.1]hept-2-yl}benzamide: m/z (ES) 482 (MH)⁺
For 8At: 4-{(1S,2S,4R)-2-[4-(pyridin-2-ylmethoxy)phenyl]bicyclo[2.2.1]hept-2-yl}-N-1,3,4-thiadiazol-2-ylbenzamide: m/z (ES) 483 (MH)⁺
For 8Ca: N-methyl-4-{1-[4-(pyridin-2-ylmethoxy)phenyl]cyclohexyl}benzamide: m/z (ES) 401 (MH)⁺
For 8Cg: N-cyclopropyl-4-{1-[4-(pyridin-2-ylmethoxy)phenyl]cyclohexyl}benzamide: m/z (ES) 427 (MH)⁺
For 8Ck: N-(2-hydroxyethyl)-4-{1-[4-(pyridin-2-ylmethoxy)phenyl]cyclohexyl}benzamide: m/z (ES) 431 (MH)⁺

TABLE 8-2
8G

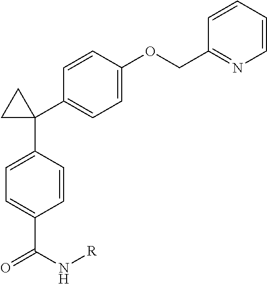

8H

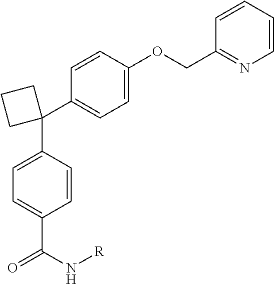

8I

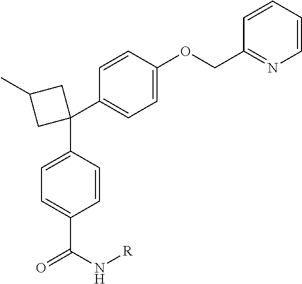

8J

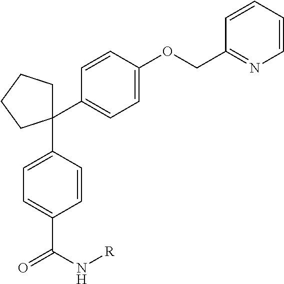

8K

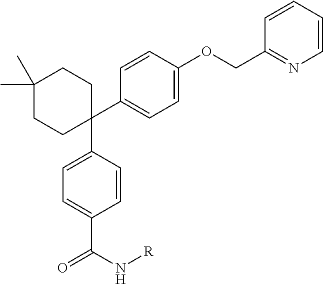

8L

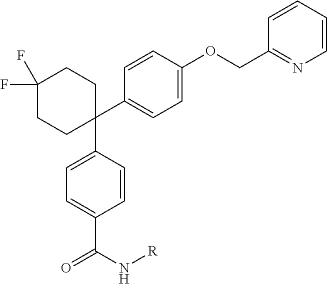

| Ex. | Ex. | Ex. | Ex. | Ex. | Ex. | |
|---|---|---|---|---|---|---|
| 8G | 8H | 8I | 8J | 8K | 8L | R |
| a | a | a | a | a | a | Me |
| b | b | b | b | b | b | i-Pr |
| c | c | c | c | c | c | t-Bu |
| d | d | d | d | d | d | Bn |
| e | e | e | e | e | e | cyclopropyl |
| f | f | f | f | f | f | —CH₂CH₂OH |
| g | g | g | g | g | g | —CH₂C(CH₃)₂OH |
| h | h | h | h | h | h | 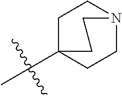 |
| i | i | i | i | i | i | 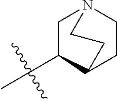 |
| j | j | j | j | j | j | 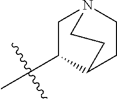 |
| k | k | k | k | k | k | 3-pyridyl |

Table 8-2. Parent Ion m/z (MH)⁺ data for compounds
For 8Gd: N-benzyl-4-{1-[4-(pyridin-2-ylmethoxy)phenyl]cyclopropyl}benzamide: m/z (ES) 435 (MH)⁺
For 8Ha: N-methyl-4-{1-[4-(pyridin-2-ylmethoxy)phenyl]cyclobutyl}benzamide: m/z (ES) 395 (M+Na)⁺
For 8Hd: N-benzyl-4-{1-[4-(pyridin-2-ylmethoxy)phenyl]cyclobutyl}benzamide: m/z (ES) 471 (M+Na)⁺
For 8He: N-cyclopropyl-4-{1-[4-(pyridin-2-ylmethoxy)phenyl]cyclobutyl}benzamide: m/z (ES) 421 (M+Na)⁺
For 8Ie: N-cyclopropyl-4-{3-methyl-1-[4-(pyridin-2-ylmethoxy)phenyl]cyclobutyl}benzam: m/z (ES) 413 (MH)⁺
For 8Ja: N-methyl-4-{1-[4-(pyridin-2-ylmethoxy)phenyl]cyclopentyl}benzamide: m/z (ES) 387 (MH)⁺
For 8Jd: N-benzyl-4-{1-[4-(pyridin-2-ylmethoxy)phenyl]cyclopentyl}benzamide: m/z (ES) 463 (MH)⁺
For 8Je: N-cyclopropyl-4-{1-[4-(pyridin-2-ylmethoxy)phenyl]cyclopentyl}benzamide: m/z (ES) 413 (MH)⁺
For 8Ka: 4-{4,4-dimethyl-1-[4-(pyridin-2-ylmethoxy)phenyl]cyclohexyl}-N-methylbenzamide: m/z (ES) 429 (MH)⁺
For 8 Kd: 4-{4,4-dimethyl-1-[4-(pyridin-2-ylmethoxy)phenyl]cyclohexyl}-N-benzylbenzamide: m/z (ES) 505 (MH)⁺
For 8Ke: 4-{4,4-dimethyl-1-[4-(pyridin-2-ylmethoxy)phenyl]cyclohexyl}-N-cyclopropylbenzamide: m/z (ES) 455 (MH)⁺
For 8La: 4-{4,4-difluoro-1-[4-(pyridin-2-ylmethoxy)phenyl]cyclohexyl}-N-methylbenzamide: m/z (ES) 437 (MH)⁺
For 8Ld: 4-{4,4-difluoro-1-[4-(pyridin-2-ylmethoxy)phenyl]cyclohexyl}-N-benzylbenzamide: m/z (ES) 513 (MH)⁺
For 8Le: 4-{4,4-difluoro-1-[4-(pyridin-2-ylmethoxy)phenyl]cyclohexyl}-N-cyclopropylbenzamide: m/z (ES) 463 (MH)⁺

### Example 9

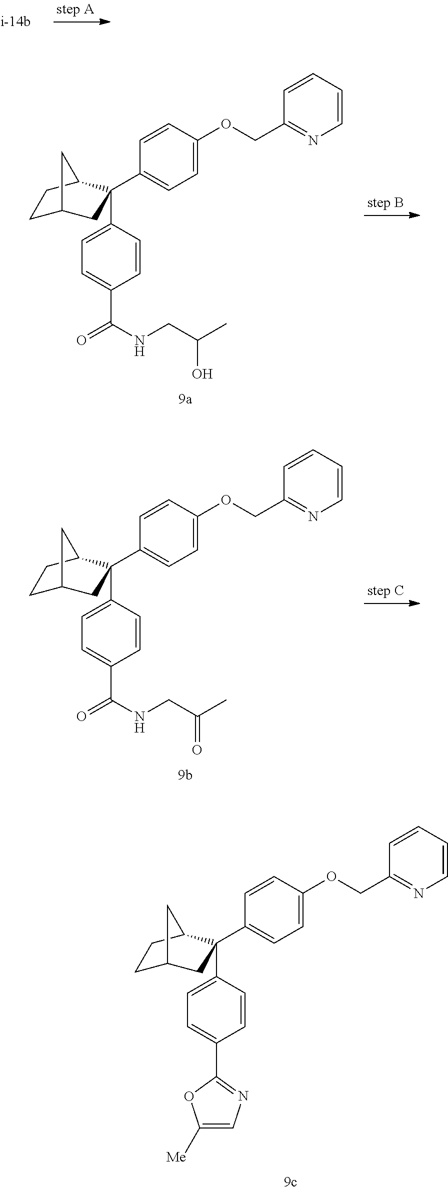

### Step A: Preparation of N-(2-hydroxypropyl)-4-{2-[4-(pyridin-2-ylmethoxy)phenyl]-bicyclo[2.2.1]-hept-2-yl}benzamide (9a)

DIPEA (1.0 mL, 5.74 mmol) was added to a stirred solution of i-14b (100 mg, 0.287 mmol), 1-aminopropan-2-ol (332 μL, 4.30 mmol), and HATU (218 mg, 0.574 mmol) in DMF (2.0 mL), and the resulting yellow solution was allowed to stir at rt overnight. The reaction mixture was diluted with EtOAc and organic layer was washed with water, saturated aqueous sodium bicarbonate solution and brine. The organic layer was dried (Na₂SO₄), concentrated in vacuo, and the resulting crude residue was purified by preparative reversed phase HPLC on YMC Pack Pro C18 stationary phase (CH₃CN/H₂O as eluent, 0.05% TFA as modifier), followed by lyophilization of the purified fractions to afford the title compound 9a. m/z (ES) 457 (MH)⁺.

### Step B: Preparation of N-(2-oxopropyl)-4-{2-[4-(pyridin-2-ylmethoxy)phenyl]-bicyclo[2.2.1]-hept-2-yl}benzamide (9b)

TPAP (5.2 mg, 0.0146 mmol) was added to a stirred suspension of powdered 4 Å molecular sieves (36.0 mg), NMO (8.5 mg, 0.109 mmol), and 9a (33.0 mg, 0.073 mmol) in DCM (500 μL), and the resulting mixture was allowed to stir at rt for 40 min. The reaction was diluted with DCM and quenched with saturated aqueous Na₂SO₃. The layers were separated, and the aqueous layer was extracted with DCM. The combined organic extracts were washed with saturated aqueous copper sulfate solution and brine, dried (Na₂SO₄) and concentrated in vacuo. The resulting crude residue was purified by flash chromatography on silica gel (gradient elution 0-5% MeOH/DCM as eluent) to afford the title compound 9b. m/z (ES) 455 (MH)⁺.

### Step C: Preparation of 2-[(4-{2-[4-(5-methyl-1,3-oxazol-2-yl)phenyl]bicyclo[2.2.1]-hept-2-yl}phenoxy)methyl]pyridine (9c)

9b (8.0 mg, 0.0176 mmol) was dissolved in thionyl chloride (1.0 mL), and the resulting mixture was heated to 50° C. for 2 h. The reaction mixture was cooled to rt, quenched by dropwise addition to a chilled solution of saturated aqueous sodium bicarbonate and extracted three times with EtOAc. The combined organic extracts were washed with brine and dried (Na₂SO₄) and concentrated in vacuo. The crude residue was purified by preparative reversed phase HPLC on YMC Pack Pro C18 stationary phase (CH₃CN/H₂O as eluent, 0.05% TFA as modifier), followed by lyophilization of the purified fractions to afford the title compound 9c. m/z (ES) 437 (MH)⁺.

Following procedures similar to those described above, the following compounds in Table 9 can be prepared:

TABLE 9
9A

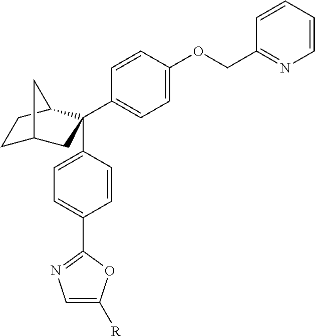

9B

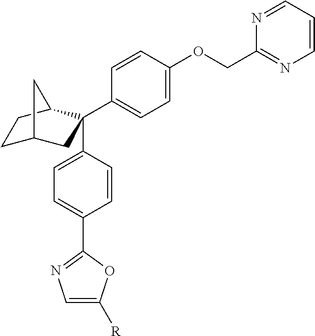

9C

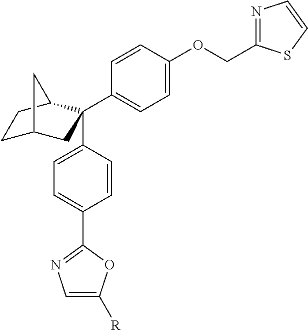

9D

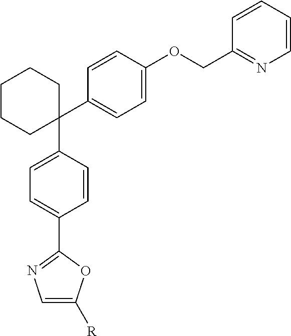

9E

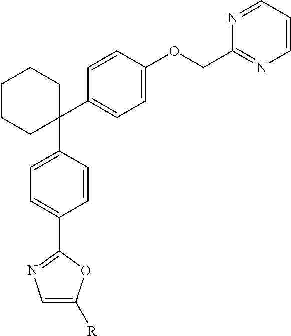

9F

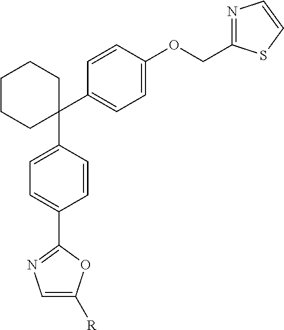

| Ex. | Ex. | Ex. | Ex. | Ex. | Ex. | |
|---|---|---|---|---|---|---|
| 9A | 9B | 9C | 9D | 9E | 9F | R |
| — | a | a | a | a | a | Me |
| b | b | b | b | b | b | Et |
| c | c | c | c | c | c | i-Pr |
| d | d | d | d | d | d | cyclopropyl |
| e | e | e | e | e | e | —C(CH₃)₂OH |

### Example 10

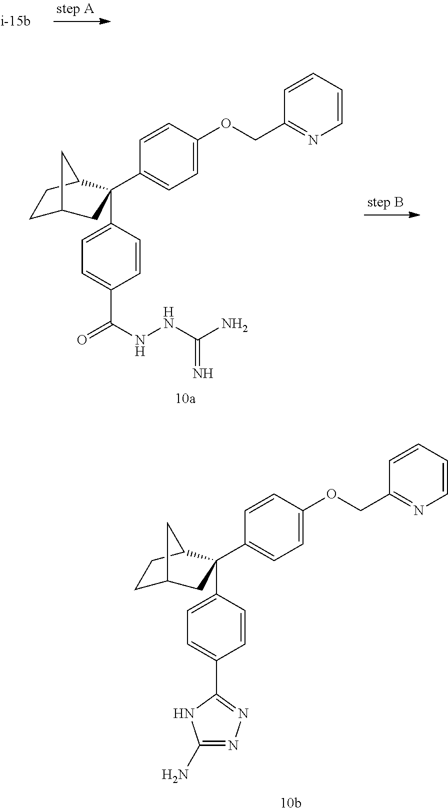

### Step A: Preparation of 2-(4-{2-[4-(pyridin-2-ylmethoxy)phenyl]bicyclo[2.2.1]-hept-2-yl}benzoyl)hydrazinecarboximidamide (10a)

0.3 N NaOH (400 mL, 0.120 mmol) was added to a suspension of S-methyl-pseudothiourea sulfate (67 mg, 0.242 mmol) and i-15b (50 mg, 0.121 mmol) in dioxane (900 μL) at rt, and the resulting mixture was allowed to stir at it overnight, then heated to 100° C. for 4 h. The reaction mixture was cooled to it, poured into water and extracted three times with EtOAc. The combined organic extracts were washed with brine, dried (Na₂SO₄) and concentrated in vacuo to afford 10b (m/z (ES) 438 (MH)⁺). 10a was collected as an insoluble solid from the aforementioned workup (m/z (ES) 456 (MH)⁺).

### Step B: Preparation of 5-(4-{2-[4-(pyridin-2-ylmethoxy)phenyl]bicyclo[2.2.1]-hept-2-yl}phenyl)-4H-1,2,4-triazol-3-amine (10b)

10a (36.9 mg, 0.081 mmol) was heated neat to 208° C. for 20 min, then cooled to rt. The crude residue was purified by preparative reversed phase HPLC on YMC Pack Pro C18 stationary phase (CH₃CN/H₂O as eluent, 0.05% TFA as modifier), followed by lyophilization of the purified fractions to afford the title compound 10b. m/z (ES) 438 (MH)⁺.

Following procedures similar to those described above, the following compounds in Table 10 can be prepared:

TABLE 10

| R | 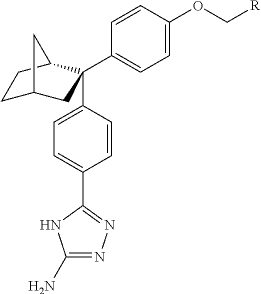 | 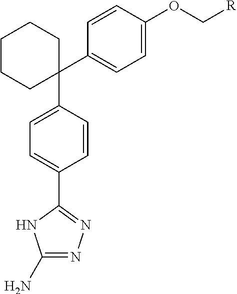 |
|---|---|---|
| 2-pyridyl | — | 10C |
| 2-pyrimidinyl | 10A | 10D |
| 2-thiazolyl | 10B | 10E |

### Example 11

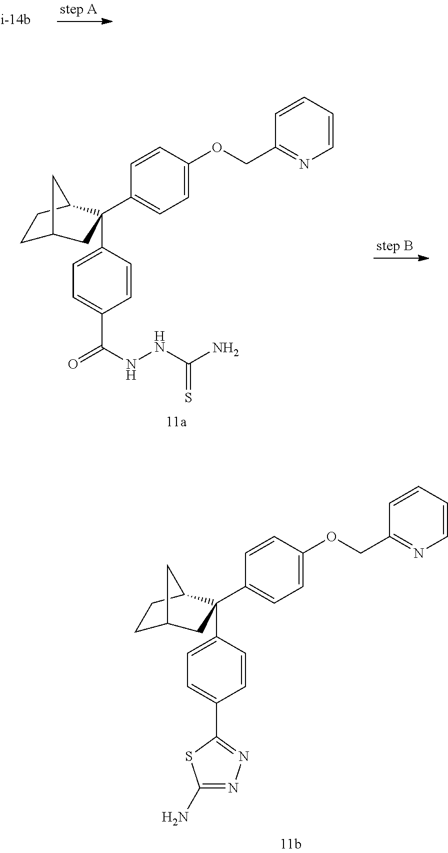

### Step A: Preparation of 2-(4-{2-[4-(pyridin-2-ylmethoxy)phenyl]bicyclo[2.2.1]-hept-2-yl}benzoyl)hydrazinecarbothioamide (11a)

DIPEA (100 μL, 0.575 mmol) was added to a stirred solution of i-14b (50.0 mg, 0.115 mmol), thiosemicarbazide (52.4 mg, 0.575 mmol), and HATU (87.4 mg, 0.23 mmol) in DMF (1.0 mL) at rt, and the resulting yellow mixture was allowed to stir at rt overnight. The mixture was poured into saturated aqueous sodium bicarbonate and extracted three times with EtOAc. The combined organic extracts were washed with water and brine, dried (Na₂SO₄) and concentrated in vacuo to afford the title compound 11a. m/z (ES) 473 (MH)⁺.

### Step B: Preparation of 5-(4-{2-[4-(pyridin-2-ylmethoxy)phenyl]bicyclo[2.2.1]-hept-2-yl}phenyl)-1,3,4-thiadiazol-2-amine (11b)

Methanesulfonic acid (8.0 μL, 0.122 mmol) was added to a stirred suspension of 11a (23.0 mg, 0.049 mmol) in toluene (450 μl), and the resulting mixture was heated to reflux for 1.5 h. The reaction mixture was poured into saturated aqueous sodium bicarbonate and was extracted once with EtOAc and three times with DCM. The combined organic extracts were washed with water and brine, dried (Na₂SO₄) and concentrated in vacuo. The resulting crude residue was purified by preparative reversed phase HPLC on YMC Pack Pro C18 stationary phase (CH₃CN/H₂O as eluent, 0.05% TFA as modifier), followed by lyophilization of the purified fractions to afford the title compound 11b. m/z (ES) 455 (MH)⁺.

Following procedures similar to those described above, the following compounds in Table 11 can be prepared:

TABLE 11

| R | 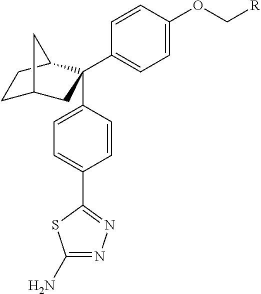 | 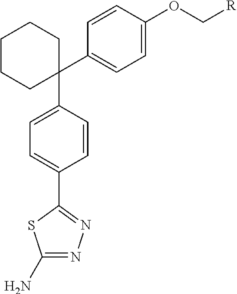 |
|---|---|---|
| 2-pyridyl | — | 11C |
| 2-pyrimidinyl | 11A | 11D |
| 2-thiazolyl | 11B | 11E |

### Example 12

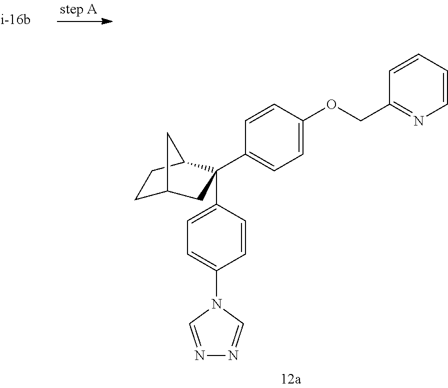

### Step A: Preparation of 2-[(4-{2-[4-(4H-1,2,4-triazol-4-yl)phenyl]bicyclo[2.2.1]-hept-2-yl}phenoxy)methyl]pyridine (12a)

i-18a (48.0 mg, 0.225 mmol) was added to a stirred solution of triethylamine (60.0 μL, 0.422 mmol), p-TSA (7.0 mg, 0.036 mmol) and i-16b (71.0 mg, 0.189 mmol) in toluene (1.5 mL), and the resulting mixture was heated to reflux for 38 h. After cooling to rt, the reaction mixture was diluted with EtOAc, and the combined organics were washed with saturated aqueous sodium bicarbonate, water and brine, dried (Na₂SO₄), and concentrated in vacuo. The resulting crude residue was purified by flash chromatography on silica gel (gradient elution; 0-5% MeOH/DCM as eluent) to afford the title compound 12a. m/z (ES) 423 (MH)⁺.

Following procedures similar to those described above, the following compounds in Table 12 can be prepared:

TABLE 12

| R | 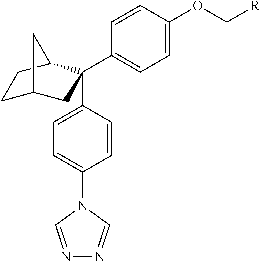 | 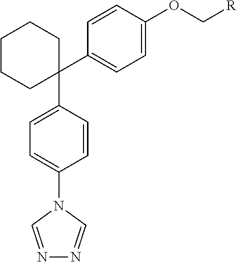 |
|---|---|---|
| 2-pyridyl | — | 12C |
| 2-pyrimidinyl | 12A | 12D |
| 2-thiazolyl | 12B | 12E |

### Example 13

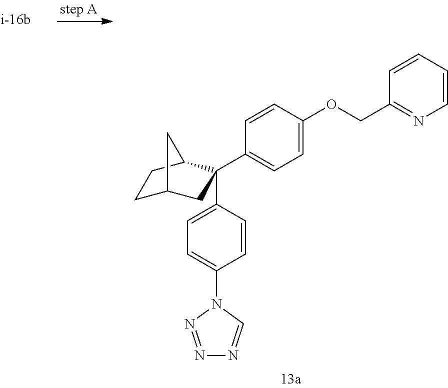

### Step A: Preparation of 2-[(4-{2-[4-(1H-tetrazol-1-yl)phenyl]bicyclo[2.2.1]-hept-2-yl}phenoxy)methyl]pyridine (13a)

Sodium azide (33.0 mg, 0.521 mmol) was added to a stirred solution of i-16b (70.0 mg, 0.189 mmol) and triethylorthoformate (125 μL, 0.750 mmol) in glacial acetic acid (450 μL), and the resulting mixture was heated to 95° C. for 1.5 h. After cooling to rt, the reaction was diluted with EtOAc, and the organics were washed with saturated aqueous sodium bicarbonate, dried (Na₂SO₄), and concentrated in vacuo. The resulting crude residue was purified by flash chromatography on silica gel (gradient elution; 0-75% EtOAc/hexanes as eluent) to afford the title compound 13a. m/z (ES) 424 (MH)⁺.

Following procedures similar to those described above, the following compounds in Table 13 can be prepared:

TABLE 13

| R | 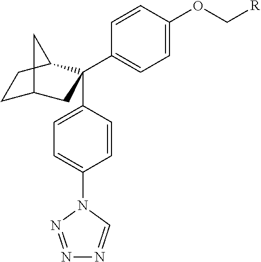 | 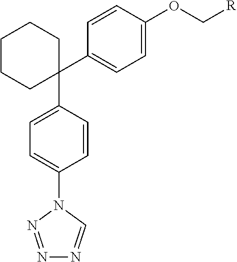 |
|---|---|---|
| 2-pyridyl | — | 13C |
| 2-pyrimidinyl | 13A | 13D |
| 2-thiazolyl | 13B | 13E |

### Example 14

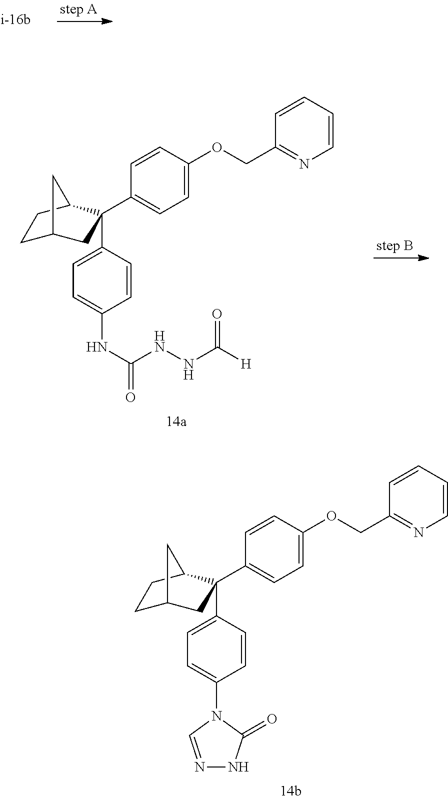

### Step A: Preparation of 2-formyl-N-(4-{2-[4-(pyridin-2-ylmethoxy)phenyl]bicyclo[2.2.1]-hept-2-yl}phenyl)hydrazinecarboxamide (14a)

Triphosgene (18.5 mg, 0.062 mmol) was added to a stirred solution of pyridine (35.0 μL, 0.438 mmol) and i-16b (50.0 mg, 0.135 mmol) in DCM (6.5 mL) at 0° C. The resulting was allowed to stir at 0° C. for 40 min, at which time formic hydrazide (37.0 mg, 0.616 mmol) and triethylamine (45.0 μL, 0.320 mmol) were added sequentially. The reaction mixture was allowed to stir at 0° C. for an additional 50 min, quenched with saturated aqueous sodium bicarbonate, and extracted three times with EtOAc. The combined organic extracts were washed with saturated aqueous sodium bicarbonate, dried (Na₂SO₄), and concentrated in vacuo. The resulting crude residue was purified by flash chromatography on silica gel (gradient elution 0-10% MeOH/DCM as eluent) to afford the title compound 14a. m/z (ES) 457 (MH)⁺.

### Step B: Preparation of 4-(4-{2-[4-(pyridin-2-ylmethoxy)phenyl]bicyclo[2.2.1]hept-2-yl}phenyl)-2,4-dihydro-3H-1,2,4-triazol-3-one (14b)

1.25 N sodium hydroxide (170 μL, 0.212 mmol) was added to a suspension of 14a (62.0 mg, 0.135 mmol) in n-butanol (1.5 mL), and the resulting mixture was heated to reflux for 4 h. After cooling to rt, the reaction mixture was diluted with EtOAc, and the organics were washed with saturated aqueous ammonium chloride and saturated aqueous sodium bicarbonate solution, dried (Na₂SO₄), and concentrated in vacuo. The resulting crude residue was purified by flash chromatography on silica gel (gradient elution; 50-100% EtOAc (1% MeOH solution)/hexanes as eluent) to afford the title compound 14b. m/z (ES) 439 (MH)⁺.

Following procedures similar to those described above, the following compounds in Table 14 can be prepared:

TABLE 14

| R | 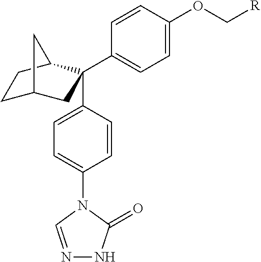 | 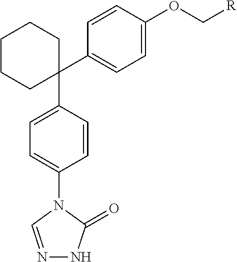 |
|---|---|---|
| 2-pyridyl | — | 14C |
| 2-pyrimidinyl | 14A | 14D |
| 2-thiazolyl | 14B | 14E |

### Example 15

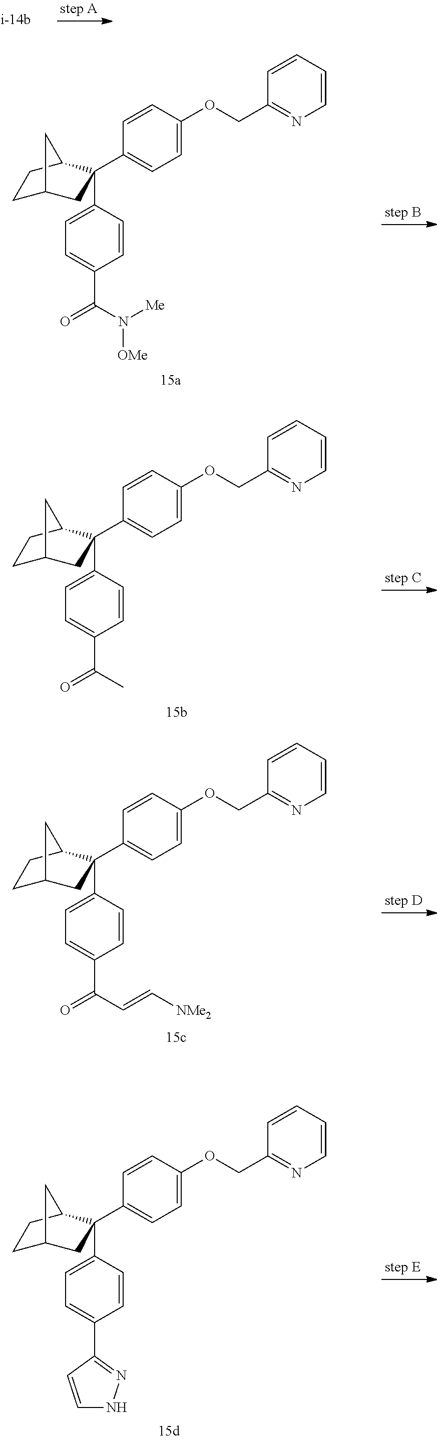

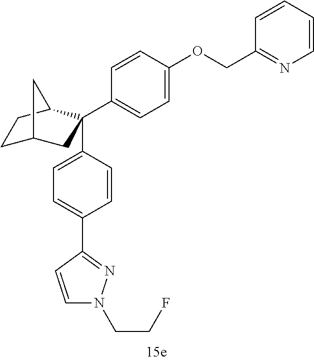

### Step A: Preparation of N-methoxy-N-methyl-4-{2-[4-(pyridin-2-ylmethoxy)phenyl]-bicyclo[2.2.1]hept-2-yl}benzamide (15a)

HATU (446 mg, 1.17 mmol) was added to a stirred solution of i-14b (256 mg, 0.587 mmol), N,O-dimethylhydroxylamine hydrochloride (86.0 mg, 0.881 mmol), and DIPEA (1.20 mL, 5.87 mmol) in DMF (6.0 mL), and the resulting mixture was allowed to stir at rt overnight. The reaction mixture was diluted with EtOAc and washed three times with water. The organic extracts were dried (MgSO₄), concentrated in vacuo, and the resulting crude residue was purified by flash chromatography on silica gel (gradient elution; 0-70% EtOAc/hexanes as eluent) to afford the title compound 15a. m/z (ES) 443 (MH)⁺.

### Step B: Preparation of 1-(4-{2-[4-(pyridin-2-ylmethoxy)phenyl]bicyclo[2.2.1]hept-2-yl}phenyl)ethanone (15b)

Methylmagnesium bromide (468 μL, of a 3.0 M diethyl ether solution, 1.40 mmol) was added to a stirred suspension of 15a (207 mg, 0.470 mmol) in THF (4.7 mL) at 0° C., and the resulting mixture was allowed to stir at 0° C. for 2 h. An additional portion of methylmagnesium bromide (156 μL, 0.470 mmol) was added, and after an additional 1 h, the reaction mixture was quenched with saturated aqueous ammonium chloride and extracted three times with EtOAc. The combined organic extracts were washed with brine, dried (MgSO₄), and concentrated in vacuo. The resulting crude residue was purified by flash chromatography on silica gel (gradient elution; 0-50% EtOAc/hexanes as eluent) to afford the title compound 15b. m/z (ES) 398 (MH)⁺.

### Step C: Preparation of (2Z)-3-(dimethylamino)-1-(4-{2-[4-(pyridin-2-ylmethoxy)phenyl]bicyclo[2.2.1]hept-2-yl}phenyl)prop-2-en-1-one (15c)

A solution of DMF-dimethylacetal (51.0 μL, 0.380 mmol) and 15b (153 mg, 0.38 mmol) in EtOH (1.0 mL) was subjected to microwave irradiation at 150° C. for 27 min. After cooling to rt, the crude mixture was purified directly by flash chromatography on silica gel (gradient elution; 0-10% MeOH (10% ammonium hydroxide solution)/DCM as eluent) to afford the title compound 15c. m/z (ES) 453 (MH)⁺.

### Step D: Preparation of 2-[(4-{2-[4-(1H-pyrazol-3-yl)phenyl]bicyclo[2.2.1]hept-2-yl}phenoxy)methyl]pyridine (15d)

Hydrazine hydrate (63.0 μL, 1.30 mmol) was added to a stirred solution of 15c (98.0 mg, 0.216 mmol) in EtOH (2.0 mL), and the resulting mixture was heated to 80° C. for 2 h. After cooling to rt, the reaction mixture was concentrated in vacuo and purified by flash chromatography on silica gel (gradient elution; 0-8% MeOH (10% ammonium hydroxide solution)/DCM as eluent) to afford the title compound 15d. m/z (ES) 422 (MH)⁺.

### Step E: Preparation of 2-{[4-(2-{4-[1-(2-fluoroethyl)-1H-pyrazol-3-yl)phenyl}bicycle-[2.2.1]hept-2-yl)phenoxy]methyl}pyridine (15e)

Sodium hydride (9.0 mg, 0.222 mmol; 60% dispersion in mineral spirits) was added to a stirred solution of 15d (78.0 mg, 0.185 mmol) in DMF (2.0 mL) at 0° C., and the resulting mixture was allowed to stir at 0° C. for 40 min. 1-Bromo-2-fluoroethane (21.0 μl, 0.277 mmol) was added, and the reaction mixture was allowed to stir at 0° C. for 3 h, quenched with brine, and extracted three times with EtOAc. The combined organic extracts were dried (MgSO₄), concentrated in vacuo, and the resulting crude residue was purified by flash chromatography on silica gel (gradient elution; 10-70% EtOAc/hexanes as eluent) to afford the title compound 15e. m/z (ES) 468 (MH)⁺.

Following procedures similar to those described above, the following compounds in Table 15 can be prepared:

TABLE 15
15A

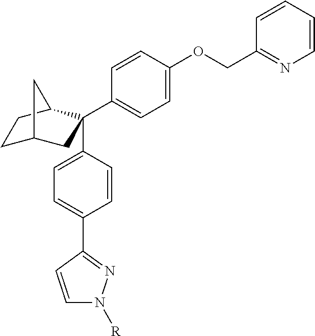

15B

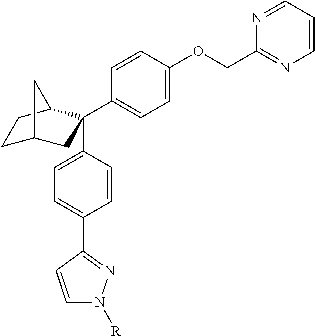

15C

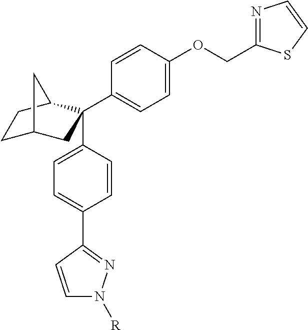

15D

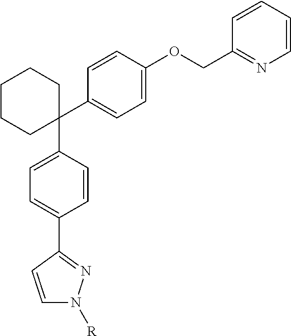

15E

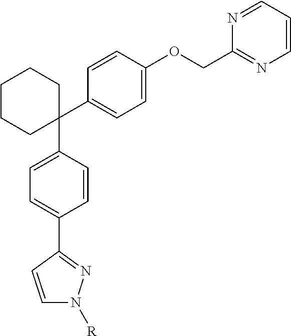

15F

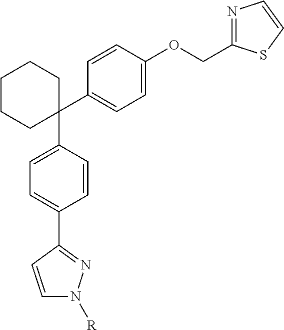

| Ex. | Ex. | Ex. | Ex. | Ex. | Ex. | |
|---|---|---|---|---|---|---|
| 15A | 15B | 15C | 15D | 15E | 15F | R |
| a | a | a | a | a | a | Me |
| b | b | b | b | b | b | Et |
| c | c | c | c | c | c | i-Pr |
| d | d | d | d | d | d | —CH₂CF₂H |
| e | e | e | e | e | e | —CH₂CF₃ |
| f | f | f | f | f | f | —CH₂C(CH₃)₂OH |
| g | g | g | g | g | g | 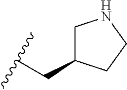 |
| h | h | h | h | h | h | 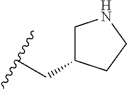 |

### Example 16

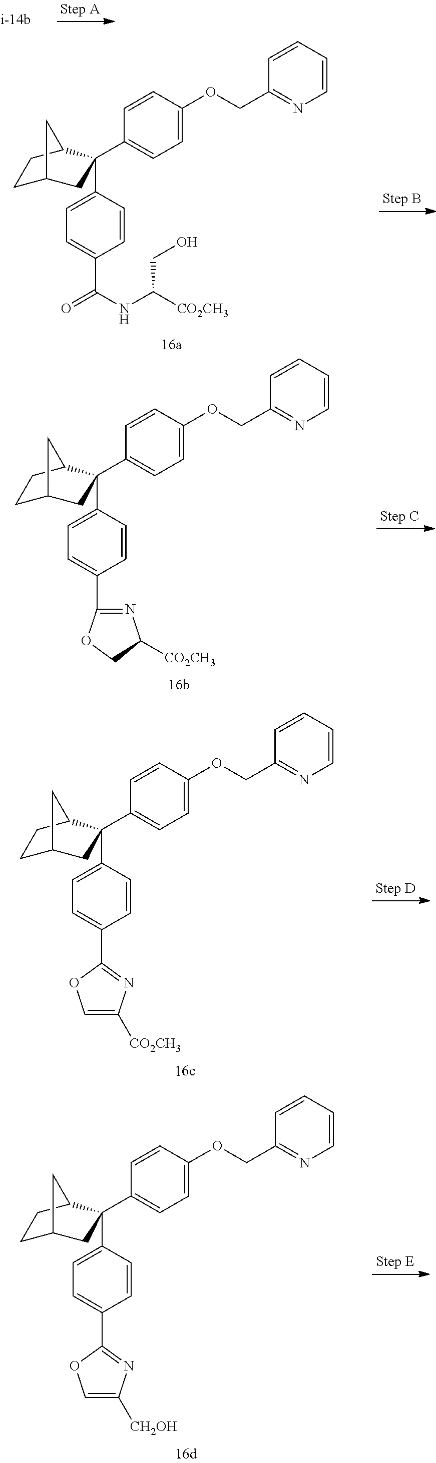

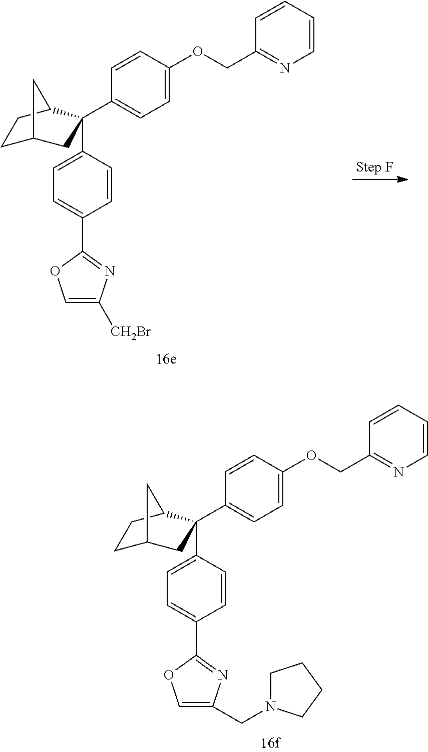

### Step A: Preparation of methyl N-(4-{(2S)-2-[4-(pyridin-2-ylmethoxy)phenyl]-bicyclo[2.2.1]hept-2-yl}benzoyl)-D-serinate (16a).

DIPEA (537 μL, 3.00 mmol) was added to a stirred solution of i-14b (300 mg, 0.750 mmol), D-serine hydrochloride (142.5 mg, 1.00 mmol), HOBT (162 mg, 1.20 mmol) and EDAC (191 mg, 1.00 mmol) in DMF (10 mL), and the resulting mixture was allowed to stir at rt for 8 h. The reaction mixture was poured into saturated aqueous ammonium chloride and extracted three times with EtOAc. The combined organic extracts were washed with water and brine, dried (MgSO₄) and concentrated in vacuo. The resulting crude residue was purified by flash chromatography on silica gel (gradient elution; 1-10% Methanol/DCM as eluent) to afford the title compound 16a. m/z (ES) 501 (MH)⁺. ¹H NMR (500 MHZ, CDCl₃): δ 8.56 (d, 1H, J=4.8 Hz), 7.68 (m, 1H), 7.66 (d, 2H, J=8.4 Hz), 7.48 (d, 1H, J=7.8 Hz), 7.32 (d, 2H, J=8.4 Hz), 7.22 (d, 2H, J=8.9 Hz), 7.19 (m, 1H), 7.07 (d, 1H, J=7.1 Hz), 6.85 (d, 2H, J=8.9 Hz), 5.13 (s, 2H), 4.83 (m, 1H), 3.97-4.06 (m, 2H), 3.79 (s, 3H), 3.18 (s, 1H), 2.40 (s, 1H), 2.32 (m, 2H), 1.72 (d, 1H, J=9.4 Hz,), 1.50 (m, 2H), 1.40 (d, 1H, J=9.6 Hz), 1.15 (m, 2H).

### Step B: Preparation of methyl (4R)-2-(4-{(2S)-2-[4-(pyridin-2-ylmethoxy)phenyl]-bicycle[2.2.1]hept-2-yl}phenyl)-4,5-dihydro-1,3-oxazole-4-carboxylate (16b)

(Diethylamino)sulfur trifluoride (116 μL, 0.720 mmol) was added dropwise to a stirred solution of 16a (300 mg, 0.600 mmol) in DCM (15.0 mL) at −78° C., and the resulting mixture was allowed to stir at −78° C. Potassium carbonate (99.5 mg, 0.720 mmol) was added, and the reaction mixture was warmed to rt, diluted with water and extracted three times with chloroform. The combined organic extracts were washed with water and brine, dried (MgSO₄) and concentrated in vacuo. The resulting crude residue was purified by flash chromatography on silica gel (gradient elution; 20-60% ethyl acetate/hexanes as eluent) to afford the title compound 16b. m/z (ES) 483 (MH)⁺. ¹H NMR (500 MHZ, CDCl₃): δ 8.60 (d, 1H, J=4.5 Hz), 7.85 (d, 2H, J=8.5 Hz), 7.72 (dt, 1H, J=1.8, 6.0 Hz), 7.51 (d, 1H, J=7.8 Hz), 7.34 (d, 2H, J=8.4 Hz), 7.23 (d, 2H, J=8.7 Hz), 7.22 (m, 1H), 6.85 (d, 2H, J=8.7 Hz), 5.16 (s, 2H), 4.93 (dd, 1H, J=7.8, 10.6 Hz), 4.67 (t, 1H, J=8.4 Hz), 4.56 (dd, 1H, J=8.7, 10.6 Hz), 3.81 (s, 3H), 3.19 (s, 1H), 2.41 (s, 1H), 2.28-2.37 (m, 2H), 1.74 (d, 1H, J=9.6 Hz), 1.50 (m, 2H), 1.40 (dd, 1H, J=1.2, 8.8 Hz), 1.18 (m, 2H).

### Step C: Preparation of methyl methyl 2-(4-{(2S)-2-[4-(pyridin-2-ylmethoxy)phenyl]bicyclo[2.2.1]hept-2-yl}phenyl)-1,3-oxazole-4-carboxylate (16c)

DBU (234 μL, 1.54 mmol) was added to a stirred solution of bromotrichloromethane (152 μL, 1.54 mmol) and 16b (185 mg, 0.384 mmol) in DCM (10.0 mL) at 0° C., and the resulting mixture was allowed to stir at 0° C. for 8 h. The reaction mixture was quenched with saturated aqueous sodium bicarbonate and extracted three times with EtOAc. The combined organic extracts were washed with brine, dried (MgSO₄), concentrated in vacuo, and the resulting crude residue was purified by flash chromatography on silica gel (gradient elution; 20-50% ethyl acetate/hexanes as eluent) to afford the title compound 16c. m/z (ES) 481 (MH)⁺. ¹H NMR (500 MHZ, CDCl₃): δ 8.60 (d, 1H, J=4.4 Hz), 8.28 (s, 1H), 7.99 (d, 2H, J=8.5 Hz), 7.72-7.74 (m, 1H), 7.53 (d, 1H, J=7.8 Hz), 7.40 (d, 2H, J=8.5 Hz), 7.26 (d, 2H, J=8.9 Hz), 7.24 (m, 1H), 6.88 (d, 2H, J=8.9 Hz), 5.17 (s, 2H), 3.97 (s, 3H), 3.21 (s, 1H), 2.43 (s, 1H), 2.32-2.37 (m, 2H), 1.75 (d, 1H, J=9.6 Hz), 1.52-1.59 (m, 2H), 1.43 (dd, 1H, J=1.2, 8.6 Hz), 1.22 (m, 2H).

### Step D: Preparation of [2-(4-{(2S)-2-[4-(pyridin-2-ylmethoxy)phenyl]bicycle-[2.2.1]hept-2-yl}phenyl)-1,3-oxazol-4-yl]methanol (16d)

Lithium aluminum hydride (19.0 mg, 0.500 mmol) was added in several portions to a stirred solution of 16c (120 mg, 0.250 mmol) in diethyl ether (2.5 mL) at 0° C., and the resulting mixture was allowed to stir at 0° C. for 30 min. The reaction mixture was quenched with 1.0 N NaOH solution, and after 20 min, the mixture was dried (MgSO₄), filtered and concentrated in vacuo to afford 16d as a white powder which was used without further purification in the subsequent reaction. m/z (ES) 453 (MH)⁺. ¹H NMR (500 MHZ, CDCl₃): δ 8.57 (d, 1H, J=4.4 Hz), 7.88 (d, 2H, J=8.4 Hz), 7.67-7.70 (m, 1H), 7.59 (s, 1H), 7.50 (d, 1H, J=8.0 Hz), 7.36 (d, 2H, J=8.4 Hz), 7.23 (d, 2H, J=8.9 Hz), 7.21 (m, 1H), 6.86 (d, 2H, J=8.9 Hz), 5.15 (s, 2H), 4.67 (s, 2H), 3.33 (s, 1H), 2.40 (s, 1H), 2.28-2.36 (m, 2H), 1.73 (d, 1H, J=9.6 Hz), 1.49-1.52 (m, 2H), 1.40 (d, 1H, J=9.5 Hz), 1.19 (m, 2H).

### Step E: Preparation of 2-{[4-((2S)-2-{4-[4-(bromomethyl)-1,3-oxazol-2-yl]phenyl}bicyclo[2.2.1]hept-2-yl)phenoxy]methyl}pyridine (16e)

Triphenylphosphine (131 mg, 0.500 mmol) was added to a stirred solution of carbon tetrabromide (166 mg, 0.500 mmol) and 16d (150 mg, 0.331 mmol) in DCM (4.0 mL) at rt, and the resulting mixture was allowed to stir at rt for 1 h. The reaction mixture was concentrated in vacuo, and the resulting crude residue was purified by flash chromatography on silica gel (gradient elution; 20-60% EtOAc/hexanes as eluent) to afford the title compound 16e. m/z (ES) 517 (MH)⁺. ¹H NMR (500 MHZ, CDCl₃): δ 8.59 (d, 1H, J=4.8 Hz), 7.91 (d, 2H, J=8.5 Hz), 7.72 (t, 1H, J=7.6 Hz), 7.67 (s, 1H), 7.52 (d, 1H, J=7.7 Hz), 7.38 (d, 2H, J=8.5 Hz), 7.24 (d, 2H, J=8.7 Hz), 7.23-7.25 (m, 1H), 6.87 (d, 2H, J=8.7 Hz), 5.17 (s, 2H), 4.45 (s, 2H), 3.20 (s, 1H), 2.42 (s, 1H), 1.07-2.38 (m, 2H), 1.75 (d, 1H, J=10.3 Hz), 1.51-1.63 (m, 2H), 1.42 (d, 1H, J=9.8 Hz), 1.20 (d, 2H, J=9.2 Hz).

### Step F: Preparation of 4-{[2-(4-(2S)-2-(4-{(2S)-2-[4-(pyridin-2-ylmethoxy)phenyl]bicycle-[2.2.1]hept-2-yl}phenyl)-1,3-oxazol-4-yl]methyl}pyrrolidine (16f)

DIPEA (28.8 μL, 0.150 mmol) was added to a mixture of 16e (51.0 mg, 0.097 mmol) and pyrrolidine (8.52 mg, 0.120 mmol) in DCM (2.0 mL), and the resulting mixture was allowed to stir at rt for 1 h. The reaction mixture was poured into water, extracted three times with EtOAc, and the combined organic extracts were washed with water and brine, dried (MgSO₄) and concentrated in vacuo. The resulting crude residue was purified by preparative TLC on silica gel (10% MeOH/DCM as eluent) to afford the title compound 16f. m/z (ES) 506 (MH)⁺. ¹H NMR (500 MHZ, CDCl₃): δ 8.59 (d 1H, J=4.8 Hz), 7.71 (m, 1H), 7.51 (d, 1H, J=7.7 Hz), 7.39 (d, 2H, J=8.5 Hz), 7.23-7.25 (m, 3H), 6.87 (d, 2H, J=8.7 Hz), 5.15 (s, 2H), 4.07 (s, 2H), 3.16-3.21 (m, 5H), 2.43 (s, 1H), 2.33-2.38 (m, 2H), 1.75 (d, 1H, J=8.9 Hz), 1.52-1.64 (m, 2H), 1.20 (d, 2H, J=9.6 Hz).

Following procedures similar to those described above, the following compounds in Table 16 can be prepared:

TABLE 16
16A

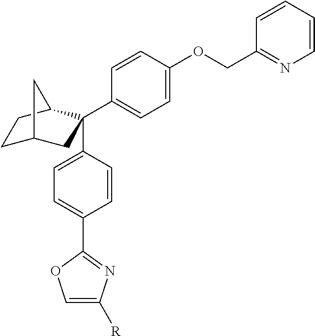

16B

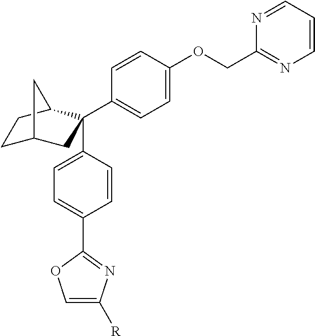

16C

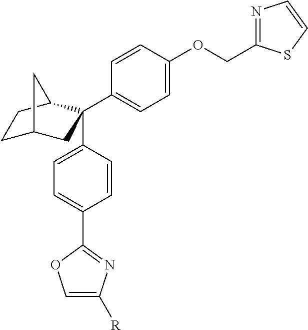

16D

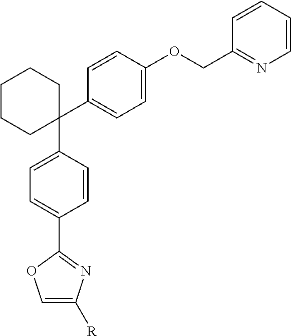

16E

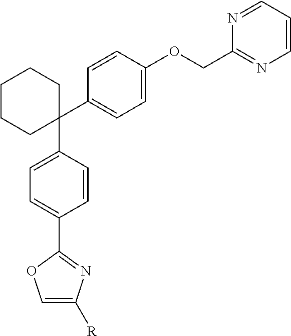

16F

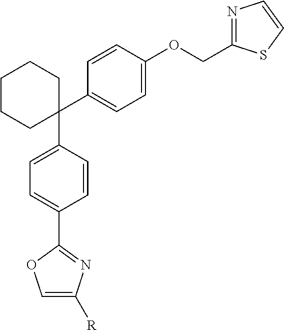

| Ex. | Ex. | Ex. | Ex. | Ex. | Ex. | |
|---|---|---|---|---|---|---|
| 16A | 16B | 16C | 16D | 16E | 16F | R |
| a | a | a | a | a | a | Me |
| b | b | b | b | b | b | Et |
| — | c | c | c | c | c | CO₂Me |
| — | d | d | d | d | d | —CH₂OH |
| e | e | e | e | e | e | —CH₂CN |
| f | f | f | f | f | f | 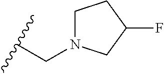 |
| g | g | g | g | g | g | 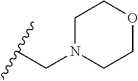 |
| h | h | h | h | h | h | 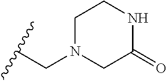 |

Table 16. Parent Ion m/z (MH)⁺ data for compounds
For 16Ag: 4-{[2-(4-{(1S,2S,4R)-2-[4-(pyridin-2-ylmethoxy)phenyl]bicyclo[2.2.1]hept-2-yl}phenyl)-1,3-oxazol-4-yl]methyl}morpholine: m/z (ES) 522 (MH)⁺
For 16Ah: 4-{[2-(4-{(1S,2S,4R)-2-[4-(pyridin-2-ylmethoxy)phenyl]bicyclo[2.2.1]hept-2-yl}phenyl)-1,3-oxazol-4-yl]methyl}piperazin-2-one: m/z (ES) 535 (MH)⁺

### Example 17

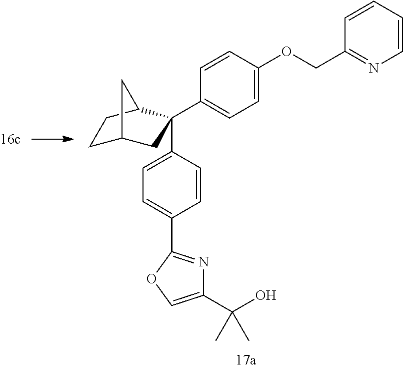

### Step A: Preparation of 2-[5-(4-{(2S)-2-[4-(pyridin-2-ylmethoxy)phenyl]bicycle-[2.2.1]hept-2-yl}phenyl)isoxazol-3-yl]propan-2-ol (17a)

Methyl lithium (135 μL of a 1.6 M THF solution, 0.216 mmol) was added to a stirred solution of 16c (26.0 mg, 0.054 mmol) in THF (1.0 mL) at −78° C., and the resulting mixture was allowed to stir at −78° C. for 10 min. The reaction mixture was quenched with saturated aqueous ammonium chloride, extracted three times with EtOAc. The combined organic extracts were concentrated in vacuo, and the resulting crude residue was purified by preparative TLC on silica gel (55% EtOAc/Hexanes as eluent) to afford the title compound 17a. m/z (ES) 481 (MH)⁺. ¹HNMR (500 MHz, CDCl₃): δ 8.59 (d 1H, J=4.3 Hz), 7.88 (d, 2H, J=8.2 Hz), 7.72 (m, 1H), 7.48-7.51 (m, 2H), 7.36 (d, 2H, J=8.2 Hz), 7.23-7.25 (m, 3H), 6.85 (d, 2H, J=8.9 Hz), 5.15 (s, 2H), 3.20 (s, 1H), 2.28-2.46 (m, 3H), 1.74 (d, 1H, J=9.2 Hz), 1.60 (s, 6H), 140 (d, 1H, J=9.4 Hz), 1.20 (m, 2H).

Following procedures similar to those described above, the following compounds in Table 17 can be prepared:

TABLE 17

| R | 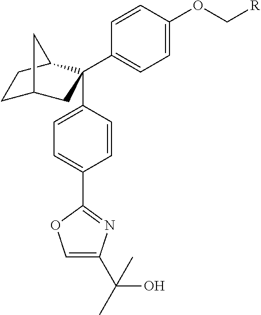 | 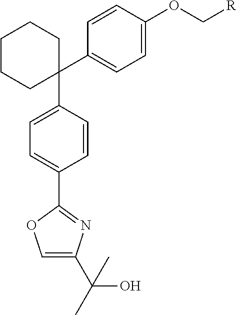 |
|---|---|---|
| 2-pyridyl | — | 17C |
| 2-pyrimidinyl | 17A | 17D |
| 2-thiazolyl | 17B | 17E |

### Example 18

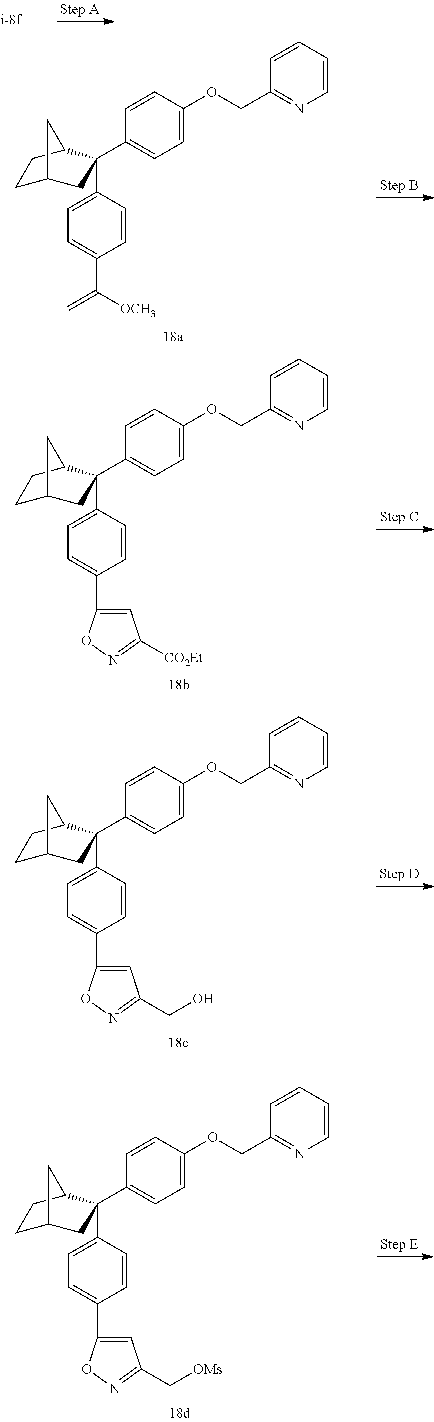

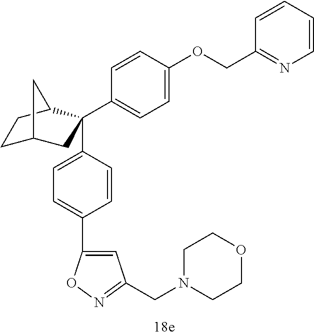

### Step A: Preparation of 2-[(4-{(2S)-2-[4-(1-methoxyvinyl)phenyl]bicyclo[2.2.1]-hept-2-yl}phenoxy)methyl]pyridine (18a)

Tebbe reagent (1.50 mL of a 0.5 M toluene solution, 0.663 mmol) was added to a stirred solution of i-8f (273 mg, 0.663 mmol) in THF (3.00 mL), and the resulting mixture was allowed to stir at for 16 h. The reaction mixture was quenched by addition of solid Al₂O₃, and the resulting suspension was filtered through Al₂O₃. The filter cake was rinsed with EtOAc and the combined filtrate was concentrated in vacuo. The crude residue containing 18a was used without purification.

### Step B: Preparation of ethyl 5-(4-{(2S)-2-[4-(pyridin-2-ylmethoxy)phenyl]bicyclo-[2.2.1]hept-2-yl}-phenyl)isoxazole-3-carboxylate (18b)

Ethyl 2-chloro-2-(hydroxyimino)acetate (303 mg, 1.99 mmol) was added to a stirred solution of 18a (crude product from Step A; 0.663 mmol, theoretical) and triethylamine (924 μL, 6.63 mmol) in THF (5.0 mL), and the resulting mixture was stirred at rt for 1 h. The reaction mixture was acidified to pH ˜1 by addition of TFA, and the resulting mixture was heated to 45° C. for 1 h, at which time, the reaction mixture was concentrated in vacuo. The resulting crude residue was redissolved in TFA (30 mL) and heated at 50° C. for an additional 1 h and concentrated in vacuo. The crude residue was purified by flash chromatography on silica gel (gradient elution; 0%-100% EtOAc/hexanes as eluent) to afford the title compound 18b. m/z (ES) 495 (MH)⁺. ¹HNMR (500 MHz, CDCl₃): δ 8.61 (d, 1H, J=4.4 Hz), 7.79 (m, 1H), 7.69 (d, 2H, J=8.5 Hz), 7.57 (d, 1H, J=7.8 Hz), 7.42 (d, 2H, J=8.5 Hz), 7.27 (d, 2H, J=8.9 Hz), 7.24 (m, 1H), 6.89 (d, 2H, J=8.9 Hz), 6.85 (s, 1H), 5.20 (s, 2H), 4.47 (q, 2H, J=7.1 Hz), 3.22 (s, 1H), 2.42 (s, 1H), 2.36-2.39 (m, 2H), 1.75 (d, 1H, J=9.6 Hz), 1.40-1.47 (m, 5H), 1.21 (m, 2H).

### Step C: Preparation of [5-(4-{(2S)-2-[4-(pyridin-2-ylmethoxy)phenyl]bicyclo[2.2.1]hept-2-yl}phenyl)isoxazol-3-yl]methanol (18c)

Lithium aluminum hydride (0.780 mL of a 1.0 M THF solution, 0.780 mmol) was added to a stirred solution of 18b (258 mg, 0.522 mmol) in Et₂O (2.0 mL) at 0° C., and the resulting mixture was allowed to stir at 0° C. for 10 min. The reaction mixture was quenched by addition of 1.0 M NaOH and diluted with EtOAc. The resulting mixture was dried (MgSO₄), filtered and concentrated in vacuo. The crude residue was purified by flash chromatography on silica gel (gradient elution; 0%-100% EtOAc/hexanes as eluent) to afford the title compound 18c. m/z (ES) 435 (MH)⁺. ¹H NMR (500 MHZ, CDCl₃): δ 8.59 (d, 1H, J=4.4 Hz), 7.88 (d, 2H, J=8.4 Hz), 7.67-7.70 (m, 1H), 7.51 (d, 1H, J=8.0 Hz), 7.38 (d, 2H, J=8.4 Hz), 7.19-7.24 (m, 3H), 6.87 (d, 2H, J=8.9 Hz), 6.51 (s, 1H), 5.16 (s, 2H), 4.80 (s 2H), 3.20 (s, 1H), 2.40 (s, 1H), 2.28-2.35 (m, 2H), 1.74 (m, 1H), 1.50-1.54 (m, 2H), 1.41 (d, 1H, J=9.5 Hz), 1.20 (m, 2H).

### Step D: Preparation of [5-(4-{(2S)-2-[4-(pyridin-2-ylmethoxy)phenyl]bicyclo[2.2.1]hept-2-yl}phenyl)isoxazol-3-yl]methyl methanesulfonate (18d)

Following the procedure described in Step E of Scheme 5, methanesulfonate 18d can be prepared.

### Step E Preparation of 4-{[5-(4-{(2S)-2-[4-(pyridin-2-ylmethoxy)phenyl]bicycle[2.2.1]-hept-2-yl}phenyl)isoxazol-3-yl]methyl}morpholine (18e)

Following the procedure described in Step F of Scheme 5, morpholine 18e can be prepared.

Following procedures similar to those described above, the following compounds in Table 18 can be prepared:

TABLE 18
18A

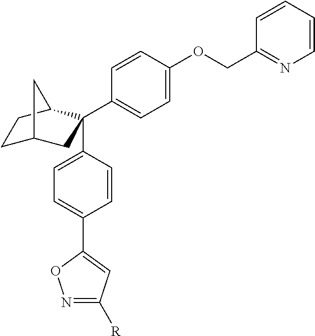

18B

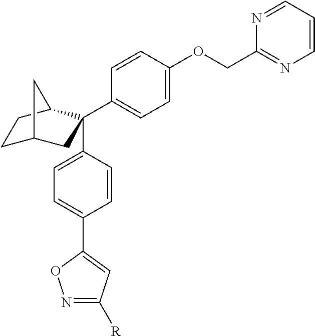

18C

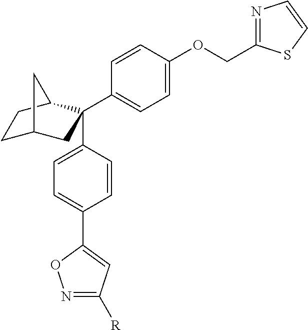

18D

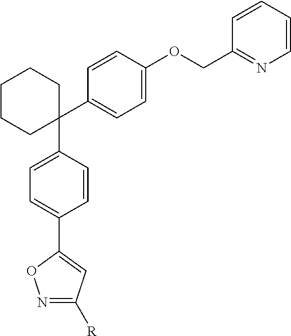

18E

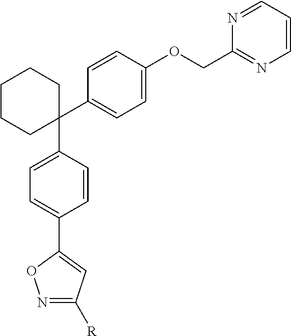

18F

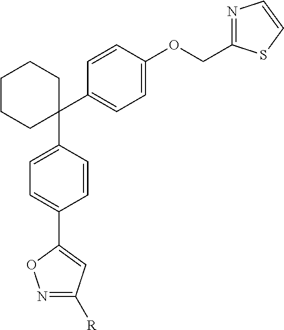

| Ex. | Ex. | Ex. | Ex. | Ex. | Ex. | |
|---|---|---|---|---|---|---|
| 18A | 18B | 18C | 18D | 18E | 18F | R |
| a | a | a | a | a | a | Me |
| b | b | b | b | b | b | Et |
| — | c | c | c | c | c | CO₂Me |
| — | d | d | d | d | d | —CH₂OH |
| e | e | e | e | e | e | 1-hydroxycyclopropyl |
| f | f | f | f | f | f | 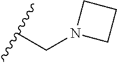 |
| g | g | g | g | g | g | 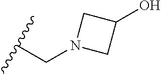 |
| h | h | h | h | h | h | 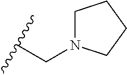 |
| i | i | i | i | i | i | 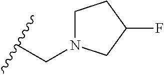 |

### Example 19

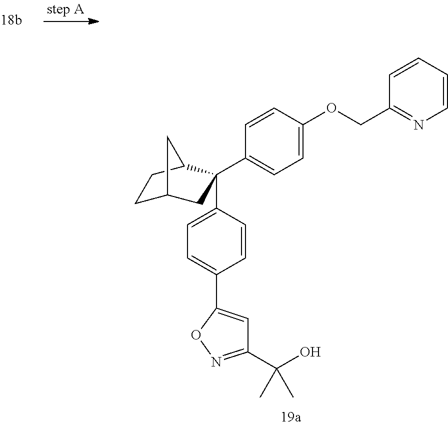

### Step A: Preparation of 2-[5-(4-{(2S)-2-[4-(pyridin-2-ylmethoxy)phenyl]-bicyclo[2.2.1]hept-2-yl}phenyl)isoxazol-3-yl]propan-2-ol (19a)

Methylmagnesium bromide (334 μL, of a 1.4 M (3:1) toluene:THF solution, 0.468 mmol) was added to a stirred solution of 18b (56.0 mg, 0.117 mmol) in THF (2.0 mL) at 0° C., and the resulting mixture was allowed to stir at 0° C. for 1 h. The reaction was quenched with saturated aqueous ammonium chloride and extracted three times with EtOAc. The Combined organic layers were concentrated and the resulting mixture was purified directly by preparative TLC on silica gel (65% EtOAc/Hexanes as a eluent) to afford the title compound 19a. m/z (ES) 481 (MH)⁺.

Following procedures similar to those described above, the following compounds in Table 19 can be prepared:

TABLE 19

| R | 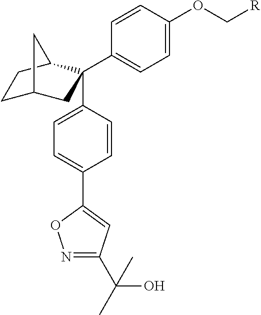 | 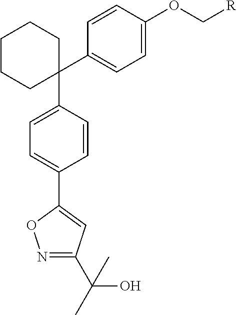 |
|---|---|---|
| 2-pyridyl | — | 19C |
| 2-pyrimidinyl | 19A | 19D |
| 2-thiazolyl | 19B | 19E |

### Example 20

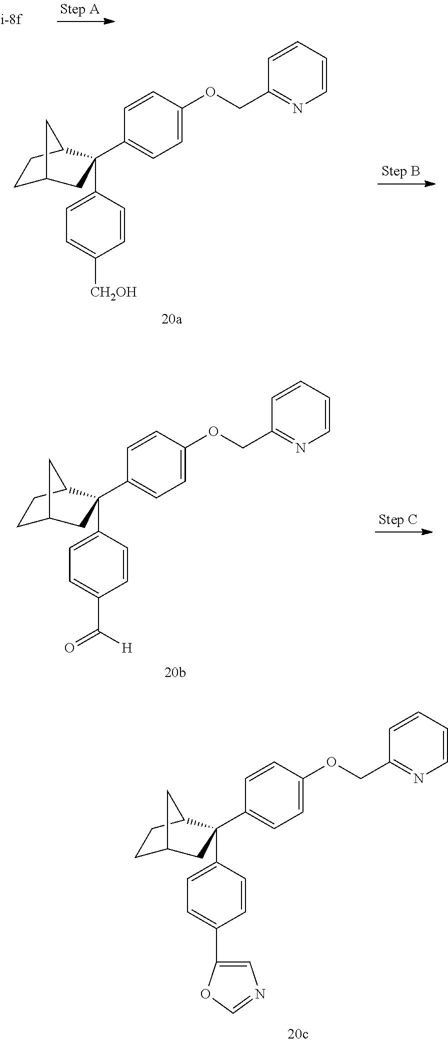

### Step A: preparation of 2 (4-{(2S)-2-[4-(pyridin-2-ylmethoxy)phenyl]bicyclo[2.2.1]hept-2-yl}phenyl)methanol (20a)

Lithium aluminum hydride (54.0 mg, 1.42 mmol) was added in several portions to a stirred solution of i-8f (293 mg, 0.708 mmol) in diethyl ether (5.0 mL) at 0° C., and the resulting mixture was allowed to stir at 0° C. for 30 min. The reaction was quenched with 1.0 N NaOH, and after stirring for an additional 20 min, the mixture was dried (MgSO₄), filtered and concentrated in vacuo to afford 20a as a colorless oil which was used without further purification. m/z (ES) 386 (MH)⁺. ¹H NMR (500 MHZ, CDCl₃): δ 8.56 (d, 1H, J=4.5 Hz), 7.70 (t, 1H, J=7.6 Hz), 7.51 (d, 1H, J=7.8 Hz), 7.20-7.28 (m, 7H), 6.85 (d, 2H, J=8.5 Hz), 5.14 (s, 2H), 4.62 (d, 1H, J=5.5 Hz), 3.19 (s, 1H), 2.39 (s, 1H), 2.30 (m, 2H), 1.72 (d, 1H, J=9.4 Hz), 1.50 (m, 2H), 1.39 (d, 1H, J=9.6 Hz), 1.18 (m, 2H).

### Step B: Preparation of 4-{(2S)-2-[4-(pyridin-2-ylmethoxy)phenyl]bicyclo[2.2.1]hept-2-yl}benzaldehyde (20b)

Celite® (500 mg) was added to a stirred suspension of manganese (IV) oxide (500 mg, excess) and 20a (crude product from Step A; 0.708 mmol, theoretical) in DCM (12.0 mL), and the resulting mixture was allowed to stir at rt for 4 h. The reaction mixture was filtered, and the solids were washed copiously with EtOAc. The combined filterate was concentrated in vacuo, and the resulting crude residue was purified by flash chromatography on silica gel (gradient elution; 5-30% EtOAc/Hexanes as eluent) to afford the title compound 20b. m/z (ES) 384 (MH)⁺. ¹H NMR (500 MHZ, CDCl₃): δ 9.95 (s, 1H), 8.60 (d, 1H, J=4.6 Hz), 7.71 (t, 1H, J=7.6 Hz), 7.51 (d, 1H, J=8.0 Hz), 7.47 (d, 2H, J=7.3 Hz), 7.20-7.28 (m, 3H), 6.87 (d, 2H, J=8.9 Hz), 5.16 (s, 2H), 3.23 (s, 1H), 2.36 (s, 1H), 2.34 (s, 1H), 1.75 (d, 1H, J=9.8 Hz), 1.54 (m, 2H), 1.42 (d, 1H, J=9.8 Hz), 1.18 (m, 2H).

### Step C: Preparation of 2-[(4-{(2S)-2-[4-(1,3-oxazol-5-yl)phenyl]bicyclo[2.2.1]hept-2-yl}phenoxy)methyl]pyridine (20c)

p-Toluenesulfonylmethyl isocyanide (61.0 mg, 0.313 mmol) was added to a stirred suspension of 20b (119 mg, 0.310 mmol) and potassium carbonate (43.0 mg, 0.312 mmol) in MeOH (3.0 mL), and the resulting mixture was refluxed in a gas tight vessel for 18 h. After cooling to rt, the reaction mixture was poured into water and extracted with EtOAc. The organic extracts were washed with brine, dried (MgSO₄) and concentrated. The resulting crude residue was purified by flash chromatography on silica gel (gradient elution; 10-50% EtOAc/Hexanes as eluent) to afford the title compound 20c, m/z (ES) 423 (MH)⁺. ¹H NMR (500 MHZ, CDCl₃): δ 8.60 (d, 1H, J=4.6 Hz), 7.89 (s, 1H), 7.71 (t, 1H, J=7.6 Hz), 7.51-7.54 (m, 3H), 7.35 (d, 2H, J=8.4 Hz), 7.20-7.28 (m, 3H), 6.88 (d, 2H, J=8.8 Hz), 5.16 (s, 2H), 3.20 (s, 1H), 2.42 (s, 1H), 2.33 (s, 2H), 1.75 (d, 1H, J=9.6 Hz), 1.53 (m, 2H), 1.42 (d, 1H, J=9.6 Hz), 1.22 (m, 2H).

Following procedures similar to those described above, the following compounds in Table 20 can be prepared:

TABLE 20

| R | 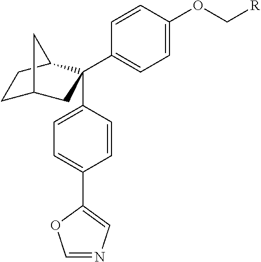 | 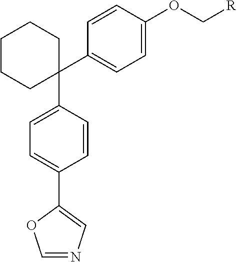 |
|---|---|---|
| 2-pyridyl | — | 20C |
| 2-pyrimidinyl | 20A | 20D |
| 2-thiazolyl | 20B | 20E |

### Example 21

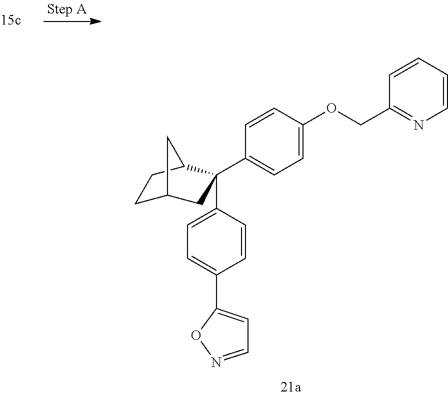

### Step C: Preparation of 2-({4-[(2S)-2-(4-isoxazol-5-ylphenyl)bicyclo[2.2.1]hept-2-yl]phenoxy}methyl)pyridine (21a)

A thick-walled pressure tube was charged with 15c (34.0 mg, 0.075 mmol), hydroxylamine hydrochloride (12.0 mg, 0.173 mmol) and ethanol (1.0 mL), and the resulting mixture was irradiated in a microwave apparatus (300W) at 110° C. for 2 h. After cooling to rt, the reaction mixture was concentrated in vacuo, and the resulting crude residue was purified by preparative TLC on silica gel (40% EtOAc/Hexanes as eluent) to afford the title compound 21a. m/z (ES) 423 (MH)⁺.

Following procedures similar to those described above, the following compounds in Table 21 can be prepared:

TABLE 21

| R | 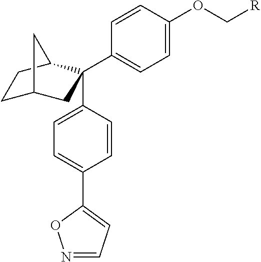 | 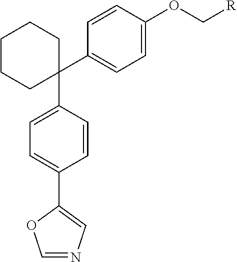 |
|---|---|---|
| 2-pyridyl | — | 21C |
| 2-pyrimidinyl | 21A | 21D |
| 2-thiazolyl | 21B | 21E |

### Example 22

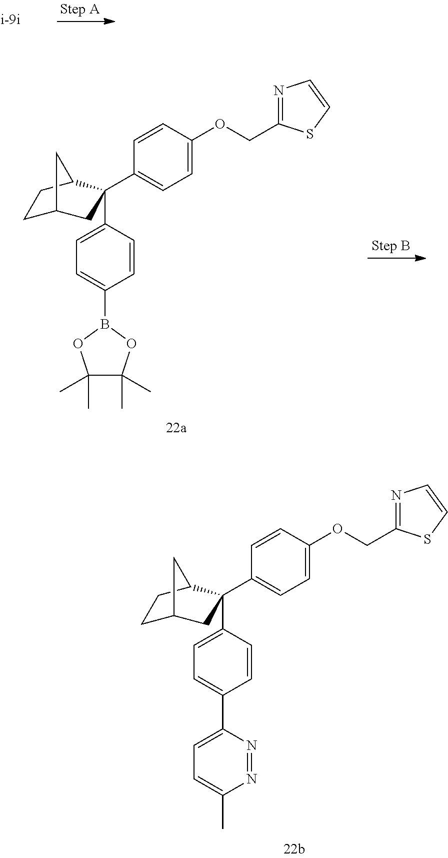

### Step A: Preparation of 2-[(4-{2-[4-(4,4,5,5,tetramethyl-1,3,2-dioxaborolan-2-yl)phenyl]-bicyclo[2.2.1]hept-2-yl}phenoxy)methyl]-1,3-thiazole (22a)

i-9i (74 mg, 0.152 mmol), bis(pinacolato)diboron (42 mg, 0.167 mmol), potassium acetate (45 mg, 0.456 mmol), and [1,1′-bis(diphenylphosphino)ferrocene]dichloropalladium (3.4 mg, 0.00456 mmol) were combined in DMSO (1.5 mL), degassed, flushed with nitrogen, heated to 80° C., and allowed to stir at 80° C. overnight. After cooling to rt, the reaction mixture was poured into saturated aqueous sodium bicarbonate and extracted three times with EtOAc. The combined organic extracts were washed with water and brine, dried (Na₂SO₄), and concentrated. The resulting crude residue was purified by flash chromatography on silica gel (gradient elution; 0-28% EtOAc/Hexanes as eluent) to afford the title compound 22a. m/z (ES) 488 (MH)⁺.

### Step B: Preparation of 3-methyl-6-(4-{2-[4-(1,3-thiazol-2-ylmethoxy)phenyl]bicyclo-[2.2.1]hept-2-yl}phenyl)pyridazine (22b)

Sodium carbonate (115 μL of a 2.0 M aqueous solution, 0.230 mmol) was added to a stirred solution of 3-chloro-6-methylpyridazine (22.0 mg, 0.173 mmol) and 22a (56.0 mg, 0.115 mmol) in toluene (0.5 mL) and EtOH (1.0 mL). The resultant mixture was degassed and back flushed with nitrogen three times, at which time, tetrakis(triphenylphosphine)palladium (17.0 mg, 0.016 mmol) was added, and the resulting mixture heated to reflux overnight. The reaction mixture was poured into saturated aqueous sodium bicarbonate and extracted three times with EtOAc. The combined organic extracts were washed with water and brine, dried (Na₂SO₄), and concentrated in vacuo. The resulting crude residue was purified by preparative reversed phase HPLC on YMC Pack Pro C18 stationary phase (CH₃CN/H₂O as eluent, 0.05% TFA as modifier), followed by lyophilization of the purified fractions to afford the title compound 22b. m/z (ES) 454 (MH)⁺.

Following procedures similar to those described above, the following compounds in Table 22 can be prepared:

TABLE 22
22A

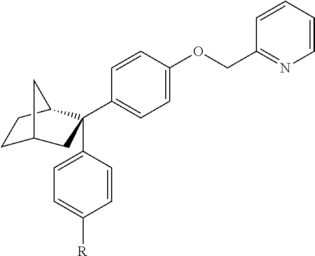

22B

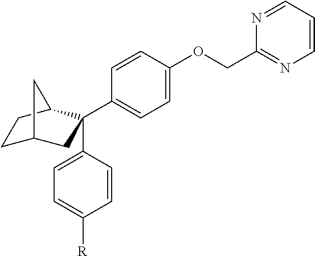

22C

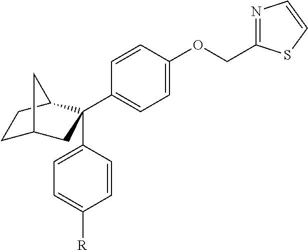

22D

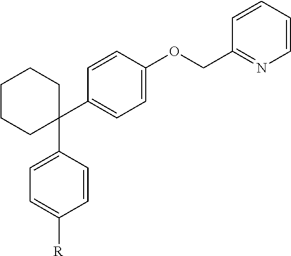

22E

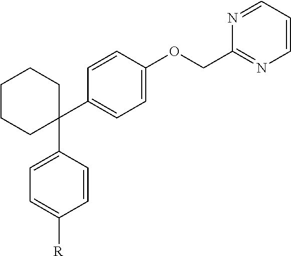

22F

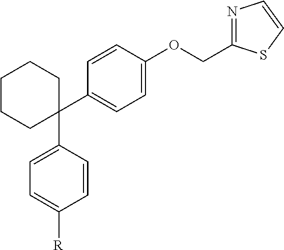

22G

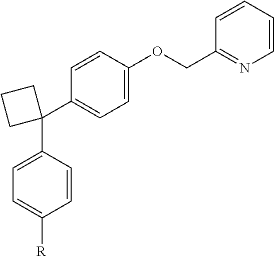

22H

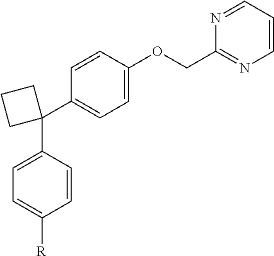

22I

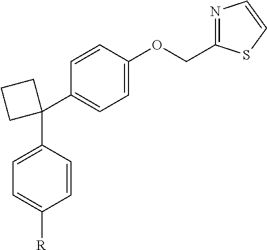

| Ex. | Ex. | Ex. | Ex. | Ex. | Ex. | Ex. | Ex. | Ex. | |
|---|---|---|---|---|---|---|---|---|---|
| 22A | 22B | 22C | 22D | 22E | 22F | 22G | 22H | 22I | R |
| a | a | a | a | a | a | a | a | a | 2-pyridyl |
| b | b | b | b | b | b | b | b | b | 3-pyridyl |
| c | c | c | c | c | c | c | c | c | 4-pyridyl |
| d | d | d | d | d | d | d | d | d | 2-pyrimidinyl |
| e | e | e | e | e | e | e | e | e | 5-pyrimidinyl |
| f | f | f | f | f | f | f | f | f | 6-F-3-pyridyl |
| g | g | g | g | g | g | g | g | g | 6-amino-3-pyridyl |
| h | h | h | h | h | h | h | h | h | 6-methoxy-3-pyridyl |
| i | i | i | i | i | i | i | i | i | 6-hydroxy-3-pyridyl |
| j | j | j | j | j | j | j | j | j | 3-pyridazinyl |
| — | k | k | k | k | k | k | k | k | 6-methyl-3-pyridazinyl |
| l | l | l | l | l | l | l | l | l | 6-methoxy-3-pyridazinyl |
| m | m | m | m | m | m | m | m | m | 6-trifluoromethy1-3- |
| | | | | | | | | | pyridazinyl |
| n | n | n | n | n | n | n | n | n | 6-chloro-3-pyridazinyl |
| o | o | o | o | o | o | o | o | o | 6-methylsulfonyl-3- |
| | | | | | | | | | pyridazinyl |
| p | p | p | p | p | p | p | p | p | 5-amino-2-pyrazinyl |
| q | q | q | q | q | q | q | q | q | 2-amino-4-pyrimidinyl |
| r | r | r | r | r | r | r | r | r | 2-amino-5-pyrimidinyl |

Table 22. Parent Ion m/z (MH)⁺ data for compounds
For 22Cd: 2-(4-{(1S,2S,4R)-2-[4-(1,3-thiazol-2-ylmethoxy)phenyl]bicyclo[2.2.1]hept-2-yl}-phenyl)pyrimidine: m/z (ES) 449 (MH)⁺
For 22 Db: 2-({4-[1-(4-pyridin-3-ylphenyl)cyclohexyl]phenoxy}methyl)pyridine: m/z (ES) 421 (MH)⁺
For 22Dc: 2-({4-[1-(4-pyridin-4-ylphenyl)cyclohexyl]phenoxy}methyl)pyridine: m/z (ES) 421 (MH)⁺
For 22Dk: 3-methyl-6-(4-{1-[4-(pyridin-2-ylmethoxy)phenyl]cyclohexyl}phenyl)pyridazine: m/z (ES) 436 (MH)⁺
For 22Dl: 3-methoxy-6-(4-{1-[4-(pyridin-2-ylmethoxy)phenyl]cyclohexyl}phenyl)pyridazine: m/z (ES) 452 (MH)⁺
For 22Ga: 2-({4-[1-(4-pyridin-2-ylphenyl)cyclobutyl]phenoxy}methyl)pyridine: m/z (ES) 393 (MH)⁺
For 22 Gb: 2-({4-[1-(4-pyridin-3-ylphenyl)cyclobutyl]phenoxy}methyl)pyridine: m/z (ES) 393 (MH)⁺
For 22Gc: 2-({4-[1-(4-pyridin-4-ylphenyl)cyclobutyl]phenoxy}methyl)pyridine: m/z (ES) 393 (MH)⁺
For 22Gk: 3-methyl-6-(4-{1-[4-(pyridin-2-ylmethoxy)phenyl]cyclobutyl}phenyl)pyridazine: m/z (ES) 408 (MH)⁺
For 22Gl: 3-methoxy-6-(4-{1-[4-(pyridin-2-ylmethoxy)phenyl]cyclobutyl}phenyl)pyridazine: m/z (ES) 424 (MH)⁺

### Example 23

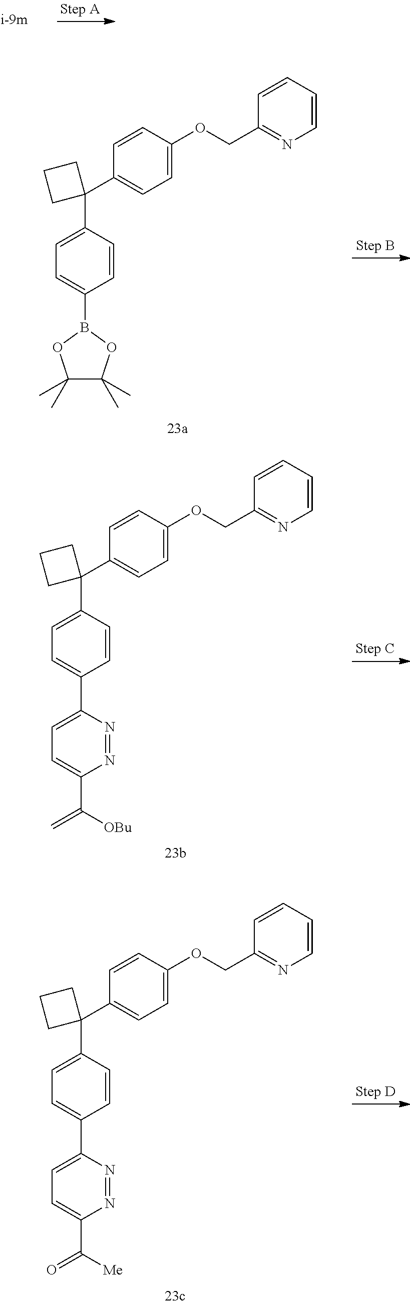

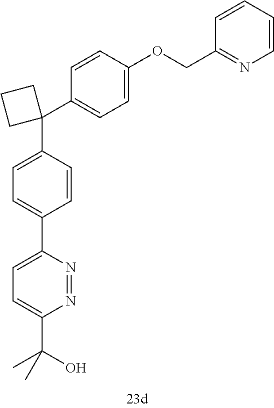

### Step A: Preparation of 2-[(4-{1-[4-(4,4,5,5-tetramethyl-1,3,2-dioxaborolan-2-yl)phenyl]cyclobutyl}phenoxy)methyl]pyridine (23a)

Following Step A of Example 22, 23a was obtained. m/z (ES) 442 (MH)⁺.

### Step B: Preparation of 3-(1-butoxyvinyl)-6-(4-{1-[4-(pyridin-2-ylmethoxy)phenyl]cyclobutyl}phenyl)pyridazine (23b)

[1,1′-Bis(diphenylphosphino)ferrocene]dichloropalladium(II) (7.4 mg, 0.0091 mmol) was added to a stirred solution of 23a (40.0 mg, 0.091 mmol), i-19a (23.1 mg, 0.109 mmol), and 2.0 M aqueous sodium carbonate (91.0 μL, 0.181 mmol) in EtOH (1.0 mL) and toluene (0.25 mL) in a thick-walled pressure tube. The resulting mixture was degassed with a stream of nitrogen gas and heated to 80° C. for 4 h. After cooling to rt, the reaction mixture was filtered through a column of Celite®, and the solids were rinsed exhaustively with EtOAc. The combined filtrate was washed with water and brine, dried (Na₂SO₄), and concentrated in vacuo. The resulting crude residue was purified by flash chromatography on silica gel (gradient elution; 0-30% ethyl acetate/hexanes as eluent) to afford the title compound 23b. m/z (ES) 492 (MH)⁺.

### Step C: Preparation of 1-[6-(4-{1-[4-(pyridin-2-ylmethoxy)-phenyl]cyclobutyl}-phenyl)pyridazin-3-yl]ethanone (23c)

2.0 N HCl (3.0 mL) was added to a stirred solution of 23b (23.0 mg, 0.047 mmol) in EtOH (2.0 mL) at 0° C., and the resulting mixture was allowed to stir at 0° C. for 30 min. The reaction mixture was quenched with saturated aqueous sodium bicarbonate and brine, and extracted three times with EtOAc. The combined organic extracts were washed with brine, dried (Na₂SO₄), and concentrated in vacuo. The resulting crude product was used in the subsequent step without further purification. m/z (ES) 436 (MH)⁺.

### Step D: Preparation of 2-[6-(4-{1-[4-(pyridin-2-ylmethoxy)phenyl]cyclobutyl}-phenyl)pyridazin-3-yl]propan-2-ol (23d)

Methylmagnesium bromide (46.0 μL of a 1.4 M (3:1) toluene:THF solution, 0.064 mmol) was added to a stirred solution of 23c (20.0 mg, 0.046 mmol) in THF (0.5 mL) and diethyl ether (1.0 mL) at 0° C. The resulting mixture was allowed to stir at 0° C. for 1.25 h, quenched with saturated aqueous ammonium chloride, and extracted three times with EtOAc. The combined organic extracts were washed with brine, dried (Na₂SO₄), concentrated in vacuo, and the resulting crude residue was purified by preparative reversed phase HPLC on YMC Pack Pro C18 stationary phase (CH₃CN/H₂O as eluent, 0.05% TFA as modifier), followed by lyophilization of the purified fractions to afford the title compound 23d. m/z (ES) 452 (MH)⁺.

Following procedures similar to those described above, the following compounds in Table 23 can be prepared:

TABLE 23

| R | 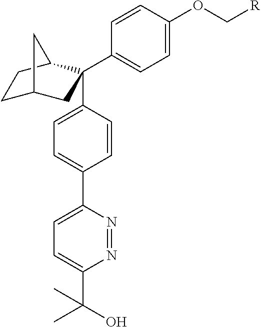 | 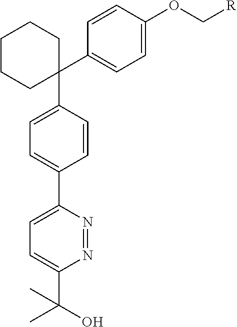 | 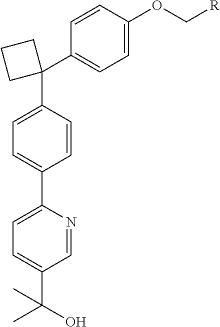 |
|---|---|---|---|
| 2-pyridyl | 23A | 23D | — |
| 2-pyrimidinyl | 23B | 23E | 23G |
| 2-thiazolyl | 23C | 23F | 23H |

Table 23. Parent Ion m/z (MH)⁺ data for compounds
For 23D: 2-[6-(4-{1-[4-(pyridin-2-ylmethoxy)phenyl]cyclohexyl}phenyl)pyridazin-3-yl]propan-2-ol: m/z (ES) 427 (MH)⁺

### Example 24

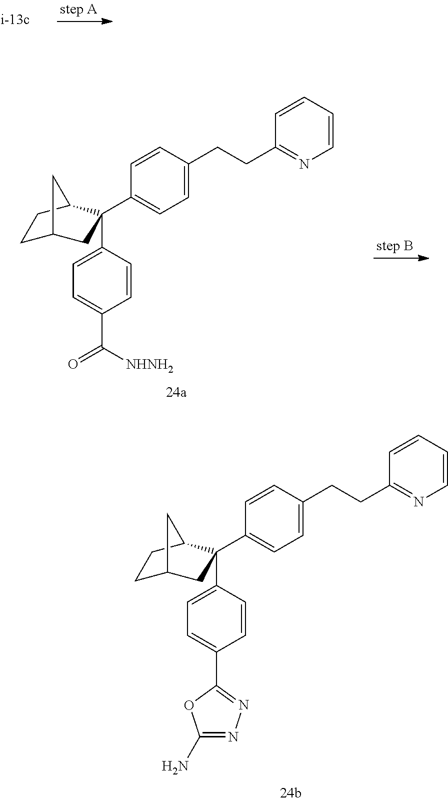

### Step A: Preparation of 4-{(1S,2S,4R)-2-[4-(2-pyridin-2-ylethyl)phenyl]bicyclo[2.2.1]hept-2-yl}benzohydrazide (24a)

Compound 24a was prepared following procedures as previously described in step A of scheme i-15.

### Step B: Preparation of 5-(4-{(1S,2S,4R)-2-[4-(2-pyridin-2-ylethyl)phenyl]bicyclo[2.2.1]-hept-2-yl}phenyl)-1,3,4-oxadiazol-2-amine (24b)

Compound 24b was prepared following procedures as previously described in step A of Example 2. m/z (ES) 437 (MH)⁺.

Following procedures similar to those described above, the following compounds in Table 24 can be prepared:

TABLE 24

| R | 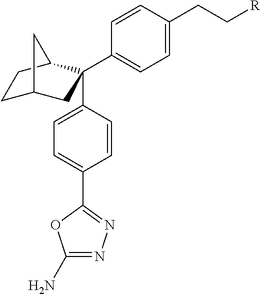 | 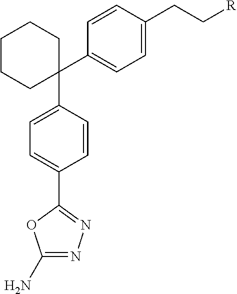 |
|---|---|---|
| 2-pyridyl | — | 24C |
| 2-pyrimidinyl | 24A | 24D |
| 2-thiazolyl | 24B | 24E |

### Example 25

Following procedures described previously in Schemes i-11, i-12, and i-14, as well as Examples 1, 2, 8, 22, and 23, the compounds in Table 25 can be prepared.

TABLE 25
25A

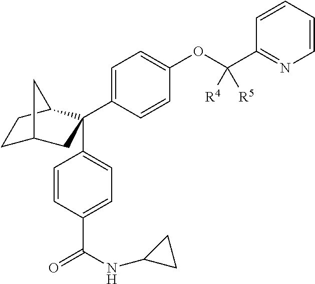

25B

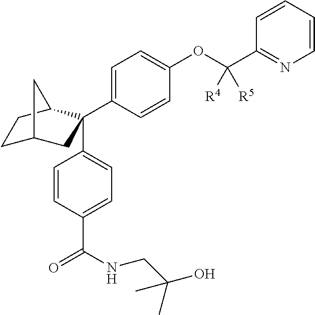

25C

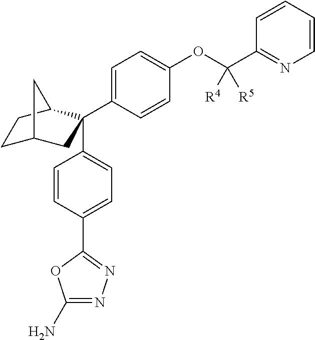

25D

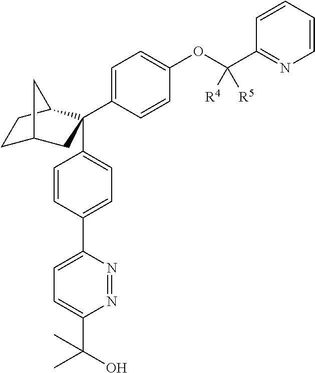

25E

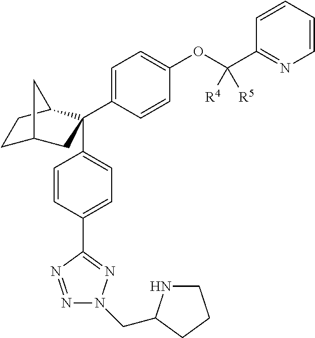

| Ex. | Ex. | Ex. | Ex. | Ex. | | |
|---|---|---|---|---|---|---|
| 25A | 25B | 25C | 25D | 25E | R⁴ | R⁵ |
| a | a | a | a | a | H | Me |
| b | b | b | b | b | H | Et |
| c | c | c | c | c | H | i-Pr |
| d | d | d | d | d | H | CF₃ |
| e | e | e | e | e | H | CO₂H |
| f | f | f | f | f | H | CO₂Me |
| g | g | g | g | g | H | CONH₂ |
| h | h | h | h | h | Me | Me |
| i | i | i | i | i | Me | CO₂Me |

Table 25. Parent Ion m/z (MH)⁺ data for compounds
For 25Aa: N-cyclopropyl-4-{(1S,2S,4R)-2-[4-(1-pyridin-2-ylethoxy)phenyl]bicyclo[2.2.1]hept-2-yl}benzamide: m/z (ES) 453 (MH)⁺
For 25Ab: N-cyclopropyl-4-{(1S,2S,4R)-2-[4-(1-pyridin-2-ylpropoxy)phenyl]bicyclo[2.2.1]hept-2-yl}benzamide: m/z (ES) 467 (MH)⁺
For 25Ac: N-cyclopropyl-4-{(1S,2S,4R)-2-[4-(2-methyl-1-pyridin-2-ylpropoxy)phenyl]bicyclo-[2.2.1]hept-2-yl}benzamide: m/z (ES) 481 (MH)⁺
For 25Ad: N-cyclopropyl-4-{(1S,2S,)-2-[4-(2,2,2-trifluoro-1-pyridin-2-ylethoxy)phenyl]bicyclo-[2.2.1]hept-2-yl}benzamide: m/z (ES) 507 (MH)⁺
For 25Ae: [4-((1S,2S,4R)-2-{4-[(cyclopropylamino)carbonyl]phenyl}bicyclo[2.2.1]hept-2-yl)phenoxy]pyridin-2-yl)acetic acid: m/z (ES) 483 (MH)⁺
For 25Af: methyl[4-((1S,2S,4R)-2-{4-[(cyclopropylamino)carbonyl]phenyl}bicyclo[2.2.1]hept-2-yl)phenoxy](pyridin-2-yl)acetate: m/z (ES) 497 (MH)⁺
For 25Ag: 4-{(1S,2S,4R)-2-[4-(2-amino-2-oxo-1-pyridin-2-ylethoxy)phenyl]bicyclo[2.2.1]hept-2-yl}-N-cyclopropylbenzamide: m/z (ES) 482 (MH)⁺
For 25Ai: methyl 2-[4-((1S,2S,4R)-2-{4-[(cyclopropylamino)carbonyl]phenyl}bicyclo[2.2.1]-hept-2-yl)phenoxy]-2-pyridin-2-ylpropanoate: m/z (ES) 511 (MH)⁺
For 25Ba: N-(2-hydroxy-2-methylpropyl)-4-{(1S,2S,4R)-2-[4-(1-pyridin-2-ylethoxy)phenyl]-bicyclo[2.2.1]hept-2-yl}benzamide: m/z (ES) 485 (MH)⁺
For 25Bb: N-(2-hydroxy-2-methylpropyl)-4-{(1S,2S,4R)-2-[4-(1-pyridin-2-ylpropoxy)phenyl]-bicyclo[2.2.1]hept-2-yl}benzamide: m/z (ES) 499 (MH)⁺
For 25Ca: 5-(4-{(1S,2S,4R)-2-[4-(1-pyridin-2-ylethoxy)phenyl]bicyclo[2.2.1]hept-2-yl}phenyl)-1,3,4-oxadiazol-2-amine: m/z (ES) 453 (MH)⁺
For 25Cb: 5-(4-{(1S,2S,4R)-2-[4-(1-pyridin-2-ylpropoxy)phenyl]bicyclo[2.2.1]hept-2-yl}-phenyl)-1,3,4-oxadiazol-2-amine: m/z (ES) 467 (MH)⁺
For 25Ch: 5-(4-{(1S,2S,4R)-2-[4-(1-methyl-1-pyridin-2-ylethoxy)phenyl]bicyclo[2.2.1]hept-2-yl}-phenyl)-1,3,4-oxadiazol-2-amine: m/z (ES) 467 (MH)⁺

### Example 26

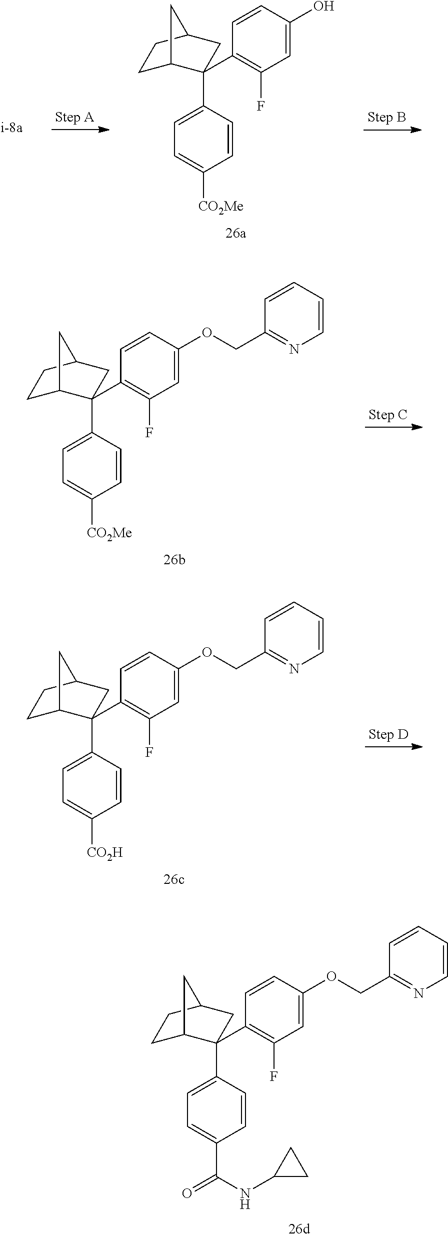

### Preparation of N-cyclopropyl-4-{2-[2-fluoro-4-(pyridin-2-ylmethoxy)phenyl]bicyclo[2.2.1]hept-2-yl}benzamide (26d)

Following procedures described in Schemes i-4, i-8, and i-14, as well as Example 8, compound 26d was prepared. m/z (ES) 457 (MH)⁺.

Following procedures described in the schemes and examples cited above, as well as Scheme i-15 and Examples 1, 2, 22, and 23, the compounds in Table 26 can be prepared:

### Table 26

TABLE 26
26A

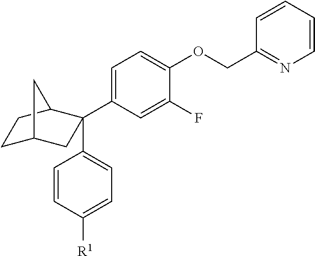

26B

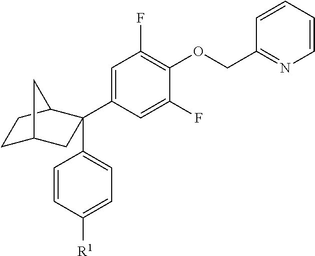

26C

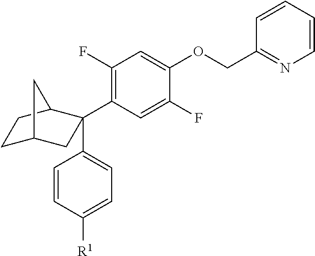

26D

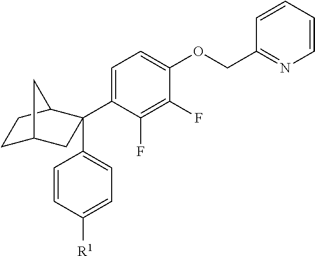

26E

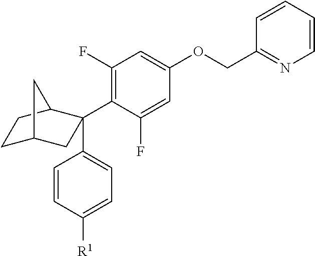

| Ex. | Ex. | Ex. | Ex. | Ex. | |
|---|---|---|---|---|---|
| 26A | 26B | 26C | 26D | 26E | R¹ |
| a | a | a | a | a | 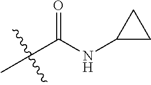 |
| b | b | b | b | b | 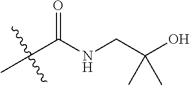 |
| c | c | c | c | c | 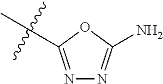 |
| d | d | d | d | d | 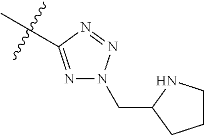 |
| e | e | e | e | e | 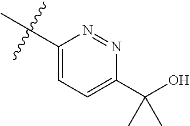 |

Table 26. Parent Ion m/z (MH)⁺ data for compounds
For 26Aa: N-cyclopropyl-4-{2-[3-fluoro-4-(pyridin-2-ylmethoxy)phenyl]bicyclo[2.2.1]hept-2-yl}benzamide: m/z (ES) 457 (MH)⁺
For 26Ca: N-cyclopropyl-4-{2-[2,5-difluoro-4-(pyridin-2-ylmethoxy)phenyl]bicyclo[2.2.1]hept-2-yl}benzamide: m/z (ES) 475 (MH)⁺
For 26Da: N-cyclopropyl-4-{2-[2,3-difluoro-4-(pyridin-2-ylmethoxy)phenyl]bicyclo[2.2.1]hept-2-yl}benzamide: m/z (ES) 475 (MH)⁺
For 26Ea: N-cyclopropyl-4-{2-[2,6-difluoro-4-(pyridin-2-ylmethoxy)phenyl]bicyclo[2.2.1]hept-2-yl}benzamide: m/z (ES) 475 (MH)⁺

### Example 27

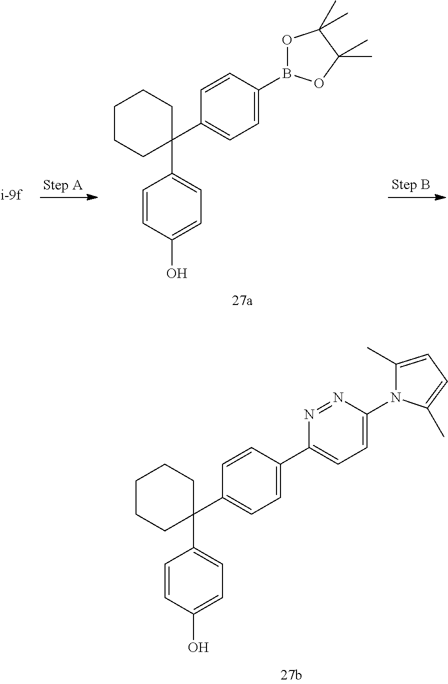

### Step A: Preparation of 4-{1-[4-(4,4,5,5-tetramethyl-1,3,2-dioxaborolan-2-yl)phenyl]-cyclohexyl}phenol (27a)

Compound 27a can be prepared following the procedures described in example 22, step A, substituting intermediate i-9f for intermediate i-9i.

### Step B: Preparation of 4-(1-{4-[6-(2,5-dimethyl-1H-pyrrol-1-yl)pyridazin-3-yl]phenyl}-cyclohexyl)phenol (27b)

Compound 27b can be prepared from 27a and i-20a following the procedures described in example 22, step B, substituting compound 27a for compound 22a.

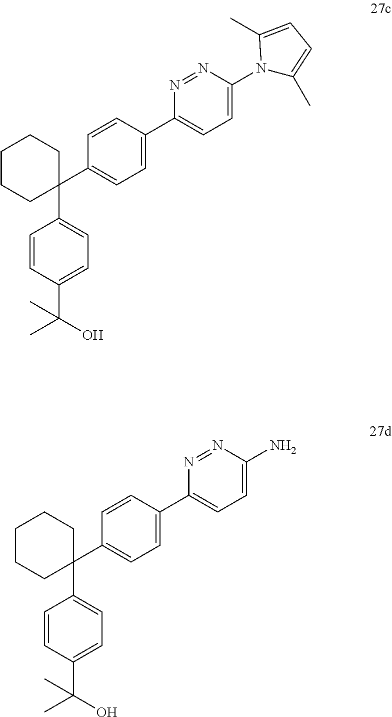

### Preparation of 2 2-[4-(1-{4-[6-(2,5-dimethyl-1H-pyrrol-1-yl)pyridazin-3-yl]phenyl}cyclohexyl)phenyl]propan-2-ol (27c)

Compound 27c can be prepared following the procedures described in scheme i-5, step C, substituting compound 27b for intermediate i-5b. The product of this reaction can be carried forward following procedures as described in scheme i-5, step D, substituting it for i-5c. The product of this reaction can be subsequently carried forward following procedures as described in example 23, step D, substituting it for compound 23C, to afford the title compound 27c.

### Preparation of 2-(4-{1-[4-(6-aminopyridazin-3-yl)phenyl]cyclohexyl}phenyl)propan-2-ol (27d)

A stirred solution of hydroxylamine hydrochloride (10.0 equiv.), triethylamine (3.00 equiv) and 31c (1.00 equiv.) in (2:1) EtOH:water (0.20 M final solute concentration) is irradiated at 120° C. in a microwave reactor for 30 min. After cooling to rt, the reaction mixture is poured into satd. aq. sodium bicarbonate and extracted with EtOAc. The combined organic extracts are dried, filtered and concentrated in vacuo. The crude residue is purified by preparative reversed phase HPLC on YMC Pack Pro C18 stationary phase (CH₃CN/H₂O as eluent, 0.05% TFA as modifier) to afford the title compound 27d.

Following procedures similar to those described in Example 27 and the preceding schemes, the following additional compounds represented in Table 27 can be prepared:

TABLE 27
27A

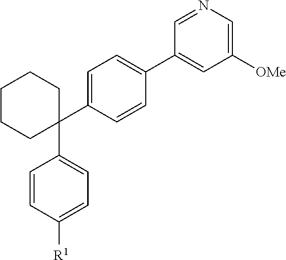

27B

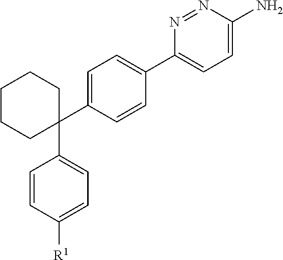

27C

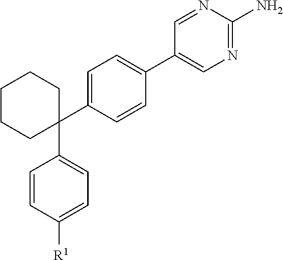

27A

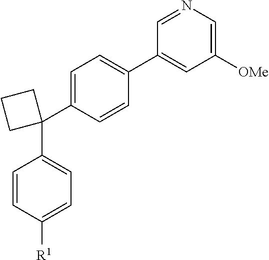

27E

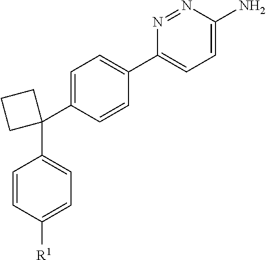

27F

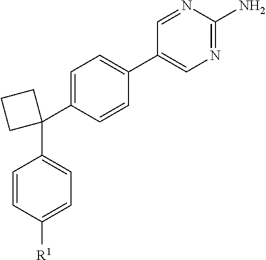

| Ex. | Ex. | Ex. | Ex. | Ex. | Ex. | |
|---|---|---|---|---|---|---|
| #27A | #27B | #27C | #27D | #27E | #27F | R¹ |
| a | a | a | a | a | a | CO₂Me |
| b | — | b | b | b | b | 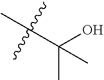 |
| c | c | c | c | c | c | CN |
| d | d | d | d | d | d | 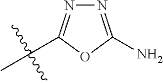 |
| e | e | e | e | e | e | 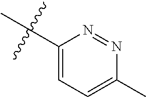 |
| f | f | f | f | f | f | 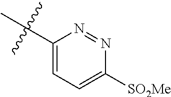 |
| g | g | g | g | g | g | 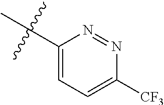 |
| h | h | h | h | h | h | 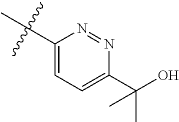 |

### FLAP Binding Assay

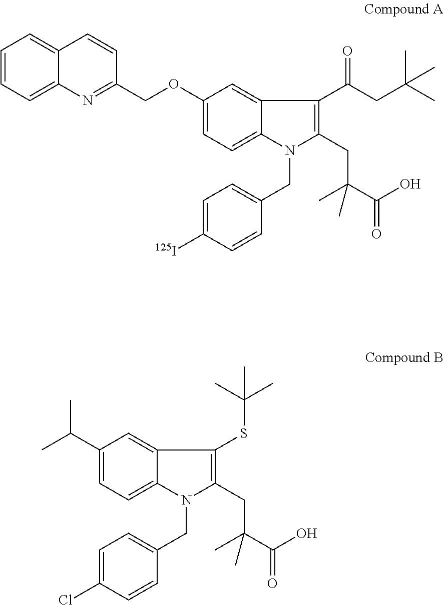

A 100,000×g pellet from human leukocyte 10,000×g supernatants (1) is the source of FLAP. The 100,000×g pellet membranes were resuspended in Tris-Tween assay buffer (100 mM Tris HCl pH 7.4, 140 mM NaCl, 2 mM EDTA, 0.5 mM dithiothreitol, 5% glycerol, 0.05% Tween 20) to yield a final protein concentration of 50 μg to 150 μg/ml. Aliquots (100 μl) of membrane suspension were added to 12 mm×75 mm polypropylene tubes containing 100 μl Tris-Tween assay buffer, 30,000 cpm of Compound A in 5 μl MeOH:assay buffer (1:1), and 2 μl dimethyl sulfoxide or competitor (i.e., the compound to be tested) in dimethyl sulfoxide. Compound B (10 μM final concentration) was used to determine non-specific binding. After a 20 minute incubation at room temperature, tube contents were diluted to 4 ml with cold 0.1 M Tris HCl pH 7.4, 0.05% Tween 20 wash buffer and the membranes were collected by filtration of GFB filters presoaked in the wash buffer. Tubes and filters were rinsed with 2×4 ml aliquots of cold wash buffer. Filters were transferred to 12 mm×3.5 mm polystyrene tubes for determination of radioactivity by gamma-scintillation counting.

Specific binding is defined as total binding minus non-specific binding. Total binding was Compound A bound to membranes in the absence of competitor; non-specific binding was Compound A bound in the presence of 10 μM Compound B. Preparation of Compound A is described in reference 1, below. The IC₅₀ values were obtained by computer analysis (see reference 2, below) of the experimental data. Representative compounds of the invention were determined to have an IC₅₀<50 nM.

### REFERENCES

1. Charleson, S., Prasti, P., Leger, S., Gillard, J. W, Vickers, P. J., Mancini, J. A., Charleson, P., Guay, J., Ford-Hutchinson, A. W., and Evans, J. F. (1992) Characterization of a 5-lipoxygenase-activating protein binding assay: correlation of affinity for 5-lipoxygenase-activating protein with leukotriene synthesis inhibition. Mol Pharmacol 41:873-879.
2. Kinetic, E B D A, Ligand, Lowry: A collection of Radioligand Binding Analysis Programs by G. A. McPherson. Elsevier-BIOSOFT.

While the invention has been described with reference to certain particular embodiments thereof, numerous alternative embodiments will be apparent to those skilled in the art from the teachings described herein. All patents, patent applications and publications cited herein are incorporated by reference in their entirety.

1. A compound having the formula I 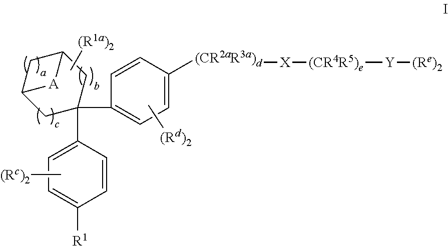
and the pharmaceutically acceptable salts thereof wherein:
a is an integer selected from 0, 1, 2 and 3;
b and c are each integers independently selected from 0, 1 and 2;
A represents a methylene or ethylene group;
each R^{1a} is independently selected from the group consisting of: —H, —F, —C₁₋₆alkyl, —OH, —OC₁₋₆alkyl, -fluoroC₁₋₆alkyl, -fluoro C₁₋₆alkoxy, —N(R^{a})₂ and C₁₋₆alkylN(R^{a})₂,
or one R^{1a} group can represent oxo and the other is as previously defined;
R¹ is selected from the group consisting of:
(a) a 5-membered aromatic or partially unsaturated heterocyclic ring containing 2 to 4 heteroatoms selected from N, S and O, wherein the heterocyclic ring is optionally substituted with one or more of R⁶;
(b) a 6-membered aromatic or partially unsaturated heterocyclic ring containing 1 to 2 heteroatoms selected from N and O, wherein the heterocyclic ring is optionally substituted with one or more of R⁶;
(c) an 8-membered aromatic or partially unsaturated ortho-fused bicyclic ring system containing 3-5 heteroatoms selected from one sulfur and 2-4 of nitrogen wherein one carbon in the ring is optionally substituted with a group selected from ═O, ═S, —SMe, —NH₂, —CF₃, —Cl, —C₁₋₄alkyl and C₁₋₄alkyl substituted with a group selected from —NH₂, —OH, —OC₁₋₄alkyl, —CN and 1-3 of fluoro;
(d) a 9-membered aromatic or partially unsaturated ortho-fused bicyclic ring system containing 3-4 nitrogen atoms, wherein one carbon in the ring is optionally substituted with a group selected from ═O, ═S, —SMe, —NH₂, —CF₃, —Cl, pyrrolidinyl, tetrahydrofuranyl, —C₁₋₄alkyl and C₁₋₄alkyl substituted with a group selected from —NH₂, —OH, —OC₁₋₄alkyl, —CN, 1-3 of fluoro and piperidinyl;
(e) —C₁₋₆alkyl, —C₂₋₆alkenyl, and —C₂₋₆alkynyl, said alkyl, alkenyl and alkynyl groups being optionally substituted with R¹² and optionally substituted with R¹³;
(f) —C₃₋₆cycloalkyl optionally substituted with 1-3 substituents selected from the group consisting of fluoro, —NH₂, —OH and —C₁₋₃alkyl optionally substituted with 1-3 of fluoro;
(g) —O—R^{6a} wherein R^{6a} is selected from the group consisting of (1) —C₁₋₆alkyl optionally substituted with R¹² and optionally substituted with R¹³, (2) —C₃₋₆cycloalkyl optionally substituted with R¹² and optionally substituted with R¹³ and (3) —C₂₋₆alkyl-R¹⁰; and
(h) —H, —OH, —CN, —CO₂R^{4a}, —C(O)NR⁷R⁸, —NR⁷R⁸, —NR^{b}SOₚR^{a}, —NR^{b}C(O)R^{a}, —NR^{b}C(O)NR^{a}R^{b}, —S(O)ₚR^{a}, and —S(O)ₚNR^{a}R^{b};
p is an integer selected from 0, 1 and 2;
X is selected from the group consisting of a bond, —O—, —S— and —C(R¹⁴)₂—;
R^{4a} is selected from the group consisting of —H, —C₁₋₆alkyl and —C₃₋₆cycloalkyl;
R⁶ is selected from the group consisting of (a) —C₁₋₆alkyl optionally substituted with one or more substituents selected from the group consisting of —OH, —NH₂, —N(CH₃)₂, NH(C═O)O^{t}Bu, —CN, fluoro, and —O—C₁₋₄alkyl optionally substituted with one or more substituents selected from the group consisting of —OH, phenyl and fluoro, (b) —C₁₋₆alkyl-R¹⁰, (c) —OC₁₋₆alkyl optionally substituted with one or more substituents selected from the group consisting of —OH, —NH₂ and fluoro, (d) —C₃₋₆cycloalkyl optionally substituted with one or more substituents selected from the group consisting of methyl, —OH, —NH₂, —NH(C═O)O^{t}Bu, —CF₃ and fluoro, (e) —NR⁷R⁸, (f) —SO₂C₁₋₃alkyl, (g) —(CH₂)₀₋₃CO₂—R⁸, (h) —OH, (i) ═O (oxo), (j) —SH, (k) ═S, (l) —SMe, (m) —Cl, (n) 1-5 of fluoro, (O) —CF₃, (p) —CN and (q) R¹⁰;
R⁷ is selected from the group consisting of (a) —H, (b) —C₁₋₆alkyl optionally substituted with one or more substituents selected from the group consisting of —F, —CN, —NH₂ and —OH, (c) —C₃₋₆cycloalkyl optionally substituted with one or more substituents selected from the group consisting of methyl, —CF₃, —F, —NH₂ and —OH, (d) —COC₁₋₆alkyl optionally substituted with one or more substituents selected from the group consisting of —F and —OH, (e) —C(O)OC₁₋₆alkyl optionally substituted with one or more substituents selected from the group consisting of methyl, phenyl, —CF₃, —F and —OH, (f) a 4-6 membered saturated heterocyclic ring containing one N and/or one O, wherein the ring is bonded to the nitrogen in —NR⁷R⁸ through a carbon atom in the ring, and wherein the ring is optionally substituted with one or more substituents selected from the group consisting of methyl, —CF₃, —F, CH₂CH₂F, CH₂CHF₂, CH₂CF₃, —NH₂ and —OH, and wherein the ring is optionally bridged by a —CH₂CH₂— group, and (g) a 5-membered heterocyclic ring comprising one, two or three heteroatoms selected from N, O and S, (h) a 5-membered aromatic or partially unsaturated heterocyclic ring containing 2 to 4 heteroatoms selected from N, S and O, wherein the heterocyclic ring is optionally substituted with one or more of R⁶, (i) a 6-membered aromatic or partially unsaturated heterocyclic ring containing 1 to 2 heteroatoms selected from N and O, wherein the heterocyclic ring is optionally substituted with one or more of R⁶;
R⁸ is selected from the group consisting of (a) —H, (b) —C₁₋₆alkyl optionally substituted with one or more substituents selected from the group consisting of —F, —NH₂ and —OH, and (c) —C₃₋₆cycloalkyl optionally substituted with one or more substituents selected from the group consisting of methyl, —CF₃, —F, —NH₂ and —OH;
R¹⁰ is a heterocyclic ring selected from the group consisting of (a) azetidinyl optionally substituted with one or more of methyl, —F and —OH, (b) pyrrolidinyl optionally substituted with one or more of methyl, —F and —OH, (c) piperidinyl optionally substituted with one or more of methyl, —F and —OH, (d) piperazinyl optionally substituted with ═O, and (e) morpholinyl optionally substituted with one or more of methyl and —F; and
Y is selected from the group consisting of (a) a 5-membered aromatic or partially unsaturated heterocyclic ring containing 1 to 4 heteroatoms selected from 1 to 4 of N and zero to 1 of S, wherein the heterocyclic ring is optionally substituted with R¹¹, (b) a 6-membered aromatic or partially unsaturated heterocyclic ring containing 1 to 2 N heteroatoms, wherein the heterocyclic ring is optionally substituted with R¹¹, (c) a 9-membered bicyclic aromatic or partially unsaturated heterocyclic ring containing 1 to 4 N heteroatoms, wherein the heterocyclic ring is optionally substituted with R¹¹ and (d) a 10-membered bicyclic aromatic or partially unsaturated heterocyclic ring containing 1 to 4 N heteroatoms, wherein the heterocyclic ring is optionally substituted with R¹¹; and
R¹¹ is selected from the group consisting of —F, —NH₂, —OH, —OC₃₋₄cycloalkyl, —C₁₋₃alkyl optionally substituted with 1-3 fluoro, and —OC₁₋₃alkyl optionally substituted with phenyl or 1-3 fluoro.
R¹² is selected from the group consisting of: —CO₂R^{4a}, —C(O)NR⁷R⁸, —N(R^{a})₂, —NR^{b}SOₚR^{a}, —NR^{b}C(O)R^{a}, —NR^{b}C(O)NR^{a}R^{b}, —S(O)ₚNR^{a}R^{b}, —S(O)ₚR^{a}, —F, —CF₃, phenyl, Het and Z¹,
R¹³ is selected from the group consisting of —OH, —NH₂ and 1-5 of —F;
R¹⁴ is selected from the group consisting of —H and —C₁₋₄alkyl optionally substituted with 1-3 fluoro groups;
each R^{a} is independently selected from the group consisting of
a) —H,
b) —C₁₋₆alkyl, —C₂₋₆alkenyl and —C₂₋₆alkynyl, wherein each is optionally substituted with 1-2 substituents selected from the group consisting of: —OH, —OC₁₋₄alkyl, —CN, —NH₂, —NHC₁₋₄alkyl, and —N(C₁₋₄alkyl)₂, and —CF₃, and optionally with 1-3 of fluoro,
c) —C₃₋₆cycloalkyl, optionally substituted by 1-2 substituents selected from the group consisting of: —C₁₋₄alkyl, —OH, —OC₁₋₄alkyl, —CN, —NH₂, —NHC₁₋₄alkyl, and —N(C₁₋₄alkyl)₂, and —CF₃, and optionally with 1-3 of fluoro,
d) Het and Het-C₁₋₄alkylene-, the Het moieties being optionally substituted on carbon with 1-2 substituents selected from the group consisting of —F, —OH, —CO₂H, —C₁₋₄alkyl, —CO₂C₁₋₄alkyl, —OC₁₋₄alkyl, —NH₂, —NHC₁₋₄alkyl, —N(C₁₋₄alkyl)₂, —NHC(O)C₁₋₄alkyl, oxo, —C(O)NHC₁₋₄alkyl and —C(O)N(C₁₋₄alkyl)₂; and optionally substituted on nitrogen when present with a group selected from —C₁₋₄alkyl and —C₁₋₄acyl; and the alkylene portion of Het-C₁₋₄alkylene- being optionally substituted with a member selected from the group consisting of —OH, —CN, —OC₁₋₄alkyl, —NH₂, —NHC₁₋₄alkyl, —N(C₁₋₄alkyl)₂ and 1-3 of fluoro,
e) Z² and Z²—C₁₋₄alkylene-, the alkylene portion of Z²—C₁₋₄alkylene- being optionally substituted with a substituent selected from the group consisting of —OH, —CN, —OC₁₋₄alkyl, —NH₂, —NHC₁₋₄alkyl, —N(C₁₋₄alkyl)₂ and 1-3 of fluoro;
each R^{b} is independently selected from the group consisting of —H and —C₁₋₃alkyl optionally substituted with 1-2 members selected from the group consisting of NH₂, —OH, —F, —CN and —CF₃;
R^{c}, R^{d}, and R^{e} are each independently selected from —H, —F, —Cl, —Br, —C₁₋₆alkyl, —CN, —OH, —OC₁₋₆alkyl, -fluoroC₁₋₆alkyl, -fluoroC₁₋₆alkoxy, —N(R^{f})₂ or —C₁₋₆alkylN(R^{f})₂, where C₁₋₆alkyl and OC₁₋₆alkyl are optionally substituted by 1-3 of fluoro;
each R^{f} is independently selected from the group consisting of —H and
(a) —C₁₋₁₀alkyl, —C₃₋₁₀alkenyl, or —C₃₋₁₀alkynyl, optionally substituted with 1-3 fluoro groups or 1-2 members selected from the group consisting of: —OH, —OC₁₋₆alkyl, —CN, —NH₂, —NHC₁₋₄alkyl, and —N(C₁₋₄alkyl)₂;
(b) Aryl or Ar—C₁₋₆alkylene-, the aryl portions being optionally substituted with 1-2 of —C₁₋₆alkyl, —CN, —OH, —OC₁₋₆alkyl, -fluoroC₁₋₆alkyl, -fluoroC₁₋₆alkoxy, —C₁₋₆alkyl-NH₂, —C₁₋₆alkylNHC₁₋₄alkyl, —C₁₋₆alkylN(C₁₋₄alkyl)₂, —NH₂, —NHC₁₋₄alkyl, —N(C₁₋₄alkyl)₂, —NHC(O)C₁₋₄alkyl, —C(O)NHC₁₋₄alkyl, —C(O)N(C₁₋₄alkyl)₂, —CO₂H and —CO₂C₁₋₆alkyl groups, and 1-3 —F, —Cl or —Br groups; and
the alkylene portion of Ar—C₁₋₆alkylene- being optionally substituted with —OH, —OC₁₋₆alkyl, —NH₂, —NHC₁₋₄alkyl, —N(C₁₋₄alkyl)₂, and 1-3 fluoro groups;
(c) Hetcy or Hetcy-C₁₋₆alkylene-, each being optionally substituted on carbon with 1-2 members selected from the group consisting of: —F, —OH, —CO₂H, —C₁₋₆alkyl, —CO₂C₁₋₆alkyl, —OC₁₋₆alkyl, —NH₂, —NHC₁₋₄alkyl, —N(C₁₋₄alkyl)₂, —NHC(O)C₁₋₄alkyl, oxo, —C(O)NHC₁₋₄alkyl and —C(O)N(C₁₋₄alkyl)₂; and optionally substituted on nitrogen when present with —C₁₋₆alkyl or —C₁₋₆acyl; and
the alkylene portion of Hetcy-C₁₋₆alkylene- being optionally substituted with 1-2 of: —F, —OH, —OC₁₋₆alkyl, —NH₂, —NHC₁₋₄alkyl and —N(C₁₋₄alkyl)₂;
(d) HAR or HAR-C₁₋₆alkylene-, said HAR and HAR portion of HAR-C₁₋₆alkylene- being substituted with 1-2 members selected from the group consisting of: —F, —Cl, —Br, —C₁₋₆alkyl, —CN, —OH, —OC₁₋₆alkyl, -fluoroC₁₋₆alkyl, -fluoroC₁₋₆alkoxy NH₂, —NHC₁₋₄alkyl, —N(C₁₋₄alkyl)₂, —NHC(O)C₁₋₄alkyl, —C(O)NHC₁₋₄alkyl, —C(O)N(C₁₋₄alkyl)₂, —CO₂H, —CO₂C₁₋₆alkyl; and
the alkylene portion of HAR-C₁₋₆alkylene- being optionally substituted with 1-2 of: —F, —OH, —OC₁₋₆alkyl, —NH₂, —NHC₁₋₄alkyl and —N(C₁₋₄alkyl)₂;
Het is selected from the group consisting of azetidinyl, pyrrolidinyl, piperidinyl, piperazinyl, morpholinyl, tetraydrofuranyl and \tilde{β}lactamyl, \tilde{δ}lactamyl, γ-lactamyl and tetrahydropyranyl;
Z¹ is selected from the group consisting of:
a) Z²,
b) an 8-membered aromatic or partially unsaturated ortho-fused bicyclic ring system containing 3-5 heteroatoms selected from one sulfur and 2-4 of nitrogen wherein one carbon in the ring is optionally substituted with a group selected from ═O, ═S, —SMe, —NH₂, —CF₃, —Cl, —C₁₋₄alkyl and C₁₋₄alkyl substituted with a group selected from —NH₂, —OH, —OC₁₋₄alkyl, —CN and 1-3 of fluoro, and
c) a 9-membered aromatic or partially unsaturated ortho-fused bicyclic ring system containing 3-4 nitrogen atoms, wherein one carbon in the ring is optionally substituted with a group selected from ═O, ═S, —SMe, —NH₂, —CF₃, —Cl, —C₁₋₄alkyl and C₁₋₄alkyl substituted with a group selected from —NH₂, —OH, —OC₁₋₄alkyl, —CN and 1-3 of fluoro; and
Z² is selected from the group consisting of:
a) a 5-membered aromatic or partially unsaturated heterocyclic ring containing 2-4 nitrogen atoms, wherein one nitrogen in the ring is optionally substituted with a group selected from —C₁₋₄alkyl and —C₁₋₄alkyl substituted with a group selected from —NH₂, —OH, —CN and 1-3 of fluoro, and one carbon in the ring is optionally substituted with a group selected from ═O, ═S, —SMe, —NH₂, —CF₃, —Cl, —C₁₋₄alkyl, —C₁₋₄alkyl substituted with a group selected from —NH₂, —OH, —OC₁₋₄alkyl, —CN and 1-3 of fluoro, and —OC₁₋₄alkyl optionally substituted by —OH or 1-3 of fluoro;
b) a 5-membered aromatic or partially unsaturated heterocyclic ring containing 2-3 heteroatoms selected from one oxygen or one sulfur and 1-2 of nitrogen, wherein one nitrogen in the ring is optionally substituted with a group selected from C₁₋₄alkyl and C₁₋₄alkyl substituted with a group selected from —NH₂, —OH, —CN and 1-3 of fluoro, and one carbon in the ring is optionally substituted with a group selected from ═O, ═S, —SMe, —NH₂, —CF₃, —Cl, C₁₋₄alkyl optionally substituted with a group selected from —NH₂, —OH, —OC₁₋₄alkyl, —CN and 1-3 of fluoro, and —OC₁₋₄alkyl optionally substituted by —OH or 1-3 of fluoro; and
c) a 6-membered aromatic or partially unsaturated heterocyclic ring containing 1-2 nitrogen atoms, wherein one nitrogen in the ring is optionally substituted with a group selected from —C₁₋₄alkyl and —C₁₋₄alkyl substituted with a group selected from —NH₂, —OH, —CN and 1-3 of fluoro, and one carbon in the ring is optionally substituted with a group selected from ═O, ═S, —SMe, —NH₂, —CF₃, —Cl, —C₁₋₄alkyl, —C₁₋₄alkyl substituted with a group selected from —NH₂, —OH, —OC₁₋₄alkyl, —CN and 1-3 of fluoro, and —OC₁₋₄alkyl optionally substituted by —OH or 1-3 of fluoro;
d and e are each integers independently selected from 0, 1, and 2, such that the sum of d plus e is 0 to 4;
R^{2a}, R^{3a}, R⁴ and R⁵ are each independently selected from the group consisting of —H, —C₁₋₆alkyl, —OC₁₋₆alkyl, —OH, -fluoro, -fluoroC₁₋₆alkyl, -fluoroC₁₋₆alkoxy, —N(R^{f})₂, where R^{f} is as hereinbefore defined, and none or one of CR^{2a}R^{3a} and none or one of CR⁴R⁵ can represent a group selected from carbonyl, thiocarbonyl, C═NR^{f} and a 3- to 7-membered cycloalkyl ring.

2. A pharmaceutical composition comprising a therapeutically effective amount of a compound of claim 1.

3. A method for the treatment of a disease or condition mediated by FLAP which comprises administering a compound of claim 1 to a patient in need thereof.

4. (canceled)

5. A compound of claim 1 having the formula (Ia): 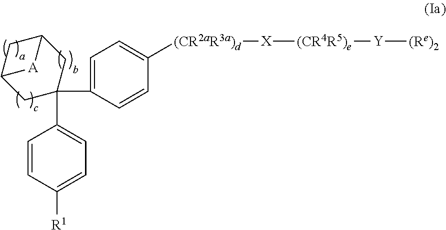
and pharmaceutically acceptable salts thereof, wherein R¹, R^{2a}, R^{3a}, R⁴, R⁵, R^{e}, a, b, c, d, e, A, X and Y are as defined in claim 1.

6. A compound of claim 1 having the formula (Ia-1): 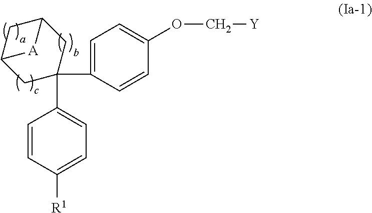
pharmaceutically acceptable salts thereof, wherein R¹, a, b, c, A and Y are as defined in claim 1

7. A compound of claim 6 wherein R¹ is a 5-membered aromatic or partially unsaturated heterocyclic ring containing 2 to 4 heteroatoms selected from N, S and O, wherein the heterocyclic ring is optionally substituted with one or more of R⁶.

8. A compound of claim 6 wherein R¹ is selected from oxazole, isoxazole, oxadiazole, thiadiazole, triazole, pyrazole, and tetrazole, wherein each of these groups includes its corresponding partially saturated derivative, and wherein each group is optionally substituted with one or more of R⁶.

9. A compound of claim 6 wherein R¹ is a 6-membered aromatic or partially unsaturated heterocyclic ring containing 1 to 2 nitrogen atoms, wherein the heterocyclic ring is optionally substituted with one or more of R⁶.

10. A compound of claim 6 wherein R¹ is pyridyl, pyrazinyl or pyridazinyl optionally substituted with one or more of R⁶. In another subset of formula (Ia-1) Y is selected from pyridyl, pyrimidinyl and thiazolyl, each optionally substituted with R¹¹.

11. A compound of claim 6 wherein Y is selected from pyridyl, pyrimidinyl and thiazolyl each optionally substituted with R¹¹, and R¹ is a 5-membered aromatic or partially unsaturated heterocyclic ring containing 2 to 4 heteroatoms selected from N, S and O, wherein the heterocyclic ring is optionally substituted with one or more of R⁶.

12. A compound of claim 1 having the formula (Ia-1a): 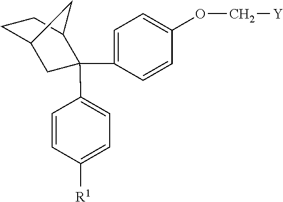
pharmaceutically acceptable salts thereof, wherein R¹ and Y are as defined in claim 1.

13. A compound of claim 12 wherein R¹ is a 5-membered aromatic or partially unsaturated heterocyclic ring containing 2 to 4 heteroatoms selected from N, S and O, wherein the heterocyclic ring is optionally substituted with one or more of R⁶.

14. A compound of claim 12 wherein R¹ is selected from oxazole, isoxazole, oxadiazole, thiadiazole, triazole, pyrazole, and tetrazole, wherein each of these groups includes its corresponding partially saturated derivative, and wherein each group is optionally substituted with one or more of R⁶.

15. A compound of claim 12 wherein R¹ is a 6-membered aromatic or partially unsaturated heterocyclic ring containing 1 to 2 nitrogen atoms, wherein the heterocyclic ring is optionally substituted with one or more of R⁶.

16. A compound of claim 12 wherein R¹ is pyridyl, pyrazinyl or pyridazinyl optionally substituted with one or more of R⁶.

17. A compound of claim 12 wherein Y is selected from pyridyl, pyrimidinyl and thiazolyl, each optionally substituted with R¹¹.

18. A compound of claim 12 wherein Y is selected from pyridyl, pyrimidinyl and thiazolyl each optionally substituted with R¹¹ and R¹ is a 5-membered aromatic or partially unsaturated heterocyclic ring containing 2 to 4 heteroatoms selected from N, S and O, wherein the heterocyclic ring is optionally substituted with one or more of R⁶.
